SEQUENCE LISTING (799 sequences)

SEQ ID NO: 1 - protein, 12 aa, Artificial Sequence
  Description of Artificial Sequence Synthetic peptide
RRWIRVAVILRV

SEQ ID NO: 2 - protein, 12 aa, Artificial Sequence
  Description of Artificial Sequence Synthetic peptide
VRLIWAVRIWRR

SEQ ID NO: 3 - protein, 10 aa, Artificial Sequence
  Description of Artificial Sequence Synthetic peptide
VRLIVRIWRR

SEQ ID NO: 4 - protein, 10 aa, Artificial Sequence
  Description of Artificial Sequence Synthetic peptide
RFKRVARVIW

SEQ ID NO: 5 - protein, 12 aa, Artificial Sequence
  Description of Artificial Sequence Synthetic peptide
FKKVIVIRRWFI

SEQ ID NO: 6 - protein, 12 aa, Artificial Sequence
  Description of Artificial Sequence Synthetic peptide
RRWIRVAVILRV

SEQ ID NO: 7 - protein, 12 aa, Artificial Sequence
  Description of Artificial Sequence Synthetic peptide
KRIRWVILWRQV

SEQ ID NO: 8 - protein, 12 aa, Artificial Sequence
  Description of Artificial Sequence Synthetic peptide
RRINRVIWRWRK

SEQ ID NO: 9 - protein, 12 aa, Artificial Sequence
  Description of Artificial Sequence Synthetic peptide
RIVWVRIRRWFV

SEQ ID NO: 10 - protein, 12 aa, Artificial Sequence
  Description of Artificial Sequence Synthetic peptide
RIVRVRIARLQV

SEQ ID NO: 11 - protein, 12 aa, Artificial Sequence
  Description of Artificial Sequence Synthetic peptide
RIVWVRIRRLQV

SEQ ID NO: 12 - protein, 12 aa, Artificial Sequence
  Description of Artificial Sequence Synthetic peptide
RIVWVRIRRWQV

SEQ ID NO: 13 - protein, 8 aa, Artificial Sequence
  Description of Artificial Sequence Synthetic peptide
RFRIRVRR

SEQ ID NO: 14 - protein, 10 aa, Artificial Sequence
  Description of Artificial Sequence Synthetic peptide
VRVAVRIWRR

SEQ ID NO: 15 - protein, 12 aa, Artificial Sequence
  Description of Artificial Sequence Synthetic peptide
VRLIPAVRIWRR

SEQ ID NO: 16 - protein, 12 aa, Artificial Sequence
  Description of Artificial Sequence Synthetic peptide
KQFRIRVRVWIK

SEQ ID NO: 17 - protein, 12 aa, Artificial Sequence
  Description of Artificial Sequence Synthetic peptide
VRLIRIWVRIWR

SEQ ID NO: 18 - protein, 11 aa, Artificial Sequence
  Description of Artificial Sequence Synthetic peptide
RFKVAVRIWRR

SEQ ID NO: 19 - protein, 12 aa, Artificial Sequence
  Description of Artificial Sequence Synthetic peptide
VRLIRAVRIWRR

SEQ ID NO: 20 - protein, 12 aa, Artificial Sequence
  Description of Artificial Sequence Synthetic peptide
IRWRIRVRVRWI

SEQ ID NO: 21 - protein, 12 aa, Artificial Sequence
  Description of Artificial Sequence Synthetic peptide
VRLRIRWRKLWV

SEQ ID NO: 22 - protein, 12 aa, Artificial Sequence
  Description of Artificial Sequence Synthetic peptide
GRLIVAVRIWRR

SEQ ID NO: 23 - protein, 12 aa, Artificial Sequence
  Description of Artificial Sequence Synthetic peptide
VGLIVAVRIWRR

SEQ ID NO: 24 - protein, 12 aa, Artificial Sequence
  Description of Artificial Sequence Synthetic peptide
VRGIVAVRIWRR

SEQ ID NO: 25 - protein, 12 aa, Artificial Sequence
  Description of Artificial Sequence Synthetic peptide
VRLGVAVRIWRR

SEQ ID NO: 26 - protein, 12 aa, Artificial Sequence
  Description of Artificial Sequence Synthetic peptide
VRLIGAVRIWRR

SEQ ID NO: 27 - protein, 12 aa, Artificial Sequence
  Description of Artificial Sequence Synthetic peptide
VRLIVGVRIWRR

SEQ ID NO: 28 - protein, 12 aa, Artificial Sequence
  Description of Artificial Sequence Synthetic peptide
VRLIVAGRIWRR

SEQ ID NO: 29 - protein, 12 aa, Artificial Sequence
  Description of Artificial Sequence Synthetic peptide
VRLIVAVGIWRR

SEQ ID NO: 30 - protein, 12 aa, Artificial Sequence
  Description of Artificial Sequence Synthetic peptide
VRLIVAVRGWRR

SEQ ID NO: 31 - protein, 12 aa, Artificial Sequence
  Description of Artificial Sequence Synthetic peptide
VRLIVAVRIGRR

SEQ ID NO: 32 - protein, 12 aa, Artificial Sequence
  Description of Artificial Sequence Synthetic peptide
VRLIVAVRIWGR

SEQ ID NO: 33 - protein, 12 aa, Artificial Sequence
  Description of Artificial Sequence Synthetic peptide
VRLIVAVRIWRG

SEQ ID NO: 34 - protein, 12 aa, Artificial Sequence
  Description of Artificial Sequence Synthetic peptide
ARLIVAVRIWRR

SEQ ID NO: 35 - protein, 12 aa, Artificial Sequence
  Description of Artificial Sequence Synthetic peptide
VALIVAVRIWRR

SEQ ID NO: 36 - protein, 12 aa, Artificial Sequence
  Description of Artificial Sequence Synthetic peptide
VRAIVAVRIWRR

SEQ ID NO: 37 - protein, 12 aa, Artificial Sequence
  Description of Artificial Sequence Synthetic peptide
VRLAVAVRIWRR

SEQ ID NO: 38 - protein, 12 aa, Artificial Sequence
  Description of Artificial Sequence Synthetic peptide
VRLIAAVRIWRR

SEQ ID NO: 39 - protein, 12 aa, Artificial Sequence
  Description of Artificial Sequence Synthetic peptide
VRLIVAARIWRR

SEQ ID NO: 40 - protein, 12 aa, Artificial Sequence
  Description of Artificial Sequence Synthetic peptide
VRLIVAVAIWRR

SEQ ID NO: 41 - protein, 12 aa, Artificial Sequence
  Description of Artificial Sequence Synthetic peptide
VRLIVAVRAWRR

SEQ ID NO: 42 - protein, 12 aa, Artificial Sequence
  Description of Artificial Sequence Synthetic peptide
VRLIVAVRIARR

SEQ ID NO: 43 - protein, 12 aa, Artificial Sequence
  Description of Artificial Sequence Synthetic peptide
VRLIVAVRIWAR

SEQ ID NO: 44 - protein, 12 aa, Artificial Sequence
  Description of Artificial Sequence Synthetic peptide
VRLIVAVRIWRA

SEQ ID NO: 45 - protein, 12 aa, Artificial Sequence
  Description of Artificial Sequence Synthetic peptide
RRLIVAVRIWRR

SEQ ID NO: 46 - protein, 12 aa, Artificial Sequence
  Description of Artificial Sequence Synthetic peptide
VRRIVAVRIWRR

SEQ ID NO: 47 - protein, 12 aa, Artificial Sequence
  Description of Artificial Sequence Synthetic peptide
VRLRVAVRIWRR

SEQ ID NO: 48 - protein, 12 aa, Artificial Sequence
  Description of Artificial Sequence Synthetic peptide
VRLIRAVRIWRR

SEQ ID NO: 49 - protein, 12 aa, Artificial Sequence
  Description of Artificial Sequence Synthetic peptide
VRLIVRVRIWRR

SEQ ID NO: 50 - protein, 12 aa, Artificial Sequence
  Description of Artificial Sequence Synthetic peptide
VRLIVARRIWRR

SEQ ID NO: 51 - protein, 12 aa, Artificial Sequence
  Description of Artificial Sequence Synthetic peptide
VRLIVAVRRWRR

SEQ ID NO: 52 - protein, 12 aa, Artificial Sequence
  Description of Artificial Sequence Synthetic peptide
VRLIVAVRIRRR

SEQ ID NO: 53 - protein, 12 aa, Artificial Sequence
  Description of Artificial Sequence Synthetic peptide
KRLIVAVRIWRR

SEQ ID NO: 54 - protein, 12 aa, Artificial Sequence
  Description of Artificial Sequence Synthetic peptide
VKLIVAVRIWRR

SEQ ID NO: 55 - protein, 12 aa, Artificial Sequence
  Description of Artificial Sequence Synthetic peptide
VRKIVAVRIWRR

SEQ ID NO: 56 - protein, 12 aa, Artificial Sequence
  Description of Artificial Sequence Synthetic peptide
VRLKVAVRIWRR

SEQ ID NO: 57 - protein, 12 aa, Artificial Sequence
  Description of Artificial Sequence Synthetic peptide
VRLIKAVRIWRR

SEQ ID NO: 58 - protein, 12 aa, Artificial Sequence
  Description of Artificial Sequence Synthetic peptide
VRLIVKVRIWRR

SEQ ID NO: 59 - protein, 12 aa, Artificial Sequence
  Description of Artificial Sequence Synthetic peptide
VRLIVAKRIWRR

SEQ ID NO: 60 - protein, 12 aa, Artificial Sequence
  Description of Artificial Sequence Synthetic peptide
VRLIVAVKIWRR

SEQ ID NO: 61 - protein, 12 aa, Artificial Sequence
  Description of Artificial Sequence Synthetic peptide
VRLIVAVRKWRR

SEQ ID NO: 62 - protein, 12 aa, Artificial Sequence
  Description of Artificial Sequence Synthetic peptide
VRLIVAVRIKRR

SEQ ID NO: 63 - protein, 12 aa, Artificial Sequence
  Description of Artificial Sequence Synthetic peptide
VRLIVAVRIWKR

SEQ ID NO: 64 - protein, 12 aa, Artificial Sequence
  Description of Artificial Sequence Synthetic peptide
VRLIVAVRIWRK

SEQ ID NO: 65 - protein, 12 aa, Artificial Sequence
  Description of Artificial Sequence Synthetic peptide
LRLIVAVRIWRR

SEQ ID NO: 66 - protein, 12 aa, Artificial Sequence
  Description of Artificial Sequence Synthetic peptide
VLLIVAVRIWRR

SEQ ID NO: 67 - protein, 12 aa, Artificial Sequence
  Description of Artificial Sequence Synthetic peptide
VRLLVAVRIWRR

SEQ ID NO: 68 - protein, 12 aa, Artificial Sequence
  Description of Artificial Sequence Synthetic peptide
VRLILAVRIWRR

SEQ ID NO: 69 - protein, 12 aa, Artificial Sequence
  Description of Artificial Sequence Synthetic peptide
VRLIVLVRIWRR

SEQ ID NO: 70 - protein, 12 aa, Artificial Sequence
  Description of Artificial Sequence Synthetic peptide
VRLIVALRIWRR

SEQ ID NO: 71 - protein, 12 aa, Artificial Sequence
  Description of Artificial Sequence Synthetic peptide
VRLIVAVLIWRR

SEQ ID NO: 72 - protein, 12 aa, Artificial Sequence
  Description of Artificial Sequence Synthetic peptide
VRLIVAVRLWRR

SEQ ID NO: 73 - protein, 12 aa, Artificial Sequence
  Description of Artificial Sequence Synthetic peptide
VRLIVAVRILRR

SEQ ID NO: 74 - protein, 12 aa, Artificial Sequence
  Description of Artificial Sequence Synthetic peptide
VRLIVAVRIWLR

SEQ ID NO: 75 - protein, 12 aa, Artificial Sequence
  Description of Artificial Sequence Synthetic peptide
VRLIVAVRIWRL

SEQ ID NO: 76 - protein, 12 aa, Artificial Sequence
  Description of Artificial Sequence Synthetic peptide
IRLIVAVRIWRR

SEQ ID NO: 77 - protein, 12 aa, Artificial Sequence
  Description of Artificial Sequence Synthetic peptide
VILIVAVRIWRR

SEQ ID NO: 78 - protein, 12 aa, Artificial Sequence
  Description of Artificial Sequence Synthetic peptide
VRIIVAVRIWRR

SEQ ID NO: 79 - protein, 12 aa, Artificial Sequence
  Description of Artificial Sequence Synthetic peptide
VRLIIAVRIWRR

SEQ ID NO: 80 - protein, 12 aa, Artificial Sequence
  Description of Artificial Sequence Synthetic peptide
VRLIVIVRIWRR

SEQ ID NO: 81 - protein, 12 aa, Artificial Sequence
  Description of Artificial Sequence Synthetic peptide
VRLIVAIRIWRR

SEQ ID NO: 82 - protein, 12 aa, Artificial Sequence
  Description of Artificial Sequence Synthetic peptide
VRLIVAVIIWRR

SEQ ID NO: 83 - protein, 12 aa, Artificial Sequence
  Description of Artificial Sequence Synthetic peptide
VRLIVAVRIIRR

SEQ ID NO: 84 - protein, 12 aa, Artificial Sequence
  Description of Artificial Sequence Synthetic peptide
VRLIVAVRIWIR

SEQ ID NO: 85 - protein, 12 aa, Artificial Sequence
  Description of Artificial Sequence Synthetic peptide
VRLIVAVRIWRI

SEQ ID NO: 86 - protein, 12 aa, Artificial Sequence
  Description of Artificial Sequence Synthetic peptide
VVLIVAVRIWRR

SEQ ID NO: 87 - protein, 12 aa, Artificial Sequence
  Description of Artificial Sequence Synthetic peptide
VRVIVAVRIWRR

SEQ ID NO: 88 - protein, 12 aa, Artificial Sequence
  Description of Artificial Sequence Synthetic peptide
VRLVVAVRIWRR

SEQ ID NO: 89 - protein, 12 aa, Artificial Sequence
  Description of Artificial Sequence Synthetic peptide
VRLIVVVRIWRR

SEQ ID NO: 90 - protein, 12 aa, Artificial Sequence
  Description of Artificial Sequence Synthetic peptide
VRLIVAVVIWRR

SEQ ID NO: 91 - protein, 12 aa, Artificial Sequence
  Description of Artificial Sequence Synthetic peptide
VRLIVAVRVWRR

SEQ ID NO: 92 - protein, 12 aa, Artificial Sequence
  Description of Artificial Sequence Synthetic peptide
VRLIVAVRIVRR

SEQ ID NO: 93 - protein, 12 aa, Artificial Sequence
  Description of Artificial Sequence Synthetic peptide
VRLIVAVRIWVR

SEQ ID NO: 94 - protein, 12 aa, Artificial Sequence
  Description of Artificial Sequence Synthetic peptide
VRLIVAVRIWRV

SEQ ID NO: 95 - protein, 12 aa, Artificial Sequence
  Description of Artificial Sequence Synthetic peptide
WRLIVAVRIWRR

SEQ ID NO: 96 - protein, 12 aa, Artificial Sequence
  Description of Artificial Sequence Synthetic peptide
VWLIVAVRIWRR

SEQ ID NO: 97 - protein, 12 aa, Artificial Sequence
  Description of Artificial Sequence Synthetic peptide
VRWIVAVRIWRR

SEQ ID NO: 98 - protein, 12 aa, Artificial Sequence
  Description of Artificial Sequence Synthetic peptide
VRLWVAVRIWRR

SEQ ID NO: 99 - protein, 12 aa, Artificial Sequence
  Description of Artificial Sequence Synthetic peptide
VRLIWAVRIWRR

SEQ ID NO: 100 - protein, 12 aa, Artificial Sequence
  Description of Artificial Sequence Synthetic peptide
VRLIVWVRIWRR

SEQ ID NO: 101 - protein, 12 aa, Artificial Sequence
  Description of Artificial Sequence Synthetic peptide
VRLIVAWRIWRR

SEQ ID NO: 102 - protein, 12 aa, Artificial Sequence
  Description of Artificial Sequence Synthetic peptide
VRLIVAVWIWRR

SEQ ID NO: 103 - protein, 12 aa, Artificial Sequence
  Description of Artificial Sequence Synthetic peptide
VRLIVAVRWWRR

SEQ ID NO: 104 - protein, 12 aa, Artificial Sequence
  Description of Artificial Sequence Synthetic peptide
VRLIVAVRIWWR

SEQ ID NO: 105 - protein, 12 aa, Artificial Sequence
  Description of Artificial Sequence Synthetic peptide
VRLIVAVRIWRW

SEQ ID NO: 106 - protein, 12 aa, Artificial Sequence
  Description of Artificial Sequence Synthetic peptide
QRLIVAVRIWRR

SEQ ID NO: 107 - protein, 12 aa, Artificial Sequence
  Description of Artificial Sequence Synthetic peptide
VQLIVAVRIWRR

SEQ ID NO: 108 - protein, 12 aa, Artificial Sequence
  Description of Artificial Sequence Synthetic peptide
VRQIVAVRIWRR

SEQ ID NO: 109 - protein, 12 aa, Artificial Sequence
  Description of Artificial Sequence Synthetic peptide
VRLQVAVRIWRR

SEQ ID NO: 110 - protein, 12 aa, Artificial Sequence
  Description of Artificial Sequence Synthetic peptide
VRLIQAVRIWRR

SEQ ID NO: 111 - protein, 12 aa, Artificial Sequence
  Description of Artificial Sequence Synthetic peptide
VRLIVQVRIWRR

SEQ ID NO: 112 - protein, 12 aa, Artificial Sequence
  Description of Artificial Sequence Synthetic peptide
VRLIVAQRIWRR

SEQ ID NO: 113 - protein, 12 aa, Artificial Sequence
  Description of Artificial Sequence Synthetic peptide
VRLIVAVQIWRR

SEQ ID NO: 114 - protein, 12 aa, Artificial Sequence
  Description of Artificial Sequence Synthetic peptide
VRLIVAVRQWRR

SEQ ID NO: 115 - protein, 12 aa, Artificial Sequence
  Description of Artificial Sequence Synthetic peptide
VRLIVAVRIQRR

SEQ ID NO: 116 - protein, 12 aa, Artificial Sequence
  Description of Artificial Sequence Synthetic peptide
VRLIVAVRIWQR

SEQ ID NO: 117 - protein, 12 aa, Artificial Sequence
  Description of Artificial Sequence Synthetic peptide
VRLIVAVRIWRQ

SEQ ID NO: 118 - protein, 12 aa, Artificial Sequence
  Description of Artificial Sequence Synthetic peptide
GQRWLIVWRIRK

SEQ ID NO: 119 - protein, 12 aa, Artificial Sequence
  Description of Artificial Sequence Synthetic peptide
VGRWLIVWRIRK

SEQ ID NO: 120 - protein, 12 aa, Artificial Sequence
  Description of Artificial Sequence Synthetic peptide
VQGWLIVWRIRK

SEQ ID NO: 121 - protein, 12 aa, Artificial Sequence
  Description of Artificial Sequence Synthetic peptide
VQRGLIVWRIRK

SEQ ID NO: 122 - protein, 12 aa, Artificial Sequence
  Description of Artificial Sequence Synthetic peptide
VQRWGIVWRIRK

SEQ ID NO: 123 - protein, 12 aa, Artificial Sequence
  Description of Artificial Sequence Synthetic peptide
VQRWLGVWRIRK

SEQ ID NO: 124 - protein, 12 aa, Artificial Sequence
  Description of Artificial Sequence Synthetic peptide
VQRWLIGWRIRK

SEQ ID NO: 125 - protein, 12 aa, Artificial Sequence
  Description of Artificial Sequence Synthetic peptide
VQRWLIVGRIRK

SEQ ID NO: 126 - protein, 12 aa, Artificial Sequence
  Description of Artificial Sequence Synthetic peptide
VQRWLIVWGIRK

SEQ ID NO: 127 - protein, 12 aa, Artificial Sequence
  Description of Artificial Sequence Synthetic peptide
VQRWLIVWRGRK

SEQ ID NO: 128 - protein, 12 aa, Artificial Sequence
  Description of Artificial Sequence Synthetic peptide
VQRWLIVWRIGK

SEQ ID NO: 129 - protein, 12 aa, Artificial Sequence
  Description of Artificial Sequence Synthetic peptide
VQRWLIVWRIRG

SEQ ID NO: 130 - protein, 12 aa, Artificial Sequence
  Description of Artificial Sequence Synthetic peptide
AQRWLIVWRIRK

SEQ ID NO: 131 - protein, 12 aa, Artificial Sequence
  Description of Artificial Sequence Synthetic peptide
VARWLIVWRIRK

SEQ ID NO: 132 - protein, 12 aa, Artificial Sequence
  Description of Artificial Sequence Synthetic peptide
VQAWLIVWRIRK

SEQ ID NO: 133 - protein, 12 aa, Artificial Sequence
  Description of Artificial Sequence Synthetic peptide
VQRALIVWRIRK

SEQ ID NO: 134 - protein, 12 aa, Artificial Sequence
  Description of Artificial Sequence Synthetic peptide
VQRWAIVWRIRK

SEQ ID NO: 135 - protein, 12 aa, Artificial Sequence
  Description of Artificial Sequence Synthetic peptide
VQRWLAVWRIRK

SEQ ID NO: 136 - protein, 12 aa, Artificial Sequence
  Description of Artificial Sequence Synthetic peptide
VQRWLIAWRIRK

SEQ ID NO: 137 - protein, 12 aa, Artificial Sequence
  Description of Artificial Sequence Synthetic peptide
VQRWLIVARIRK

SEQ ID NO: 138 - protein, 12 aa, Artificial Sequence
  Description of Artificial Sequence Synthetic peptide
VQRWLIVWAIRK

SEQ ID NO: 139 - protein, 12 aa, Artificial Sequence
  Description of Artificial Sequence Synthetic peptide
VQRWLIVWRARK

SEQ ID NO: 140 - protein, 12 aa, Artificial Sequence
  Description of Artificial Sequence Synthetic peptide
VQRWLIVWRIAK

SEQ ID NO: 141 - protein, 12 aa, Artificial Sequence
  Description of Artificial Sequence Synthetic peptide
VQRWLIVWRIRA

SEQ ID NO: 142 - protein, 12 aa, Artificial Sequence
  Description of Artificial Sequence Synthetic peptide
RQRWLIVWRIRK

SEQ ID NO: 143 - protein, 12 aa, Artificial Sequence
  Description of Artificial Sequence Synthetic peptide
VRRWLIVWRIRK

SEQ ID NO: 144 - protein, 12 aa, Artificial Sequence
  Description of Artificial Sequence Synthetic peptide
VQRRLIVWRIRK

SEQ ID NO: 145 - protein, 12 aa, Artificial Sequence
  Description of Artificial Sequence Synthetic peptide
VQRWRIVWRIRK

SEQ ID NO: 146 - protein, 12 aa, Artificial Sequence
  Description of Artificial Sequence Synthetic peptide
VQRWLRVWRIRK

SEQ ID NO: 147 - protein, 12 aa, Artificial Sequence
  Description of Artificial Sequence Synthetic peptide
VQRWLIRWRIRK

SEQ ID NO: 148 - protein, 12 aa, Artificial Sequence
  Description of Artificial Sequence Synthetic peptide
VQRWLIVRRIRK

SEQ ID NO: 149 - protein, 12 aa, Artificial Sequence
  Description of Artificial Sequence Synthetic peptide
VQRWLIVWRRRK

SEQ ID NO: 150 - protein, 12 aa, Artificial Sequence
  Description of Artificial Sequence Synthetic peptide
VQRWLIVWRIRR

SEQ ID NO: 151 - protein, 12 aa, Artificial Sequence
  Description of Artificial Sequence Synthetic peptide
KQRWLIVWRIRK

SEQ ID NO: 152 - protein, 12 aa, Artificial Sequence
  Description of Artificial Sequence Synthetic peptide
VKRWLIVWRIRK

SEQ ID NO: 153 - protein, 12 aa, Artificial Sequence
  Description of Artificial Sequence Synthetic peptide
VQKWLIVWRIRK

SEQ ID NO: 154 - protein, 12 aa, Artificial Sequence
  Description of Artificial Sequence Synthetic peptide
VQRKLIVWRIRK

SEQ ID NO: 155 - protein, 12 aa, Artificial Sequence
  Description of Artificial Sequence Synthetic peptide
VQRWKIVWRIRK

SEQ ID NO: 156 - protein, 12 aa, Artificial Sequence
  Description of Artificial Sequence Synthetic peptide
VQRWLKVWRIRK

SEQ ID NO: 157 - protein, 12 aa, Artificial Sequence
  Description of Artificial Sequence Synthetic peptide
VQRWLIKWRIRK

SEQ ID NO: 158 - protein, 12 aa, Artificial Sequence
  Description of Artificial Sequence Synthetic peptide
VQRWLIVKRIRK

SEQ ID NO: 159 - protein, 12 aa, Artificial Sequence
  Description of Artificial Sequence Synthetic peptide
VQRWLIVWKIRK

SEQ ID NO: 160 - protein, 12 aa, Artificial Sequence
  Description of Artificial Sequence Synthetic peptide
VQRWLIVWRKRK

SEQ ID NO: 161 - protein, 12 aa, Artificial Sequence
  Description of Artificial Sequence Synthetic peptide
VQRWLIVWRIKK

SEQ ID NO: 162 - protein, 12 aa, Artificial Sequence
  Description of Artificial Sequence Synthetic peptide
LQRWLIVWRIRK

SEQ ID NO: 163 - protein, 12 aa, Artificial Sequence
  Description of Artificial Sequence Synthetic peptide
VLRWLIVWRIRK

SEQ ID NO: 164 - protein, 12 aa, Artificial Sequence
  Description of Artificial Sequence Synthetic peptide
VQLWLIVWRIRK

SEQ ID NO: 165 - protein, 12 aa, Artificial Sequence
  Description of Artificial Sequence Synthetic peptide
VQRLLIVWRIRK

SEQ ID NO: 166 - protein, 12 aa, Artificial Sequence
  Description of Artificial Sequence Synthetic peptide
VQRWLLVWRIRK

SEQ ID NO: 167 - protein, 12 aa, Artificial Sequence
  Description of Artificial Sequence Synthetic peptide
VQRWLILWRIRK

SEQ ID NO: 168 - protein, 12 aa, Artificial Sequence
  Description of Artificial Sequence Synthetic peptide
VQRWLIVLRIRK

SEQ ID NO: 169 - protein, 12 aa, Artificial Sequence
  Description of Artificial Sequence Synthetic peptide
VQRWLIVWLIRK

SEQ ID NO: 170 - protein, 12 aa, Artificial Sequence
  Description of Artificial Sequence Synthetic peptide
VQRWLIVWRLRK

SEQ ID NO: 171 - protein, 12 aa, Artificial Sequence
  Description of Artificial Sequence Synthetic peptide
VQRWLIVWRILK

SEQ ID NO: 172 - protein, 12 aa, Artificial Sequence
  Description of Artificial Sequence Synthetic peptide
VQRWLIVWRIRL

SEQ ID NO: 173 - protein, 12 aa, Artificial Sequence
  Description of Artificial Sequence Synthetic peptide
IQRWLIVWRIRK

SEQ ID NO: 174 - protein, 12 aa, Artificial Sequence
  Description of Artificial Sequence Synthetic peptide
VIRWLIVWRIRK

SEQ ID NO: 175 - protein, 12 aa, Artificial Sequence
  Description of Artificial Sequence Synthetic peptide
VQIWLIVWRIRK

SEQ ID NO: 176 - protein, 12 aa, Artificial Sequence
  Description of Artificial Sequence Synthetic peptide
VQRILIVWRIRK

SEQ ID NO: 177 - protein, 12 aa, Artificial Sequence
  Description of Artificial Sequence Synthetic peptide
VQRWIIVWRIRK

SEQ ID NO: 178 - protein, 12 aa, Artificial Sequence
  Description of Artificial Sequence Synthetic peptide
VQRWLIIWRIRK

SEQ ID NO: 179 - protein, 12 aa, Artificial Sequence
  Description of Artificial Sequence Synthetic peptide
VQRWLIVIRIRK

SEQ ID NO: 180 - protein, 12 aa, Artificial Sequence
  Description of Artificial Sequence Synthetic peptide
VQRWLIVWIIRK

SEQ ID NO: 181 - protein, 12 aa, Artificial Sequence
  Description of Artificial Sequence Synthetic peptide
VQRWLIVWRIIK

SEQ ID NO: 182 - protein, 12 aa, Artificial Sequence
  Description of Artificial Sequence Synthetic peptide
VQRWLIVWRIRI

SEQ ID NO: 183 - protein, 12 aa, Artificial Sequence
  Description of Artificial Sequence Synthetic peptide
VVRWLIVWRIRK

SEQ ID NO: 184 - protein, 12 aa, Artificial Sequence
  Description of Artificial Sequence Synthetic peptide
VQVWLIVWRIRK

SEQ ID NO: 185 - protein, 12 aa, Artificial Sequence
  Description of Artificial Sequence Synthetic peptide
VQRVLIVWRIRK

SEQ ID NO: 186 - protein, 12 aa, Artificial Sequence
  Description of Artificial Sequence Synthetic peptide
VQRWVIVWRIRK

SEQ ID NO: 187 - protein, 12 aa, Artificial Sequence
  Description of Artificial Sequence Synthetic peptide
VQRWLVVWRIRK

SEQ ID NO: 188 - protein, 12 aa, Artificial Sequence
  Description of Artificial Sequence Synthetic peptide
VQRWLIVVRIRK

SEQ ID NO: 189 - protein, 12 aa, Artificial Sequence
  Description of Artificial Sequence Synthetic peptide
VQRWLIVWVIRK

SEQ ID NO: 190 - protein, 12 aa, Artificial Sequence
  Description of Artificial Sequence Synthetic peptide
VQRWLIVWRVRK

SEQ ID NO: 191 - protein, 12 aa, Artificial Sequence
  Description of Artificial Sequence Synthetic peptide
VQRWLIVWRIVK

SEQ ID NO: 192 - protein, 12 aa, Artificial Sequence
  Description of Artificial Sequence Synthetic peptide
VQRWLIVWRIRV

SEQ ID NO: 193 - protein, 12 aa, Artificial Sequence
  Description of Artificial Sequence Synthetic peptide
WQRWLIVWRIRK

SEQ ID NO: 194 - protein, 12 aa, Artificial Sequence
  Description of Artificial Sequence Synthetic peptide
VWRWLIVWRIRK

SEQ ID NO: 195 - protein, 12 aa, Artificial Sequence
  Description of Artificial Sequence Synthetic peptide
VQWWLIVWRIRK

SEQ ID NO: 196 - protein, 12 aa, Artificial Sequence
  Description of Artificial Sequence Synthetic peptide
VQRWWIVWRIRK

SEQ ID NO: 197 - protein, 12 aa, Artificial Sequence
  Description of Artificial Sequence Synthetic peptide
VQRWLWVWRIRK

SEQ ID NO: 198 - protein, 12 aa, Artificial Sequence
  Description of Artificial Sequence Synthetic peptide
VQRWLIWWRIRK

SEQ ID NO: 199 - protein, 12 aa, Artificial Sequence
  Description of Artificial Sequence Synthetic peptide
VQRWLIVWWIRK

SEQ ID NO: 200 - protein, 12 aa, Artificial Sequence
  Description of Artificial Sequence Synthetic peptide
VQRWLIVWRWRK

SEQ ID NO: 201 - protein, 12 aa, Artificial Sequence
  Description of Artificial Sequence Synthetic peptide
VQRWLIVWRIWK

SEQ ID NO: 202 - protein, 12 aa, Artificial Sequence
  Description of Artificial Sequence Synthetic peptide
VQRWLIVWRIRW

SEQ ID NO: 203 - protein, 12 aa, Artificial Sequence
  Description of Artificial Sequence Synthetic peptide
QQRWLIVWRIRK

SEQ ID NO: 204 - protein, 12 aa, Artificial Sequence
  Description of Artificial Sequence Synthetic peptide
VQQWLIVWRIRK

SEQ ID NO: 205 - protein, 12 aa, Artificial Sequence
  Description of Artificial Sequence Synthetic peptide
VQRQLIVWRIRK

SEQ ID NO: 206 - protein, 12 aa, Artificial Sequence
  Description of Artificial Sequence Synthetic peptide
VQRWQIVWRIRK

SEQ ID NO: 207 - protein, 12 aa, Artificial Sequence
  Description of Artificial Sequence Synthetic peptide
VQRWLQVWRIRK

SEQ ID NO: 208 - protein, 12 aa, Artificial Sequence
  Description of Artificial Sequence Synthetic peptide
VQRWLIQWRIRK

SEQ ID NO: 209 - protein, 12 aa, Artificial Sequence
  Description of Artificial Sequence Synthetic peptide
VQRWLIVQRIRK

SEQ ID NO: 210 - protein, 12 aa, Artificial Sequence
  Description of Artificial Sequence Synthetic peptide
VQRWLIVWQIRK

SEQ ID NO: 211 - protein, 12 aa, Artificial Sequence
  Description of Artificial Sequence Synthetic peptide
VQRWLIVWRQRK

SEQ ID NO: 212 - protein, 12 aa, Artificial Sequence
  Description of Artificial Sequence Synthetic peptide
VQRWLIVWRIQK

SEQ ID NO: 213 - protein, 12 aa, Artificial Sequence
  Description of Artificial Sequence Synthetic peptide
VQRWLIVWRIRQ

SEQ ID NO: 214 - protein, 12 aa, Artificial Sequence
  Description of Artificial Sequence Synthetic peptide
GQLRIRVAVIRA

SEQ ID NO: 215 - protein, 12 aa, Artificial Sequence
  Description of Artificial Sequence Synthetic peptide
VGLRIRVAVIRA

SEQ ID NO: 216 - protein, 12 aa, Artificial Sequence
  Description of Artificial Sequence Synthetic peptide
VQGRIRVAVIRA

SEQ ID NO: 217 - protein, 12 aa, Artificial Sequence
  Description of Artificial Sequence Synthetic peptide
VQLGIRVAVIRA

SEQ ID NO: 218 - protein, 12 aa, Artificial Sequence
  Description of Artificial Sequence Synthetic peptide
VQLRGRVAVIRA

SEQ ID NO: 219 - protein, 12 aa, Artificial Sequence
  Description of Artificial Sequence Synthetic peptide
VQLRIGVAVIRA

SEQ ID NO: 220 - protein, 12 aa, Artificial Sequence
  Description of Artificial Sequence Synthetic peptide
VQLRIRGAVIRA

SEQ ID NO: 221 - protein, 12 aa, Artificial Sequence
  Description of Artificial Sequence Synthetic peptide
VQLRIRVGVIRA

SEQ ID NO: 222 - protein, 12 aa, Artificial Sequence
  Description of Artificial Sequence Synthetic peptide
VQLRIRVAGIRA

SEQ ID NO: 223 - protein, 12 aa, Artificial Sequence
  Description of Artificial Sequence Synthetic peptide
VQLRIRVAVGRA

SEQ ID NO: 224 - protein, 12 aa, Artificial Sequence
  Description of Artificial Sequence Synthetic peptide
VQLRIRVAVIGA

SEQ ID NO: 225 - protein, 12 aa, Artificial Sequence
  Description of Artificial Sequence Synthetic peptide
VQLRIRVAVIRG

SEQ ID NO: 226 - protein, 12 aa, Artificial Sequence
  Description of Artificial Sequence Synthetic peptide
AQLRIRVAVIRA

SEQ ID NO: 227 - protein, 12 aa, Artificial Sequence
  Description of Artificial Sequence Synthetic peptide
VALRIRVAVIRA

SEQ ID NO: 228 - protein, 12 aa, Artificial Sequence
  Description of Artificial Sequence Synthetic peptide
VQARIRVAVIRA

SEQ ID NO: 229 - protein, 12 aa, Artificial Sequence
  Description of Artificial Sequence Synthetic peptide
VQLAIRVAVIRA

SEQ ID NO: 230 - protein, 12 aa, Artificial Sequence
  Description of Artificial Sequence Synthetic peptide
VQLRARVAVIRA

SEQ ID NO: 231 - protein, 12 aa, Artificial Sequence
  Description of Artificial Sequence Synthetic peptide
VQLRIAVAVIRA

SEQ ID NO: 232 - protein, 12 aa, Artificial Sequence
  Description of Artificial Sequence Synthetic peptide
VQLRIRAAVIRA

SEQ ID NO: 233 - protein, 12 aa, Artificial Sequence
  Description of Artificial Sequence Synthetic peptide
VQLRIRVAAIRA

SEQ ID NO: 234 - protein, 12 aa, Artificial Sequence
  Description of Artificial Sequence Synthetic peptide
VQLRIRVAVARA

SEQ ID NO: 235 - protein, 12 aa, Artificial Sequence
  Description of Artificial Sequence Synthetic peptide
VQLRIRVAVIAA

SEQ ID NO: 236 - protein, 12 aa, Artificial Sequence
  Description of Artificial Sequence Synthetic peptide
RQLRIRVAVIRA

SEQ ID NO: 237 - protein, 12 aa, Artificial Sequence
  Description of Artificial Sequence Synthetic peptide
VRLRIRVAVIRA

SEQ ID NO: 238 - protein, 12 aa, Artificial Sequence
  Description of Artificial Sequence Synthetic peptide
VQRRIRVAVIRA

SEQ ID NO: 239 - protein, 12 aa, Artificial Sequence
  Description of Artificial Sequence Synthetic peptide
VQLRRRVAVIRA

SEQ ID NO: 240 - protein, 12 aa, Artificial Sequence
  Description of Artificial Sequence Synthetic peptide
VQLRIRRAVIRA

SEQ ID NO: 241 - protein, 12 aa, Artificial Sequence
  Description of Artificial Sequence Synthetic peptide
VQLRIRVRVIRA

SEQ ID NO: 242 - protein, 12 aa, Artificial Sequence
  Description of Artificial Sequence Synthetic peptide
VQLRIRVARIRA

SEQ ID NO: 243 - protein, 12 aa, Artificial Sequence
  Description of Artificial Sequence Synthetic peptide
VQLRIRVAVRRA

SEQ ID NO: 244 - protein, 12 aa, Artificial Sequence
  Description of Artificial Sequence Synthetic peptide
VQLRIRVAVIRR

SEQ ID NO: 245 - protein, 12 aa, Artificial Sequence
  Description of Artificial Sequence Synthetic peptide
KQLRIRVAVIRA

SEQ ID NO: 246 - protein, 12 aa, Artificial Sequence
  Description of Artificial Sequence Synthetic peptide
VKLRIRVAVIRA

SEQ ID NO: 247 - protein, 12 aa, Artificial Sequence
  Description of Artificial Sequence Synthetic peptide
VQKRIRVAVIRA

SEQ ID NO: 248 - protein, 12 aa, Artificial Sequence
  Description of Artificial Sequence Synthetic peptide
VQLKIRVAVIRA

SEQ ID NO: 249 - protein, 12 aa, Artificial Sequence
  Description of Artificial Sequence Synthetic peptide
VQLRKRVAVIRA

SEQ ID NO: 250 - protein, 12 aa, Artificial Sequence
  Description of Artificial Sequence Synthetic peptide
VQLRIKVAVIRA

SEQ ID NO: 251 - protein, 12 aa, Artificial Sequence
  Description of Artificial Sequence Synthetic peptide
VQLRIRKAVIRA

SEQ ID NO: 252 - protein, 12 aa, Artificial Sequence
  Description of Artificial Sequence Synthetic peptide
VQLRIRVKVIRA

SEQ ID NO: 253 - protein, 12 aa, Artificial Sequence
  Description of Artificial Sequence Synthetic peptide
VQLRIRVAKIRA

SEQ ID NO: 254 - protein, 12 aa, Artificial Sequence
  Description of Artificial Sequence Synthetic peptide
VQLRIRVAVKRA

SEQ ID NO: 255 - protein, 12 aa, Artificial Sequence
  Description of Artificial Sequence Synthetic peptide
VQLRIRVAVIKA

SEQ ID NO: 256 - protein, 12 aa, Artificial Sequence
  Description of Artificial Sequence Synthetic peptide
VQLRIRVAVIRK

SEQ ID NO: 257 - protein, 12 aa, Artificial Sequence
  Description of Artificial Sequence Synthetic peptide
LQLRIRVAVIRA

SEQ ID NO: 258 - protein, 12 aa, Artificial Sequence
  Description of Artificial Sequence Synthetic peptide
VLLRIRVAVIRA

SEQ ID NO: 259 - protein, 12 aa, Artificial Sequence
  Description of Artificial Sequence Synthetic peptide
VQLLIRVAVIRA

SEQ ID NO: 260 - protein, 12 aa, Artificial Sequence
  Description of Artificial Sequence Synthetic peptide
VQLRLRVAVIRA

SEQ ID NO: 261 - protein, 12 aa, Artificial Sequence
  Description of Artificial Sequence Synthetic peptide
VQLRILVAVIRA

SEQ ID NO: 262 - protein, 12 aa, Artificial Sequence
  Description of Artificial Sequence Synthetic peptide
VQLRIRLAVIRA

SEQ ID NO: 263 - protein, 12 aa, Artificial Sequence
  Description of Artificial Sequence Synthetic peptide
VQLRIRVLVIRA

SEQ ID NO: 264 - protein, 12 aa, Artificial Sequence
  Description of Artificial Sequence Synthetic peptide
VQLRIRVALIRA

SEQ ID NO: 265 - protein, 12 aa, Artificial Sequence
  Description of Artificial Sequence Synthetic peptide
VQLRIRVAVLRA

SEQ ID NO: 266 - protein, 12 aa, Artificial Sequence
  Description of Artificial Sequence Synthetic peptide
VQLRIRVAVILA

SEQ ID NO: 267 - protein, 12 aa, Artificial Sequence
  Description of Artificial Sequence Synthetic peptide
VQLRIRVAVIRL

SEQ ID NO: 268 - protein, 12 aa, Artificial Sequence
  Description of Artificial Sequence Synthetic peptide
IQLRIRVAVIRA

SEQ ID NO: 269 - protein, 12 aa, Artificial Sequence
  Description of Artificial Sequence Synthetic peptide
VILRIRVAVIRA

SEQ ID NO: 270 - protein, 12 aa, Artificial Sequence
  Description of Artificial Sequence Synthetic peptide
VQIRIRVAVIRA

SEQ ID NO: 271 - protein, 12 aa, Artificial Sequence
  Description of Artificial Sequence Synthetic peptide
VQLIIRVAVIRA

SEQ ID NO: 272 - protein, 12 aa, Artificial Sequence
  Description of Artificial Sequence Synthetic peptide
VQLRIIVAVIRA

SEQ ID NO: 273 - protein, 12 aa, Artificial Sequence
  Description of Artificial Sequence Synthetic peptide
VQLRIRIAVIRA

SEQ ID NO: 274 - protein, 12 aa, Artificial Sequence
  Description of Artificial Sequence Synthetic peptide
VQLRIRVIVIRA

SEQ ID NO: 275 - protein, 12 aa, Artificial Sequence
  Description of Artificial Sequence Synthetic peptide
VQLRIRVAIIRA

SEQ ID NO: 276 - protein, 12 aa, Artificial Sequence
  Description of Artificial Sequence Synthetic peptide
VQLRIRVAVIIA

SEQ ID NO: 277 - protein, 12 aa, Artificial Sequence
  Description of Artificial Sequence Synthetic peptide
VQLRIRVAVIRI

SEQ ID NO: 278 - protein, 12 aa, Artificial Sequence
  Description of Artificial Sequence Synthetic peptide
VVLRIRVAVIRA

SEQ ID NO: 279 - protein, 12 aa, Artificial Sequence
  Description of Artificial Sequence Synthetic peptide
VQVRIRVAVIRA

SEQ ID NO: 280 - protein, 12 aa, Artificial Sequence
  Description of Artificial Sequence Synthetic peptide
VQLVIRVAVIRA

SEQ ID NO: 281 - protein, 12 aa, Artificial Sequence
  Description of Artificial Sequence Synthetic peptide
VQLRVRVAVIRA

SEQ ID NO: 282 - protein, 12 aa, Artificial Sequence
  Description of Artificial Sequence Synthetic peptide
VQLRIVVAVIRA

SEQ ID NO: 283 - protein, 12 aa, Artificial Sequence
  Description of Artificial Sequence Synthetic peptide
VQLRIRVVVIRA

SEQ ID NO: 284 - protein, 12 aa, Artificial Sequence
  Description of Artificial Sequence Synthetic peptide
VQLRIRVAVVRA

SEQ ID NO: 285 - protein, 12 aa, Artificial Sequence
  Description of Artificial Sequence Synthetic peptide
VQLRIRVAVIVA

SEQ ID NO: 286 - protein, 12 aa, Artificial Sequence
  Description of Artificial Sequence Synthetic peptide
VQLRIRVAVIRV

SEQ ID NO: 287 - protein, 12 aa, Artificial Sequence
  Description of Artificial Sequence Synthetic peptide
WQLRIRVAVIRA

SEQ ID NO: 288 - protein, 12 aa, Artificial Sequence
  Description of Artificial Sequence Synthetic peptide
VWLRIRVAVIRA

SEQ ID NO: 289 - protein, 12 aa, Artificial Sequence
  Description of Artificial Sequence Synthetic peptide
VQWRIRVAVIRA

SEQ ID NO: 290 - protein, 12 aa, Artificial Sequence
  Description of Artificial Sequence Synthetic peptide
VQLWIRVAVIRA

SEQ ID NO: 291 - protein, 12 aa, Artificial Sequence
  Description of Artificial Sequence Synthetic peptide
VQLRWRVAVIRA

SEQ ID NO: 292 - protein, 12 aa, Artificial Sequence
  Description of Artificial Sequence Synthetic peptide
VQLRIWVAVIRA

SEQ ID NO: 293 - protein, 12 aa, Artificial Sequence
  Description of Artificial Sequence Synthetic peptide
VQLRIRWAVIRA

SEQ ID NO: 294 - protein, 12 aa, Artificial Sequence
  Description of Artificial Sequence Synthetic peptide
VQLRIRVWVIRA

SEQ ID NO: 295 - protein, 12 aa, Artificial Sequence
  Description of Artificial Sequence Synthetic peptide
VQLRIRVAWIRA

SEQ ID NO: 296 - protein, 12 aa, Artificial Sequence
  Description of Artificial Sequence Synthetic peptide
VQLRIRVAVWRA

SEQ ID NO: 297 - protein, 12 aa, Artificial Sequence
  Description of Artificial Sequence Synthetic peptide
VQLRIRVAVIWA

SEQ ID NO: 298 - protein, 12 aa, Artificial Sequence
  Description of Artificial Sequence Synthetic peptide
VQLRIRVAVIRW

SEQ ID NO: 299 - protein, 12 aa, Artificial Sequence
  Description of Artificial Sequence Synthetic peptide
QQLRIRVAVIRA

SEQ ID NO: 300 - protein, 12 aa, Artificial Sequence
  Description of Artificial Sequence Synthetic peptide
VQQRIRVAVIRA

SEQ ID NO: 301 - protein, 12 aa, Artificial Sequence
  Description of Artificial Sequence Synthetic peptide
VQLQIRVAVIRA

SEQ ID NO: 302 - protein, 12 aa, Artificial Sequence
  Description of Artificial Sequence Synthetic peptide
VQLRQRVAVIRA

SEQ ID NO: 303 - protein, 12 aa, Artificial Sequence
  Description of Artificial Sequence Synthetic peptide
VQLRIQVAVIRA

SEQ ID NO: 304 - protein, 12 aa, Artificial Sequence
  Description of Artificial Sequence Synthetic peptide
VQLRIRQAVIRA

SEQ ID NO: 305 - protein, 12 aa, Artificial Sequence
  Description of Artificial Sequence Synthetic peptide
VQLRIRVQVIRA

SEQ ID NO: 306 - protein, 12 aa, Artificial Sequence
  Description of Artificial Sequence Synthetic peptide
VQLRIRVAQIRA

SEQ ID NO: 307 - protein, 12 aa, Artificial Sequence
  Description of Artificial Sequence Synthetic peptide
VQLRIRVAVQRA

SEQ ID NO: 308 - protein, 12 aa, Artificial Sequence
  Description of Artificial Sequence Synthetic peptide
VQLRIRVAVIQA

SEQ ID NO: 309 - protein, 12 aa, Artificial Sequence
  Description of Artificial Sequence Synthetic peptide
VQLRIRVAVIRQ

SEQ ID NO: 310 - protein, 12 aa, Artificial Sequence
  Description of Artificial Sequence Synthetic peptide
VRLIVAVWRIRK

SEQ ID NO: 311 - protein, 12 aa, Artificial Sequence
  Description of Artificial Sequence Synthetic peptide
VRLIVAVAVIRA

SEQ ID NO: 312 - protein, 12 aa, Artificial Sequence
  Description of Artificial Sequence Synthetic peptide
VQRWLIVRIWRR

SEQ ID NO: 313 - protein, 12 aa, Artificial Sequence
  Description of Artificial Sequence Synthetic peptide
VQRWLIVAVIRA

SEQ ID NO: 314 - protein, 12 aa, Artificial Sequence
  Description of Artificial Sequence Synthetic peptide
VQLRIRVRIWRR

SEQ ID NO: 315 - protein, 12 aa, Artificial Sequence
  Description of Artificial Sequence Synthetic peptide
VQLRIRVWRIRK

SEQ ID NO: 316 - protein, 12 aa, Artificial Sequence
  Description of Artificial Sequence Synthetic peptide
VRIWRRVQRWLI

SEQ ID NO: 317 - protein, 12 aa, Artificial Sequence
  Description of Artificial Sequence Synthetic peptide
VAVIRAVRLIVA

SEQ ID NO: 318 - protein, 12 aa, Artificial Sequence
  Description of Artificial Sequence Synthetic peptide
VRIWRRVQRWLI

SEQ ID NO: 319 - protein, 12 aa, Artificial Sequence
  Description of Artificial Sequence Synthetic peptide
VAVIRAVQRWLI

SEQ ID NO: 320 - protein, 12 aa, Artificial Sequence
  Description of Artificial Sequence Synthetic peptide
VRIWRRVQLRIR

SEQ ID NO: 321 - protein, 12 aa, Artificial Sequence
  Description of Artificial Sequence Synthetic peptide
VWRIRKVQLRIR

SEQ ID NO: 322 - protein, 12 aa, Artificial Sequence
  Description of Artificial Sequence Synthetic peptide
VRIWRRVRLIVA

SEQ ID NO: 323 - protein, 12 aa, Artificial Sequence
  Description of Artificial Sequence Synthetic peptide
VWRIRKVQRWLI

SEQ ID NO: 324 - protein, 12 aa, Artificial Sequence
  Description of Artificial Sequence Synthetic peptide
VAVIRAVQLRIR

SEQ ID NO: 325 - protein, 12 aa, Artificial Sequence
  Description of Artificial Sequence Synthetic peptide
VRLILIVWRIRK

SEQ ID NO: 326 - protein, 12 aa, Artificial Sequence
  Description of Artificial Sequence Synthetic peptide
VRLIIRVAVIRA

SEQ ID NO: 327 - protein, 12 aa, Artificial Sequence
  Description of Artificial Sequence Synthetic peptide
VQRWVAVRIWRR

SEQ ID NO: 328 - protein, 12 aa, Artificial Sequence
  Description of Artificial Sequence Synthetic peptide
VQRWIRVAVIRA

SEQ ID NO: 329 - protein, 12 aa, Artificial Sequence
  Description of Artificial Sequence Synthetic peptide
VQLRVAVRIWRR

SEQ ID NO: 330 - protein, 12 aa, Artificial Sequence
  Description of Artificial Sequence Synthetic peptide
VQLRLIVWRIRK

SEQ ID NO: 331 - protein, 12 aa, Artificial Sequence
  Description of Artificial Sequence Synthetic peptide
VRLIVAVRRIRK

SEQ ID NO: 332 - protein, 12 aa, Artificial Sequence
  Description of Artificial Sequence Synthetic peptide
VRLIVAVRVIRA

SEQ ID NO: 333 - protein, 12 aa, Artificial Sequence
  Description of Artificial Sequence Synthetic peptide
VQRWLIVWIWRR

SEQ ID NO: 334 - protein, 12 aa, Artificial Sequence
  Description of Artificial Sequence Synthetic peptide
VQRWLIVWVIRA

SEQ ID NO: 335 - protein, 12 aa, Artificial Sequence
  Description of Artificial Sequence Synthetic peptide
VQLRIRVAIWRR

SEQ ID NO: 336 - protein, 12 aa, Artificial Sequence
  Description of Artificial Sequence Synthetic peptide
VQLRIRVARIRK

SEQ ID NO: 337 - protein, 12 aa, Artificial Sequence
  Description of Artificial Sequence Synthetic peptide
RRWIRVAVILRV

SEQ ID NO: 338 - protein, 12 aa, Artificial Sequence
  Description of Artificial Sequence Synthetic peptide
KRIRWVILWRQV

SEQ ID NO: 339 - protein, 12 aa, Artificial Sequence
  Description of Artificial Sequence Synthetic peptide
ARIVAVRIRLQV

SEQ ID NO: 340 - protein, 12 aa, Artificial Sequence
  Description of Artificial Sequence Synthetic peptide
AVILRVRRWIRV

SEQ ID NO: 341 - protein, 12 aa, Artificial Sequence
  Description of Artificial Sequence Synthetic peptide
ILWRQVKRIRWV

SEQ ID NO: 342 - protein, 12 aa, Artificial Sequence
  Description of Artificial Sequence Synthetic peptide
RIRLQVARIVAV

SEQ ID NO: 343 - protein, 12 aa, Artificial Sequence
  Description of Artificial Sequence Synthetic peptide
GRWIRVAVILRV

SEQ ID NO: 344 - protein, 12 aa, Artificial Sequence
  Description of Artificial Sequence Synthetic peptide
RGWIRVAVILRV

SEQ ID NO: 345 - protein, 12 aa, Artificial Sequence
  Description of Artificial Sequence Synthetic peptide
RRGIRVAVILRV

SEQ ID NO: 346 - protein, 12 aa, Artificial Sequence
  Description of Artificial Sequence Synthetic peptide
RRWGRVAVILRV

SEQ ID NO: 347 - protein, 12 aa, Artificial Sequence
  Description of Artificial Sequence Synthetic peptide
RRWIGVAVILRV

SEQ ID NO: 348 - protein, 12 aa, Artificial Sequence
  Description of Artificial Sequence Synthetic peptide
RRWIRGAVILRV

SEQ ID NO: 349 - protein, 12 aa, Artificial Sequence
  Description of Artificial Sequence Synthetic peptide
RRWIRVGVILRV

SEQ ID NO: 350 - protein, 12 aa, Artificial Sequence
  Description of Artificial Sequence Synthetic peptide
RRWIRVAGILRV

SEQ ID NO: 351 - protein, 12 aa, Artificial Sequence
  Description of Artificial Sequence Synthetic peptide
RRWIRVAVGLRV

SEQ ID NO: 352 - protein, 12 aa, Artificial Sequence
  Description of Artificial Sequence Synthetic peptide
RRWIRVAVIGRV

SEQ ID NO: 353 - protein, 12 aa, Artificial Sequence
  Description of Artificial Sequence Synthetic peptide
RRWIRVAVILGV

SEQ ID NO: 354 - protein, 12 aa, Artificial Sequence
  Description of Artificial Sequence Synthetic peptide
RRWIRVAVILRG

SEQ ID NO: 355 - protein, 12 aa, Artificial Sequence
  Description of Artificial Sequence Synthetic peptide
ARWIRVAVILRV

SEQ ID NO: 356 - protein, 12 aa, Artificial Sequence
  Description of Artificial Sequence Synthetic peptide
RAWIRVAVILRV

SEQ ID NO: 357 - protein, 12 aa, Artificial Sequence
  Description of Artificial Sequence Synthetic peptide
RRAIRVAVILRV

SEQ ID NO: 358 - protein, 12 aa, Artificial Sequence
  Description of Artificial Sequence Synthetic peptide
RRWARVAVILRV

SEQ ID NO: 359 - protein, 12 aa, Artificial Sequence
  Description of Artificial Sequence Synthetic peptide
RRWIAVAVILRV

SEQ ID NO: 360 - protein, 12 aa, Artificial Sequence
  Description of Artificial Sequence Synthetic peptide
RRWIRAAVILRV

SEQ ID NO: 361 - protein, 12 aa, Artificial Sequence
  Description of Artificial Sequence Synthetic peptide
RRWIRVAAILRV

SEQ ID NO: 362 - protein, 12 aa, Artificial Sequence
  Description of Artificial Sequence Synthetic peptide
RRWIRVAVALRV

SEQ ID NO: 363 - protein, 12 aa, Artificial Sequence
  Description of Artificial Sequence Synthetic peptide
RRWIRVAVIARV

SEQ ID NO: 364 - protein, 12 aa, Artificial Sequence
  Description of Artificial Sequence Synthetic peptide
RRWIRVAVILAV

SEQ ID NO: 365 - protein, 12 aa, Artificial Sequence
  Description of Artificial Sequence Synthetic peptide
RRWIRVAVILRA

SEQ ID NO: 366 - protein, 12 aa, Artificial Sequence
  Description of Artificial Sequence Synthetic peptide
RRRIRVAVILRV

SEQ ID NO: 367 - protein, 12 aa, Artificial Sequence
  Description of Artificial Sequence Synthetic peptide
RRWRRVAVILRV

SEQ ID NO: 368 - protein, 12 aa, Artificial Sequence
  Description of Artificial Sequence Synthetic peptide
RRWIRRAVILRV

SEQ ID NO: 369 - protein, 12 aa, Artificial Sequence
  Description of Artificial Sequence Synthetic peptide
RRWIRVRVILRV

SEQ ID NO: 370 - protein, 12 aa, Artificial Sequence
  Description of Artificial Sequence Synthetic peptide
RRWIRVARILRV

SEQ ID NO: 371 - protein, 12 aa, Artificial Sequence
  Description of Artificial Sequence Synthetic peptide
RRWIRVAVRLRV

SEQ ID NO: 372 - protein, 12 aa, Artificial Sequence
  Description of Artificial Sequence Synthetic peptide
RRWIRVAVIRRV

SEQ ID NO: 373 - protein, 12 aa, Artificial Sequence
  Description of Artificial Sequence Synthetic peptide
RRWIRVAVILRR

SEQ ID NO: 374 - protein, 12 aa, Artificial Sequence
  Description of Artificial Sequence Synthetic peptide
KRWIRVAVILRV

SEQ ID NO: 375 - protein, 12 aa, Artificial Sequence
  Description of Artificial Sequence Synthetic peptide
RKWIRVAVILRV

SEQ ID NO: 376 - protein, 12 aa, Artificial Sequence
  Description of Artificial Sequence Synthetic peptide
RRKIRVAVILRV

SEQ ID NO: 377 - protein, 12 aa, Artificial Sequence
  Description of Artificial Sequence Synthetic peptide
RRWKRVAVILRV

SEQ ID NO: 378 - protein, 12 aa, Artificial Sequence
  Description of Artificial Sequence Synthetic peptide
RRWIKVAVILRV

SEQ ID NO: 379 - protein, 12 aa, Artificial Sequence
  Description of Artificial Sequence Synthetic peptide
RRWIRKAVILRV

SEQ ID NO: 380 - protein, 12 aa, Artificial Sequence
  Description of Artificial Sequence Synthetic peptide
RRWIRVKVILRV

SEQ ID NO: 381 - protein, 12 aa, Artificial Sequence
  Description of Artificial Sequence Synthetic peptide
RRWIRVAKILRV

SEQ ID NO: 382 - protein, 12 aa, Artificial Sequence
  Description of Artificial Sequence Synthetic peptide
RRWIRVAVKLRV

SEQ ID NO: 383 - protein, 12 aa, Artificial Sequence
  Description of Artificial Sequence Synthetic peptide
RRWIRVAVIKRV

SEQ ID NO: 384 - protein, 12 aa, Artificial Sequence
  Description of Artificial Sequence Synthetic peptide
RRWIRVAVILKV

SEQ ID NO: 385 - protein, 12 aa, Artificial Sequence
  Description of Artificial Sequence Synthetic peptide
RRWIRVAVILRK

SEQ ID NO: 386 - protein, 12 aa, Artificial Sequence
  Description of Artificial Sequence Synthetic peptide
VRWIRVAVILRV

SEQ ID NO: 387 - protein, 12 aa, Artificial Sequence
  Description of Artificial Sequence Synthetic peptide
RVWIRVAVILRV

SEQ ID NO: 388 - protein, 12 aa, Artificial Sequence
  Description of Artificial Sequence Synthetic peptide
RRVIRVAVILRV

SEQ ID NO: 389 - protein, 12 aa, Artificial Sequence
  Description of Artificial Sequence Synthetic peptide
RRWVRVAVILRV

SEQ ID NO: 390 - protein, 12 aa, Artificial Sequence
  Description of Artificial Sequence Synthetic peptide
RRWIVVAVILRV

SEQ ID NO: 391 - protein, 12 aa, Artificial Sequence
  Description of Artificial Sequence Synthetic peptide
RRWIRVVVILRV

SEQ ID NO: 392 - protein, 12 aa, Artificial Sequence
  Description of Artificial Sequence Synthetic peptide
RRWIRVAVVLRV

SEQ ID NO: 393 - protein, 12 aa, Artificial Sequence
  Description of Artificial Sequence Synthetic peptide
RRWIRVAVIVRV

SEQ ID NO: 394 - protein, 12 aa, Artificial Sequence
  Description of Artificial Sequence Synthetic peptide
RRWIRVAVILVV

SEQ ID NO: 395 - protein, 12 aa, Artificial Sequence
  Description of Artificial Sequence Synthetic peptide
IRWIRVAVILRV

SEQ ID NO: 396 - protein, 12 aa, Artificial Sequence
  Description of Artificial Sequence Synthetic peptide
RIWIRVAVILRV

SEQ ID NO: 397 - protein, 12 aa, Artificial Sequence
  Description of Artificial Sequence Synthetic peptide
RRIIRVAVILRV

SEQ ID NO: 398 - protein, 12 aa, Artificial Sequence
  Description of Artificial Sequence Synthetic peptide
RRWIIVAVILRV

SEQ ID NO: 399 - protein, 12 aa, Artificial Sequence
  Description of Artificial Sequence Synthetic peptide
RRWIRIAVILRV

SEQ ID NO: 400 - protein, 12 aa, Artificial Sequence
  Description of Artificial Sequence Synthetic peptide
RRWIRVIVILRV

SEQ ID NO: 401 - protein, 12 aa, Artificial Sequence
  Description of Artificial Sequence Synthetic peptide
RRWIRVAIILRV

SEQ ID NO: 402 - protein, 12 aa, Artificial Sequence
  Description of Artificial Sequence Synthetic peptide
RRWIRVAVIIRV

SEQ ID NO: 403 - protein, 12 aa, Artificial Sequence
  Description of Artificial Sequence Synthetic peptide
RRWIRVAVILIV

SEQ ID NO: 404 - protein, 12 aa, Artificial Sequence
  Description of Artificial Sequence Synthetic peptide
RRWIRVAVILRI

SEQ ID NO: 405 - protein, 12 aa, Artificial Sequence
  Description of Artificial Sequence Synthetic peptide
LRWIRVAVILRV

SEQ ID NO: 406 - protein, 12 aa, Artificial Sequence
  Description of Artificial Sequence Synthetic peptide
RLWIRVAVILRV

SEQ ID NO: 407 - protein, 12 aa, Artificial Sequence
  Description of Artificial Sequence Synthetic peptide
RRLIRVAVILRV

SEQ ID NO: 408 - protein, 12 aa, Artificial Sequence
  Description of Artificial Sequence Synthetic peptide
RRWLRVAVILRV

SEQ ID NO: 409 - protein, 12 aa, Artificial Sequence
  Description of Artificial Sequence Synthetic peptide
RRWILVAVILRV

SEQ ID NO: 410 - protein, 12 aa, Artificial Sequence
  Description of Artificial Sequence Synthetic peptide
RRWIRLAVILRV

SEQ ID NO: 411 - protein, 12 aa, Artificial Sequence
  Description of Artificial Sequence Synthetic peptide
RRWIRVLVILRV

SEQ ID NO: 412 - protein, 12 aa, Artificial Sequence
  Description of Artificial Sequence Synthetic peptide
RRWIRVALILRV

SEQ ID NO: 413 - protein, 12 aa, Artificial Sequence
  Description of Artificial Sequence Synthetic peptide
RRWIRVAVLLRV

SEQ ID NO: 414 - protein, 12 aa, Artificial Sequence
  Description of Artificial Sequence Synthetic peptide
RRWIRVAVILLV

SEQ ID NO: 415 - protein, 12 aa, Artificial Sequence
  Description of Artificial Sequence Synthetic peptide
RRWIRVAVILRL

SEQ ID NO: 416 - protein, 12 aa, Artificial Sequence
  Description of Artificial Sequence Synthetic peptide
WRWIRVAVILRV

SEQ ID NO: 417 - protein, 12 aa, Artificial Sequence
  Description of Artificial Sequence Synthetic peptide
RWWIRVAVILRV

SEQ ID NO: 418 - protein, 12 aa, Artificial Sequence
  Description of Artificial Sequence Synthetic peptide
RRWWRVAVILRV

SEQ ID NO: 419 - protein, 12 aa, Artificial Sequence
  Description of Artificial Sequence Synthetic peptide
RRWIWVAVILRV

SEQ ID NO: 420 - protein, 12 aa, Artificial Sequence
  Description of Artificial Sequence Synthetic peptide
RRWIRWAVILRV

SEQ ID NO: 421 - protein, 12 aa, Artificial Sequence
  Description of Artificial Sequence Synthetic peptide
RRWIRVWVILRV

SEQ ID NO: 422 - protein, 12 aa, Artificial Sequence
  Description of Artificial Sequence Synthetic peptide
RRWIRVAWILRV

SEQ ID NO: 423 - protein, 12 aa, Artificial Sequence
  Description of Artificial Sequence Synthetic peptide
RRWIRVAVWLRV

SEQ ID NO: 424 - protein, 12 aa, Artificial Sequence
  Description of Artificial Sequence Synthetic peptide
RRWIRVAVIWRV

SEQ ID NO: 425 - protein, 12 aa, Artificial Sequence
  Description of Artificial Sequence Synthetic peptide
RRWIRVAVILWV

SEQ ID NO: 426 - protein, 12 aa, Artificial Sequence
  Description of Artificial Sequence Synthetic peptide
RRWIRVAVILRW

SEQ ID NO: 427 - protein, 12 aa, Artificial Sequence
  Description of Artificial Sequence Synthetic peptide
QRWIRVAVILRV

SEQ ID NO: 428 - protein, 12 aa, Artificial Sequence
  Description of Artificial Sequence Synthetic peptide
RQWIRVAVILRV

SEQ ID NO: 429 - protein, 12 aa, Artificial Sequence
  Description of Artificial Sequence Synthetic peptide
RRQIRVAVILRV

SEQ ID NO: 430 - protein, 12 aa, Artificial Sequence
  Description of Artificial Sequence Synthetic peptide
RRWQRVAVILRV

SEQ ID NO: 431 - protein, 12 aa, Artificial Sequence
  Description of Artificial Sequence Synthetic peptide
RRWIQVAVILRV

SEQ ID NO: 432 - protein, 12 aa, Artificial Sequence
  Description of Artificial Sequence Synthetic peptide
RRWIRQAVILRV

SEQ ID NO: 433 - protein, 12 aa, Artificial Sequence
  Description of Artificial Sequence Synthetic peptide
RRWIRVQVILRV

SEQ ID NO: 434 - protein, 12 aa, Artificial Sequence
  Description of Artificial Sequence Synthetic peptide
RRWIRVAQILRV

SEQ ID NO: 435 - protein, 12 aa, Artificial Sequence
  Description of Artificial Sequence Synthetic peptide
RRWIRVAVQLRV

SEQ ID NO: 436 - protein, 12 aa, Artificial Sequence
  Description of Artificial Sequence Synthetic peptide
RRWIRVAVIQRV

SEQ ID NO: 437 - protein, 12 aa, Artificial Sequence
  Description of Artificial Sequence Synthetic peptide
RRWIRVAVILQV

SEQ ID NO: 438 - protein, 12 aa, Artificial Sequence
  Description of Artificial Sequence Synthetic peptide
RRWIRVAVILRQ

SEQ ID NO: 439 - protein, 12 aa, Artificial Sequence
  Description of Artificial Sequence Synthetic peptide
GQWRRIRVWVIR

SEQ ID NO: 440 - protein, 12 aa, Artificial Sequence
  Description of Artificial Sequence Synthetic peptide
VGWRRIRVWVIR

SEQ ID NO: 441 - protein, 12 aa, Artificial Sequence
  Description of Artificial Sequence Synthetic peptide
VQGRRIRVWVIR

SEQ ID NO: 442 - protein, 12 aa, Artificial Sequence
  Description of Artificial Sequence Synthetic peptide
VQWGRIRVWVIR

SEQ ID NO: 443 - protein, 12 aa, Artificial Sequence
  Description of Artificial Sequence Synthetic peptide
VQWRGIRVWVIR

SEQ ID NO: 444 - protein, 12 aa, Artificial Sequence
  Description of Artificial Sequence Synthetic peptide
VQWRRGRVWVIR

SEQ ID NO: 445 - protein, 12 aa, Artificial Sequence
  Description of Artificial Sequence Synthetic peptide
VQWRRIGVWVIR

SEQ ID NO: 446 - protein, 12 aa, Artificial Sequence
  Description of Artificial Sequence Synthetic peptide
VQWRRIRGWVIR

SEQ ID NO: 447 - protein, 12 aa, Artificial Sequence
  Description of Artificial Sequence Synthetic peptide
VQWRRIRVGVIR

SEQ ID NO: 448 - protein, 12 aa, Artificial Sequence
  Description of Artificial Sequence Synthetic peptide
VQWRRIRVWGIR

SEQ ID NO: 449 - protein, 12 aa, Artificial Sequence
  Description of Artificial Sequence Synthetic peptide
VQWRRIRVWVGR

SEQ ID NO: 450 - protein, 12 aa, Artificial Sequence
  Description of Artificial Sequence Synthetic peptide
VQWRRIRVWVIG

SEQ ID NO: 451 - protein, 12 aa, Artificial Sequence
  Description of Artificial Sequence Synthetic peptide
AQWRRIRVWVIR

SEQ ID NO: 452 - protein, 12 aa, Artificial Sequence
  Description of Artificial Sequence Synthetic peptide
VAWRRIRVWVIR

SEQ ID NO: 453 - protein, 12 aa, Artificial Sequence
  Description of Artificial Sequence Synthetic peptide
VQARRIRVWVIR

SEQ ID NO: 454 - protein, 12 aa, Artificial Sequence
  Description of Artificial Sequence Synthetic peptide
VQWARIRVWVIR

SEQ ID NO: 455 - protein, 12 aa, Artificial Sequence
  Description of Artificial Sequence Synthetic peptide
VQWRAIRVWVIR

SEQ ID NO: 456 - protein, 12 aa, Artificial Sequence
  Description of Artificial Sequence Synthetic peptide
VQWRRARVWVIR

SEQ ID NO: 457 - protein, 12 aa, Artificial Sequence
  Description of Artificial Sequence Synthetic peptide
VQWRRIAVWVIR

SEQ ID NO: 458 - protein, 12 aa, Artificial Sequence
  Description of Artificial Sequence Synthetic peptide
VQWRRIRAWVIR

SEQ ID NO: 459 - protein, 12 aa, Artificial Sequence
  Description of Artificial Sequence Synthetic peptide
VQWRRIRVAVIR

SEQ ID NO: 460 - protein, 12 aa, Artificial Sequence
  Description of Artificial Sequence Synthetic peptide
VQWRRIRVWAIR

SEQ ID NO: 461 - protein, 12 aa, Artificial Sequence
  Description of Artificial Sequence Synthetic peptide
VQWRRIRVWVAR

SEQ ID NO: 462 - protein, 12 aa, Artificial Sequence
  Description of Artificial Sequence Synthetic peptide
VQWRRIRVWVIA

SEQ ID NO: 463 - protein, 12 aa, Artificial Sequence
  Description of Artificial Sequence Synthetic peptide
RQWRRIRVWVIR

SEQ ID NO: 464 - protein, 12 aa, Artificial Sequence
  Description of Artificial Sequence Synthetic peptide
VRWRRIRVWVIR

SEQ ID NO: 465 - protein, 12 aa, Artificial Sequence
  Description of Artificial Sequence Synthetic peptide
VQRRRIRVWVIR

SEQ ID NO: 466 - protein, 12 aa, Artificial Sequence
  Description of Artificial Sequence Synthetic peptide
VQWRRRRVWVIR

SEQ ID NO: 467 - protein, 12 aa, Artificial Sequence
  Description of Artificial Sequence Synthetic peptide
VQWRRIRRWVIR

SEQ ID NO: 468 - protein, 12 aa, Artificial Sequence
  Description of Artificial Sequence Synthetic peptide
VQWRRIRVRVIR

SEQ ID NO: 469 - protein, 12 aa, Artificial Sequence
  Description of Artificial Sequence Synthetic peptide
VQWRRIRVWRIR

SEQ ID NO: 470 - protein, 12 aa, Artificial Sequence
  Description of Artificial Sequence Synthetic peptide
VQWRRIRVWVRR

SEQ ID NO: 471 - protein, 12 aa, Artificial Sequence
  Description of Artificial Sequence Synthetic peptide
KQWRRIRVWVIR

SEQ ID NO: 472 - protein, 12 aa, Artificial Sequence
  Description of Artificial Sequence Synthetic peptide
VKWRRIRVWVIR

SEQ ID NO: 473 - protein, 12 aa, Artificial Sequence
  Description of Artificial Sequence Synthetic peptide
VQKRRIRVWVIR

SEQ ID NO: 474 - protein, 12 aa, Artificial Sequence
  Description of Artificial Sequence Synthetic peptide
VQWKRIRVWVIR

SEQ ID NO: 475 - protein, 12 aa, Artificial Sequence
  Description of Artificial Sequence Synthetic peptide
VQWRKIRVWVIR

SEQ ID NO: 476 - protein, 12 aa, Artificial Sequence
  Description of Artificial Sequence Synthetic peptide
VQWRRKRVWVIR

SEQ ID NO: 477 - protein, 12 aa, Artificial Sequence
  Description of Artificial Sequence Synthetic peptide
VQWRRIKVWVIR

SEQ ID NO: 478 - protein, 12 aa, Artificial Sequence
  Description of Artificial Sequence Synthetic peptide
VQWRRIRKWVIR

SEQ ID NO: 479 - protein, 12 aa, Artificial Sequence
  Description of Artificial Sequence Synthetic peptide
VQWRRIRVKVIR

SEQ ID NO: 480 - protein, 12 aa, Artificial Sequence
  Description of Artificial Sequence Synthetic peptide
VQWRRIRVWKIR

SEQ ID NO: 481 - protein, 12 aa, Artificial Sequence
  Description of Artificial Sequence Synthetic peptide
VQWRRIRVWVKR

SEQ ID NO: 482 - protein, 12 aa, Artificial Sequence
  Description of Artificial Sequence Synthetic peptide
VQWRRIRVWVIK

SEQ ID NO: 483 - protein, 12 aa, Artificial Sequence
  Description of Artificial Sequence Synthetic peptide
VVWRRIRVWVIR

SEQ ID NO: 484 - protein, 12 aa, Artificial Sequence
  Description of Artificial Sequence Synthetic peptide
VQVRRIRVWVIR

SEQ ID NO: 485 - protein, 12 aa, Artificial Sequence
  Description of Artificial Sequence Synthetic peptide
VQWVRIRVWVIR

SEQ ID NO: 486 - protein, 12 aa, Artificial Sequence
  Description of Artificial Sequence Synthetic peptide
VQWRVIRVWVIR

SEQ ID NO: 487 - protein, 12 aa, Artificial Sequence
  Description of Artificial Sequence Synthetic peptide
VQWRRVRVWVIR

SEQ ID NO: 488 - protein, 12 aa, Artificial Sequence
  Description of Artificial Sequence Synthetic peptide
VQWRRIVVWVIR

SEQ ID NO: 489 - protein, 12 aa, Artificial Sequence
  Description of Artificial Sequence Synthetic peptide
VQWRRIRVVVIR

SEQ ID NO: 490 - protein, 12 aa, Artificial Sequence
  Description of Artificial Sequence Synthetic peptide
VQWRRIRVWVVR

SEQ ID NO: 491 - protein, 12 aa, Artificial Sequence
  Description of Artificial Sequence Synthetic peptide
VQWRRIRVWVIV

SEQ ID NO: 492 - protein, 12 aa, Artificial Sequence
  Description of Artificial Sequence Synthetic peptide
IQWRRIRVWVIR

SEQ ID NO: 493 - protein, 12 aa, Artificial Sequence
  Description of Artificial Sequence Synthetic peptide
VIWRRIRVWVIR

SEQ ID NO: 494 - protein, 12 aa, Artificial Sequence
  Description of Artificial Sequence Synthetic peptide
VQIRRIRVWVIR

SEQ ID NO: 495 - protein, 12 aa, Artificial Sequence
  Description of Artificial Sequence Synthetic peptide
VQWIRIRVWVIR

SEQ ID NO: 496 - protein, 12 aa, Artificial Sequence
  Description of Artificial Sequence Synthetic peptide
VQWRIIRVWVIR

SEQ ID NO: 497 - protein, 12 aa, Artificial Sequence
  Description of Artificial Sequence Synthetic peptide
VQWRRIIVWVIR

SEQ ID NO: 498 - protein, 12 aa, Artificial Sequence
  Description of Artificial Sequence Synthetic peptide
VQWRRIRIWVIR

SEQ ID NO: 499 - protein, 12 aa, Artificial Sequence
  Description of Artificial Sequence Synthetic peptide
VQWRRIRVIVIR

SEQ ID NO: 500 - protein, 12 aa, Artificial Sequence
  Description of Artificial Sequence Synthetic peptide
VQWRRIRVWIIR

SEQ ID NO: 501 - protein, 12 aa, Artificial Sequence
  Description of Artificial Sequence Synthetic peptide
VQWRRIRVWVII

SEQ ID NO: 502 - protein, 12 aa, Artificial Sequence
  Description of Artificial Sequence Synthetic peptide
LQWRRIRVWVIR

SEQ ID NO: 503 - protein, 12 aa, Artificial Sequence
  Description of Artificial Sequence Synthetic peptide
VLWRRIRVWVIR

SEQ ID NO: 504 - protein, 12 aa, Artificial Sequence
  Description of Artificial Sequence Synthetic peptide
VQLRRIRVWVIR

SEQ ID NO: 505 - protein, 12 aa, Artificial Sequence
  Description of Artificial Sequence Synthetic peptide
VQWLRIRVWVIR

SEQ ID NO: 506 - protein, 12 aa, Artificial Sequence
  Description of Artificial Sequence Synthetic peptide
VQWRLIRVWVIR

SEQ ID NO: 507 - protein, 12 aa, Artificial Sequence
  Description of Artificial Sequence Synthetic peptide
VQWRRLRVWVIR

SEQ ID NO: 508 - protein, 12 aa, Artificial Sequence
  Description of Artificial Sequence Synthetic peptide
VQWRRILVWVIR

SEQ ID NO: 509 - protein, 12 aa, Artificial Sequence
  Description of Artificial Sequence Synthetic peptide
VQWRRIRLWVIR

SEQ ID NO: 510 - protein, 12 aa, Artificial Sequence
  Description of Artificial Sequence Synthetic peptide
VQWRRIRVLVIR

SEQ ID NO: 511 - protein, 12 aa, Artificial Sequence
  Description of Artificial Sequence Synthetic peptide
VQWRRIRVWLIR

SEQ ID NO: 512 - protein, 12 aa, Artificial Sequence
  Description of Artificial Sequence Synthetic peptide
VQWRRIRVWVLR

SEQ ID NO: 513 - protein, 12 aa, Artificial Sequence
  Description of Artificial Sequence Synthetic peptide
VQWRRIRVWVIL

SEQ ID NO: 514 - protein, 12 aa, Artificial Sequence
  Description of Artificial Sequence Synthetic peptide
WQWRRIRVWVIR

SEQ ID NO: 515 - protein, 12 aa, Artificial Sequence
  Description of Artificial Sequence Synthetic peptide
VWWRRIRVWVIR

SEQ ID NO: 516 - protein, 12 aa, Artificial Sequence
  Description of Artificial Sequence Synthetic peptide
VQWWRIRVWVIR

SEQ ID NO: 517 - protein, 12 aa, Artificial Sequence
  Description of Artificial Sequence Synthetic peptide
VQWRWIRVWVIR

SEQ ID NO: 518 - protein, 12 aa, Artificial Sequence
  Description of Artificial Sequence Synthetic peptide
VQWRRWRVWVIR

SEQ ID NO: 519 - protein, 12 aa, Artificial Sequence
  Description of Artificial Sequence Synthetic peptide
VQWRRIWVWVIR

SEQ ID NO: 520 - protein, 12 aa, Artificial Sequence
  Description of Artificial Sequence Synthetic peptide
VQWRRIRWWVIR

SEQ ID NO: 521 - protein, 12 aa, Artificial Sequence
  Description of Artificial Sequence Synthetic peptide
VQWRRIRVWWIR

SEQ ID NO: 522 - protein, 12 aa, Artificial Sequence
  Description of Artificial Sequence Synthetic peptide
VQWRRIRVWVWR

SEQ ID NO: 523 - protein, 12 aa, Artificial Sequence
  Description of Artificial Sequence Synthetic peptide
VQWRRIRVWVIW

SEQ ID NO: 524 - protein, 12 aa, Artificial Sequence
  Description of Artificial Sequence Synthetic peptide
QQWRRIRVWVIR

SEQ ID NO: 525 - protein, 12 aa, Artificial Sequence
  Description of Artificial Sequence Synthetic peptide
VQQRRIRVWVIR

SEQ ID NO: 526 - protein, 12 aa, Artificial Sequence
  Description of Artificial Sequence Synthetic peptide
VQWQRIRVWVIR

SEQ ID NO: 527 - protein, 12 aa, Artificial Sequence
  Description of Artificial Sequence Synthetic peptide
VQWRQIRVWVIR

SEQ ID NO: 528 - protein, 12 aa, Artificial Sequence
  Description of Artificial Sequence Synthetic peptide
VQWRRQRVWVIR

SEQ ID NO: 529 - protein, 12 aa, Artificial Sequence
  Description of Artificial Sequence Synthetic peptide
VQWRRIQVWVIR

SEQ ID NO: 530 - protein, 12 aa, Artificial Sequence
  Description of Artificial Sequence Synthetic peptide
VQWRRIRQWVIR

SEQ ID NO: 531 - protein, 12 aa, Artificial Sequence
  Description of Artificial Sequence Synthetic peptide
VQWRRIRVQVIR

SEQ ID NO: 532 - protein, 12 aa, Artificial Sequence
  Description of Artificial Sequence Synthetic peptide
VQWRRIRVWQIR

SEQ ID NO: 533 - protein, 12 aa, Artificial Sequence
  Description of Artificial Sequence Synthetic peptide
VQWRRIRVWVQR

SEQ ID NO: 534 - protein, 12 aa, Artificial Sequence
  Description of Artificial Sequence Synthetic peptide
VQWRRIRVWVIQ

SEQ ID NO: 535 - protein, 12 aa, Artificial Sequence
  Description of Artificial Sequence Synthetic peptide
GRIRWVILWRQV

SEQ ID NO: 536 - protein, 12 aa, Artificial Sequence
  Description of Artificial Sequence Synthetic peptide
KGIRWVILWRQV

SEQ ID NO: 537 - protein, 12 aa, Artificial Sequence
  Description of Artificial Sequence Synthetic peptide
KRGRWVILWRQV

SEQ ID NO: 538 - protein, 12 aa, Artificial Sequence
  Description of Artificial Sequence Synthetic peptide
KRIGWVILWRQV

SEQ ID NO: 539 - protein, 12 aa, Artificial Sequence
  Description of Artificial Sequence Synthetic peptide
KRIRGVILWRQV

SEQ ID NO: 540 - protein, 12 aa, Artificial Sequence
  Description of Artificial Sequence Synthetic peptide
KRIRWGILWRQV

SEQ ID NO: 541 - protein, 12 aa, Artificial Sequence
  Description of Artificial Sequence Synthetic peptide
KRIRWVGLWRQV

SEQ ID NO: 542 - protein, 12 aa, Artificial Sequence
  Description of Artificial Sequence Synthetic peptide
KRIRWVIGWRQV

SEQ ID NO: 543 - protein, 12 aa, Artificial Sequence
  Description of Artificial Sequence Synthetic peptide
KRIRWVILGRQV

SEQ ID NO: 544 - protein, 12 aa, Artificial Sequence
  Description of Artificial Sequence Synthetic peptide
KRIRWVILWGQV

SEQ ID NO: 545 - protein, 12 aa, Artificial Sequence
  Description of Artificial Sequence Synthetic peptide
KRIRWVILWRGV

SEQ ID NO: 546 - protein, 12 aa, Artificial Sequence
  Description of Artificial Sequence Synthetic peptide
KRIRWVILWRQG

SEQ ID NO: 547 - protein, 12 aa, Artificial Sequence
  Description of Artificial Sequence Synthetic peptide
ARIRWVILWRQV

SEQ ID NO: 548 - protein, 12 aa, Artificial Sequence
  Description of Artificial Sequence Synthetic peptide
KAIRWVILWRQV

SEQ ID NO: 549 - protein, 12 aa, Artificial Sequence
  Description of Artificial Sequence Synthetic peptide
KRARWVILWRQV

SEQ ID NO: 550 - protein, 12 aa, Artificial Sequence
  Description of Artificial Sequence Synthetic peptide
KRIAWVILWRQV

SEQ ID NO: 551 - protein, 12 aa, Artificial Sequence
  Description of Artificial Sequence Synthetic peptide
KRIRAVILWRQV

SEQ ID NO: 552 - protein, 12 aa, Artificial Sequence
  Description of Artificial Sequence Synthetic peptide
KRIRWAILWRQV

SEQ ID NO: 553 - protein, 12 aa, Artificial Sequence
  Description of Artificial Sequence Synthetic peptide
KRIRWVALWRQV

SEQ ID NO: 554 - protein, 12 aa, Artificial Sequence
  Description of Artificial Sequence Synthetic peptide
KRIRWVIAWRQV

SEQ ID NO: 555 - protein, 12 aa, Artificial Sequence
  Description of Artificial Sequence Synthetic peptide
KRIRWVILARQV

SEQ ID NO: 556 - protein, 12 aa, Artificial Sequence
  Description of Artificial Sequence Synthetic peptide
KRIRWVILWAQV

SEQ ID NO: 557 - protein, 12 aa, Artificial Sequence
  Description of Artificial Sequence Synthetic peptide
KRIRWVILWRAV

SEQ ID NO: 558 - protein, 12 aa, Artificial Sequence
  Description of Artificial Sequence Synthetic peptide
KRIRWVILWRQA

SEQ ID NO: 559 - protein, 12 aa, Artificial Sequence
  Description of Artificial Sequence Synthetic peptide
RRIRWVILWRQV

SEQ ID NO: 560 - protein, 12 aa, Artificial Sequence
  Description of Artificial Sequence Synthetic peptide
KRRRWVILWRQV

SEQ ID NO: 561 - protein, 12 aa, Artificial Sequence
  Description of Artificial Sequence Synthetic peptide
KRIRRVILWRQV

SEQ ID NO: 562 - protein, 12 aa, Artificial Sequence
  Description of Artificial Sequence Synthetic peptide
KRIRWRILWRQV

SEQ ID NO: 563 - protein, 12 aa, Artificial Sequence
  Description of Artificial Sequence Synthetic peptide
KRIRWVRLWRQV

SEQ ID NO: 564 - protein, 12 aa, Artificial Sequence
  Description of Artificial Sequence Synthetic peptide
KRIRWVIRWRQV

SEQ ID NO: 565 - protein, 12 aa, Artificial Sequence
  Description of Artificial Sequence Synthetic peptide
KRIRWVILRRQV

SEQ ID NO: 566 - protein, 12 aa, Artificial Sequence
  Description of Artificial Sequence Synthetic peptide
KRIRWVILWRRV

SEQ ID NO: 567 - protein, 12 aa, Artificial Sequence
  Description of Artificial Sequence Synthetic peptide
KRIRWVILWRQR

SEQ ID NO: 568 - protein, 12 aa, Artificial Sequence
  Description of Artificial Sequence Synthetic peptide
KKIRWVILWRQV

SEQ ID NO: 569 - protein, 12 aa, Artificial Sequence
  Description of Artificial Sequence Synthetic peptide
KRKRWVILWRQV

SEQ ID NO: 570 - protein, 12 aa, Artificial Sequence
  Description of Artificial Sequence Synthetic peptide
KRIKWVILWRQV

SEQ ID NO: 571 - protein, 12 aa, Artificial Sequence
  Description of Artificial Sequence Synthetic peptide
KRIRKVILWRQV

SEQ ID NO: 572 - protein, 12 aa, Artificial Sequence
  Description of Artificial Sequence Synthetic peptide
KRIRWKILWRQV

SEQ ID NO: 573 - protein, 12 aa, Artificial Sequence
  Description of Artificial Sequence Synthetic peptide
KRIRWVKLWRQV

SEQ ID NO: 574 - protein, 12 aa, Artificial Sequence
  Description of Artificial Sequence Synthetic peptide
KRIRWVIKWRQV

SEQ ID NO: 575 - protein, 12 aa, Artificial Sequence
  Description of Artificial Sequence Synthetic peptide
KRIRWVILKRQV

SEQ ID NO: 576 - protein, 12 aa, Artificial Sequence
  Description of Artificial Sequence Synthetic peptide
KRIRWVILWKQV

SEQ ID NO: 577 - protein, 12 aa, Artificial Sequence
  Description of Artificial Sequence Synthetic peptide
KRIRWVILWRKV

SEQ ID NO: 578 - protein, 12 aa, Artificial Sequence
  Description of Artificial Sequence Synthetic peptide
KRIRWVILWRQK

SEQ ID NO: 579 - protein, 12 aa, Artificial Sequence
  Description of Artificial Sequence Synthetic peptide
VRIRWVILWRQV

SEQ ID NO: 580 - protein, 12 aa, Artificial Sequence
  Description of Artificial Sequence Synthetic peptide
KVIRWVILWRQV

SEQ ID NO: 581 - protein, 12 aa, Artificial Sequence
  Description of Artificial Sequence Synthetic peptide
KRVRWVILWRQV

SEQ ID NO: 582 - protein, 12 aa, Artificial Sequence
  Description of Artificial Sequence Synthetic peptide
KRIVWVILWRQV

SEQ ID NO: 583 - protein, 12 aa, Artificial Sequence
  Description of Artificial Sequence Synthetic peptide
KRIRVVILWRQV

SEQ ID NO: 584 - protein, 12 aa, Artificial Sequence
  Description of Artificial Sequence Synthetic peptide
KRIRWVVLWRQV

SEQ ID NO: 585 - protein, 12 aa, Artificial Sequence
  Description of Artificial Sequence Synthetic peptide
KRIRWVIVWRQV

SEQ ID NO: 586 - protein, 12 aa, Artificial Sequence
  Description of Artificial Sequence Synthetic peptide
KRIRWVILVRQV

SEQ ID NO: 587 - protein, 12 aa, Artificial Sequence
  Description of Artificial Sequence Synthetic peptide
KRIRWVILWVQV

SEQ ID NO: 588 - protein, 12 aa, Artificial Sequence
  Description of Artificial Sequence Synthetic peptide
KRIRWVILWRVV

SEQ ID NO: 589 - protein, 12 aa, Artificial Sequence
  Description of Artificial Sequence Synthetic peptide
IRIRWVILWRQV

SEQ ID NO: 590 - protein, 12 aa, Artificial Sequence
  Description of Artificial Sequence Synthetic peptide
KIIRWVILWRQV

SEQ ID NO: 591 - protein, 12 aa, Artificial Sequence
  Description of Artificial Sequence Synthetic peptide
KRIIWVILWRQV

SEQ ID NO: 592 - protein, 12 aa, Artificial Sequence
  Description of Artificial Sequence Synthetic peptide
KRIRIVILWRQV

SEQ ID NO: 593 - protein, 12 aa, Artificial Sequence
  Description of Artificial Sequence Synthetic peptide
KRIRWIILWRQV

SEQ ID NO: 594 - protein, 12 aa, Artificial Sequence
  Description of Artificial Sequence Synthetic peptide
KRIRWVIIWRQV

SEQ ID NO: 595 - protein, 12 aa, Artificial Sequence
  Description of Artificial Sequence Synthetic peptide
KRIRWVILIRQV

SEQ ID NO: 596 - protein, 12 aa, Artificial Sequence
  Description of Artificial Sequence Synthetic peptide
KRIRWVILWIQV

SEQ ID NO: 597 - protein, 12 aa, Artificial Sequence
  Description of Artificial Sequence Synthetic peptide
KRIRWVILWRIV

SEQ ID NO: 598 - protein, 12 aa, Artificial Sequence
  Description of Artificial Sequence Synthetic peptide
KRIRWVILWRQI

SEQ ID NO: 599 - protein, 12 aa, Artificial Sequence
  Description of Artificial Sequence Synthetic peptide
LRIRWVILWRQV

SEQ ID NO: 600 - protein, 12 aa, Artificial Sequence
  Description of Artificial Sequence Synthetic peptide
KLIRWVILWRQV

SEQ ID NO: 601 - protein, 12 aa, Artificial Sequence
  Description of Artificial Sequence Synthetic peptide
KRLRWVILWRQV

SEQ ID NO: 602 - protein, 12 aa, Artificial Sequence
  Description of Artificial Sequence Synthetic peptide
KRILWVILWRQV

SEQ ID NO: 603 - protein, 12 aa, Artificial Sequence
  Description of Artificial Sequence Synthetic peptide
KRIRLVILWRQV

SEQ ID NO: 604 - protein, 12 aa, Artificial Sequence
  Description of Artificial Sequence Synthetic peptide
KRIRWLILWRQV

SEQ ID NO: 605 - protein, 12 aa, Artificial Sequence
  Description of Artificial Sequence Synthetic peptide
KRIRWVLLWRQV

SEQ ID NO: 606 - protein, 12 aa, Artificial Sequence
  Description of Artificial Sequence Synthetic peptide
KRIRWVILLRQV

SEQ ID NO: 607 - protein, 12 aa, Artificial Sequence
  Description of Artificial Sequence Synthetic peptide
KRIRWVILWLQV

SEQ ID NO: 608 - protein, 12 aa, Artificial Sequence
  Description of Artificial Sequence Synthetic peptide
KRIRWVILWRLV

SEQ ID NO: 609 - protein, 12 aa, Artificial Sequence
  Description of Artificial Sequence Synthetic peptide
KRIRWVILWRQL

SEQ ID NO: 610 - protein, 12 aa, Artificial Sequence
  Description of Artificial Sequence Synthetic peptide
WRIRWVILWRQV

SEQ ID NO: 611 - protein, 12 aa, Artificial Sequence
  Description of Artificial Sequence Synthetic peptide
KWIRWVILWRQV

SEQ ID NO: 612 - protein, 12 aa, Artificial Sequence
  Description of Artificial Sequence Synthetic peptide
KRWRWVILWRQV

SEQ ID NO: 613 - protein, 12 aa, Artificial Sequence
  Description of Artificial Sequence Synthetic peptide
KRIWWVILWRQV

SEQ ID NO: 614 - protein, 12 aa, Artificial Sequence
  Description of Artificial Sequence Synthetic peptide
KRIRWWILWRQV

SEQ ID NO: 615 - protein, 12 aa, Artificial Sequence
  Description of Artificial Sequence Synthetic peptide
KRIRWVWLWRQV

SEQ ID NO: 616 - protein, 12 aa, Artificial Sequence
  Description of Artificial Sequence Synthetic peptide
KRIRWVIWWRQV

SEQ ID NO: 617 - protein, 12 aa, Artificial Sequence
  Description of Artificial Sequence Synthetic peptide
KRIRWVILWWQV

SEQ ID NO: 618 - protein, 12 aa, Artificial Sequence
  Description of Artificial Sequence Synthetic peptide
KRIRWVILWRWV

SEQ ID NO: 619 - protein, 12 aa, Artificial Sequence
  Description of Artificial Sequence Synthetic peptide
KRIRWVILWRQW

SEQ ID NO: 620 - protein, 12 aa, Artificial Sequence
  Description of Artificial Sequence Synthetic peptide
QRIRWVILWRQV

SEQ ID NO: 621 - protein, 12 aa, Artificial Sequence
  Description of Artificial Sequence Synthetic peptide
KQIRWVILWRQV

SEQ ID NO: 622 - protein, 12 aa, Artificial Sequence
  Description of Artificial Sequence Synthetic peptide
KRQRWVILWRQV

SEQ ID NO: 623 - protein, 12 aa, Artificial Sequence
  Description of Artificial Sequence Synthetic peptide
KRIQWVILWRQV

SEQ ID NO: 624 - protein, 12 aa, Artificial Sequence
  Description of Artificial Sequence Synthetic peptide
KRIRQVILWRQV

SEQ ID NO: 625 - protein, 12 aa, Artificial Sequence
  Description of Artificial Sequence Synthetic peptide
KRIRWQILWRQV

SEQ ID NO: 626 - protein, 12 aa, Artificial Sequence
  Description of Artificial Sequence Synthetic peptide
KRIRWVQLWRQV

SEQ ID NO: 627 - protein, 12 aa, Artificial Sequence
  Description of Artificial Sequence Synthetic peptide
KRIRWVIQWRQV

SEQ ID NO: 628 - protein, 12 aa, Artificial Sequence
  Description of Artificial Sequence Synthetic peptide
KRIRWVILQRQV

SEQ ID NO: 629 - protein, 12 aa, Artificial Sequence
  Description of Artificial Sequence Synthetic peptide
KRIRWVILWQQV

SEQ ID NO: 630 - protein, 12 aa, Artificial Sequence
  Description of Artificial Sequence Synthetic peptide
KRIRWVILWRQQ

SEQ ID NO: 631 - protein, 12 aa, Artificial Sequence
  Description of Artificial Sequence Synthetic peptide
RRWIRVILWRQV

SEQ ID NO: 632 - protein, 12 aa, Artificial Sequence
  Description of Artificial Sequence Synthetic peptide
RRWIRVRVWVIR

SEQ ID NO: 633 - protein, 12 aa, Artificial Sequence
  Description of Artificial Sequence Synthetic peptide
KRIRWVAVILRV

SEQ ID NO: 634 - protein, 12 aa, Artificial Sequence
  Description of Artificial Sequence Synthetic peptide
KRIRWVRVWVIR

SEQ ID NO: 635 - protein, 12 aa, Artificial Sequence
  Description of Artificial Sequence Synthetic peptide
VQWRRIAVILRV

SEQ ID NO: 636 - protein, 12 aa, Artificial Sequence
  Description of Artificial Sequence Synthetic peptide
VQWRRIILWRQV

SEQ ID NO: 637 - protein, 12 aa, Artificial Sequence
  Description of Artificial Sequence Synthetic peptide
AVILRVKRIRWV

SEQ ID NO: 638 - protein, 12 aa, Artificial Sequence
  Description of Artificial Sequence Synthetic peptide
RVWVIRRRWIRV

SEQ ID NO: 639 - protein, 12 aa, Artificial Sequence
  Description of Artificial Sequence Synthetic peptide
AVILRVKRIRWV

SEQ ID NO: 640 - protein, 12 aa, Artificial Sequence
  Description of Artificial Sequence Synthetic peptide
RVWVIRKRIRWV

SEQ ID NO: 641 - protein, 12 aa, Artificial Sequence
  Description of Artificial Sequence Synthetic peptide
AVILRVVQWRRI

SEQ ID NO: 642 - protein, 12 aa, Artificial Sequence
  Description of Artificial Sequence Synthetic peptide
ILWRQVVQWRRI

SEQ ID NO: 643 - protein, 12 aa, Artificial Sequence
  Description of Artificial Sequence Synthetic peptide
AVILRVRRWIRV

SEQ ID NO: 644 - protein, 12 aa, Artificial Sequence
  Description of Artificial Sequence Synthetic peptide
ILWRQVKRIRWV

SEQ ID NO: 645 - protein, 12 aa, Artificial Sequence
  Description of Artificial Sequence Synthetic peptide
RVWVIRVQWRRI

SEQ ID NO: 646 - protein, 12 aa, Artificial Sequence
  Description of Artificial Sequence Synthetic peptide
RRWIWVILWRQV

SEQ ID NO: 647 - protein, 12 aa, Artificial Sequence
  Description of Artificial Sequence Synthetic peptide
RRWIRIRVWVIR

SEQ ID NO: 648 - protein, 12 aa, Artificial Sequence
  Description of Artificial Sequence Synthetic peptide
KRIRRVAVILRV

SEQ ID NO: 649 - protein, 12 aa, Artificial Sequence
  Description of Artificial Sequence Synthetic peptide
KRIRRIRVWVIR

SEQ ID NO: 650 - protein, 12 aa, Artificial Sequence
  Description of Artificial Sequence Synthetic peptide
VQWRRVAVILRV

SEQ ID NO: 651 - protein, 12 aa, Artificial Sequence
  Description of Artificial Sequence Synthetic peptide
VQWRWVILWRQV

SEQ ID NO: 652 - protein, 12 aa, Artificial Sequence
  Description of Artificial Sequence Synthetic peptide
RRWIRVAVWRQV

SEQ ID NO: 653 - protein, 12 aa, Artificial Sequence
  Description of Artificial Sequence Synthetic peptide
RRWIRVAVWVIR

SEQ ID NO: 654 - protein, 12 aa, Artificial Sequence
  Description of Artificial Sequence Synthetic peptide
KRIRWVILILRV

SEQ ID NO: 655 - protein, 12 aa, Artificial Sequence
  Description of Artificial Sequence Synthetic peptide
KRIRWVILWVIR

SEQ ID NO: 656 - protein, 12 aa, Artificial Sequence
  Description of Artificial Sequence Synthetic peptide
VQWRRIRVILRV

SEQ ID NO: 657 - protein, 12 aa, Artificial Sequence
  Description of Artificial Sequence Synthetic peptide
VQWRRIRVWRQV

SEQ ID NO: 658 - protein, 12 aa, Artificial Sequence
  Description of Artificial Sequence Synthetic peptide
VRLIVAVRIWRR

SEQ ID NO: 659 - protein, 12 aa, Artificial Sequence
  Description of Artificial Sequence Synthetic peptide
VQRWLIVWRIRK

SEQ ID NO: 660 - protein, 12 aa, Artificial Sequence
  Description of Artificial Sequence Synthetic peptide
RIVWVRIRRWQV

SEQ ID NO: 661 - protein, 12 aa, Artificial Sequence
  Description of Artificial Sequence Synthetic peptide
VRIWRRVRLIVA

SEQ ID NO: 662 - protein, 12 aa, Artificial Sequence
  Description of Artificial Sequence Synthetic peptide
VWRIRKVQRWLI

SEQ ID NO: 663 - protein, 12 aa, Artificial Sequence
  Description of Artificial Sequence Synthetic peptide
IRRWQVRIVWVR

SEQ ID NO: 664 - protein, 12 aa, Artificial Sequence
  Description of Artificial Sequence Synthetic peptide
VFLRRIRVIVIR

SEQ ID NO: 665 - protein, 12 aa, Artificial Sequence
  Description of Artificial Sequence Synthetic peptide
VFWRRIRVWVIR

SEQ ID NO: 666 - protein, 12 aa, Artificial Sequence
  Description of Artificial Sequence Synthetic peptide
VQLRAIRVRVIR

SEQ ID NO: 667 - protein, 12 aa, Artificial Sequence
  Description of Artificial Sequence Synthetic peptide
VQLRRIRVWVIR

SEQ ID NO: 668 - protein, 12 aa, Artificial Sequence
  Description of Artificial Sequence Synthetic peptide
VQWRAIRVRVIR

SEQ ID NO: 669 - protein, 12 aa, Artificial Sequence
  Description of Artificial Sequence Synthetic peptide
VQWRRIRVWVIR

SEQ ID NO: 670 - protein, 9 aa, Artificial Sequence
  Description of Artificial Sequence Synthetic peptide
VQLRIRVAV

SEQ ID NO: 671 - protein, 12 aa, Artificial Sequence
  Description of Artificial Sequence Synthetic peptide
VQRWLIVWRIRK

SEQ ID NO: 672 - protein, 12 aa, Artificial Sequence
  Description of Artificial Sequence Synthetic peptide
VQLRIRVAVIRA

SEQ ID NO: 673 - protein, 12 aa, Artificial Sequence
  Description of Artificial Sequence Synthetic peptide
VRLIVAVRIWRR

SEQ ID NO: 674 - protein, 12 aa, Artificial Sequence
  Description of Artificial Sequence Synthetic peptide
VRLRIRWWVLRK

SEQ ID NO: 675 - protein, 9 aa, Artificial Sequence
  Description of Artificial Sequence Synthetic peptide
VRLRIRVAV

SEQ ID NO: 676 - protein, 9 aa, Artificial Sequence
  Description of Artificial Sequence Synthetic peptide
IRVRVIWRK

SEQ ID NO: 677 - protein, 7 aa, Artificial Sequence
  Description of Artificial Sequence Synthetic peptide
VIWIRWR

SEQ ID NO: 678 - protein, 7 aa, Artificial Sequence
  Description of Artificial Sequence Synthetic peptide
WIVIWRR

SEQ ID NO: 679 - protein, 7 aa, Artificial Sequence
  Description of Artificial Sequence Synthetic peptide
IRWVIRW

SEQ ID NO: 680 - protein, 9 aa, Artificial Sequence
  Description of Artificial Sequence Synthetic peptide
FRRWWKWFK

SEQ ID NO: 681 - protein, 9 aa, Artificial Sequence
  Description of Artificial Sequence Synthetic peptide
RKWIWRWFL

SEQ ID NO: 682 - protein, 12 aa, Artificial Sequence
  Description of Artificial Sequence Synthetic peptide
RLERIVVIRVAR

SEQ ID NO: 683 - protein, 12 aa, Artificial Sequence
  Description of Artificial Sequence Synthetic peptide
RLAGIVVIRVAR

SEQ ID NO: 684 - protein, 12 aa, Artificial Sequence
  Description of Artificial Sequence Synthetic peptide
RLARIVKIRVAR

SEQ ID NO: 685 - protein, 9 aa, Artificial Sequence
  Description of Artificial Sequence Synthetic peptide
VRLRIRVAV

SEQ ID NO: 686 - protein, 13 aa, Artificial Sequence
  Description of Artificial Sequence Synthetic peptide
ILRWKWRWWVWRR

SEQ ID NO: 687 - protein, 7 aa, Artificial Sequence
  Description of Artificial Sequence Synthetic peptide
IWVIWRR

SEQ ID NO: 688 - protein, 9 aa, Artificial Sequence
  Description of Artificial Sequence Synthetic peptide
VQLRIRVAV

SEQ ID NO: 689 - protein, 12 aa, Artificial Sequence
  Description of Artificial Sequence Synthetic peptide
RRWVVWRIVQRR

SEQ ID NO: 690 - protein, 12 aa, Artificial Sequence
  Description of Artificial Sequence Synthetic peptide
IRWRIRVWVRRI

SEQ ID NO: 691 - protein, 12 aa, Artificial Sequence
  Description of Artificial Sequence Synthetic peptide
VRLWIRVAVIRA

SEQ ID NO: 692 - protein, 9 aa, Artificial Sequence
  Description of Artificial Sequence Synthetic peptide
VQWRIRVRV

SEQ ID NO: 693 - protein, 13 aa, Artificial Sequence
  Description of Artificial Sequence Synthetic peptide
ILPWWWPWWPWRR

SEQ ID NO: 694 - protein, 13 aa, Artificial Sequence
  Description of Artificial Sequence Synthetic peptide
ILVWKWRWWVWRR

SEQ ID NO: 695 - protein, 12 aa, Artificial Sequence
  Description of Artificial Sequence Synthetic peptide
RLWRIVVIRVKR

SEQ ID NO: 696 - protein, 7 aa, Artificial Sequence
  Description of Artificial Sequence Synthetic peptide
KIWVRWK

SEQ ID NO: 697 - protein, 12 aa, Artificial Sequence
  Description of Artificial Sequence Synthetic peptide
RLWRIVVIRVAR

SEQ ID NO: 698 - protein, 9 aa, Artificial Sequence
  Description of Artificial Sequence Synthetic peptide
RWTISFKRS

SEQ ID NO: 699 - protein, 12 aa, Artificial Sequence
  Description of Artificial Sequence Synthetic peptide
VRLRIRVAVRRA

SEQ ID NO: 700 - protein, 51 aa, Artificial Sequence
  Description of Artificial Sequence Synthetic polypeptide
RRWRIVVIRVRRRRWRIVVIRVRRRRWRIVVIRVRRRRWRIVVIRVRRKKK

SEQ ID NO: 701 - protein, 12 aa, Artificial Sequence
  Description of Artificial Sequence Synthetic peptide
VQWRRIRVWVIR

SEQ ID NO: 702 - protein, 12 aa, Artificial Sequence
  Description of Artificial Sequence Synthetic peptide
VRFRIRVRVWIK

SEQ ID NO: 703 - protein, 13 aa, Artificial Sequence
  Description of Artificial Sequence Synthetic peptide
ILVWKWVWWVWRR

SEQ ID NO: 704 - protein, 12 aa, Artificial Sequence
  Description of Artificial Sequence Synthetic peptide
HQFRFRFRVRRK

SEQ ID NO: 705 - protein, 12 aa, Artificial Sequence
  Description of Artificial Sequence Synthetic peptide
KKQVSRVKVWRK

SEQ ID NO: 706 - protein, 12 aa, Artificial Sequence
  Description of Artificial Sequence Synthetic peptide
LVRAIQVRAVIR

SEQ ID NO: 707 - protein, 8 aa, Artificial Sequence
  Description of Artificial Sequence Synthetic peptide
RRWIVWIR

SEQ ID NO: 708 - protein, 9 aa, Artificial Sequence
  Description of Artificial Sequence Synthetic peptide
VRLRIRVAV

SEQ ID NO: 709 - protein, 12 aa, Artificial Sequence
  Description of Artificial Sequence Synthetic peptide
QRLRIRVAVIRA

SEQ ID NO: 710 - protein, 9 aa, Artificial Sequence
  Description of Artificial Sequence Synthetic peptide
VRFRIRVRV

SEQ ID NO: 711 - protein, 8 aa, Artificial Sequence
  Description of Artificial Sequence Synthetic peptide
RQVIVRRW

SEQ ID NO: 712 - protein, 7 aa, Artificial Sequence
  Description of Artificial Sequence Synthetic peptide
FRIRVRF

SEQ ID NO: 713 - protein, 26 aa, Artificial Sequence
  Description of Artificial Sequence Synthetic peptide
KWKSFIKKLTSAAKKVVTTAKPLISS

SEQ ID NO: 714 - protein, 12 aa, Artificial Sequence
  Description of Artificial Sequence Synthetic peptide
VQLRIRVAVIRA

SEQ ID NO: 715 - protein, 12 aa, Artificial Sequence
  Description of Artificial Sequence Synthetic peptide
VQLRIRVWVIRK

SEQ ID NO: 716 - protein, 12 aa, Artificial Sequence
  Description of Artificial Sequence Synthetic peptide
KQFRIRVRVIRK

SEQ ID NO: 717 - protein, 12 aa, Artificial Sequence
  Description of Artificial Sequence Synthetic peptide
RGARIVVIRVAR

SEQ ID NO: 718 - protein, 8 aa, Artificial Sequence
  Description of Artificial Sequence Synthetic peptide
RFRIRVRV

SEQ ID NO: 719 - protein, 9 aa, Artificial Sequence
  Description of Artificial Sequence Synthetic peptide
LRIRVIVWR

SEQ ID NO: 720 - protein, 12 aa, Artificial Sequence
  Description of Artificial Sequence Synthetic peptide
RRWRIVVIRVRR

SEQ ID NO: 721 - protein, 12 aa, Artificial Sequence
  Description of Artificial Sequence Synthetic peptide
IVVWRRQLVKNK

SEQ ID NO: 722 - protein, 12 aa, Artificial Sequence
  Description of Artificial Sequence Synthetic peptide
RLARIVVFRVAR

SEQ ID NO: 723 - protein, 12 aa, Artificial Sequence
  Description of Artificial Sequence Synthetic peptide
RFWKVRVKYIRF

SEQ ID NO: 724 - protein, 12 aa, Artificial Sequence
  Description of Artificial Sequence Synthetic peptide
AIRVVRARLVRR

SEQ ID NO: 725 - protein, 12 aa, Artificial Sequence
  Description of Artificial Sequence Synthetic peptide
VRWRIRVAVIRA

SEQ ID NO: 726 - protein, 12 aa, Artificial Sequence
  Description of Artificial Sequence Synthetic peptide
IVWKIKRWWVGR

SEQ ID NO: 727 - protein, 9 aa, Artificial Sequence
  Description of Artificial Sequence Synthetic peptide
KRWIVKWVK

SEQ ID NO: 728 - protein, 12 aa, Artificial Sequence
  Description of Artificial Sequence Synthetic peptide
HQWRIRVAVRRH

SEQ ID NO: 729 - protein, 9 aa, Artificial Sequence
  Description of Artificial Sequence Synthetic peptide
IRVWVLRQR

SEQ ID NO: 730 - protein, 12 aa, Artificial Sequence
  Description of Artificial Sequence Synthetic peptide
RLARIVVIRVRR

SEQ ID NO: 731 - protein, 9 aa, Artificial Sequence
  Description of Artificial Sequence Synthetic peptide
RIKWIVRFR

SEQ ID NO: 732 - protein, 9 aa, Artificial Sequence
  Description of Artificial Sequence Synthetic peptide
KQFRIRVRV

SEQ ID NO: 733 - protein, 13 aa, Artificial Sequence
  Description of Artificial Sequence Synthetic peptide
ILKKWPWWPWRRK

SEQ ID NO: 734 - protein, 12 aa, Artificial Sequence
  Description of Artificial Sequence Synthetic peptide
GLARIVVIRVAR

SEQ ID NO: 735 - protein, 9 aa, Artificial Sequence
  Description of Artificial Sequence Synthetic peptide
RIRVIVLKK

SEQ ID NO: 736 - protein, 12 aa, Artificial Sequence
  Description of Artificial Sequence Synthetic peptide
VFWRRIRVWVIR

SEQ ID NO: 737 - protein, 9 aa, Artificial Sequence
  Description of Artificial Sequence Synthetic peptide
RVRWYRIFY

SEQ ID NO: 738 - protein, 12 aa, Artificial Sequence
  Description of Artificial Sequence Synthetic peptide
KRWRIRVRVIRK

SEQ ID NO: 739 - protein, 12 aa, Artificial Sequence
  Description of Artificial Sequence Synthetic peptide
RLRRIVVIRVAR

SEQ ID NO: 740 - protein, 12 aa, Artificial Sequence
  Description of Artificial Sequence Synthetic peptide
VRLIVKVRIRRR

SEQ ID NO: 741 - protein, 12 aa, Artificial Sequence
  Description of Artificial Sequence Synthetic peptide
VRVIVKVRIRRR

SEQ ID NO: 742 - protein, 12 aa, Artificial Sequence
  Description of Artificial Sequence Synthetic peptide
VRWIVKVRIRRR

SEQ ID NO: 743 - protein, 12 aa, Artificial Sequence
  Description of Artificial Sequence Synthetic peptide
RRLIVKVRIWRR

SEQ ID NO: 744 - protein, 12 aa, Artificial Sequence
  Description of Artificial Sequence Synthetic peptide
RRWIVKVRIRRR

SEQ ID NO: 745 - protein, 12 aa, Artificial Sequence
  Description of Artificial Sequence Synthetic peptide
KWRLLIRWRIQK

SEQ ID NO: 746 - protein, 12 aa, Artificial Sequence
  Description of Artificial Sequence Synthetic peptide
KQRWLIRWRIRK

SEQ ID NO: 747 - protein, 12 aa, Artificial Sequence
  Description of Artificial Sequence Synthetic peptide
VQLRIRVKVIRK

SEQ ID NO: 748 - protein, 12 aa, Artificial Sequence
  Description of Artificial Sequence Synthetic peptide
WQLRIRVKVIRK

SEQ ID NO: 749 - protein, 12 aa, Artificial Sequence
  Description of Artificial Sequence Synthetic peptide
WQRVRRVKVIRK

SEQ ID NO: 750 - protein, 37 aa, Artificial Sequence
  Description of Artificial Sequence Synthetic polypeptide
LLGDFFRKSKEKIGKEFKRIVQRIKDFLRNLVPRTES

SEQ ID NO: 751 - protein, 11 aa, Artificial Sequence
  Description of Artificial Sequence Synthetic peptide
ILRWPWPWRRK

SEQ ID NO: 752 - protein, 21 aa, Artificial Sequence
  Description of Artificial Sequence Synthetic peptide
KWKLFKKIFKRIVQRIKDFLR

SEQ ID NO: 753 - protein, 13 aa, Artificial Sequence
  Description of Artificial Sequence Synthetic peptide
ILPWKWPWWPWRR

SEQ ID NO: 754 - protein, 9 aa, Artificial Sequence
  Description of Artificial Sequence Synthetic peptide
FQVVKIKVR

SEQ ID NO: 755 - protein, 9 aa, Artificial Sequence
  Description of Artificial Sequence Synthetic peptide
KRWWKWIRW

SEQ ID NO: 756 - protein, 9 aa, Artificial Sequence
  Description of Artificial Sequence Synthetic peptide
IWVIRRVWR

SEQ ID NO: 757 - protein, 7 aa, Artificial Sequence
  Description of Artificial Sequence Synthetic peptide
IRWVIRW

SEQ ID NO: 758 - protein, 12 aa, Artificial Sequence
  Description of Artificial Sequence Synthetic peptide
RLARIVVIRVAR

SEQ ID NO: 759 - protein, 12 aa, Artificial Sequence
  Description of Artificial Sequence Synthetic peptide
RLARIVVIRVAG

SEQ ID NO: 760 - protein, 12 aa, Artificial Sequence
  Description of Artificial Sequence Synthetic peptide
VRLIRAVRAWRV

SEQ ID NO: 761 - protein, 12 aa, Artificial Sequence
  Description of Artificial Sequence Synthetic peptide
IFWRRIVIVKKF

SEQ ID NO: 762 - protein, 9 aa, Artificial Sequence
  Description of Artificial Sequence Synthetic peptide
WRWRVRVWR

SEQ ID NO: 763 - protein, 13 aa, Artificial Sequence
  Description of Artificial Sequence Synthetic peptide
KSRIVPAIPVSLL

SEQ ID NO: 764 - protein, 12 aa, Artificial Sequence
  Description of Artificial Sequence Synthetic peptide
KQFRIRVRVWIK

SEQ ID NO: 765 - protein, 12 aa, Artificial Sequence
  Description of Artificial Sequence Synthetic peptide
VRWARVARILRV

SEQ ID NO: 766 - protein, 12 aa, Artificial Sequence
  Description of Artificial Sequence Synthetic peptide
LIQRIRVRNIVK

SEQ ID NO: 767 - protein, 9 aa, Artificial Sequence
  Description of Artificial Sequence Synthetic peptide
LRIRWIFKR

SEQ ID NO: 768 - protein, 12 aa, Artificial Sequence
  Description of Artificial Sequence Synthetic peptide
RLARIVPIRVAR

SEQ ID NO: 769 - protein, 12 aa, Artificial Sequence
  Description of Artificial Sequence Synthetic peptide
KRIVRRLVARIV

SEQ ID NO: 770 - protein, 12 aa, Artificial Sequence
  Description of Artificial Sequence Synthetic peptide
KRFRIRVRWVIK

SEQ ID NO: 771 - protein, 12 aa, Artificial Sequence
  Description of Artificial Sequence Synthetic peptide
RLARICVIRVAR

SEQ ID NO: 772 - protein, 9 aa, Artificial Sequence
  Description of Artificial Sequence Synthetic peptide
VFRIRVRVR

SEQ ID NO: 773 - protein, 8 aa, Artificial Sequence
  Description of Artificial Sequence Synthetic peptide
VRIVRRVI

SEQ ID NO: 774 - protein, 12 aa, Artificial Sequence
  Description of Artificial Sequence Synthetic peptide
RLARRVVIRVAR

SEQ ID NO: 775 - protein, 9 aa, Artificial Sequence
  Description of Artificial Sequence Synthetic peptide
WNRVKWIRR

SEQ ID NO: 776 - protein, 12 aa, Artificial Sequence
  Description of Artificial Sequence Synthetic peptide
RLRRIVVIRVRR

SEQ ID NO: 777 - protein, 8 aa, Artificial Sequence
  Description of Artificial Sequence Synthetic peptide
RIWVIWRR

SEQ ID NO: 778 - protein, 9 aa, Artificial Sequence
  Description of Artificial Sequence Synthetic peptide
IVVRRVIRK

SEQ ID NO: 779 - protein, 7 aa, Artificial Sequence
  Description of Artificial Sequence Synthetic peptide
RWWRIVI

SEQ ID NO: 780 - protein, 7 aa, Artificial Sequence
  Description of Artificial Sequence Synthetic peptide
IVWIWRR

SEQ ID NO: 781 - protein, 9 aa, Artificial Sequence
  Description of Artificial Sequence Synthetic peptide
KRWWKWWRR

SEQ ID NO: 782 - protein, 11 aa, Artificial Sequence
  Description of Artificial Sequence Synthetic peptide
RLARIVVIRVA

SEQ ID NO: 783 - protein, 9 aa, Artificial Sequence
  Description of Artificial Sequence Synthetic peptide
VLIRWNGKK

SEQ ID NO: 784 - protein, 9 aa, Artificial Sequence
  Description of Artificial Sequence Synthetic peptide
KRFRIRVRV

SEQ ID NO: 785 - protein, 7 aa, Artificial Sequence
  Description of Artificial Sequence Synthetic peptide
WIRVIRW

SEQ ID NO: 786 - protein, 12 aa, Artificial Sequence
  Description of Artificial Sequence Synthetic peptide
KQFRNRLRIVKK

SEQ ID NO: 787 - protein, 12 aa, Artificial Sequence
  Description of Artificial Sequence Synthetic peptide
RRVIVKKFRIRR

SEQ ID NO: 788 - protein, 7 aa, Artificial Sequence
  Description of Artificial Sequence Synthetic peptide
IIRRWWV

SEQ ID NO: 789 - protein, 12 aa, Artificial Sequence
  Description of Artificial Sequence Synthetic peptide
VQWRAIRVRVIR

SEQ ID NO: 790 - protein, 12 aa, Artificial Sequence
  Description of Artificial Sequence Synthetic peptide
VQLRAIRVRVIR

SEQ ID NO: 791 - protein, 12 aa, Artificial Sequence
  Description of Artificial Sequence Synthetic peptide
RIVRVRAIRWQV

SEQ ID NO: 792 - protein, 12 aa, Artificial Sequence
  Description of Artificial Sequence Synthetic peptide
RIVIVRIRRLFV

SEQ ID NO: 793 - protein, 9 aa, Artificial Sequence
  Description of Artificial Sequence Synthetic peptide
FRIRVRVIR

SEQ ID NO: 794 - protein, 12 aa, Artificial Sequence
  Description of Artificial Sequence Synthetic peptide
VRLIVKVRIWRR

SEQ ID NO: 795 - protein, 7 aa, Artificial Sequence
  Description of Artificial Sequence Synthetic peptide
VRRIWRR

SEQ ID NO: 796 - protein, 8 aa, Artificial Sequence
  Description of Artificial Sequence Synthetic peptide
VRFRIWRR

SEQ ID NO: 797 - protein, 12 aa, Artificial Sequence
  Description of Artificial Sequence Synthetic peptide
VRRIVRVLIRWA

SEQ ID NO: 798 - protein, 12 aa, Artificial Sequence
  Description of Artificial Sequence Synthetic peptide
RVLIRVARRVIW

SEQ ID NO: 799 - protein, 12 aa, Artificial Sequence
  Description of Artificial Sequence Synthetic peptide
VRLIRVWRVIRK

(19) United States
(12) Patent Application Publication
(10) Pub. No.: US 2016/0289287 A1
(43) Pub. Date: Oct. 6, 2016

(54) SMALL CATIONIC ANTI-BIOFILM AND IDR PEPTIDES

(71) Applicant: THE UNIVERSITY OF BRITISH COLUMBIA, Vancouver (CA)

(72) Inventors: Robert E.W. Hancock, Vancouver (CA); Cesar de la Fuente Nunez, Vancouver (CA); Jason Kindrachuk, Vancouver (CA); Havard Jenssen, Vancouver (CA); Joerg Overhage, Vancouver (CA); Evan Haney, Vancouver (CA)

(73) Assignee: The University of British Columbia, Vancouver, BC (CA)

(21) Appl. No.: 14/915,193

(22) PCT Filed: Aug. 27, 2014
(86) PCT No.: PCT/US14/52993

§ 371 Date: Feb. 26, 2016

Related U.S. Application Data
(60) Provisional application No. 61/870,655, filed on Aug. 27, 2013.

(51) Int. Cl. C07K 14/47; C07K 7/06; C07K 7/08

(52) U.S. Cl. CPC C07K 14/4723; C07K 7/08; C07K 7/06; A61K 38/00

(57) ABSTRACT

The present invention relates generally to peptides and more specifically to anti-biofilm and immunomodulatory peptides.

### FIELD

The present invention relates generally to peptides, especially protease resistant peptides, and more specifically to anti-biofilm and immunomodulatory IDR peptides.

### BACKGROUND

The treatment of bacterial infections with antibiotics is one of the mainstays of human medicine. Unfortunately the effectiveness of antibiotics has become limited due to an increase in bacterial antibiotic resistance in the face of a decreasing efforts and success in discovery of new classes of antibiotics. Today, infectious diseases are the second leading cause of death worldwide and the largest cause of premature deaths and loss of work productivity in industrialized countries. Nosocomial bacterial infections that are resistant to therapy result in annual costs of more than $2 billion and account for more than 100,000 direct and indirect deaths in North America alone, whereas a major complication of microbial diseases, namely sepsis, annually accounts for 750,000 cases and 210,000 deaths in North America and 5 million worldwide.

A major limitation in antibiotic development has been difficulties in finding new structures with equivalent properties to the conventional antibiotics, namely low toxicity for the host and a broad spectrum of action against bacterial pathogens. Recent novel antibiotic classes, including the oxazolidinones (linezolid), the streptogramins (synercid) and the glycolipopeptides (daptomycin) are all only active against Gram positive pathogens. One promising set of compounds is the cationic antimicrobial peptides that are mimics of peptides produced by virtually all complex organisms ranging from plants and insects to humans as a major component of their innate defenses against infection. Cationic antimicrobial peptides, found in most species of life, represent a good template for a new generation of antimicrobials. They kill both Gram negative and Gram positive microorganisms rapidly and directly, do not easily select mutants, work against common clinically-resistant bacteria such as methicillin-resistant Staphylococcus aureus (MRSA) and vancomycin resistant Enterococcus (VRE), show a synergistic effect with conventional antibiotics, and can often activate host innate immunity without displaying immunogenicity (Hancock R E W. 2001. Cationic peptides: effectors in innate immunity and novel antimicrobials. Lancet Infectious Diseases 1, 156-164; Fjell C D, Hiss J A, Hancock R E W and Schneider G. 2012. Designing antimicrobial peptides: Form follows function. Nature Rev. Drug Discov. 11:37-51). Moreover, some peptide seem to counteract some of the more harmful aspects of inflammation (e.g. sepsis, endotoxaemia), which is extremely important since rapid killing of bacteria and subsequent liberation of bacterial components such as LPS or peptidoglycan can induce fatal immune dysregulation (Jarisch-Herxheimer reaction) (Gough M, Hancock R E W, Kelly N M. 1996. Anti-endotoxic potential of cationic peptide antimicrobials. Infect. Immun 64, 4922-4927) and stimulate anti-infective immunity (Hilchie A L, K Wuerth, and R E W Hancock. 2013 Immune modulation by multifaceted cationic host defence (antimicrobial) peptides. Nature Chem. Biol. 9:761-8). Thus they offered at least two separate approaches to treating infections with uses as broad spectrum anti-infectives and/or as adjuvants that selectively enhance aspects of innate immunity while suppressing potentially harmful inflammation. Although there is great hope for such peptides there is clearly much room for improvement [Hancock, R. E. W., A. Nijnik and D. J. Philpott. 2012. Modulating immunity as a therapy for bacterial infections. Nature Rev. Microbiol. 10:243-254; Fjell C D, et al. 2012. Nat. Rev. Drug Discov. 11:37-51.].

Biofilm infections are especially recalcitrant to conventional antibiotic treatment, and are a major problem in trauma patients, including military personnel with major injuries [Høiby, N., et al. 2011. The clinical impact of bacterial biofilms. International J Oral Science 3:55-65.; Antunes, L C M and R B R Ferreira. 2011. Biofilms and bacterial virulence. Reviews Med Microbiol 22:12-16.]. Microbial biofilms are surface-associated bacterial communities that grow in a protective polymeric matrix. The biofilm-mode of growth is a major lifestyle for bacteria in natural, industrial and clinical settings; indeed they are associated with 65% or more of all clinical infections. In the clinic, bacterial growth as biofilms, renders them difficult to treat with conventional antibiotics, and can result in as much as a 1000-fold decrease in susceptibility to antimicrobial agents, due to differentiation of bacteria within the biofilm, poor antibiotic penetration into the biofilm, and the stationary phase growth of bacteria underlying the surface layer. There are very few compounds developed that have activity against bacterial biofilms, unlike the peptides described here.

In 2008, our group made the breakthrough observation that the 37 amino acid human host defense peptide LL-37 was able to both prevent the development of biofilms and promote dissociation of existing biofilms [Overhage, J., A. Campisano, M. Bains, E. C. W. Torfs, B. H. A. Rehm, and R. E. W. Hancock. 2008. The human host defence peptide LL-37 prevents bacterial biofilm formation. Infect. Immun. 76:4176-4182]; a property that was apparently shared by a subset of the natural antimicrobial peptides (e.g., bovine indolicidin), but not by other cationic host defense peptides (e.g., polymyxin). Mechanistically it was demonstrated that LL-37 likely entered bacteria at sub-inhibitory concentrations and altered the transcription of dozens of genes leading to decreased bacterial attachment, increased twitching motility, and decreases in the quorum sensing systems (Las and Rhl). Since this time anti-biofilm activity has been confirmed by several other investigators and extended to certain other peptides [e.g. Amer L. S., B. M. Bishop, and M. L. van Hoek. 2010. Antimicrobial and antibiofilm activity of cathelicidins and short, synthetic peptides against Francisella. Biochem Biophys Res Commun 396:246-51.], although none of these appear to be as active as the best peptides described here, virtually all of them are much larger and are thus not as cost effective, and none contained D-amino acids and are thus protease resistant.

Armed with knowledge of the anti-biofilm activity of cationic peptides, we screened a library of peptides and demonstrated that peptides as small as 9 amino acids in length were active against P. aeruginosa [de la Fuente-Núñez, C., V. Korolik, M. Bains, U. Nguyen, E. B. M. Breidenstein, S. Horsman, S. Lewenza, L. Burrows and R. E. W. Hancock. 2012 Inhibition of bacterial biofilm formation and swarming motility by a small synthetic cationic peptide. Antimicrob. Agents Chemother. 56:2696-2704.]. These studies clearly showed that antimicrobial and anti-biofilm properties were independently determined. For example, the 9 amino acid long peptide 1037 had very good anti-biofilm activity (IC₅₀=5 μg/ml), but essentially no antimicrobial activity against biofilm cells (MIC=304 μg/ml), whereas the related peptide HH10 had very good antimicrobial activity (MIC=0.8 μg/ml), but was devoid of anti-biofilm activity. Intriguingly, we found that these peptides also work to break down Campylobacter, Burkholderia and Listeria biofilms, suggesting a shared mechanism in these very different pathogens, which has now been deciphered and is presented for the first time herein. It is worthy of note that Burkholderia is completely resistant to the antibiotic action against free swimming cells, of antimicrobial peptides, again confirming the independence of antimicrobial and anti-biofilm activity. Thus the structure:activity relationships for the different types of activities of cationic peptides do not correspond such that it is possible to make an antimicrobial peptide with no anti-biofilm activity (de la Fuente-Núñez C, et al. 2012 Inhibition of bacterial biofilm formation and swarming motility by a small synthetic cationic peptide. Antimicrob. Agents Chemother. 56:2696-2704) or an immune modulator peptide with no antimicriobial activity vs. planktonic bacteria (M. G., E. Dullaghan, N. Mookherjee, N. Glavas, M. Waldbrook, A. Thompson, A. Wang, K. Lee, S. Doria, P. Hamill, J. Yu, Y. Li, O. Donini, M. M. Guarna, B. B. Finlay, J. R. North, and R. E. W. Hancock. 2007. An anti-infective peptide that selectively modulates the innate immune response. Nature Biotech. 25: 465-472), although the data described herein show that it is possible to make peptides with both immunomodulatory and anti-biofilm activity.

Thus this invention relates to peptides that have broad spectrum activity against biofilms (but nearly always weaker activity against so-called planktonic, free-swimming cells) including especially protease-resistant peptides. The peptides of the invention often have immunomodulatory activity that can occur in conjunction with anti-biofilm activity or in place of this activity. Ideally a peptide of the invention will contain both activities.

The innate immune system is a highly effective and evolved general defense system that involves a variety of effector functions including phagocytic cells, complement, etc., but is generally incompletely understood. Elements of innate immunity are always present at low levels and are activated very rapidly when stimulated by pathogens, acting to prevent these pathogens from causing disease. Generally speaking many known innate immune responses are “triggered” by the binding of microbial signaling molecules, like lipopolysaccharide (LPS), to pattern recognition receptors such as Toll-like receptors (TLR) on the surface of host cells. Many of the effector functions of innate immunity are grouped together in the inflammatory response. However, too severe an inflammatory response can result in effects that are harmful to the body, and, in an extreme case, sepsis and potentially death can occur; indeed sepsis occurs in approximately 750,000 patients in North America annually with 210,000 deaths. Thus, a therapeutic intervention to boost innate immunity, which is based on stimulation of TLR signaling (for example using a TLR agonist), has the potential disadvantage that it could stimulate a potentially harmful inflammatory response and/or exacerbate the natural inflammatory response to infection.

Natural cationic host defense peptides (also known as antimicrobial peptides) are crucial molecules in host defenses against pathogenic microbe challenge. It has been hypothesized that since their direct antimicrobial activity is compromised by physiological salt concentrations (e.g. the 150 mM NaCl and 2 mM MgCl₂+CaCl₂ salt concentrations in blood), their most important activities are immunomodulatory (Bowdish D M E, Davidson D J, and Hancock R E W. 2005. A re-evaluation of the role of host defence peptides in mammalian immunity. Current Protein Pept. Sci. 6:35-51).

We have described in the past, a broad series of synthetic so-called innate defence regulator (IDR) peptides, as mimics of natural host defence peptides, which act to treat infections and inflammation in animal models. Although some IDR peptides are able to weakly kill planktonic bacteria, quantitative structure-activity relationship studies have suggested that antimicrobial and immunomodulatory activities are independently determined The activity of IDR peptides against biofilms, either in vitro or in vivo, was unknown prior to the discovery reported here.

The host defence and IDR peptides have many anti-infective immunomodulatory activities other than direct microbial killing, leading us and others to propose that such activities play a key role in innate immunity, including the suppression of acute inflammation and stimulation of protective immunity against a variety of pathogens [Hancock R E W, and Sahl H G. 2006. Antimicrobial and host-defence peptides as novel anti-infective therapeutic strategies. Nature Biotech. 24:1551-1557.]. To demonstrate that synthetic variants of these peptides can protect without direct killing (i.e., by selectively modulating innate immunity), we created a bovine peptide homolog, innate defense regulator peptide (IDR)-1, which had absolutely no direct antibiotic activity, but was protective by both local and systemic administration in mouse models of infection with major Gram-positive and -negative pathogens, including MRSA, vancomycin-resistant Enterococcus (VRE), and Salmonella [Scott, et al. 2007. Nature Biotech. 25: 465-472.]. Protection by IDR-1 was prevented by in vivo depletion of monocytes and macrophages, but not neutrophils or lymphocytes indicating that the former were key effector cells. Gene and protein expression analysis in human and mouse monocytes and macrophages indicated that IDR-1 acted through mitogen-activated protein (MAP) kinase and other signaling pathways, to enhance the levels of monocyte chemokines while reducing pro-inflammatory cytokine responses. More recent work has demonstrated new more effective IDR peptides that protect in numerous animal models including E. coli, Salmonella, MRSA, VRE, multi-drug resistant tuberculosis, cystic fibrosis (CF), cerebral malaria, and perinatal brain injury from hypoxia-ischemia-LPS challenge (preterm brith model) and also have wound healing and vaccineadjuvant properties [Nijnik A., L. Madera, S. Ma, M. Waldbrook, M. Elliott, S. C. Mullaly, J. Kindrachuk, H. Jenssen, R. E. W. Hancock. 2010. Synthetic cationic peptide IDR-1002 provides protection against bacterial infections through chemokine induction and enhanced leukocyte recruitment. J. Immunol. 184:2539-2550.; Turner-Brannen, E., K.-Y. Choi, D. N. D. Lippert, J. P. Cortens, R. E. W. Hancock, H. El-Gabalawy and N. Mookherjee. 2011. Modulation of IL-1β-induced inflammatory responses by a synthetic cationic innate defence regulator peptide, IDR-1002, in synovial fibroblasts. Arthritis Res. Ther. 13:R129.; Madera, L., and R. E. W. Hancock. 2012. Synthetic immunomodulatory peptide IDR-1002 enhances monocyte migration and adhesion on fibronectin. J. Innate Immun. 4:553-568.; Achtman, A. H., S. Pilat, C. W. Law, D. J. Lynn, L. Janot, M. Mayer, S. Ma, J. Kindrachuk, B. B. Finlay, F. S. L. Brinkman, G. K. Smyth, R. E. W. Hancock and L. Schofield. 2012. Effective adjunctive therapy by an innate defense regulatory peptide in a pre-clinical model of severe malaria. Science Transl. Med. 4:135ra64.; Rivas-Santiago, B., J. E. Castañeda-Delgado, C. E. Rivas Santiago, M. Waldbrook, I. González-Curiel, J. C. León-Contreras, A. Enciso-Moreno, V. del Villar, J. Méndez-Ramos, R. E. W. Hancock, R. Hernandez-Pando. 2013. Ability of innate defence regulator peptides IDR-1002, IDR-HH2 and IDR-1018 to protect against Mycobacterium tuberculosis infections in animal models. PLoS One 8:e59119.; Mayer, M. L., C. J. Blohmke, R. Falsafi, C. D. Fjell, L. Madera, S. E. Turvey, and R. E. W. Hancock. 2013. Rescue of dysfunctional autophagy by IDR-1018 attenuates hyperinflammatory responses from cystic fibrosis cells. J. Immunol. 190:1227-1238.; Niyonsaba, F., L. Madera, K. Okumura, H. Ogawa, and R. E. W. Hancock. 2013. The innate defense regulator peptides IDR-HH2, IDR-1002 and IDR-1018 modulate human neutrophil functions. J. Leukocyte Biol. in press PMID: 23616580.; Bolouri, H., K. Sävman, W. Wang, A. Thomas, N. Maurer, E. Dullaghan, C. D. Fjell, H. Hagberg, R. E. W. Hancock, K. L. Brown, and C. Mallard. 2014. Innate defence regulator peptide 1018 protects against perinatal brain injury. Ann. Neurol. 75:395-410; Kindrachuk, J., H. Jenssen, M. Elliott, R. Townsend, A. Nijnik, S. F. Lee, V. Gerdts, L. A. Babiuk, S. A. Halperin and R. E. W. Hancock. 2009. A novel vaccine adjuvant comprised of a synthetic innate defence regulator peptide and CpG oligonucleotide links innate and adaptive immunity. Vaccine 27:4662-4671.; Polewicz, M., A. Gracia, S. Garlapati, J. van Kessel, S. Strom, S. A. Halperin, R. E. W. Hancock, A. A. Potter, L. A. Babiuk, and V. Gerdts. 2013. Novel vaccine formulations against pertussis offer earlier onset of immunity and provide protection in the presence of maternal antibodies. Vaccine. 2013 PMID: 23684829.; Steinstraesser, L., T. Hirsch, M. Schulte, M. Kueckelhaus, F. Jacobsen, E. A. Mersch, I. Stricker, N. Afacan, H. Jenssen, R. E. W. Hancock and J. Kindrachuk. 2012. Innate defense regulator peptide 1018 in wound healing and wound infection. PLoS ONE 7:e39373.].

The common features, small size, and linearity make the peptides of this invention ideal candidates for semi-random design methods such as Spot peptide synthesis on cellulose membranes. The field of chemoinformatics involves computer-aided identification of new lead structures and their optimization into drug candidates (Engel T. Basic Overview of Chemoinformatics. Journal of Chemical Information and Modelling, 46:2267-2277, 2006). One of the most broadly used chemoinformatics approaches is called Quantitative Structure-Activity Relationship (QSAR) modeling, which seeks to relate structural characteristics of a molecule (known as descriptors) to its measurable properties, such as biological activity. QSAR analysis has found a broad application in antimicrobial discovery. In a series of pilot studies we have utilized a variety of QSAR descriptors in combination with the approaches of the Artificial Intelligence to successfully predict antimicrobial activity of cationic antimicrobial peptides (Cherkasov, A., K. Hilpert, H. Jenssen, C. D. Fjell, M. Waldbrook, S. C. Mullaly, R. Volkmer and R. E. W. Hancock. 2009. Use of artificial intelligence in the design of small peptide antibiotics effective against a broad spectrum of highly antibiotic resistant Superbugs. ACS Chemical Biol. 4:65-74.).

The present invention is based on the observation that certain peptide sequences, representing a few hundred of the more than 10²¹ possible 12 amino-acid sequences, have potent anti-biofilm activity or immunomodulatory activity or both. Exemplary peptides of the invention include peptides with their carboxyl terminus residue carboxy-amidated having the amino acid sequences of SEQ ID NOS:1-749, and analogs, derivatives, enantiomers, unamidated and truncated variants, and conservative variations thereof.

The invention also provides a method of inhibiting the growth of or causing dispersal of bacteria in a biofilm including contacting the biofilm with an inhibiting effective amount of at least one peptide of the invention alone, or in combination with at least one antibiotic. Classes of antibiotics that can be used in synergistic therapy with the peptides of the invention include, but are not limited to, aminoglycosides, β-lactams, fluoroquinolones, vancomycin, and macrolides.

The invention further provides a method of modulating the innate immune response of human cells in a manner that enhances the production of a protective immune response while not inducing or inhibiting the potentially harmful proinflammatory response.

The invention further provides polynucleotides that encode the peptides of the invention. Exemplary polynucleotides encode peptides having the amino acid sequences of SEQ ID NOS:1-749, and analogs, derivatives and conservative variations thereof.

The invention further provides a method of identifying an antibiofilm peptide having 8 to 12 amino acids. The method includes contacting under conditions sufficient for antimicrobial activity, a test peptide with a microbe that will form or has formed one or more surface-associated biofilm colonies, and detecting a reduced amount of biofilm as compared to amount of biofilm in the absence of the test peptide. In one embodiment, the peptide is synthesized on, or attached to, a solid support. The peptides of the invention will retain anti-biofilm activity when cleaved from the solid support or retain activity when still associated with the solid support. The microbe can be a Gram negative bacterium, such as Pseudomonas aeruginosa, Escherichia coli, Salmonella enteritidis ssp. Typhimurium, Acinetobacter baumanii, Burkholderia spp., Klebsiella pneumoniae, Enterobacter sp., or Campylobacter spp. In another embodiment, the microbe can be a Gram positive bacterium, such as Staphylococcus aureus, Staphylococcus epidermidis, or Enterococcus faecalis. The detection can include detecting residual bacteria by confocal microscopy of coverslips with adhered bacteria in flow cells, after specific staining, or by measuring residual bacteria adherent to the plastic surface of a microtiter plate by removing free swimming (planktonic) bacteria and staining residual bacteria with crystal violet.

In another embodiment, the invention provides agents that are capable of selectively enhancing innate immunity by contacting cells containing one or more genes that encode a polypeptide involved in innate immunity and protection against an infection, with the agent of interest, wherein expression of the one or more genes or polypeptides in the presence of the agent is modulated as compared with expression of the one or more genes or polypeptides in the absence of the agent, and wherein the modulated expression results in enhancement of innate immunity. In one aspect, the invention includes agents identified by the methods. In another aspect, the agent does not stimulate a septic reaction, but does stimulate the expression of one or more genes or polypeptides involved in protective immunity. Exemplary but non-limiting genes or polypeptides which are increased in expression include MCP1, MCP3 and Gro-α.

In another embodiment, the invention provides agents that selectively suppress the proinflammatory response of cells containing a polynucleotide or polynucleotides that encode a polypeptide involved in innate immunity. The method includes contacting the cells with microbes, or TLR ligands and agonists derived from those microbes, and further contacting the cells with an agent of interest, wherein the agent decreases the expression of a proinflammatory gene encoding the polynucleotide or polypeptide as compared with expression of the proinflammatory gene or polypeptide in the absence of the agent. In one aspect, the modulated expression results in suppression of proinflammatory and septic responses. Preferably, the agent does not stimulate a sepsis reaction in a subject. Exemplary, but non-limiting proinflammatory genes include TNFα.

The invention further provides a method of protecting medical devices from colonization with pathogenic biofilm-forming bacteria by coating at least one peptide of the invention on the surface of the medical device.

### SUMMARY

In a first aspect, disclosed herein is an isolated antibiofilm or immunomodulatory peptide having 7 to 12 amino acids, wherein the peptide has an amino acid sequence of SEQ ID NOS: 1-749, or analogs, derivatives, enantiomers, amidated and unamidated variations and conservative variations thereof.

In some embodiments of this aspect, disclosed herein is an isolated polynucleotide that encodes this peptide.

In some embodiments, the peptide can comprise any contiguous sequence of amino acids having the formula: AA1-AA2-AA3-AA4-AA5-AA6-AA7-AA8-AA9-AA10-AA11-AA12 and containing only the residues K, R, F, L, I, A, W and no more than a single Q or G residue.

In a second aspect, disclosed herein is a polypeptide X1-A-X2 or a functional variant or mimetic thereof, wherein A represents at least one peptide having an amino acid sequence of SEQ ID NOS: 1-749 or analogs, derivatives, enantiomers, amidated and unamidated variations and conservative variations thereof; and wherein each X1 and X2 independently of one another represents any amino acid sequence of n amino acids, n varying from 0 to 50, and n being identical or different in X1 and X2.

In some embodiments of this polypeptide, the functional variant or mimetic is a conservative amino acid substitution or peptide mimetic substitution. In some embodiments of this polypeptide, the functional variant has about 66% or greater amino acid identity. Truncation of amino acids from the N or C termini or from both can create these mimetics. In some embodiments of this polypeptide, the amino acids are non-natural amino acid equivalents. In some embodiments of this polypeptide, n is zero.

In a third aspect, disclosed herein is a method of inhibiting the growth of bacterial biofilms comprising contacting a bacterial biofilm with an inhibiting effective amount of a peptide having an amino acid sequence of SEQ ID NOS: 1-749, or any combination thereof, or analogs, derivatives, enantiomers, amidated and unamidated variations and conservative variations thereof.

In some embodiments of this aspect, the bacterium is Gram positive. In some embodiments of this aspect, the bacterium is Staphylococcus aureus, Staphylococcus epidermidis, or Enterococcus faecalis. In some embodiments of this aspect, the bacterium is Gram negative. In some embodiments of this aspect, the bacterium is Pseudomonas aeruginosa, Escherichia coli, Salmonella enteritidis ssp Typhimurium, Acinetobacter baummanii, Klebsiella pneumoniae, Enterobacter sp., Campylobacter or Burkholderia cepacia complex.

In some embodiments of this aspect, the contacting comprises a peptide in combination with at least one antibiotic. In some embodiments of this aspect, the antibiotic is selected from the group consisting of aminoglycosides, β-lactams, quinolones, and glycopeptides. In some embodiments of this aspect, the antibiotic is selected from the group consisting of amikacin, gentamicin, kanamycin, netilmicin, tobramycin, streptomycin, azithromycin, clarithromycin, erythromycin, erythromycin estolate/ethyl-succinate/gluceptate/lactobionate/stearate, penicillin G, penicillin V, methicillin, nafcillin, oxacillin, cloxacillin, dicloxacillin, ampicillin, amoxicillin, ticarcillin, carbenicillin, mezlocillin, azlocillin, piperacillin, cephalothin, cefazolin, cefaclor, cefamandole, cefoxitin, cefuroxime, cefonicid, cefmetazole, cefotetan, cefprozil, loracarbef, cefetamet, cefoperazone, cefotaxime, ceftizoxime, ceftriaxone, ceftazidime, cefepime, cefixime, cefpodoxime, cefsulodin, imipenem, aztreonam, fleroxacin, nalidixic acid, norfloxacin, ciprofloxacin, ofloxacin, enoxacin, lomefloxacin, cinoxacin, doxycycline, minocycline, tetracycline, vancomycin, chloramphenicol, clindamycin, trimethoprim, sulfamethoxazole, nitrofurantoin, rifampin and mupirocin and teicoplanin.

In some embodiments of this aspect, the peptide is bound to a solid support. In some embodiments, the peptide is bound covalently or noncovalently. In some embodiments of this aspect, the solid support is a medical device.

In some embodiments of the first aspect, the peptide is capable of selectively enhancing innate immunity as determined by contacting a cell containing one or more genes that encode a polypeptide involved in innate immunity and protection against an infection, with the peptide of interest, wherein expression of the one or more genes or polypeptides in the presence of the peptide is modulated as compared with expression of the one or more genes or polypeptides in the absence of the peptide, and wherein the modulated expression results in enhancement of innate immunity. In further embodiments, the peptide does not stimulate a septic reaction. In further embodiments, the peptide stimulates expression of the one or more genes or proteins, thereby selectively enhancing innate immunity. In further embodiments, the one or more genes or proteins encode chemokines or interleukins that attract immune cells. In further embodiments, the one or more genes are selected from the group consisting of MCP-1, MCP-3, and Gro-α.

In some embodiments of the first aspect, the peptide selectively suppresses proinflammatory responses, whereby the peptide can contact a cell treated with an inflammatory stimulus and containing a polynucleotide or polynucleotides that encode a polypeptide involved in inflammation and sepsis and which is normally upregulated in response to this inflammatory stimulus, and wherein the peptides suppresses the expression of this gene or polypeptide as compared with expression of the inflammatory gene in the absence of the peptide and wherein the modulated expression results in enhancement of innate immunity. In further embodiments, the peptide inhibits the inflammatory or septic response. In further embodiments, the peptide blocks the inflammatory or septic response. In further embodiments, the peptide inhibits the expression of a pro-inflammatory gene or molecule. In further embodiments, the peptide inhibits the expression of TNF-α. In further embodiments, the inflammation is induced by a microbe or a microbial ligand acting on a Toll-like receptor. In further embodiments, the microbial ligand is a bacterial endotoxin or lipopolysaccharide.

In a fourth aspect, disclosed herein is an isolated immunomodulatory polypeptide X1-A-X2, or a functional variant or mimetic thereof, wherein A represents at least one peptide having an amino acid sequence of SEQ ID NOS: 1-749 or analogs, derivatives, enantiomers, amidated and unamidated variations and conservative variations thereof each X1 and X2 independently of one another represents any amino acid sequence of n amino acids, n varying from 0 to 5, and n being identical or different in X1 and X2.

In some embodiments of this aspect, the functional variant or mimetic is a conservative amino acid substitution or peptide mimetic substitution. In some embodiments of this aspect, the functional variant has about 70% or greater amino acid sequence identity to X1-A-X2.

In a fifth aspect, disclosed herein is method of inhibiting the growth of bacterial biofilms comprising contacting the bacterial biofilm with an inhibiting effective amount of a peptide having an amino acid sequence of aspects one or four, or any combination thereof, or analogs, derivatives, enantiomers, amidated and unamidated variations and conservative variations thereof.

In some embodiments of this aspect, the bacterium is Gram positive. In some embodiments of this aspect, the bacterium is Staphylococcus aureus, Staphylococcus epidermidis, or Enterococcus faecaelis.

In some embodiments of this aspect, the bacterium is Gram negative. In some embodiments of this aspect, the bacterium is Pseudomonas aeruginosa, Escherichia coli, Salmonella enteritidis ssp Typhimurium, Acinetobacter baummanii, Klebsiella pneumoniae, Campylobacter, or Burkholderia cepacia complex.

In some embodiments of this aspect, the contacting comprises a peptide in combination with at least one antibiotic. In some embodiments, the antibiotic is selected from the group consisting of aminoglycosides, β-lactams, quinolones, and glycopeptides.

In some embodiments, the antibiotic is selected from the group consisting of amikacin, gentamicin, kanamycin, netilmicin, tobramycin, streptomycin, azithromycin, clarithromycin, erythromycin, erythromycin estolate/ethyl-succinate/gluceptate/lactobionate/stearate, penicillin G, penicillin V, methicillin, nafcillin, oxacillin, cloxacillin, dicloxacillin, ampicillin, amoxicillin, ticarcillin, carbenicillin, mezlocillin, azlocillin, piperacillin, cephalothin, cefazolin, cefaclor, cefamandole, cefoxitin, cefuroxime, cefonicid, cefmetazole, cefotetan, cefprozil, loracarbef, cefetamet, cefoperazone, cefotaxime, ceftizoxime, ceftriaxone, ceftazidime, cefepime, cefixime, cefpodoxime, cefsulodin, imipenem, aztreonam, fleroxacin, nalidixic acid, norfloxacin, ciprofloxacin, ofloxacin, enoxacin, lomefloxacin, cinoxacin, doxycycline, minocycline, tetracycline, vancomycin, chloramphenicol, clindamycin, trimethoprim, sulfamethoxazole, nitrofurantoin, rifampin and mupirocin and teicoplanin.

In some embodiments of this aspect, the peptide is bound to a solid support. In some embodiments, the peptide is bound covalently or noncovalently. In some embodiments of this aspect, the solid support is a medical device.

In some embodiments of the first or fourth aspects, the peptide is capable of selectively enhancing innate immunity as determined by contacting a cell containing one or more genes that encode a polypeptide involved in innate immunity and protection against an infection, with the peptide of interest, wherein expression of the one or more genes or polypeptides in the presence of the peptide is modulated as compared with expression of the one or more genes or polypeptides in the absence of the peptide, and wherein the modulated expression results in enhancement of innate immunity.

In some embodiments of this aspect, the peptide does not stimulate a septic reaction.

In some embodiments of this aspect, the peptide stimulates expression of the one or more genes or proteins, thereby selectively enhancing innate immunity. In some embodiments, the one or more genes or proteins encode chemokines or interleukins that attract immune cells. In some embodiments, the one or more genes are selected from the group consisting of MCP-1, MCP-3, and Gro-α.

In some embodiments of the first or fourth aspects, the peptide selectively suppresses proinflammatory responses, whereby the peptide can contact a cell treated with an inflammatory stimulus and containing a polynucleotide or polynucleotides that encode a polypeptide involved in inflammation and sepsis and which is normally upregulated in response to this inflammatory stimulus, and wherein the peptides suppresses the expression of this gene or polypeptide as compared with expression of the inflammatory gene in the absence of the peptide and wherein the modulated expression results in enhancement of innate immunity.

In some embodiments, the peptide inhibits the inflammatory or septic response. In some embodiments, the peptide inhibits the expression of a pro-inflammatory gene or molecule. In some embodiments, the peptide inhibits the expression of TNF-α. In some embodiments, the inflammation is induced by a microbe or amicrobial ligand acting on a Toll-like receptor. In some embodiments, the microbial ligand is a bacterial endotoxin or lipopolysaccharide.

In a sixth aspect, disclosed herein is isolated molecule that has anti-biofilm activity by virtue of inhibiting (p)ppGpp synthesis or causing (p)ppGpp degradation. In some embodiments, the molecule is a peptide. In some embodiments, the peptide has 7 to 12 amino acids, where the peptide has an amino acid sequence of SEQ ID NOS: 1-749, or analogs, derivatives, enantiomers, amidated and unamidated variations and conservative variations thereof.

### BRIEF DESCRIPTION OF THE DRAWINGS

FIG. 1. Identification of new anti-biofilm peptides active against P. aeruginosa using the microtiter plate screening method with crystal violet staining. Demonstration that the D-L- and retro-inverso derivatives of peptide sequences have differential activity. As a control peptide 1037 was utilized [de la Fuente Nunez et al. 2011].

FIG. 2: Activity of DJK5 when added during P. aeruginosa biofilm formation or to pre-existing biofilms. P. aeruginosa was grown in minimal medium in continuous-culture flow cells. Channels were inoculated with 0.5 ml of early-stationary-phase cultures and incubated without flow for 4 h at 23° C. Flow of medium across the biofilm was then started (with or without added DJK5 at 10 μg/ml), with a mean flow of 0.3 ml/min, corresponding to a laminar flow with a Reynolds number of 5. Peptide DJK5 was added either at the initiation of the flow (i.e. during biofilm formation), or after two days (pre-existing biofilms). Biofilms were stained and visualized using the live/dead BacLight bacterial viability kit (Molecular probes Inc.). Live SYT09-stained cells (green) and dead propidium iodide-stained (red) cells were visualized with a Leica TCS microscope using appropriate optical filters. Overlapping stains were revealed as yellow looking cells. All experiments were done in two or more replicates with very similar results.

FIG. 3. Activity of DJK6 when added during S. aureus biofilm formation at 2.5 μg/ml. Experiments were done as described in the FIG. 2 legend. Live SYT09-stained cells (green) and dead propidium iodide-stained (red) cells were visualized with a Leica TCS microscope using appropriate optical filters.

FIG. 4: Activity of 1018 when added during biofilm formation by diverse bacteria or to pre-existing biofilms. Experiments were done as described in the FIG. 2 legend. Observations were as follows: E. coli: 3 days old control→structured biofilm; Added peptide at time zero→Few live planktonic cells; Treatment on 2 days pre-formed biofilm, treated by 1018 for the third day→Structured biofilm, but many cells are dead. Acinetobacter baumanii: Control 3 days-old biofilm→biofilm less structured than other bacteria; Added peptide at time zero→No live planktonic cells; Treatment on 2 days pre-formed biofilm, treated by 1018 for the third day→More cells than in the inhibition samples, but no aggregates. Klebsiella pneumoniae: Control 3 days-old biofilm→biofilm microcolonies; Added peptide at time zero→Mostly dead cells; Treatment on 2 days pre-formed biofilm, treated by 1018 for the third day→Mostly dead cells.

FIG. 5: Activity of 1018 when added during biofilm formation by diverse bacteria or to pre-existing biofilms. Experiments were done as described in the FIG. 2 legend. Observations were as follows: Staphylococcus aureus: Control 3 days-old biofilm→biofilm aggregates; Added peptide at time zero→few live cells; Treatment on 2 days pre-formed biofilm, treated by 1018 for the third day→few live cells. Salmonella enterica serovar Typhimurium: Control 3 days-old biofilm→biofilm aggregates; Added peptide at time zero→Some planktonic cells; Treatment on 2 days pre-formed biofilm, treated by 1018 for the third day→some dispersion, relatively few dead cells. Burkholderia cenocepacia: 3 days old control→biofilm microcolonies; Added peptide at time zero→Live cells but no microcolonies; Treatment on 2 days pre-formed biofilm, treated by 1018 for the third day→Some dead cells but no microcolonies.

FIG. 6: Activity of 1018 when added during biofilm formation by Burkholderia cepacia complex clinical isolates. This assay was performed in microtiter plates as described in the legend to FIG. 1.

FIG. 7: Synergy between peptides and antibiotics for inhibition of biofilm growth in flow cells. Minimal Biofilm Inhibitory Concentrations (MBIC) for P. aeruginosa: Ciprofloxacin=1.0 μg/ml; peptide 1018=24 μg/ml; peptide DJK5=0.5 μg/ml; MBICs for E. coli: Tobramycin=6.4 μg/ml; 1018=32 μg/ml; DJK5=0.5 μg/ml.

FIG. 8. Peptide synergy with ciprofloxacin vs. P. aeruginosa at the minimal biofilm eradication concentration in flow cells.

FIG. 9. Peptide synergy with tobramycin and ceftazidime vs. P. aeruginosa at the minimal biofilm eradication concentration in flow cells.

FIG. 10. Peptide 1018 affects events involved in the formation and dispersal of biofilms. (A) Peptide 1018 prevents initial attachment of planktonic bacteria to surfaces. The number of attached cells was analyzed by measuring absorbance at 595 nm. Statistical significance was determined using one-way ANOVA (where *** p<0.001). (B) 1018 significantly inhibited swimming and swarming motilities and stimulated twitching motility. (C) Congo red assays showing the effect of subinhibitory levels of 1018 (15 μg/mL) on Congo red binding. (D) Effect of 10 μg/mL 1018 on expression of biofilm-related genes.

FIG. 11. (p)ppGpp is essential for biofilm development in both Gram negative and Gram positive bacteria. (a) Mutants lacking the ability to synthesize (p)ppGpp did not develop biofilms in flow cells. Overproduction of ppGpp, either by exogenous addition of serine hydroxamate (SHX) (b) or relA overexpression (c) triggered biofilm development. (d) (p)ppGpp synthetases relA and spoT were up-regulated in biofilm cells compared to planktonic cells as determined by qRT-PCR.

FIG. 12. Stimulation of biofilm development by SHX. Biofilm development was induced below certain threshold levels of SHX and repressed above such levels (as seen here in the case of A. baumannii). Biofilms were stained and visualized using SYTO9 and examined by confocal laser scanning microscope. Each panel shows xy, yz and xz dimensions.

FIG. 13. Stimulation of biofilm development by relA overexpression. (p)ppGpp stimulation by increasing concentrations of IPTG correlated with the extent of induction of biofilm formation in E. coli. Each panel shows xy, yz and xz dimensions.

FIG. 14. (p)ppGpp overproduction led to peptide resistance and the peptide blocked (p)ppGpp production. (a) Both mutations in genes responsible for (p)ppGpp synthesis and treatment with peptide 1018 led to filamentation and cell death of bacteria grown under biofilm conditions in flow cells. (b, c) Overproduction of (p)ppGpp either by adding SHX (b) or overexpressing relA (c) led to peptide resistance. (d) Anti-biofilm peptide 1018 directly prevented (p)ppGpp production.

FIG. 15: Peptides also inhibit swarming motility of Pseudomonas aeruginosa PA14 and PAO1 and Burkholderia cenocepacia.

FIG. 16. Protection by an anti-biofilm peptide in a model of Pseudomonas aeruginosa biofilm infection in Drosophila. Protection was equivalent to 5 μg/ml tobramycin (not shown). The inset shows the in vivo biofilm growth mode of Pseudomonas in this model. The model and its validation was described in Mulcahy H., L. Charron-Mazenod, and S. Lewenza. 2008. Extracellular DNA chelates cations and induces antibiotic resistance in Pseudomonas aeruginosa biofilms. PLoS Pathog 4: e1000213.

FIG. 17. Protection by an anti-biofilm IDR peptide 1018 in a model of Citrobacter rodentium infection (mimics, in mice, enteropathogenic E. coli infections of man). A C. rodentium stain, tagged with a lux cassette to enable it to produce light, was infected into mice four hours after the addition of peptide. After 7 days mice were imaged with a CCD camera to observe visible light and the color scale to the right indicates the intensity (proportional to the number of bacteria) according to color. Peptide treated mice showed no residual bacteria while saline treated mice demonstrated heavy infection in the gastrointestinal tract (likely due to formation of a biofilm).

FIG. 17A. Protection by an anti-biofilm peptide in a Pseudomonas aeruginosa surface abrasion biofilm model. CD1 Mice were anesthetized, shaved on their backs and abrasions made with a nail file. For each abrasion, 10⁸ CFU/10 μl of Pseudomonas (PA14 Lux) was added to the abrasion and treated (left hand mice) or not (right hand mice) at time zero with DJK5 (200 μg/mouse resuspended at 20 mg/ml in water). After 24 and 48 hours of infection, mice were anesthetized via inhalation of aerosolized isoflurane mixed with oxygen and imaged using a Xenogen Imaging System 100 (Xenogen, Hopkinton, Mass.) to detect luminescent bacteria (which requires a bacterial energy source such that only live bacteria demonstrate luminescence). The experimental design had 2 controls and 2 DJK5-treated mice per cage, and significant variability was observed in the 8 mice used in these studies, although all treated mice had no bacteria. Top Figures: Normal mice; Bottom Figures: Results in cyclophosphamide treated (neutropenic) mice, which makes the biofilm last longer. Control mice had to be sacrificed after 2 days when they had reached the humane end-point. NB. an ROI of 1,000=5×10⁶ bacteria.

FIG. 18. Lack of cytotoxicity of immunomodulatory peptides against human peripheral blood mononuclear cells as determined by the low release of cytosolic lactate dehydrogenase.

FIG. 19. High production of anti-infective chemokine MCP-1 by human peripheral blood mononuclear cells treated with peptides, as determined by ELISA after 24 hours of stimulation.

FIG. 20. Ability of peptides to knockdown pro-inflammatory cytokine TNFα production by human PBMCs in response to bacterial LPS treatment as determined by ELISA after 24 hours.

FIG. 21. Ability of 10 μg/ml of peptides in combination with 20 or 5 μg/ml of the known adjuvant poly inosine:cytosine [poly(I:C)] to synergize to increase MCP-1 production, a known adjuvant property [see Kindrachuk, J., H. Jenssen, M. Elliott, R. Townsend, A. Nijnik, S. F. Lee, V. Gerdts, L. A. Babiuk, S. A. Halperin and R. E. W. Hancock. 2009. A novel vaccine adjuvant comprised of a synthetic innate defence regulator peptide and CpG oligonucleotide links innate and adaptive immunity. Vaccine 27:4662-4671].

### DETAILED DESCRIPTION

### A. Introduction

Peptides can be synthesized in solid phase, or as an array of peptides made in parallel on cellulose sheets (Frank, R. Spot synthesis: an easy technique for the positionally addressable, parallel chemical synthesis on a membrane support. Tetrahedron. 1992 48, 9217-9232) or by solution phase chemistry, and both of the first two methods were applied here. We previously adapted these methods, especially Spot synthesis, to create a large number of variants through sequence scrambling, truncations and systematic modifications of peptide sequence, and used a luciferase-based screen to investigate their ability to kill Pseudomonas aeruginosa planktonic cells (Hilpert K, Volkmer-Engert R, Walter T, Hancock R E W. High-throughput generation of small antibacterial peptides with improved activity. Nature Biotech 23:1008-1012, 2005). This permitted us to screen hundreds of 12-mer peptides based on the sequence of the bovine analog Bac2A and determine optimal amino acid substitutions, and using combinations of amino acid substitutions to define peptides of both 8 to 12 amino acids in length that had excellent broad spectrum antimicrobial activity against planktonic bacteria. We did not test the peptides vs. biofilms as we suspected they would be inactive since it is well understood that biofilms are highly resistant to conventional antibiotics (Stewart, P. S., and J. W. Costerton. 2001. Antibiotic resistance of bacteria in biofilms. Lancet 358:135-138.; Høiby, N., T. Bjarnsholt, M Givskov., S. Molin, O. Ciofu. 2010. Antibiotic resistance of bacterial biofilms. International Journal of Antimicrobial 35:322-32.).

To date screens for new anti-biofilm peptides and for new IDR peptides have been very limited. Using the procedures described above, we have been able to screen a much broader range of peptides starting from new templates. It has permitted a systematic and detailed investigation of the determinants of peptide activity in very small peptides. Thus we have been able to identify novel and potent anti-biofilm agents, existing IDR peptides that have unreported anti-biofilm activities, new IDR peptides and novel peptides with both anti-biofilm and IDR activities. Thus these peptides collectively have action against biofilms and the potential to favorably resolve infections.

The peptides of the invention retain activities in the typical media used to test in vitro antibiotic activity and/or tissue culture medium used to examine immunomodulatory activity, making them candidates for clinical therapeutic usage; in contrast most directly antimicrobial peptides are antagonized by physiological levels of salts.

The invention provides a number of methods, reagents, and compounds that can be used for inhibiting microbial infections or biofilm growth. It is to be understood that this invention is not limited to particular methods, reagents, compounds, compositions, or biological systems, which can, of course, vary. It is also to be understood that the terminology used herein is for the purpose of describing particular embodiments only, and is not intended to be limiting. As used in this specification and the appended claims, the singular forms “a”, “an”, and “the” include plural referents unless the content clearly dictates otherwise. Thus, for example, reference to “a peptide” includes a combination of two or more peptides, and the like.

“About” as used herein when referring to a measurable value such as an amount, a temporal duration, and the like, is meant to encompass variations of ±20% or ±10%, more preferably ±5%, even more preferably ±1%, and still more preferably ±0.1% from the specified value, as such variations are appropriate to perform the disclosed methods.

Unless defined otherwise, all technical and scientific terms used herein have the same meaning as commonly understood by one of ordinary skill in the art to which the invention pertains. Although any methods and materials similar or equivalent to those described herein can be used in the practice for testing of the present invention, the preferred materials and methods are described herein. In describing and claiming the present invention, the following terminology will be used.

“Antimicrobial” as used herein means that the peptides of the present invention inhibit, prevent, or destroy the growth or proliferation of planktonic (free swimming) microbes such as bacteria, fungi, viruses, parasites or the like. Anti-biofilm relates to the ability to destroy, inhibit the growth of, or encourage the dispersal of, biofilms of living organisms.

“Selective enhancement of innate immunity” or “immunomodulatory” as used herein means that the peptides of the invention are able to upregulate, in mammalian cells, genes and molecules that are natural components of the innate immune response and assist in the resolution of infections without excessive increases, or with actual decreases, of pro-inflammatory cytokines like TNFα that can cause potentially harmful inflammation and thus initiate a sepsis reaction in a subject. The peptides do not stimulate a septic reaction, but do stimulate expression of the one or more genes encoding chemokines or interleukins that attract immune cells including MCP-1, MCP-3, and CXCL-1. The peptides may also possess anti-sepsis activity including an ability to reduce the expression of TNFα in response to bacterial ligands like LPS.

The “amino acid” residues identified herein are in the natural L-configuration or isomeric D-configuration. In keeping with standard polypeptide nomenclature, J. Biol. Chem., 243:3557-59, (1969), abbreviations for amino acid residues are as shown in the following table.

| 1-Letter | 3-Letter | Amino Acid |
|---|---|---|
| Y | Tyr | L-tyrosine |
| G | Gly | L-glycine |
| F | Phe | L-phenylalanine |
| M | Met | L-methionine |
| A | Ala | L-alanine |
| S | Ser | L-serine |
| I | Ile | L-isoleucine |
| L | Leu | L-leucine |
| T | Thr | L-threonine |
| V | Val | L-valine |
| P | Pro | L-proline |
| K | Lys | L-lysine |
| H | His | L-histidine |
| Q | Gin | L-glutamine |
| E | Glu | L-glutamic acid |
| W | Trp | L-tryptohan |
| R | Arg | L-arginine |
| D | Asp | L-aspartic acid |
| N | Asn | L-asparagine |
| C | Cys | L-cysteine |

It should be noted that all amino acid residue sequences are represented herein by formulae whose left to right orientation is in the conventional direction of amino-terminus to carboxy-terminus. Also all peptides are modified at the carboxy-terminus to remove the negative charge, often through amidation, esterification, acylation or the like.

Particularly favored amino acids include A, R, L, I, V, K, W, G, and Q.

### B. Peptides

The invention provides an isolated peptide with anti-biofilm and/or immunomodulatory activity. Exemplary peptides of the invention have an amino acid sequence including those listed in Table 1, and analogs, derivatives, enantiomers, amidated and unamidated versions, variations and conservative variations thereof, wherein the peptides have anti-biofilm and/or immunomodulatory activity. The peptides of the invention include SEQ ID NOS:1-739, as well as the broader groups of peptides having conservative substitutions, and conservative variations thereof.

“Isolated” when used in reference to a peptide, refers to a peptide substantially free of proteins, lipids, nucleic acids, for example, with which it might be naturally associated. Those of skill in the art can make similar substitutions to achieve peptides with similar or greater antibiofilm or immunomodulatory activity. For example, the invention includes the peptides depicted in SEQ ID NOS:1-749, as well as analogs or derivatives thereof, as long as the bioactivity (e.g., antimicrobial) of the peptide remains. Minor modifications of the primary amino acid sequence of the peptides of the invention may result in peptides that have substantially equivalent activity as compared to the specific peptides described herein. Such modifications may be deliberate, as by site-specific substitutions or may be spontaneous. All of the peptides produced by these modifications are included herein as long as the biological activity of the original peptide still exists.

Further, deletion of one or more amino acids can also result in a modification of the structure of the resultant molecule without significantly altering its biological activity. This can lead to the development of a smaller active molecule that would also have utility. For example, amino or carboxy terminal amino acids that may not be required for biological activity of the particular peptide can be removed. Peptides of the invention include any analog, homolog, mutant, isomer or derivative of the peptides disclosed in the present invention, so long as the bioactivity as described herein remains. All peptides are synthesized using L or D form amino acids, however, mixed peptides containing both L- and D-form amino acids can be synthetically produced. In addition, C-terminal derivatives can be produced, such as C-terminal amidates, C-terminal acylates, and C-terminal methyl and acetyl esters, in order to increase the anti-biofilm or immunomodulatory activity of a peptide of the invention. The peptide can be synthesized such that the sequence is reversed whereby the last amino acid in the sequence becomes the first amino acid, and the penultimate amino acid becomes the second amino acid, and so on.

In certain embodiments, the peptides of the invention include peptide analogs and peptide mimetics. Indeed, the peptides of the invention include peptides having any of a variety of different modifications, including those described herein.

Peptide analogs of the invention are generally designed and produced by chemical modifications of a lead peptide, including, e.g., any of the particular peptides described herein, such as any of the following sequences disclosed in the tables. The present invention clearly establishes that these peptides in their entirety and derivatives created by modifying any side chains of the constituent amino acids have the ability to inhibit, prevent, or destroy the growth or proliferation of microbes such as bacteria, fungi, viruses, parasites or the like. The present invention further encompasses polypeptides up to about 50 amino acids in length that include the amino acid sequences and functional variants or peptide mimetics of the sequences described herein.

In another embodiment, a peptide of the present invention is a pseudopeptide. Pseudopeptides or amide bond surrogates refers to peptides containing chemical modifications of some (or all) of the peptide bonds. The introduction of amide bond surrogates not only decreases peptide degradation but also may significantly modify some of the biochemical properties of the peptides, particularly the conformational flexibility and hydrophobicity.

To improve or alter the characteristics of the peptides of the present invention, protein engineering can be employed. Recombinant DNA technology known to those skilled in the art can be used to create novel mutant proteins or muteins including single or multiple amino acid substitutions, deletions, additions, or fusion proteins. Such modified polypeptides can show, e.g., increased/decreased biological activity or increased/decreased stability. In addition, they can be purified in higher yields and show better solubility than the corresponding natural polypeptide, at least under certain purification and storage conditions. Further, the peptides of the present invention can be produced as multimers including dimers, trimers and tetramers. Multimerization can be facilitated by linkers, introduction of cysteines to permit creation of interchain disulphide bonds, or recombinantly though heterologous polypeptides such as Fc regions.

It is known in the art that one or more amino acids can be deleted from the N-terminus or C-terminus without substantial loss of biological function. See, e.g., Ron, et al., Biol Chem., 268: 2984-2988, 1993. Accordingly, the present invention provides polypeptides having one or more residues deleted from the amino terminus. Similarly, many examples of biologically functional C-terminal deletion mutants are known (see, e.g., Dobeli, et al., 1988). Accordingly, the present invention provides polypeptides having one or more residues deleted from the carboxy terminus. The invention also provides polypeptides having one or more amino acids deleted from both the amino and the carboxyl termini as described below.

Other mutants in addition to N- and C-terminal deletion forms of the protein discussed above are included in the present invention. Thus, the invention further includes variations of the polypeptides that show substantial anti-biofilm and/or immunomodulatory activity. Such mutants include deletions, insertions, inversions, repeats, and substitutions selected according to general rules known in the art so as to have little effect on activity.

There are two main approaches for studying the tolerance of an amino acid sequence to change, see, Bowie, et al., Science, 247: 1306-1310, 1994. The first method relies on the process of evolution, in which mutations are either accepted or rejected by natural selection. The second approach uses genetic engineering to introduce amino acid changes at specific positions of a cloned gene and selections or screens to identify sequences that maintain functionality. These studies have revealed that proteins are surprisingly tolerant of amino acid substitutions. Similarly the effects of such changes can easily be assessed by employing artificial neural networks and quantitative structure activity analyses [Cherkasov et al, 2009].

Typically seen as conservative substitutions are the replacements, one for another, among the aliphatic amino acids Ala, Val, Leu, and Ile; interchange of the hydroxyl residues Ser and Thr, exchange of the acidic residues Asp and Glu, substitution between the amide residues Asn and Gln, exchange of the basic residues Lys and Arg, and replacements among the aromatic residues Phe, Tyr and Trp. Thus, the peptide of the present invention can be, for example: (i) one in which one or more of the amino acid residues are substituted with a conserved or non-conserved amino acid residue (preferably a conserved amino acid residue) and such substituted amino acid residue can or cannot be one encoded by the genetic code; or (ii) one in which one or more of the amino acid residues includes a substituent group; or (iii) one in which the polypeptide is fused with another compound, such as a compound to increase the half-life of the polypeptide (for example, polyethylene glycol); or (iv) one in which the additional amino acids are fused to the above form of the polypeptide, such as an IgG Fc fusion region peptide or leader or secretory sequence or a sequence which is employed for purification of the above form of the polypeptide or a pro-protein sequence.

Thus, the peptides of the present invention can include one or more amino acid substitutions, deletions, or additions, either from natural mutations or human manipulation. As indicated, changes are preferably of a minor nature, such as conservative amino acid substitutions that do not significantly affect the folding or activity of the peptide. The following groups of amino acids represent equivalent changes: (1) Gln, Asn; (2) Ser, Thr; (3) Val, Ile, Leu, Met, Ala, Phe; (4) Lys, Arg, His; (5) Phe, Tyr, Trp.

Arginine and/or lysine can be substituted with other basic non-natural amino acids including ornithine, citrulline, homoarginine, Nδ-[1-(4,4-dimethyl-2,6-dioxocyclohexylidene)-ethyl-L-ornithine, Nε-methyltrityl-L-lysine, and diamino-butyrate although many other mimetic residues are available. Tryptophan residues can be substituted for homo-tryptophan, bromotryptophan and fluorotryptophan. The term “conservative variation” also includes the use of a substituted amino acid in place of an unsubstituted parent amino acid provided that the substituted polypeptide at least retains most of the activity of the unsubstituted parent peptide. Such conservative substitutions are within the definition of the classes of the peptides of the invention.

The present invention is further directed to fragments of the peptides of the present invention. More specifically, the present invention embodies purified, isolated, and recombinant peptides comprising at least any one integer between 6 and 504 (or the length of the peptides amino acid residues minus 1 if the length is less than 1000) of consecutive amino acid residues. Preferably, the fragments are at least 6, preferably at least 7 to 11, more preferably 12 consecutive amino acids of a peptide of the present invention.

In addition, it should be understood that in certain embodiments, the peptides of the present invention include two or more modifications, including, but not limited to those described herein. By taking into the account the features of the peptide drugs on the market or under current development, it is clear that most of the peptides successfully stabilized against proteolysis consist of a mixture of several types of the above-described modifications. This conclusion is understood in the light of the knowledge that many different enzymes are implicated in peptide degradation.

### C. Peptides, Peptide Variants, and Peptide Mimetics

“Polypeptide,” “peptide” and “protein” are used interchangeably herein to refer to a polymer of amino acid residues. The terms apply to amino acid polymers in which one or more amino acid residue is an artificial chemical mimetic of a corresponding naturally occurring amino acid, as well as to naturally occurring amino acid polymers and non-naturally occurring amino acid polymer. Amino acid mimetics refers to chemical compounds that have a structure that is different from the general chemical structure of a natural amino acid, but which function in a manner similar to a naturally occurring amino acid. Non-natural residues are well described in the scientific and patent literature; a few exemplary non-natural compositions useful as mimetics of natural amino acid residues and guidelines are described below. Mimetics of aromatic amino acids can be generated by replacing by, e.g., D- or L-naphylalanine; D- or L-phenylglycine; D- or L-2 thieneylalanine; D- or L-1, -2,3-, or 4-pyreneylalanine; D- or L-3 thieneylalanine; D- or L-(2-pyridinyl)-alanine; D- or L-(3-pyridinyl)-alanine; D- or L-(2-pyrazinyl)-alanine; D- or L-(4-isopropyl)-phenylglycine; D-(trifluoromethyl)-phenylglycine; D-(trifluoromethyl)-phenylalanine; D-p-fluoro-phenylalanine; D- or L-p-biphenylphenylalanine; K- or L-p-methoxy-biphenylphenylalanine; D- or L-2-indole(alkyl)alanines; and, D- or L-alkylainines, where alkyl can be substituted or unsubstituted methyl, ethyl, propyl, hexyl, butyl, pentyl, isopropyl, iso-butyl, sec-isotyl, iso-pentyl, or a non-acidic amino acids. Aromatic rings of a non-natural amino acid include, e.g., thiazolyl, thiophenyl, pyrazolyl, benzimidazolyl, naphthyl, furanyl, pyrrolyl, and pyridyl aromatic rings.

“Peptide” as used herein includes peptides that are conservative variations of those peptides specifically exemplified herein. “Conservative variation” as used herein denotes the replacement of an amino acid residue by another, biologically similar residue, as discussed elsewhere herein. “Cationic” as is used to refer to any peptide that possesses sufficient positively charged amino acids to have a pI (isoelectric point) greater than about 9.0.

The biological activity of the peptides can be determined by standard methods known to those of skill in the art, such as “minimal biofilm inhibitory concentration (MBIC)” or “minimal biofilm eradication concentration (MBEC)” assays described in the present examples, whereby the lowest concentration causing reduction or eradication of biofilms is observed for a given period of time and recorded as the MBIC or MBEC respectively.

The peptides and polypeptides of the invention, as defined above, include all “mimetic” and “peptidomimetic” forms. The terms “mimetic” and “peptidomimetic” refer to a synthetic chemical compound that has substantially the same structural and/or functional characteristics of the polypeptides of the invention. The mimetic can be either entirely composed of synthetic, non-natural analogues of amino acids, or, is a chimeric molecule of partly natural peptide amino acids and partly non-natural analogs of amino acids. The mimetic can also incorporate any number of natural amino-acid conservative substitutions as long as such substitutions do not substantially alter the mimetic's structure and/or activity. As with polypeptides of the invention that are conservative variants, routine experimentation will determine whether a mimetic is within the scope of the invention, i.e., that its structure and/or function is not substantially altered. Thus, a mimetic composition is within the scope of the invention if it has anti-biofilm or immunomodulatory activity.

Polypeptide mimetic compositions can also contain any combination of non-natural structural components, which are typically from three structural groups: a) residue linkage groups other than the natural amide bond (“peptide bond”) linkages; b) non-natural residues in place of naturally occurring amino acid residues; or c) residues that induce secondary structural mimicry, i.e., to induce or stabilize a secondary structure, e.g., a beta turn, gamma turn, beta sheet, alpha helix conformation, and the like. For example, a polypeptide can be characterized as a mimetic when all or some of its residues are joined by chemical means other than natural peptide bonds. Individual peptidomimetic residues can be joined by peptide bonds, other chemical bonds or coupling means, such as, e.g., glutaraldehyde, N-hydroxysuccinimide esters, bifunctional maleimides, N,N′-dicyclohexylcarbodiimide (DCC) or N,N′-diisopropylcarbodiimide (DIC). Linking groups that can be an alternative to the traditional amide bond (“peptide bond”) linkages include, e.g., ketomethylene (e.g., —C(═O)—CH₂— for —C(═O)—NH—), aminomethylene (CH₂—NH), ethylene, olefin (CH═CH), ether (CH₂—O), thioether (CH₂—S), tetrazole (CN₄—), thiazole, retroamide, thioamide, or ester (see, e.g., Spatola (1983) in Chemistry and Biochemistry of Amino Acids, Peptides and Proteins, Vol. 7, pp 267-357, “Peptide Backbone Modifications,” Marcell Dekker, NY).

Mimetics of acidic amino acids can be generated by substitution by, e.g., non-carboxylate amino acids while maintaining a negative charge such as e.g. (phosphono)alanine; sulfated threonine. Carboxyl side groups (e.g., aspartyl or glutamyl) can also be selectively modified by reaction with carbodiimides (R′—N—C—N—R′) such as, e.g., 1-cyclohexyl-3 (2-morpholin-yl-(4-ethyl) carbodiimide or 1-ethyl-3 (4-azonia-4,4-dimetholpentyl) carbodiimide. Aspartyl or glutamyl can also be converted to asparaginyl and glutaminyl residues by reaction with ammonium ions.

Mimetics of basic amino acids can be generated by substitution with, e.g., (in addition to lysine and arginine) the amino acids ornithine, or citrulline or the side chain diaminobenzoate. Asparaginyl and glutaminyl residues can be deaminated to the corresponding aspartyl or glutamyl residues.

Arginine residue mimetics can be generated by reacting arginyl with, e.g., one or more conventional reagents, including, e.g., phenylglyoxal, 2,3-butanedione, 1,2-cyclohexanedione, or ninhydrin, preferably under alkaline conditions. Tyrosine residue mimetics can be generated by reacting tyrosyl with, e.g., aromatic diazonium compounds or tetranitromethane. N-acetylimidizol and tetranitromethane can be used to form 0-acetyl tyrosyl species and 3-nitro derivatives, respectively. Cysteine residue mimetics can be generated by reacting cysteinyl residues with, e.g., alpha-haloacetates such as 2-chloroacetic acid or chloroacetamide and corresponding amines; to give carboxymethyl or carboxyamidomethyl derivatives. Cysteine residue mimetics can also be generated by reacting cysteinyl residues with, e.g., bromo-trifluoroacetone, alpha-bromo-beta-(5-imidozoyl) propionic acid; chloroacetyl phosphate, N-alkylmaleimides, 3-nitro-2-pyridyl disulfide; methyl 2-pyridyl disulfide; p-chloromercuribenzoate; 2-chloromercuri-4 nitrophenol; or, chloro-7-nitrobenzo-oxa-1,3-diazole. Lysine mimetics can be generated (and amino terminal residues can be altered) by reacting lysinyl with, e.g., succinic or other carboxylic acid anhydrides. Lysine and other alpha-amino-containing residue mimetics can also be generated by reaction with imidoesters, such as methyl picolinimidate, pyridoxal phosphate, pyridoxal, chloroborohydride, trinitrobenzenesulfonic acid, O-methylisourea, 2,4, pentanedione, and transamidase-catalyzed reactions with glyoxylate. Mimetics of methionine can be generated by reaction with, e.g., methionine sulfoxide. Histidine residue mimetics can be generated by reacting histidyl with, e.g., diethylprocarbonate or para-bromophenacyl bromide. Other mimetics include, e.g., those generated by hydroxylation of lysine; phosphorylation of the hydroxyl groups of seryl or threonyl residues; methylation of the alpha-amino groups of lysine, arginine and histidine; acetylation of the N-terminal amine; methylation of main chain amide residues or substitution with N-methyl amino acids; or amidation of C-terminal carboxyl groups.

A component of a peptide of the invention can also be replaced by an amino acid (or peptidomimetic residue) of the opposite chirality. Thus, any amino acid naturally occurring in the L-configuration (which can also be referred to as the R or S, depending upon the structure of the chemical entity) can be replaced with the amino acid of the same chemical structural type or a peptidomimetic, but of the opposite chirality, referred to as the D-amino acid, but which can additionally be referred to as the R- or S-form, and vice versa.

The invention also provides peptides that are “substantially identical” to an exemplary peptide of the invention. A “substantially identical” amino acid sequence is a sequence that differs from a reference sequence by one or more conservative or non-conservative amino acid substitutions, deletions, or insertions, particularly when such a substitution occurs at a site that is not the active site of the molecule, and provided that the polypeptide essentially retains its functional properties. A conservative amino acid substitution, for example, substitutes one amino acid for another of the same class (e.g., substitution of one hydrophobic amino acid, such as isoleucine, valine, leucine, or methionine, for another, or substitution of one polar amino acid for another, such as substitution of arginine for lysine, glutamic acid for aspartic acid or glutamine for asparagine). One or more amino acids can be deleted, for example, from an anti-biofilm or immunomodulatory polypeptide having anti-biofilm or immunomodulatory activity of the invention, resulting in modification of the structure of the polypeptide, without significantly altering its biological activity. For example, amino- or carboxyl-terminal, or internal, amino acids that are not required for antimicrobial activity can be removed.

The skilled artisan will recognize that individual synthetic residues and peptides incorporating these mimetics can be synthesized using a variety of procedures and methodologies, which are well described in the scientific and patent literature, e.g., Organic Syntheses Collective Volumes, Gilman, et al. (Eds) John Wiley & Sons, Inc., NY. Peptides and peptide mimetics of the invention can also be synthesized using combinatorial methodologies. Various techniques for generation of peptide and peptidomimetic libraries are well known, and include, e.g., multipin, tea bag, and split-couple-mix techniques; see, e.g., al-Obeidi, Mol. Biotechnol. 9: 205-223, 1998; Hruby, Curr. Opin. Chem. Biol. 1: 114-119, 1997; Ostergaard, Mol. Divers. 3: 17-27, 1997; Ostresh, Methods Enzymol. 267: 220-234, 1996. Modified peptides of the invention can be further produced by chemical modification methods, see, e.g., Belousov, Nucleic Acids Res. 25: 3440-3444, 1997; Frenkel, Free Radic. Biol. Med. 19: 373-380, 1995; Blommers, Biochemistry 33: 7886-7896, 1994.

Peptides and polypeptides of the invention can be isolated from natural sources, be synthetic, or be recombinantly generated polypeptides. Peptides and proteins can be recombinantly expressed in vitro or in vivo. The peptides and polypeptides of the invention can be made and isolated using any method known in the art. Polypeptide and peptides of the invention can also be synthesized, whole or in part, using chemical methods well known in the art. See e.g., Caruthers, Nucleic Acids Res. Symp. Ser. 215-223, 1980; Horn, Nucleic Acids Res. Symp. Ser. 225-232, 1980; Banga, Therapeutic Peptides and Proteins, Formulation, Processing and Delivery Systems Technomic Publishing Co., Lancaster, Pa., 1995. For example, peptide synthesis can be performed using various solid-phase techniques (see e.g., Roberge, Science 269: 202, 1995; Merrifield, Methods Enzymol. 289: 3-13, 1997) and automated synthesis can be achieved, e.g., using the ABI 431A Peptide Synthesizer (Perkin Elmer) in accordance with the instructions provided by the manufacturer.

Peptides of the invention can be synthesized by such commonly used methods as t-BOC or FMOC protection of alpha-amino groups. Both methods involve stepwise syntheses whereby a single amino acid is added at each step starting from the C terminus of the peptide (See, Coligan, et al., Current Protocols in Immunology, Wiley Interscience, 1991, Unit 9). Peptides of the invention can also be synthesized by the well known solid phase peptide synthesis methods described in Merrifield, J. Am. Chem. Soc., 85:2149, (1962), and Stewart and Young, Solid Phase Peptides Synthesis, (Freeman, San Francisco, 1969, pp. 27-62), using a copoly(styrene-divinylbenzene) containing 0.1-1.0 mMol amines/g polymer. On completion of chemical synthesis, the peptides can be deprotected and cleaved from the polymer by treatment with liquid HF-10% anisole for about ¼-1 hours at 0° C. After evaporation of the reagents, the peptides are extracted from the polymer with 1% acetic acid solution which is then lyophilized to yield the crude material. This can normally be purified by such techniques as gel filtration on Sephadex G-15 using 5% acetic acid as a solvent. Lyophilization of appropriate fractions of the column will yield the homogeneous peptide or peptide derivatives, which can then be characterized by such standard techniques as amino acid analysis, thin layer chromatography, high performance liquid chromatography, ultraviolet absorption spectroscopy, molar rotation, solubility, and quantitated by the solid phase Edman degradation.

Analogs, polypeptide fragment of anti-biofilm or immunomodulatory protein having anti-biofilm or immunomodulatory activity, are generally designed and produced by chemical modifications of a lead peptide, including, e.g., any of the particular peptides described herein, such as any of the sequences including SEQ ID NOS:1-749.

As contemplated by this invention, “polypeptide” includes those having one or more chemical modification relative to another polypeptide, i.e., chemically modified polypeptides. The polypeptide from which a chemically modified polypeptide is derived may be a wildtype protein, a functional variant protein or a functional variant polypeptide, or polypeptide fragments thereof; an antibody or other polypeptide ligand according to the invention including without limitation single-chain antibodies, crystalline proteins and polypeptide derivatives thereof; or polypeptide ligands prepared according to the disclosure. Preferably, the chemical modification(s) confer(s) or improve(s) desirable attributes of the polypeptide but does not substantially alter or compromise the biological activity thereof. Desirable attributes include but are limited to increased shelf-life; enhanced serum or other in vivo stability; resistance to proteases; and the like. Such modifications include by way of non-limiting example N-terminal acetylation, glycosylation, and biotinylation.

An effective approach to confer resistance to peptidases acting on the N-terminal or C-terminal residues of a polypeptide is to add chemical groups at the polypeptide termini, such that the modified polypeptide is no longer a substrate for the peptidase. One such chemical modification is glycosylation of the polypeptides at either or both termini. Certain chemical modifications, in particular N-terminal glycosylation, have been shown to increase the stability of polypeptides in human serum (Powell et al., Pharma. Res. 10: 1268-1273, 1993). Other chemical modifications which enhance serum stability include, but are not limited to, the addition of an N-terminal alkyl group, consisting of a lower alkyl of from 1 to 20 carbons, such as an acetyl group, and/or the addition of a C-terminal amide or substituted amide group.

The presence of an N-terminal D-amino acid increases the serum stability of a polypeptide that otherwise contains L-amino acids, because exopeptidases acting on the N-terminal residue cannot utilize a D-amino acid as a substrate. Similarly, the presence of a C-terminal D-amino acid also stabilizes a polypeptide, because serum exopeptidases acting on the C-terminal residue cannot utilize a D-amino acid as a substrate. With the exception of these terminal modifications, the amino acid sequences of polypeptides with N-terminal and/or C-terminal D-amino acids are usually identical to the sequences of the parent L-amino acid polypeptide.

The terms “identical” or percent “identity”, in the context of two or peptide sequences, refers to two or more sequences or subsequences that are the same or have a specified percentage of amino acid residues or nucleotides that are the same (i.e., about 65% identity, preferably 75%, 85%, 90%, or higher identity over a specified region (e.g., nucleotide sequence encoding a peptide described herein or amino acid sequence), when compared and aligned for maximum correspondence over a comparison window or designated region) as measured using Muscle multiple alignment sequence comparison algorithms (http://www.bioinformatics.nl/tools/muscle.html) or by manual alignment and visual inspection. Such sequences are then said to be “substantially identical.” In some preferred embodiments, the identity is 87%. The term also includes sequences that have deletions and/or additions, as well as those that have substitutions as long as at least two thirds of the amino acids can be aligned. As described below, the preferred algorithms can account for gaps and the like. Preferably, for small peptides like those of the invention, identity exists over a region that is at least about 6 amino acids in length.

For peptide sequence comparison, typically one sequence acts as a reference sequence, to which test sequences are compared. When using a sequence comparison algorithm, test and reference sequences are entered into a computer in FASTA format and alignment is performed. Preferably, default program parameters can be used, or alternative parameters can be designated. The sequence comparison algorithm then aligns the sequences enabling a calculation of the percent sequence identities for the test sequences relative to the reference sequence, based on the program parameters.

### E. Polypeptide Mimetic

In general, a polypeptide mimetic (“peptidomimetic”) is a molecule that mimics the biological activity of a polypeptide but is no longer peptidic in chemical nature. By strict definition, a peptidomimetic is a molecule that contains no peptide bonds (that is, amide bonds between amino acids). However, the term peptidomimetic is sometimes used to describe molecules that are no longer completely peptidic in nature, such as pseudo-peptides, semi-peptides and peptoids. Examples of some peptidomimetics by the broader definition (where part of a polypeptide is replaced by a structure lacking peptide bonds) are described below. Whether completely or partially non-peptide, peptidomimetics according to this invention provide a spatial arrangement of reactive chemical moieties that closely resembles the three-dimensional arrangement of active groups in the polypeptide on which the peptidomimetic is based. As a result of this similar active-site geometry, the peptidomimetic has effects on biological systems that are similar to the biological activity of the polypeptide.

There are several potential advantages for using a mimetic of a given polypeptide rather than the polypeptide itself. For example, polypeptides may exhibit two undesirable attributes, i.e., poor bioavailability and short duration of action. Peptidomimetics are often small enough to be both orally active and to have a long duration of action. There are also problems associated with stability, storage and immunoreactivity for polypeptides that are not experienced with peptidomimetics.

Candidate, lead and other polypeptides having a desired biological activity can be used in the development of peptidomimetics with similar biological activities. Techniques of developing peptidomimetics from polypeptides are known. Peptide bonds can be replaced by non-peptide bonds that allow the peptidomimetic to adopt a similar structure, and therefore biological activity, to the original polypeptide. Further modifications can also be made by replacing chemical groups of the amino acids with other chemical groups of similar structure. The development of peptidomimetics can be aided by determining the tertiary structure of the original polypeptide, either free or bound to a ligand, by NMR spectroscopy, crystallography and/or computer-aided molecular modeling. These techniques aid in the development of novel compositions of higher potency and/or greater bioavailability and/or greater stability than the original polypeptide (Dean, BioEssays, 16: 683-687, 1994; Cohen and Shatzmiller, J. Mol. Graph., 11: 166-173, 1993; Wiley and Rich, Med. Res. Rev., 13: 327-384, 1993; Moore, Trends Pharmacol. Sci., 15: 124-129, 1994; Hruby, Biopolymers, 33: 1073-1082, 1993; Bugg et al., Sci. Am., 269: 92-98, 1993, all incorporated herein by reference].

Thus, through use of the methods described above, the present invention provides compounds exhibiting enhanced therapeutic activity in comparison to the polypeptides described above. The peptidomimetic compounds obtained by the above methods, having the biological activity of the above named polypeptides and similar three-dimensional structure, are encompassed by this invention. It will be readily apparent to one skilled in the art that a peptidomimetic can be generated from any of the modified polypeptides described in the previous section or from a polypeptide bearing more than one of the modifications described from the previous section. It will furthermore be apparent that the peptidomimetics of this invention can be further used for the development of even more potent non-peptidic compounds, in addition to their utility as therapeutic compounds.

Specific examples of peptidomimetics derived from the polypeptides described in the previous section are presented below. These examples are illustrative and not limiting in terms of the other or additional modifications.

Proteases act on peptide bonds. It therefore follows that substitution of peptide bonds by pseudopeptide bonds confers resistance to proteolysis. A number of pseudopeptide bonds have been described that in general do not affect polypeptide structure and biological activity. The reduced isostere pseudopeptide bond is a suitable pseudopeptide bond that is known to enhance stability to enzymatic cleavage with no or little loss of biological activity (Couder, et al., Int. J. Polypeptide Protein Res. 41: 181-184, 1993, incorporated herein by reference). Thus, the amino acid sequences of these compounds may be identical to the sequences of their parent L-amino acid polypeptides, except that one or more of the peptide bonds are replaced by an isosteric pseudopeptide bond. Preferably the most N-terminal peptide bond is substituted, since such a substitution would confer resistance to proteolysis by exopeptidases acting on the N-terminus.

To confer resistance to proteolysis, peptide bonds may also be substituted by retro-inverso pseudopeptide bonds (Dalpozzo, et al., Int. J. Polypeptide Protein Res. 41: 561-566, incorporated herein by reference). According to this modification, the amino acid sequences of the compounds may be identical to the sequences of their L-amino acid parent polypeptides, except that one or more of the peptide bonds are replaced by a retro-inverso pseudopeptide bond. Preferably the most N-terminal peptide bond is substituted, since such a substitution will confer resistance to proteolysis by exopeptidases acting on the N-terminus.

Peptoid derivatives of polypeptides represent another form of modified polypeptides that retain the important structural determinants for biological activity, yet eliminate the peptide bonds, thereby conferring resistance to proteolysis (Simon, et al., Proc. Natl. Acad. Sci. USA, 89: 9367-9371, 1992, and incorporated herein by reference). Peptoids are oligomers of N-substituted glycines. A number of N-alkyl groups have been described, each corresponding to the side chain of a natural amino acid.

### F. Polynucleotides

The invention includes polynucleotides encoding peptides of the invention. Exemplary polynucleotides encode peptides including those listed in Table 1, and analogs, derivatives, amidated variations and conservative variations thereof, wherein the peptides have antimicrobial activity. The peptides of the invention include SEQ ID NOS:1-749, as well as the broader groups of peptides having hydrophilic and hydrophobic substitutions, and conservative variations thereof.

“Isolated” when used in reference to a polynucleotide, refers to a polynucleotide substantially free of proteins, lipids, nucleic acids, for example, with which it is naturally associated. As used herein, “polynucleotide” refers to a polymer of deoxyribonucleotides or ribonucleotides, in the form of a separate fragment or as a component of a larger construct. DNA encoding a peptide of the invention can be assembled from cDNA fragments or from oligonucleotides which provide a synthetic gene which is capable of being expressed in a recombinant transcriptional unit. Polynucleotide sequences of the invention include DNA, RNA and cDNA sequences. A polynucleotide sequence can be deduced from the genetic code, however, the degeneracy of the code must be taken into account. Polynucleotides of the invention include sequences which are degenerate as a result of the genetic code. Such polynucleotides are useful for the recombinant production of large quantities of a peptide of interest, such as the peptide of SEQ ID NOS:1-749.

In the present invention, the polynucleotides encoding the peptides of the invention may be inserted into a recombinant “expression vector”. The term “expression vector” refers to a plasmid, virus or other vehicle known in the art that has been manipulated by insertion or incorporation of genetic sequences. Such expression vectors of the invention are preferably plasmids that contain a promoter sequence that facilitates the efficient transcription of the inserted genetic sequence in the host. The expression vector typically contains an origin of replication, a promoter, as well as specific genes that allow phenotypic selection of the transformed cells. For example, the expression of the peptides of the invention can be placed under control of E. coli chromosomal DNA comprising a lactose or lac operon which mediates lactose utilization by elaborating the enzyme beta-galactosidase. The lac control system can be induced by IPTG. A plasmid can be constructed to contain the lacIq repressor gene, permitting repression of the lac promoter until IPTG is added. Other promoter systems known in the art include beta lactamase, lambda promoters, the protein A promoter, and the tryptophan promoter systems. While these are the most commonly used, other microbial promoters, both inducible and constitutive, can be utilized as well. The vector contains a replicon site and control sequences which are derived from species compatible with the host cell. In addition, the vector may carry specific gene(s) which are capable of providing phenotypic selection in transformed cells. For example, the beta-lactamase gene confers ampicillin resistance to those transformed cells containing the vector with the beta-lactamase gene. An exemplary expression system for production of the peptides of the invention is described in U.S. Pat. No. 5,707,855.

Transformation of a host cell with the polynucleotide may be carried out by conventional techniques known to those skilled in the art. For example, where the host is prokaryotic, such as E. coli, competent cells that are capable of DNA uptake can be prepared from cells harvested after exponential growth and subsequently treated by the CaCl₂ method using procedures known in the art. Alternatively, MgCl₂ or RbCl could be used.

In addition to conventional chemical methods of transformation, the plasmid vectors of the invention may be introduced into a host cell by physical means, such as by electroporation or microinjection. Electroporation allows transfer of the vector by high voltage electric impulse, which creates pores in the plasma membrane of the host and is performed according to methods known in the art. Additionally, cloned DNA can be introduced into host cells by protoplast fusion, using methods known in the art.

DNA sequences encoding the peptides can be expressed in vivo by DNA transfer into a suitable host cell. “Host cells” of the invention are those in which a vector can be propagated and its DNA expressed. The term also includes any progeny of the subject host cell. It is understood that not all progeny are identical to the parental cell, since there may be mutations that occur during replication. However, such progeny are included when the terms above are used. Preferred host cells of the invention include E. coli, S. aureus and P. aeruginosa, although other Gram negative and Gram positive organisms known in the art can be utilized as long as the expression vectors contain an origin of replication to permit expression in the host.

The polynucleotide sequence encoding the peptide used according to the method of the invention can be isolated from an organism or synthesized in the laboratory. Specific DNA sequences encoding the peptide of interest can be obtained by: 1) isolation of a double-stranded DNA sequence from the genomic DNA; 2) chemical manufacture of a DNA sequence to provide the necessary codons for the peptide of interest; and 3) in vitro synthesis of a double-stranded DNA sequence by reverse transcription of mRNA isolated from a donor cell. In the latter case, a double-stranded DNA complement of mRNA is eventually formed that is generally referred to as cDNA.

The synthesis of DNA sequences is frequently the method of choice when the entire sequence of amino acid residues of the desired peptide product is known. In the present invention, the synthesis of a DNA sequence has the advantage of allowing the incorporation of codons that are more likely to be recognized by a bacterial host, thereby permitting high level expression without difficulties in translation. In addition, virtually any peptide can be synthesized, including those encoding natural peptides, variants of the same, or synthetic peptides.

When the entire sequence of the desired peptide is not known, the direct synthesis of DNA sequences is not possible and the method of choice is the formation of cDNA sequences. Among the standard procedures for isolating cDNA sequences of interest is the formation of plasmid or phage containing cDNA libraries that are derived from reverse transcription of mRNA that is abundant in donor cells that have a high level of genetic expression. When used in combination with polymerase chain reaction technology, even rare expression products can be cloned. In those cases where significant portions of the amino acid sequence of the peptide are known, the production of labeled single or double-stranded DNA or RNA probe sequences duplicating a sequence putatively present in the target cDNA may be employed in DNA/DNA hybridization procedures which are carried out on cloned copies of the cDNA which have been denatured into a single stranded form (Jay, et al., Nuc. Acid Res., 11:2325, 1983).

### G. Methods of Use

### Anti-Biofilm

The invention also provides a method of inhibiting the biofilm growth of bacteria including contacting the bacteria with an inhibiting effective amount of a peptide of the invention, including SEQ ID NOS:1-749, and analogs, derivatives, enantiomers, amidated and unamidated variations and conservative variations thereof, wherein the peptides have antibiofilm activity.

The term “contacting” refers to exposing the bacteria to the peptide so that the peptide can effectively inhibit, kill, or cause dispersal of bacteria growing in the biofilm state. Contacting may be in vitro, for example by adding the peptide to a bacterial culture to test for susceptibility of the bacteria to the peptide or acting against biofilms that grow on abiotic surfaces. Contacting may be in vivo, for example administering the peptide to a subject with a bacterial disorder, such as septic shock or infection. Contacting may further involve coating an object (e.g., medical device) such as a catheter or prosthetic device to inhibit the production of biofilms by the bacteria with which it comes into contact, thus preventing it from becoming colonized with the bacteria. “Inhibiting” or “inhibiting effective amount” refers to the amount of peptide that is required to cause an anti-biofilm bacteriostatic or bactericidal effect. Examples of bacteria that may be inhibited include Escherichia coli, Pseudomonas aeruginosa, Klebsiella pneumoniae, Salmonella enteritidis subspecies Typhimurium, Campylobacter sp., Burkholderia complex bacteria, Acinetobacter baumanii, Staphylococcus aureus, Enterococcus facaelis, Listeria monocytogenes, and oral pathogens. Other potential targets are well known to the skilled microbiologist.

The method of inhibiting the growth of biofilm bacteria may further include the addition of antibiotics for combination or synergistic therapy. Antibiotics can work by either assisting the peptide in killing bacteria in biofilms or by inhibiting bacteria released from the biofilm due to accelerated dispersal by a peptide of the invention. Those antibiotics most suitable for combination therapy can be easily tested by utilizing modified checkerboard titration assays that use the determination of Fractional Inhibitory Concentrations to assess synergy as further described below. The appropriate antibiotic administered will typically depend on the susceptibility of the biofilms, including whether the bacteria is Gram negative or Gram positive, and will be discernible by one of skill in the art. Examples of particular classes of antibiotics useful for synergistic therapy with the peptides of the invention include aminoglycosides (e.g., tobramycin), penicillins (e.g., piperacillin), cephalosporins (e.g., ceftazidime), fluoroquinolones (e.g., ciprofloxacin), carbapenems (e.g., imipenem), tetracyclines, vancomycin, polymyxins and macrolides (e.g., erythromycin and clarithromycin). The method of inhibiting the growth of bacteria may further include the addition of antibiotics for combination or synergistic therapy. The appropriate antibiotic administered will typically depend on the susceptibility of the bacteria such as whether the bacteria is Gram negative or Gram positive, or whether synergy can be demonstrated in vitro, and will be easily discernable by one of skill in the art. Further to the antibiotics listed above, typical antibiotics include aminoglycosides (amikacin, gentamicin, kanamycin, netilmicin, tobramycin, streptomycin), macrolides (azithromycin, clarithromycin, erythromycin, erythromycin estolate/ethylsuccinate/gluceptate/lactobionate/stearate), beta-lactams such as penicillins (e.g., penicillin G, penicillin V, methicillin, nafcillin, oxacillin, cloxacillin, dicloxacillin, ampicillin, amoxicillin, ticarcillin, carbenicillin, mezlocillin, azlocillin and piperacillin), or cephalosporins (e.g., cephalothin, cefazolin, cefaclor, cefamandole, cefoxitin, cefuroxime, cefonicid, cefmetazole, cefotetan, cefprozil, loracarbef, cefetamet, cefoperazone, cefotaxime, ceftizoxime, ceftriaxone, ceftazidime, cefepime, cefixime, cefpodoxime, and cefsulodin) or carbapenems (e.g., imipenem, meropenem, panipenem), or monobactams (e.g., aztreonam). Other classes of antibiotics include quinolones (e.g., fleroxacin, nalidixic acid, norfloxacin, ciprofloxacin, ofloxacin, enoxacin, lomefloxacin and cinoxacin), tetracyclines (e.g., doxycycline, minocycline, tetracycline), and glycopeptides (e.g., vancomycin, teicoplanin), for example. Other antibiotics include chloramphenicol, clindamycin, trimethoprim, sulfamethoxazole, nitrofurantoin, rifampin, linezolid, synercid, polymyxin B, colistin, colimycin, methotrexate, daptomycin, phosphonomycin and mupirocin.

The peptides and/or analogs or derivatives thereof may be administered to any host, including a human or non-human animal, in an amount effective to inhibit not only the growth of a bacterium, but also a virus, parasite or fungus. These peptides are useful as antibiofilm agents, and immunomodulatory anti-infective agents, including anti-bacterial agents, antiviral agents, and antifungal agents.

The invention further provides a method of protecting objects from bacterial colonization. Bacteria grow on many surfaces as biofilms. The peptides of the invention are active in inhibiting bacteria on surfaces. Thus, the peptides may be used for protecting objects such as medical devices from biofilm colonization with pathogenic bacteria by, coating or chemically conjugating, or by any other means, at least one peptide of the invention to the surface of the medical device. Such medical devices include indwelling catheters, prosthetic devices, and the like. Removal of bacterial biofilms from medical equipment, plumbing in hospital wards and other areas where susceptible individuals congregate and the like is also a use for peptides of the invention.

### H. Methods of Use

### Immunomodulatory

The present invention provides novel cationic peptides, characterized by a group of related sequences and generic formulas that have ability to modulate (e.g., up- and/or down regulate) polypeptide expression, thereby regulating inflammatory responses, protective immunity and/or innate immunity.

“Innate immunity” as used herein refers to the natural ability of an organism to defend itself against invasion by pathogens. Pathogens or microbes as used herein, may include, but are not limited to bacteria, fungi, parasites, and viruses. Innate immunity is contrasted with acquired/adaptive immunity in which the organism develops a defensive mechanism based substantially on antibodies and/or immune lymphocytes that is characterized by specificity, amplifiability and self vs. non-self discrimination. With innate immunity, rapid and broad, relatively nonspecific immunity is provided, molecules from other species can be functional (i.e. there is a substantial lack of self vs. non-self discrimination) and there is no immunologic memory of prior exposure. The hallmarks of innate immunity are effectiveness against a broad variety of potential pathogens, independence of prior exposure to a pathogen, and immediate effectiveness (in contrast to the specific immune response which takes days to weeks to be elicited). However agents that stimulate innate immunity can have an impact on adaptive immunity since innate immunity instructs adaptive immunity ensuring an enhanced adaptive immune response (the underlying principle that guides the selection of adjuvants that are used in vaccines to enhance vaccine responses by stimulating innate immunity). Also the effector molecules and cells of innate immunity overlap strongly with the effectors of adaptive immunity. A feature of many of the IDR peptides revealed here is their ability to selectively stimulate innate immunity, enhancing adaptive immunity to vaccine antigens.

In addition, innate immunity includes immune and inflammatory responses that affect other diseases, such as: vascular diseases: atherosclerosis, cerebral/myocardial infarction, chronic venous disease, pre-eclampsia/eclampsia, and vasculitis; neurological diseases: Alzheimer's disease, Parkinson's disease, epilepsy, and amyotrophic lateral sclerosis (ALS); respiratory diseases: asthma, pulmonary fibrosis, cystic fibrosis, chronic obstructive pulmonary disease, and acute respiratory distress syndrome; dermatologic diseases: psoriasis, acne/rosacea, chronic urticaria, and eczema; gastro-intestinal diseases: celiac disease, inflammatory bowel disease, pancreatitis, esophagitis, gastronintestinal ulceration, and fatty liver disease (alcoholic/obese); endocrine diseases: thyroiditis, paraneoplastic syndrome, type 2 diabetes, hypothyroidism and hyperthyroidism; systemic diseases: cancer, sepsis; genito/urinary diseases: chronic kidney disease, nephrotic/nephritic syndrome, benign prostatic hyperplasia, cystitis, pelvic inflammatory disease, urethritis and urethral stricture; and musculoskeletal diseases: osteoporosis, systemic lupus erythematosis; rheumatoid arthritis, inflammatory myopathy, muscular sclerosis, osteoarthritis, costal chondritis and ankylosing spondylitis.

The innate immune system prevents pathogens, in small to modest doses (i.e. introduced through dermal contact, ingestion or inhalation), from colonizing and growing to a point where they can cause life-threatening infections. The major problems with stimulating innate immunity in the past have been created by the excessive production of pro-inflammatory cytokines. Excessive inflammation is associated with detrimental pathology. Thus while the innate immune system is essential for human survival, the outcome of an overly robust and/or inappropriate immune response can paradoxically result in harmful sequelae like e.g. sepsis or chronic inflammation such as with cystic fibrosis. A feature of the IDR peptides revealed here is their ability to selectively stimulate innate immunity, enhancing protective immunity while suppressing the microbially-induced production of pro-inflammatory cytokines.

In innate immunity, the immune response is not dependent upon antigens. The innate immunity process may include the production of secretory molecules and cellular components and the recruitment and differentiation of immune cells. In innate immunity triggered by an infection, molecules on the surface of or within pathogens are recognized by receptors (for example, pattern recognition receptors such as Toll-like receptors) that have broad specificity, are capable of recognizing many pathogens, and are encoded in the germline. When cationic peptides are present in the immune response, they modify (modulate) the host response to pathogens. This change in the immune response induces the release of chemokines, which promote the recruitment of immune cells to the site of infection, enhances the differentiation of immune cells into ones that are more effective in fighting infectious organisms and repairing wounds, and at the same time suppress the potentially harmful production of pro-inflammatory cytokines.

Chemokines, or chemoattractant cytokines, are a subgroup of immune factors that mediate chemotactic and other pro-inflammatory phenomena (See, Schall, 1991, Cytokine 3:165-183). Chemokines are small molecules of approximately 70-80 residues in length and can generally be divided into two subgroups, α which have two N-terminal cysteines separated by a single amino acid (CxC) and β which have two adjacent cysteines at the N terminus (CC). RANTES, MIP-1α and MIP-1β are members of the β subgroup (reviewed by Horuk, R., 1994, Trends Pharmacol. Sci, 15:159-165; Murphy, P. M., 1994, Annu. Rev. Immunol., 12:593-633). The amino terminus of the β chemokines RANTES, MCP-1, and MCP-3 have been implicated in the mediation of cell migration and inflammation induced by these chemokines. This involvement is suggested by the observation that the deletion of the amino terminal 8 residues of MCP-1, amino terminal 9 residues of MCP-3, and amino terminal 8 residues of RANTES and the addition of a methionine to the amino terminus of RANTES, antagonize the chemotaxis, calcium mobilization and/or enzyme release stimulated by their native counterparts (Gong et al., 1996 J. Biol. Chem. 271:10521-10527; Proudfoot et al., 1996 J. Biol. Chem. 271:2599-2603). Additionally, α chemokine-like chemotactic activity has been introduced into MCP-1 via a double mutation of Tyr 28 and Arg 30 to leucine and valine, respectively, indicating that internal regions of this protein also play a role in regulating chemotactic activity (Beall et al., 1992, J. Biol. Chem. 267:3455-3459).

The monomeric forms of all chemokines characterized thus far share significant structural homology, although the quaternary structures of α and β groups are distinct. While the monomeric structures of the β and α chemokines are very similar, the dimeric structures of the two groups are completely different. An additional chemokine, lymphotactin, which has only one N terminal cysteine has also been identified and may represent an additional subgroup (γ) of chemokines (Yoshida et al., 1995, FEBS Lett. 360:155-159; and Kelner et al., 1994, Science 266:1395-1399).

Receptors for chemokines belong to the large family of G-protein coupled, 7 transmembrane domain receptors (GCR's) (See, reviews by Horuk, R., 1994, Trends Pharmacol. Sci. 15:159-165; and Murphy, P. M., 1994, Annu. Rev. Immunol. 12:593-633). Competition binding and cross-desensitization studies have shown that chemokine receptors exhibit considerable promiscuity in ligand binding. Examples demonstrating the promiscuity among β chemokine receptors include: CC CKR-1, which binds RANTES and MIP-1α (Neote et al., 1993, Cell 72: 415-425), CC CKR-4, which binds RANTES, MIP-1α, and MCP-1 (Power et al., 1995, J. Biol. Chem. 270:19495-19500), and CC CKR-5, which binds RANTES, MIP-1α, and MIP-1β (Alkhatib et al., 1996, Science, in press and Dragic et al., 1996, Nature 381:667-674). Erythrocytes possess a receptor (known as the Duffy antigen) which binds both α and β chemokines (Horuk et al., 1994, J. Biol. Chem. 269:17730-17733; Neote et al., 1994, Blood 84:44-52; and Neote et al., 1993, J. Biol. Chem. 268:12247-12249). Thus the sequence and structural homologies evident among chemokines and their receptors allows some overlap in receptor-ligand interactions.

In one aspect, the present invention provides the use of compounds including peptides of the invention to suppress potentially harmful inflammatory responses by acting directly on host cells. In this aspect, a method of identification of a polynucleotide or polynucleotides that are regulated by one or more inflammation inducing agents is provided, where the regulation is altered by a cationic peptide. Such inflammation inducing agents include, but are not limited to endotoxic lipopolysaccharide (LPS), lipoteichoic acid (LTA), flagellin, polyinosinic:polycytidylic acid (PolyIC) and/or CpG DNA or intact bacteria or viruses or other bacterial or viral components. The identification is performed by contacting the host cell with the sepsis or inflammatory inducing agents and further contacting with a cationic peptide either before, simultaneously or immediately after. The expression of the polynucleotide or polypeptide in the presence and absence of the cationic peptide is observed and a change in expression is indicative of a polynucleotide or polypeptide or pattern of polynucleotides or polypeptides that is regulated by a sepsis or inflammatory inducing agent and inhibited by a cationic peptide. In another aspect, the invention provides a polynucleotide identified by the method.

Generally, in the methods of the invention, a cationic peptide is utilized to modulate the expression of a series of polynucleotides or polypeptides that are essential in the process of inflammation or protective immunity. The pattern of polynucleotide or polypeptide expression may be obtained by observing the expression in the presence and absence of the cationic peptide. The pattern obtained in the presence of the cationic peptide is then useful in identifying additional compounds that can inhibit expression of the polynucleotide and therefore block inflammation or stimulate protective immunity. It is well known to one of skill in the art that non-peptidic chemicals and peptidomimetics can mimic the ability of peptides to bind to receptors and enzyme binding sites and thus can be used to block or stimulate biological reactions. Where an additional compound of interest provides a pattern of polynucleotide or polypeptide expression similar to that of the expression in the presence of a cationic peptide, that compound is also useful in the modulation of an innate immune response to block inflammation or stimulate protective immunity. In this manner, the cationic peptides of the invention, which are known inhibitors of inflammation and enhancers of protective immunity are useful as tools in the identification of additional compounds that inhibit sepsis and inflammation and enhance innate immunity.

As can be seen in the Examples below, peptides of the invention have an ability to reduce the expression of polynucleotides or polypeptides regulated by LPS, particularly the quintessential pro-inflammatory cytokine TNFα. High levels of endotoxins in the blood are responsible for many of the symptoms seen during a serious infection or inflammation such as fever and an elevated white blood cell count, and many of these effects reflect or are caused by high levels of induced TNFα. Endotoxin (also called lipopolysaccharide) is a component of the cell envelope of Gram negative bacteria and is a potent trigger of the pathophysiology of sepsis. The basic mechanisms of inflammation and sepsis are interrelated.

In another aspect, the invention identifies agents that enhance innate immunity. Human cells that contain a polynucleotide or polynucleotides that encode a polypeptide or polypeptides involved in innate immunity are contacted with an agent of interest. Expression of the polynucleotide is determined, both in the presence and absence of the agent. The expression is compared and of the specific modulation of expression was indicative of an enhancement of innate immunity. In another aspect, the agent does not by itself stimulate an inflammatory response as revealed by the lack of upregulation of the pro-inflammatory cytokine TNF-α. In still another aspect the agent reduces or blocks the inflammatory or septic response. In yet another aspect the agent selectively stimulates innate immunity, thus promoting an adjuvant response and enhancing adaptive immunity to vaccine antigens.

In another aspect, the invention provides methods of direct polynucleotide or polypeptide regulation by cationic peptides and the use of compounds including cationic peptides to stimulate elements of innate immunity. In this aspect, the invention provides a method of identification of a pattern of polynucleotide or polypeptide expression for identification of a compound that enhances protective innate immunity. In the method of the invention, an initial detection of a pattern of polypeptide expression for cells contacted in the presence and absence of a cationic peptide is made. The pattern resulting from polypeptide expression in the presence of the peptide represents stimulation of protective innate immunity. A pattern of polypeptide expression is then detected in the presence of a test compound, where a resulting pattern with the test compound that is similar to the pattern observed in the presence of the cationic peptide is indicative of a compound that enhances protective innate immunity. In another aspect, the invention provides compounds that are identified in the above methods. In another aspect, the compound of the invention stimulates chemokine expression. Chemokines may include, but are not limited to Gro-α, MCP-1, and MCP-3. In still another aspect, the compound is a peptide, peptidomimetic, chemical compound, or a nucleic acid molecule.

It has been shown that cationic peptides can neutralize the host response to the signaling molecules of infectious agents as well as modify the transcriptional responses of host cells, mainly by down-regulating the pro-inflammatory response and/or up-regulating the anti-inflammatory response. Example 9 shows that the cationic peptides can selectively suppress the agonist stimulated induction of the inflammation inducing cytokine TNFα in host cells. Example 6 shows that the cationic peptides can aid in the host response to pathogens by inducing the release of chemokines, which promote the recruitment of immune cells to the site of infection.

It is seen from the examples below that cationic peptides have a substantial influence on the host response to pathogens in that they assist in regulation of the host immune response by inducing selective pro-inflammatory responses that for example promote the recruitment of immune cells to the site of infection but not inducing potentially harmful pro-inflammatory cytokines. The pathology associated with infections and sepsis appears to be caused in part by a potent pro-inflammatory response to infectious agents. Peptides can aid the host in a “balanced” response to pathogens by inducing an anti-inflammatory response and suppressing certain potentially harmful pro-inflammatory responses.

### I. Treatment Regimes

The invention provides pharmaceutical compositions comprising one or a combination of antimicrobial peptides, for example, formulated together with a pharmaceutically acceptable carrier. Some compositions include a combination of multiple (e.g., two or more) peptides of the invention.

As used herein “pharmaceutically acceptable carrier” includes any and all solvents, dispersion media, coatings, detergents, emulsions, lipids, liposomes and nanoparticles, antibacterial and antifungal agents, isotonic and absorption delaying agents, and the like that are physiologically compatible. In one embodiment, the carrier is suitable for parenteral administration. Alternatively, the carrier can be suitable for intravenous, intraperitoneal, intramuscular or topical administration. In another embodiment, the carrier is suitable for oral administration. Pharmaceutically acceptable carriers include sterile aqueous solutions or dispersions and sterile powders for the extemporaneous preparation of sterile injectable solutions or dispersion. The use of such media and agents for pharmaceutically active substances is well known in the art. Except insofar as any conventional media or agent is compatible with the active compound, use thereof in the pharmaceutical compositions is contemplated. Supplementary active compounds can also be incorporated into the compositions.

A “pharmaceutically acceptable salt” refers to a salt that retains the desired biological activity of the parent compound and does not impart any undesired toxicological effects (See, e.g., Berge, et al., J. Pharm. Sci., 66: 1-19, 1977). Examples of such salts include acid addition salts and base addition salts. Acid addition salts include those derived from nontoxic inorganic acids, such as hydrochloric, nitric, phosphoric, sulfuric, hydrobromic, hydroiodic, phosphorous and the like, as well as from nontoxic organic acids such as aliphatic mono- and dicarboxylic acids, phenyl-substituted alkanoic acids, hydroxy alkanoic acids, aromatic acids, aliphatic and aromatic sulfonic acids and the like. Base addition salts include those derived from alkaline earth metals, such as sodium, potassium, magnesium, calcium and the like, as well as from nontoxic organic amines, such as N,N′-dibenzylethylenediamine, N-methylglucamine, chloroprocaine, choline, diethanolamine, ethylenediamine, procaine and the like.

In prophylactic applications, pharmaceutical compositions or medicaments are administered to a patient susceptible to, or otherwise at risk of a disease or condition (i.e., as a result of bacteria, fungi, viruses, parasites or the like) in an amount sufficient to eliminate or reduce the risk, lessen the severity, or delay the outset of the disease, including biochemical, histologic and/or behavioral symptoms of the disease, its complications and intermediate pathological phenotypes presenting during development of the disease. In therapeutic applications, compositions or medicants are administered to a patient suspected of, or already suffering from such a disease or condition in an amount sufficient to cure, or at least partially arrest, the symptoms of the disease or condition (e.g., biochemical and/or histologic), including its complications and intermediate pathological phenotypes in development of the disease or condition. An amount adequate to accomplish therapeutic or prophylactic treatment is defined as a therapeutically- or prophylactically-effective dose. In both prophylactic and therapeutic regimes, agents are usually administered in several dosages until a sufficient response has been achieved. Typically, the response is monitored and repeated dosages are given if the response starts to wane.

The pharmaceutical composition of the present invention should be sterile and fluid to the extent that the composition is deliverable by syringe. In addition to water, the carrier can be an isotonic buffered saline solution, ethanol, polyol (for example, glycerol, propylene glycol, and liquid polyetheylene glycol, and the like), and suitable mixtures thereof. Proper fluidity can be maintained, for example, by use of coating such as lecithin, by maintenance of required particle size in the case of dispersion and by use of surfactants. In many cases, it is preferable to include isotonic agents, for example, sugars, polyalcohols such as mannitol or sorbitol, and sodium chloride in the composition. Long-term absorption of the injectable compositions can be brought about by including in the composition an agent which delays absorption, for example, aluminum monostearate or gelatin.

When the active compound is suitably protected, as described above, the compound can be orally administered, for example, with an inert diluent or an assimilable edible carrier.

Pharmaceutical compositions of the invention also can be administered in combination therapy, i.e., combined with other agents. For example, in treatment of bacteria, the combination therapy can include a composition of the present invention with at least one agent or other conventional therapy.

### J. Routes of Administration

A composition of the present invention can be administered by a variety of methods known in the art. The route and/or mode of administration vary depending upon the desired results. The phrases “parenteral administration” and “administered parenterally” mean modes of administration other than enteral and topical administration, usually by injection, and includes, without limitation, intravenous, intramuscular, intraarterial, intrathecal, intracapsular, intraorbital, intracardiac, intradermal, intraperitoneal, transtracheal, subcutaneous, subcuticular, intraarticular, subcapsular, subarachnoid, intraspinal, epidural and intrasternal injection and infusion. The peptide of the invention can be administered parenterally by injection or by gradual infusion over time. The peptide can also be prepared with carriers that protect the compound against rapid release, such as a controlled release formulation, including implants, transdermal patches, and microencapsulated delivery systems. Further methods for delivery of the peptide include orally, by encapsulation in microspheres or proteinoids, by aerosol delivery to the lungs, or transdermally by iontophoresis or transdermal electroporation.

The peptides may also be delivered via transdermal or topical application. Transdermal and topical dosage forms of the invention include, but are not limited to, creams, lotions, ointments, gels, solutions, emulsions, suspensions, or other forms known to one of skill in the art. See, e.g., Remington's Pharmaceutical Sciences, 18th eds., Mack Publishing, Easton Pa. (1990); and Introduction to Pharmaceutical Dosage Forms, 4th ed., Lea & Febiger, Philadelphia (1985). Transdermal dosage forms include “reservoir type” or “matrix type” patches, which can be applied to the skin and worn for a specific period of time to permit the penetration of a desired amount of active ingredients.

Suitable excipients (e.g., carriers and diluents) and other materials that can be used to provide transdermal and topical dosage forms encompassed by this invention are well known to those skilled in the pharmaceutical arts, and will depend on the particular tissue to which a given pharmaceutical composition or dosage form will be applied. For example, typical excipients include, but are not limited to, water, acetone, ethanol, ethylene glycol, propylene glycol, butane-1,3-diol, isopropyl myristate, isopropyl palmitate, lipids, nanoparticles, mineral oil, and mixtures thereof to form lotions, tinctures, creams, emulsions, gels or ointments, which are non-toxic and pharmaceutically acceptable. Moisturizers or humectants can also be added to pharmaceutical compositions and dosage forms if desired. Examples of such additional ingredients are well known in the art. See, e.g., Remington's Pharmaceutical Sciences, 18th eds., Mack Publishing, Easton Pa. (1990).

Depending on the specific tissue to be treated, additional components may be used prior to, in conjunction with, or subsequent to treatment with active ingredients of the invention. For example, penetration enhancers can be used to assist in delivering the active ingredients to the tissue. Suitable penetration enhancers include, but are not limited to: acetone; various alcohols such as ethanol, oleyl, and tetrahydrofuryl; alkyl sulfoxides such as dimethyl sulfoxide; dimethyl acetamide; dimethyl formamide; polyethylene glycol; pyrrolidones such as polyvinylpyrrolidone; Kollidon grades (Povidone, Polyvidone); urea; and various water-soluble or insoluble sugar esters such as Tween 80 (polysorbate 80) and Span 60 (sorbitan monostearate).

To administer a peptide of the invention by certain routes of administration, it can be necessary to coat the compound with, or co-administer the compound with, a material to prevent its inactivation. The method of the invention also includes delivery systems such as microencapsulation of peptides into liposomes or a diluent. Microencapsulation also allows co-entrapment of antimicrobial molecules along with the antigens, so that these molecules, such as antibiotics, may be delivered to a site in need of such treatment in conjunction with the peptides of the invention. Liposomes in the blood stream are generally taken up by the liver and spleen. Pharmaceutically acceptable diluents include saline and aqueous buffer solutions. Liposomes include water-in-oil-in-water CGF emulsions as well as conventional liposomes (Strejan, et al., J. Neuroimmunol., 7: 27, 1984). Thus, the method of the invention is particularly useful for delivering antimicrobial peptides to such organs. Biodegradable, biocompatible polymers can be used, such as ethylene vinyl acetate, polyanhydrides, polyglycolic acid, collagen, polyorthoesters, and polylactic acid. Many methods for the preparation of such formulations are described by e.g., Sustained and Controlled Release Drug Delivery Systems, J. R. Robinson, Ed., 1978, Marcel Dekker, Inc., New York. Other methods of administration will be known to those skilled in the art.

Preparations for parenteral administration of a peptide of the invention include sterile aqueous or non-aqueous solutions, suspensions, and emulsions. Examples of non-aqueous solvents are propylene glycol, polyethylene glycol, vegetable oils such as olive oil, and injectable organic esters such as ethyl oleate. Aqueous carriers include water, alcoholic/aqueous solutions, emulsions or suspensions, including saline and buffered media. Parenteral vehicles include sodium chloride solution, Ringer's dextrose, dextrose and sodium chloride, lactated Ringer's, or fixed oils. Intravenous vehicles include fluid and nutrient replenishers, electrolyte replenishers (such as those based on Ringer's dextrose), and the like. Preservatives and other additives may also be present such as, for example, antimicrobials, anti-oxidants, chelating agents, and inert gases and the like.

Therapeutic compositions typically must be sterile, substantially isotonic, and stable under the conditions of manufacture and storage. The composition can be formulated as a solution, microemulsion, liposome, or other ordered structure suitable to high drug concentration. The carrier can be a solvent or dispersion medium containing, for example, water, ethanol, polyol (for example, glycerol, propylene glycol, and liquid polyethylene glycol, and the like), and suitable mixtures thereof. The proper fluidity can be maintained, for example, by the use of a coating such as lecithin, by the maintenance of the required particle size in the case of dispersion and by the use of surfactants. In many cases, it is preferable to include isotonic agents, for example, sugars, polyalcohols such as mannitol, sorbitol, or sodium chloride in the composition. Prolonged absorption of the injectable compositions can be brought about by including in the composition an agent that delays absorption, for example, monostearate salts and gelatin.

Sterile injectable solutions can be prepared by incorporating the active compound in the required amount in an appropriate solvent with one or a combination of ingredients enumerated above, as required, followed by sterilization microfiltration. Generally, dispersions are prepared by incorporating the active compound into a sterile vehicle that contains a basic dispersion medium and the required other ingredients from those enumerated above. In the case of sterile powders for the preparation of sterile injectable solutions, the preferred methods of preparation are vacuum drying and freeze-drying (lyophilization) that yield a powder of the active ingredient plus any additional desired ingredient from a previously sterile-filtered solution thereof. Therapeutic compositions can also be administered with medical devices known in the art. For example, in a preferred embodiment, a therapeutic composition of the invention can be administered with a needleless hypodermic injection device, such as the devices disclosed in, e.g., U.S. Pat. Nos. 5,399,163, 5,383,851, 5,312,335, 5,064,413, 4,941,880, 4,790,824, or 4,596,556. Examples of implants and modules useful in the present invention include: U.S. Pat. No. 4,487,603, which discloses an implantable micro-infusion pump for dispensing medication at a controlled rate; U.S. Pat. No. 4,486,194, which discloses a therapeutic device for administering medicants through the skin; U.S. Pat. No. 4,447,233, which discloses a medication infusion pump for delivering medication at a precise infusion rate; U.S. Pat. No. 4,447,224, which discloses a variable flow implantable infusion apparatus for continuous drug delivery; U.S. Pat. No. 4,439,196, which discloses an osmotic drug delivery system having multi-chamber compartments; and U.S. Pat. No. 4,475,196, which discloses an osmotic drug delivery system. Many other such implants, delivery systems, and modules are known.

When the peptides of the present invention are administered as pharmaceuticals, to humans and animals, they can be given alone or as a pharmaceutical composition containing, for example, 0.01 to 99.5% (or 0.1 to 90%) of active ingredient in combination with a pharmaceutically acceptable carrier.

### K. Effective Dosages

“Therapeutically effective amount” as used herein for treatment of antimicrobial related diseases and conditions refers to the amount of peptide used that is of sufficient quantity to decrease the numbers of bacteria, viruses, fungi, and parasites in the body of a subject. The dosage ranges for the administration of peptides are those large enough to produce the desired effect. The amount of peptide adequate to accomplish this is defined as a “therapeutically effective dose.” The dosage schedule and amounts effective for this use, i.e., the “dosing regimen,” will depend upon a variety of factors, including the stage of the disease or condition, the severity of the disease or condition, the general state of the patient's health, the patient's physical status, age, pharmaceutical formulation and concentration of active agent, and the like. In calculating the dosage regimen for a patient, the mode of administration also is taken into consideration. The dosage regimen must also take into consideration the pharmacokinetics, i.e., the pharmaceutical composition's rate of absorption, bioavailability, metabolism, clearance, and the like. See, e.g., the latest Remington's (Remington's Pharmaceutical Science, Mack Publishing Company, Easton, Pa.); Egleton, Peptides 18: 1431-1439, 1997; Langer Science 249: 1527-1533, 1990. The dosage regimen can be adjusted by the individual physician in the event of any contraindications.

Dosage regimens of the pharmaceutical compositions of the present invention are adjusted to provide the optimum desired response (e.g., a therapeutic response). For example, a single bolus can be administered, several divided doses can be administered over time or the dose can be proportionally reduced or increased as indicated by the exigencies of the therapeutic situation. It is especially advantageous to formulate parenteral compositions in dosage unit form for ease of administration and uniformity of dosage. Dosage unit form as used herein refers to physically discrete units suited as unitary dosages for the subjects to be treated; each unit contains a predetermined quantity of active compound calculated to produce the desired therapeutic effect in association with the required pharmaceutical carrier. The specification for the dosage unit forms of the invention are dictated by and directly dependent on (a) the unique characteristics of the active compound and the particular therapeutic effect to be achieved, and (b) the limitations inherent in the art of compounding such an active compound for the treatment of sensitivity in individuals.

Actual dosage levels of the active ingredients in the pharmaceutical compositions of the present invention can be varied so as to obtain an amount of the active ingredient which is effective to achieve the desired therapeutic response for a particular patient, composition, and mode of administration, without being toxic to the patient. The selected dosage level depends upon a variety of pharmacokinetic factors including the activity of the particular compositions of the present invention employed, or the ester, salt or amide thereof, the route of administration, the time of administration, the rate of excretion of the particular compound being employed, the duration of the treatment, other drugs, compounds and/or materials used in combination with the particular compositions employed, the age, sex, weight, condition, general health and prior medical history of the patient being treated, and like factors.

A physician or veterinarian can start doses of the compounds of the invention employed in the pharmaceutical composition at levels lower than that required to achieve the desired therapeutic effect and gradually increase the dosage until the desired effect is achieved. In general, a suitable daily dose of a compound of the invention is that amount of the compound which is the lowest dose effective to produce a therapeutic effect. Such an effective dose generally depends upon the factors described above. It is preferred that administration be intravenous, intramuscular, intraperitoneal, or subcutaneous, or administered proximal to the site of the target. If desired, the effective daily dose of a therapeutic composition can be administered as two, three, four, five, six or more sub-doses administered separately at appropriate intervals throughout the day, optionally, in unit dosage forms. While it is possible for a compound of the present invention to be administered alone, it is preferable to administer the compound as a pharmaceutical formulation (composition).

An effective dose of each of the peptides disclosed herein as potential therapeutics for use in treating microbial diseases and conditions is from about 1 μg/kg to 500 mg/kg body weight, per single administration, which can readily be determined by one skilled in the art. As discussed above, the dosage depends upon the age, sex, health, and weight of the recipient, kind of concurrent therapy, if any, and frequency of treatment. Other effective dosage range upper limits are 50 mg/kg body weight, 20 mg/kg body weight, 8 mg/kg body weight, and 2 mg/kg body weight.

The dosage and frequency of administration can vary depending on whether the treatment is prophylactic or therapeutic. In prophylactic applications, a relatively low dosage is administered at relatively infrequent intervals over a long period of time. Some patients continue to receive treatment for the rest of their lives. In therapeutic applications, a relatively high dosage at relatively short intervals is sometimes required until progression of the disease is reduced or terminated, and preferably until the patient shows partial or complete amelioration of symptoms of disease. Thereafter, the patent can be administered a prophylactic regime.

Some compounds of the invention can be formulated to ensure proper distribution in vivo. For example, the blood-brain barrier (BBB) excludes many highly hydrophilic compounds. To ensure that the therapeutic compounds of the invention cross the BBB (if desired), they can be formulated, for example, in liposomes. For methods of manufacturing liposomes, See, e.g., U.S. Pat. Nos. 4,522,811; 5,374,548; and 5,399,331. The liposomes can comprise one or more moieties which are selectively transported into specific cells or organs, thus enhance targeted drug delivery (See, e.g., Ranade, J. Clin. Pharmacol., 29: 685, 1989). Exemplary targeting moieties include folate or biotin (See, e.g., U.S. Pat. No. 5,416,016 to Low, et al.); mannosides (Umezawa, et al., Biochem. Biophys. Res. Commun., 153: 1038, 1988); antibodies (Bloeman, et al., FEBS Lett., 357: 140, 1995; Owais, et al., Antimicrob. Agents Chemother., 39: 180, 1995); surfactant protein A receptor (Briscoe, et al., Am. J. Physiol., 1233: 134, 1995), different species of which can comprise the formulations of the inventions, as well as components of the invented molecules; p120 (Schreier, et al., J. Biol. Chem., 269: 9090, 1994); See also Keinanen, et al., FEBS Lett., 346: 123, 1994; Killion, et al., Immunomethods, 4: 273, 1994. In some methods, the therapeutic compounds of the invention are formulated in liposomes; in a more preferred embodiment, the liposomes include a targeting moiety. In some methods, the therapeutic compounds in the liposomes are delivered by bolus injection to a site proximal to the tumor or infection. The composition should be fluid to the extent that easy syringability exists. It should be stable under the conditions of manufacture and storage and should be preserved against the contaminating action of microorganisms such as bacteria and fungi.

“Anti-biofilm amount” as used herein refers to an amount sufficient to achieve a biofilm-inhibiting blood concentration in the subject receiving the treatment. The anti-bacterial amount of an antibiotic generally recognized as safe for administration to a human is well known in the art, and as is known in the art, varies with the specific antibiotic and the type of bacterial infection being treated.

Because of the broad spectrum anti-biofilm properties of the peptides, they may also be used as preservatives or to prevent formation of biofilms on materials susceptible to microbial biofilm contamination. The peptides of the invention can be utilized as broad spectrum anti-biofilm agents directed toward various specific applications. Such applications include use of the peptides as preservatives for processed foods (organisms including Salmonella, Yersinia, Shigella, Pseudomonas and Listeria), either alone or in combination with antibacterial food additives such as lysozymes; as a topical agent (Pseudomonas, Streptococcus, Staphylococcus) and to kill odor producing microbes (Micrococci). The relative effectiveness of the peptides of the invention for the applications described can be readily determined by one of skill in the art by determining the sensitivity of biofilms formed by any organism to one of the peptides.

### L. Formulation

Typically, compositions are prepared as injectables, either as liquid solutions or suspensions; solid forms suitable for solution in, or suspension in, liquid vehicles prior to injection can also be prepared. The preparation also can be emulsified or encapsulated in liposomes or micro particles such as polylactide, polyglycolide, or copolymer for enhanced adjuvant effect, as discussed above. Langer, Science 249: 1527, 1990 and Hanes, Advanced Drug Delivery Reviews 28: 97-119, 1997. The agents of this invention can be administered in the form of a depot injection or implant preparation which can be formulated in such a manner as to permit a sustained or pulsatile release of the active ingredient.

Additional formulations suitable for other modes of administration include oral, intranasal, topical and pulmonary formulations, suppositories, and transdermal applications.

For suppositories, binders and carriers include, for example, polyalkylene glycols or triglycerides; such suppositories can be formed from mixtures containing the active ingredient in the range of 0.5% to 10%, preferably 1%-2%. Oral formulations include excipients, such as pharmaceutical grades of mannitol, lactose, starch, magnesium stearate, sodium saccharine, detergents like Tween or Brij, PEGylated lipids, cellulose, and magnesium carbonate. These compositions take the form of solutions, suspensions, tablets, pills, capsules, sustained release formulations or powders and contain 10%-95% of active ingredient, preferably 25%-70%.

Topical application can result in transdermal or intradermal delivery, or enable activity against local biofilm infections. Co-administration can be achieved by using the components as a mixture or as linked molecules obtained by chemical crosslinking or expression as a fusion protein.

Alternatively, transdermal delivery can be achieved using a skin patch or using transferosomes. Paul et al., Eur. J. Immunol. 25: 3521-24, 1995; Cevc et al., Biochem. Biophys. Acta 1368: 201-15, 1998.

The pharmaceutical compositions are generally formulated as sterile, substantially isotonic and in full compliance with all Good Manufacturing Practice (GMP) regulations of the U.S. Food and Drug Administration.

From the foregoing description, various modifications and changes in the compositions and methods will occur to those skilled in the art. All such modifications coming within the scope of the appended claims are intended to be included therein. Each recited range includes all combinations and sub-combinations of ranges, as well as specific numerals contained therein.

All publications and patent documents cited above are hereby incorporated by reference in their entirety for all purposes to the same extent as if each were so individually denoted.

Although the foregoing invention has been described in detail by way of example for purposes of clarity of understanding, it will be apparent to the artisan that certain changes and modifications are comprehended by the disclosure and can be practiced without undue experimentation within the scope of the appended claims, which are presented by way of illustration not limitation.

### EXEMPLARY EMBODIMENTS

### Example 1

### Materials, Methods and Peptides

Peptide Synthesis—

All peptides used in this study, as listed in Table 1, were synthesized by GenScript (Piscataway, N.J., USA), or other suitable companies, using solid phase Fmoc chemistry and purified to a purity >95% using reverse phase HPLC, or were synthesized on cellulose membranes by SPOT synthesis. Peptide mass was confirmed by mass spectrometry. SPOT peptide syntheses on cellulose were performed using a pipetting robot (Abimed, Langenfeld, Germany) and Whatman 50 cellulose membranes (Whatman, Maidstone, United Kingdom) as described previously (Kramer A, Schuster A, Reinecke U, Malin R, Volkmer-Engert R, Landgraf C, Schneider-Mergener J. 1994. Combinatorial cellulose-bound peptide libraries: screening tool for the identification of peptides that bind ligands with predefined specificity. Comp. Meth. Enzymol. 6, 388-395; Kramer A, Keitel T, Winkler K, Stocklein W, Hohne W, Schneider-Mergener J. 1997. Molecular basis for the binding promiscuity of an anti-p24 (HIV-1) monoclonal antibody. Cell 91, 799-809).

TABLE 1
List of peptides and their sequences.

| | | Sequences (all peptides are |
|---|---|---|
| Seq | | amidated; sequences with D or RI |
| ID | Peptide | in front of their names are D |
| No | name | amino acid containing) |
| 1 | HE1 | RRWIRVAVILRV |
| 2 | HE4 | VRLIWAVRIWRR |
| 3 | HE10 | VRLIVRIWRR |
| 4 | HE12 | RFKRVARVIW |
| 5 | RI1012 | FKKVIVIRRWFI |
| 6 | RI1018 | RRWIRVAVILRV |
| 7 | RI1002 | KRIRWVILWRQV |
| 8 | RI1035 | RRINRVIWRWRK |
| 9 | RIJK2 | RIVWVRIRRWFV |
| 10 | RIJK3 | RIVRVRIARLQV |
| 11 | RIJK4 | RIVWVRIRRLQV |
| 12 | RIJK6 | RIVWVRIRRWQV |
| 13 | VKJ15 | RFRIRVRR |
| 14 | EH3 | VRVAVRIWRR |
| 15 | EH4 | VRLIPAVRIWRR |
| 16 | VKJ10-4 | KQFRIRVRVWIK |
| 17 | HE5 | VRLIRIWVRIWR |
| 18 | HE11 | RFKVAVRIWRR |
| 19 | HE6 | VRLIRAVRIWRR |
| 20 | 1010RW | IRWRIRVRVRWI |
| 21 | 1020RK | VRLRIRWRKLWV |
| 22 | 1018-G1 | GRLIVAVRIWRR |
| 23 | 1018-G2 | VGLIVAVRIWRR |
| 24 | 1018-G3 | VRGIVAVRIWRR |
| 25 | 1018-G4 | VRLGVAVRIWRR |
| 26 | 1018-G5 | VRLIGAVRIWRR |
| 27 | 1018-G6 | VRLIVGVRIWRR |
| 28 | 1018-G7 | VRLIVAGRIWRR |
| 29 | 1018-G8 | VRLIVAVGIWRR |
| 30 | 1018-G9 | VRLIVAVRGWRR |
| 31 | 1018-G10 | VRLIVAVRIGRR |
| 32 | 1018-G11 | VRLIVAVRIWGR |
| 33 | 1018-G12 | VRLIVAVRIWRG |
| 34 | 1018-A1 | ARLIVAVRIWRR |
| 35 | 1018-A2 | VALIVAVRIWRR |
| 36 | 1018-A3 | VRAIVAVRIWRR |
| 37 | 1018-A4 | VRLAVAVRIWRR |
| 38 | 1018-A5 | VRLIAAVRIWRR |
| 39 | 1018-A7 | VRLIVAARIWRR |
| 40 | 1018-A8 | VRLIVAVAIWRR |
| 41 | 1018-A9 | VRLIVAVRAWRR |
| 42 | 1018-A10 | VRLIVAVRIARR |
| 43 | 1018-A11 | VRLIVAVRIWAR |
| 44 | 1018-A12 | VRLIVAVRIWRA |
| 45 | 1018-R1 | RRLIVAVRIWRR |
| 46 | 1018-R3 | VRRIVAVRIWRR |
| 47 | 1018-R4 | VRLRVAVRIWRR |
| 48 | 1018-R5 | VRLIRAVRIWRR |
| 49 | 1018-R6; | VRLIVRVRIWRR |
| | 2005 | |
| 50 | 1018-R7 | VRLIVARRIWRR |
| 51 | 1018-R9 | VRLIVAVRRWRR |
| 52 | 1018-R10; | VRLIVAVRIRRR |
| | 2002 | |
| 53 | 1018-K1 | KRLIVAVRIWRR |
| 54 | 1018-K2 | VKLIVAVRIWRR |
| 55 | 1018-K3 | VRKIVAVRIWRR |
| 56 | 1018-K4 | VRLKVAVRIWRR |
| 57 | 1018-K5 | VRLIKAVRIWRR |
| 58 | 1018-K6; | VRLIVKVRIWRR |
| | 2001 | |
| 59 | 1018-K7 | VRLIVAKRIWRR |
| 60 | 1018-K8 | VRLIVAVKIWRR |
| 61 | 1018-K9 | VRLIVAVRKWRR |
| 62 | 1018-K10 | VRLIVAVRIKRR |
| 63 | 1018-K11 | VRLIVAVRIWKR |
| 64 | 1018-K12 | VRLIVAVRIWRK |
| 65 | 1018-L1 | LRLIVAVRIWRR |
| 66 | 1018-L2 | VLLIVAVRIWRR |
| 67 | 1018-L4 | VRLLVAVRIWRR |
| 68 | 1018-L5 | VRLILAVRIWRR |
| 69 | 1018-L6 | VRLIVLVRIWRR |
| 70 | 1018-L7 | VRLIVALRIWRR |
| 71 | 1018-L8 | VRLIVAVLIWRR |
| 72 | 1018-L9 | VRLIVAVRLWRR |
| 73 | 1018-L10 | VRLIVAVRILRR |
| 74 | 1018-L11 | VRLIVAVRIWLR |
| 75 | 1018-L12 | VRLIVAVRIWRL |
| 76 | 1018-I1 | IRLIVAVRIWRR |
| 77 | 1018-I2 | VILIVAVRIWRR |
| 78 | 1018-I3 | VRIIVAVRIWRR |
| 79 | 1018-I5 | VRLIIAVRIWRR |
| 80 | 1018-I6 | VRLIVIVRIWRR |
| 81 | 1018-I7 | VRLIVAIRIWRR |
| 82 | 1018-I8 | VRLIVAVIIWRR |
| 83 | 1018-I10 | VRLIVAVRIIRR |
| 84 | 1018-I11 | VRLIVAVRIWIR |
| 85 | 1018-I12 | VRLIVAVRIWRI |
| 86 | 1018-V2 | VVLIVAVRIWRR |
| 87 | 1018-V3 | VRVIVAVRIWRR |
| 88 | 1018-V4 | VRLVVAVRIWRR |
| 89 | 1018-V6 | VRLIVVVRIWRR |
| 90 | 1018-V8 | VRLIVAVVIWRR |
| 91 | 1018-V9 | VRLIVAVRVWRR |
| 92 | 1018-V10 | VRLIVAVRIVRR |
| 93 | 1018-V11 | VRLIVAVRIWVR |
| 94 | 1018-V12 | VRLIVAVRIWRV |
| 95 | 1018-W1 | WRLIVAVRIWRR |
| 96 | 1018-W2 | VWLIVAVRIWRR |
| 97 | 1018-W3 | VRWIVAVRIWRR |
| 98 | 1018-W4 | VRLWVAVRIWRR |
| 99 | 1018-W5 | VRLIWAVRIWRR |
| 100 | 1018-W6 | VRLIVWVRIWRR |
| 101 | 1018-W7 | VRLIVAWRIWRR |
| 102 | 1018-W8 | VRLIVAVWIWRR |
| 103 | 1018-W9 | VRLIVAVRWWRR |
| 104 | 1018-W11 | VRLIVAVRIWWR |
| 105 | 1018-W12 | VRLIVAVRIWRW |
| 106 | 1018-Q1 | QRLIVAVRIWRR |
| 107 | 1018-Q2 | VQLIVAVRIWRR |
| 108 | 1018-Q3 | VRQIVAVRIWRR |
| 109 | 1018-Q4 | VRLQVAVRIWRR |
| 110 | 1018-Q5 | VRLIQAVRIWRR |
| 111 | 1018-Q6 | VRLIVQVRIWRR |
| 112 | 1018-Q7 | VRLIVAQRIWRR |
| 113 | 1018-Q8 | VRLIVAVQIWRR |
| 114 | 1018-Q9 | VRLIVAVRQWRR |
| 115 | 1018-Q10 | VRLIVAVRIQRR |
| 116 | 1018-Q11 | VRLIVAVRIWQR |
| 117 | 1018-Q12 | VRLIVAVRIWRQ |
| 118 | 1002-G1 | GQRWLIVWRIRK |
| 119 | 1002-G2 | VGRWLIVWRIRK |
| 120 | 1002-G3 | VQGWLIVWRIRK |
| 121 | 1002-G4 | VQRGLIVWRIRK |
| 122 | 1002-G5 | VQRWGIVWRIRK |
| 123 | 1002-G6 | VQRWLGVWRIRK |
| 124 | 1002-G7 | VQRWLIGWRIRK |
| 125 | 1002-G8 | VQRWLIVGRIRK |
| 126 | 1002-G9 | VQRWLIVWGIRK |
| 127 | 1002-G10 | VQRWLIVWRGRK |
| 128 | 1002-G11 | VQRWLIVWRIGK |
| 129 | 1002-G12 | VQRWLIVWRIRG |
| 130 | 1002-A1 | AQRWLIVWRIRK |
| 131 | 1002-A2 | VARWLIVWRIRK |
| 132 | 1002-A3 | VQAWLIVWRIRK |
| 133 | 1002-A4 | VQRALIVWRIRK |
| 134 | 1002-A5 | VQRWAIVWRIRK |
| 135 | 1002-A6 | VQRWLAVWRIRK |
| 136 | 1002-A7 | VQRWLIAWRIRK |
| 137 | 1002-A8 | VQRWLIVARIRK |
| 138 | 1002-A9 | VQRWLIVWAIRK |
| 139 | 1002-A10 | VQRWLIVWRARK |
| 140 | 1002-A11 | VQRWLIVWRIAK |
| 141 | 1002-Al2 | VQRWLIVWRIRA |
| 142 | 1002-R1 | RQRWLIVWRIRK |
| 143 | 1002-R2 | VRRWLIVWRIRK |
| 144 | 1002-R4 | VQRRLIVWRIRK |
| 145 | 1002-R5 | VQRWRIVWRIRK |
| 146 | 1002-R6 | VQRWLRVWRIRK |
| 147 | 1002-R7 | VQRWLIRWRIRK |
| 148 | 1002-R8 | VQRWLIVRRIRK |
| 149 | 1002-R10 | VQRWLIVWRRRK |
| 150 | 1002-R12 | VQRWLIVWRIRR |
| 151 | 1002-K1 | KQRWLIVWRIRK |
| 152 | 1002-K2 | VKRWLIVWRIRK |
| 153 | 1002-K3 | VQKWLIVWRIRK |
| 154 | 1002-K4 | VQRKLIVWRIRK |
| 155 | 1002-K5 | VQRWKIVWRIRK |
| 156 | 1002-K6 | VQRWLKVWRIRK |
| 157 | 1002-K7 | VQRWLIKWRIRK |
| 158 | 1002-K8 | VQRWLIVKRIRK |
| 159 | 1002-K9 | VQRWLIVWKIRK |
| 160 | 1002-K10 | VQRWLIVWRKRK |
| 161 | 1002-K11 | VQRWLIVWRIKK |
| 162 | 1002-L1 | LQRWLIVWRIRK |
| 163 | 1002-L2 | VLRWLIVWRIRK |
| 164 | 1002-L3 | VQLWLIVWRIRK |
| 165 | 1002-L4 | VQRLLIVWRIRK |
| 166 | 1002-L6 | VQRWLLVWRIRK |
| 167 | 1002-L7 | VQRWLILWRIRK |
| 168 | 1002-L8 | VQRWLIVLRIRK |
| 169 | 1002-L9 | VQRWLIVWLIRK |
| 170 | 1002-L10 | VQRWLIVWRLRK |
| 171 | 1002-L11 | VQRWLIVWRILK |
| 172 | 1002-L12 | VQRWLIVWRIRL |
| 173 | 1002-I1 | IQRWLIVWRIRK |
| 174 | 1002-I2 | VIRWLIVWRIRK |
| 175 | 1002-I3 | VQIWLIVWRIRK |
| 176 | 1002-I4 | VQRILIVWRIRK |
| 177 | 1002-I5 | VQRWIIVWRIRK |
| 178 | 1002-I7 | VQRWLIIWRIRK |
| 179 | 1002-I8 | VQRWLIVIRIRK |
| 180 | 1002-I9 | VQRWLIVWIIRK |
| 181 | 1002-I11 | VQRWLIVWRIIK |
| 182 | 1002-I12 | VQRWLIVWRIRI |
| 183 | 1002-V2 | VVRWLIVWRIRK |
| 184 | 1002-V3 | VQVWLIVWRIRK |
| 185 | 1002-V4 | VQRVLIVWRIRK |
| 186 | 1002-V5 | VQRWVIVWRIRK |
| 187 | 1002-V6 | VQRWLVVWRIRK |
| 188 | 1002-V8 | VQRWLIVVRIRK |
| 189 | 1002-V9 | VQRWLIVWVIRK |
| 190 | 1002-V10 | VQRWLIVWRVRK |
| 191 | 1002-V11 | VQRWLIVWRIVK |
| 192 | 1002-V12 | VQRWLIVWRIRV |
| 193 | 1002-W1 | WQRWLIVWRIRK |
| 194 | 1002-W2 | VWRWLIVWRIRK |
| 195 | 1002-W3 | VQWWLIVWRIRK |
| 196 | 1002-W5 | VQRWWIVWRIRK |
| 197 | 1002-W6 | VQRWLWVWRIRK |
| 198 | 1002-W7 | VQRWLIWWRIRK |
| 199 | 1002-W9 | VQRWLIVWWIRK |
| 200 | 1002-W10 | VQRWLIVWRWRK |
| 201 | 1002-W11 | VQRWLIVWRIWK |
| 202 | 1002-W12 | VQRWLIVWRIRW |
| 203 | 1002-Q1 | QQRWLIVWRIRK |
| 204 | 1002-Q3 | VQQWLIVWRIRK |
| 205 | 1002-Q4 | VQRQLIVWRIRK |
| 206 | 1002-Q5 | VQRWQIVWRIRK |
| 207 | 1002-Q6 | VQRWLQVWRIRK |
| 208 | 1002-Q7 | VQRWLIQWRIRK |
| 209 | 1002-Q8 | VQRWLIVQRIRK |
| 210 | 1002-Q9 | VQRWLIVWQIRK |
| 211 | 1002-Q10 | VQRWLIVWRQRK |
| 212 | 1002-Q11 | VQRWLIVWRIQK |
| 213 | 1002-Q12 | VQRWLIVWRIRQ |
| 214 | HH2-G1 | GQLRIRVAVIRA |
| 215 | HH2-G2 | VGLRIRVAVIRA |
| 216 | HH2-G3 | VQGRIRVAVIRA |
| 217 | HH2-G4 | VQLGIRVAVIRA |
| 218 | HH2-G5 | VQLRGRVAVIRA |
| 219 | HH2-G6 | VQLRIGVAVIRA |
| 220 | HH2-G7 | VQLRIRGAVIRA |
| 221 | HH2-G8 | VQLRIRVGVIRA |
| 222 | HH2-G9 | VQLRIRVAGIRA |
| 223 | HH2-G10 | VQLRIRVAVGRA |
| 224 | HH2-G11 | VQLRIRVAVIGA |
| 225 | HH2-G12 | VQLRIRVAVIRG |
| 226 | HH2-A1 | AQLRIRVAVIRA |
| 227 | HH2-A2 | VALRIRVAVIRA |
| 228 | HH2-A3 | VQARIRVAVIRA |
| 229 | HH2-A4 | VQLAIRVAVIRA |
| 230 | HH2-A5 | VQLRARVAVIRA |
| 231 | HH2-A6 | VQLRIAVAVIRA |
| 232 | HH2-A7 | VQLRIRAAVIRA |
| 233 | HH2-A9 | VQLRIRVAAIRA |
| 234 | HH2-A10 | VQLRIRVAVARA |
| 235 | HH2-A11 | VQLRIRVAVIAA |
| 236 | HH2-R1 | RQLRIRVAVIRA |
| 237 | HH2-R2 | VRLRIRVAVIRA |
| 238 | HH2-R3 | VQRRIRVAVIRA |
| 239 | HH2-R5 | VQLRRRVAVIRA |
| 240 | HH2-R7 | VQLRIRRAVIRA |
| 241 | HH2-R8 | VQLRIRVRVIRA |
| 242 | HH2-R9 | VQLRIRVARIRA |
| 243 | HH2-R10 | VQLRIRVAVRRA |
| 244 | HH2-R12 | VQLRIRVAVIRR |
| 245 | HH2-K1 | KQLRIRVAVIRA |
| 246 | HH2-K2 | VKLRIRVAVIRA |
| 247 | HH2-K3 | VQKRIRVAVIRA |
| 248 | HH2-K4 | VQLKIRVAVIRA |
| 249 | HH2-K5 | VQLRKRVAVIRA |
| 250 | HH2-K6 | VQLRIKVAVIRA |
| 251 | HH2-K7 | VQLRIRKAVIRA |
| 252 | HH2-K8 | VQLRIRVKVIRA |
| 253 | HH2-K9 | VQLRIRVAKIRA |
| 254 | HH2-K10 | VQLRIRVAVKRA |
| 255 | HH2-K11 | VQLRIRVAVIKA |
| 256 | HH2-K12 | VQLRIRVAVIRK |
| 257 | HH2-L1 | LQLRIRVAVIRA |
| 258 | HH2-L2 | VLLRIRVAVIRA |
| 259 | HH2-L4 | VQLLIRVAVIRA |
| 260 | HH2-L5 | VQLRLRVAVIRA |
| 261 | HH2-L6 | VQLRILVAVIRA |
| 262 | HH2-L7 | VQLRIRLAVIRA |
| 263 | HH2-L8 | VQLRIRVLVIRA |
| 264 | HH2-L9 | VQLRIRVALIRA |
| 265 | HH2-L10 | VQLRIRVAVLRA |
| 266 | HH2-L11 | VQLRIRVAVILA |
| 267 | HH2-L12 | VQLRIRVAVIRL |
| 268 | HH2-I1 | IQLRIRVAVIRA |
| 269 | HH2-I2 | VILRIRVAVIRA |
| 270 | HH2-I3 | VQIRIRVAVIRA |
| 271 | HH2-I4 | VQLIIRVAVIRA |
| 272 | HH2-I6 | VQLRIIVAVIRA |
| 273 | HH2-17 | VQLRIRIAVIRA |
| 274 | HH2-I8 | VQLRIRVIVIRA |
| 275 | HH2-I9 | VQLRIRVAIIRA |
| 276 | HH2-I11 | VQLRIRVAVIIA |
| 277 | HH2-I12 | VQLRIRVAVIRI |
| 278 | HH2-V2 | VVLRIRVAVIRA |
| 279 | HH2-V3 | VQVRIRVAVIRA |
| 280 | HH2-V4 | VQLVIRVAVIRA |
| 281 | HH2-V5 | VQLRVRVAVIRA |
| 282 | HH2-V6 | VQLRIVVAVIRA |
| 283 | HH2-V8 | VQLRIRVVVIRA |
| 284 | HH2-V10 | VQLRIRVAVVRA |
| 285 | HH2-V11 | VQLRIRVAVIVA |
| 286 | HH2-V12 | VQLRIRVAVIRV |
| 287 | HH2-W1 | WQLRIRVAVIRA |
| 288 | HH2-W2 | VWLRIRVAVIRA |
| 289 | HH2-W3 | VQWRIRVAVIRA |
| 290 | HH2-W4 | VQLWIRVAVIRA |
| 291 | HH2-W5 | VQLRWRVAVIRA |
| 292 | HH2-W6 | VQLRIWVAVIRA |
| 293 | HH2-W7 | VQLRIRWAVIRA |
| 294 | HH2-W8 | VQLRIRVWVIRA |
| 295 | HH2-W9 | VQLRIRVAWIRA |
| 296 | HH2-W10 | VQLRIRVAVWRA |
| 297 | HH2-W11 | VQLRIRVAVIWA |
| 298 | HH2-W12 | VQLRIRVAVIRW |
| 299 | HH2-Q1 | QQLRIRVAVIRA |
| 300 | HH2-Q3 | VQQRIRVAVIRA |
| 301 | HH2-Q4 | VQLQIRVAVIRA |
| 302 | HH2-Q5 | VQLRQRVAVIRA |
| 303 | HH2-Q6 | VQLRIQVAVIRA |
| 304 | HH2-Q7 | VQLRIRQAVIRA |
| 305 | HH2-Q8 | VQLRIRVQVIRA |
| 306 | HH2-Q9 | VQLRIRVAQIRA |
| 307 | HH2-Q10 | VQLRIRVAVQRA |
| 308 | HH2-Q11 | VQLRIRVAVIQA |
| 309 | HH2-Q12 | VQLRIRVAVIRQ |
| 310 | 1018N-02C | VRLIVAVWRIRK |
| 311 | 18N-HH2C | VRLIVAVAVIRA |
| 312 | 1002N-18C | VQRWLIVRIWRR |
| 313 | 02N-HH2C | VQRWLIVAVIRA |
| 314 | HH2N-18C | VQLRIRVRIWRR |
| 315 | HH2N-02C | VQLRIRVWRIRK |
| 316 | 1002C-18N | VRIWRRVQRWLI |
| 317 | HH2C-18N | VAVIRAVRLIVA |
| 318 | 1018C-02N | VRIWRRVQRWLI |
| 319 | HH2C-02N | VAVIRAVQRWLI |
| 320 | 18C-HH2N | VRIWRRVQLRIR |
| 321 | 02C-HH2N | VWRIRKVQLRIR |
| 322 | 18C-1018N | VRIWRRVRLIVA |
| 323 | 02C-1002N | VWRIRKVQRWLI |
| 324 | HH2C-HH2N | VAVIRAVQLRIR |
| 325 | 18N4-02C8 | VRLILIVWRIRK |
| 326 | 18N4-HH2C8 | VRLIIRVAVIRA |
| 327 | 02N4-18C8 | VQRWVAVRIWRR |
| 328 | 02N4-HH2C8 | VQRWIRVAVIRA |
| 329 | HH2N4-18C8 | VQLRVAVRIWRR |
| 330 | HH2N4-02C8 | VQLRLIVWRIRK |
| 331 | 18N8-02C4 | VRLIVAVRRIRK |
| 332 | 18N8-HH2C4 | VRLIVAVRVIRA |
| 333 | 02N8-18C4 | VQRWLIVWIWRR |
| 334 | 02N8-HH2C4 | VQRWLIVWVIRA |
| 335 | HH2N8-18C4 | VQLRIRVAIWRR |
| 336 | HH2N8-02C4 | VQLRIRVARIRK |
| 337 | 1018-I | RRWIRVAVILRV |
| 338 | 1002-I | KRIRWVILWRQV |
| 339 | HH2-I | ARIVAVRIRLQV |
| 340 | 1018C-18N-I | AVILRVRRWIRV |
| 341 | 1002C-02N-I | ILWRQVKRIRWV |
| 342 | HH2C-HH2N-I | RIRLQVARIVAV |
| 343 | RI-1018G1 | GRWIRVAVILRV |
| 344 | RI-1018G2 | RGWIRVAVILRV |
| 345 | RI-1018G3 | RRGIRVAVILRV |
| 346 | RI-1018G4 | RRWGRVAVILRV |
| 347 | RI-1018G5 | RRWIGVAVILRV |
| 348 | RI-1018G6 | RRWIRGAVILRV |
| 349 | RI-1018G7 | RRWIRVGVILRV |
| 350 | RI-1018G8 | RRWIRVAGILRV |
| 351 | RI-1018G9 | RRWIRVAVGLRV |
| 352 | RI-1018G10 | RRWIRVAVIGRV |
| 353 | RI-1018G11 | RRWIRVAVILGV |
| 354 | RI-1018G12 | RRWIRVAVILRG |
| 355 | RI-1018A1 | ARWIRVAVILRV |
| 356 | RI-1018A2 | RAWIRVAVILRV |
| 357 | RI-1018A3 | RRAIRVAVILRV |
| 358 | RI-1018A4 | RRWARVAVILRV |
| 359 | RI-1018A5 | RRWIAVAVILRV |
| 360 | RI-1018A6 | RRWIRAAVILRV |
| 361 | RI-1018A8 | RRWIRVAAILRV |
| 362 | RI-1018A9 | RRWIRVAVALRV |
| 363 | RI-1018A10 | RRWIRVAVIARV |
| 364 | RI-1018A11 | RRWIRVAVILAV |
| 365 | RI-1018Al2 | RRWIRVAVILRA |
| 366 | RI-1018R3 | RRRIRVAVILRV |
| 367 | RI-1018R4 | RRWRRVAVILRV |
| 368 | RI-1018R6 | RRWIRRAVILRV |
| 369 | RI-1018R7 | RRWIRVRVILRV |
| 370 | RI-1018R8 | RRWIRVARILRV |
| 371 | RI-1018R9 | RRWIRVAVRLRV |
| 372 | RI-1018R10 | RRWIRVAVIRRV |
| 373 | RI-1018R12 | RRWIRVAVILRR |
| 374 | RI-1018K1 | KRWIRVAVILRV |
| 375 | RI-1018K2 | RKWIRVAVILRV |
| 376 | RI-1018K3 | RRKIRVAVILRV |
| 377 | RI-1018K4 | RRWKRVAVILRV |
| 378 | RI-1018K5 | RRWIKVAVILRV |
| 379 | RI-1018K6 | RRWIRKAVILRV |
| 380 | RI-1018K7 | RRWIRVKVILRV |
| 381 | RI-1018K8 | RRWIRVAKILRV |
| 382 | RI-1018K9 | RRWIRVAVKLRV |
| 383 | RI-1018K10 | RRWIRVAVIKRV |
| 384 | RI-1018K11 | RRWIRVAVILKV |
| 385 | RI-1018K12 | RRWIRVAVILRK |
| 386 | RI-1018V1 | VRWIRVAVILRV |
| 387 | RI-1018V2 | RVWIRVAVILRV |
| 388 | RI-1018V3 | RRVIRVAVILRV |
| 389 | RI-1018V4 | RRWVRVAVILRV |
| 390 | RI-1018V5 | RRWIVVAVILRV |
| 391 | RI-1018V7 | RRWIRVVVILRV |
| 392 | RI-1018V9 | RRWIRVAVVLRV |
| 393 | RI-1018V10 | RRWIRVAVIVRV |
| 394 | RI-1018V11 | RRWIRVAVILVV |
| 395 | RI-1018I1 | IRWIRVAVILRV |
| 396 | RI-1018I2 | RIWIRVAVILRV |
| 397 | RI-1018I3 | RRIIRVAVILRV |
| 398 | RI-1018I5 | RRWIIVAVILRV |
| 399 | RI-1018I6 | RRWIRIAVILRV |
| 400 | RI-1018I7 | RRWIRVIVILRV |
| 401 | RI-1018I8 | RRWIRVAIILRV |
| 402 | RI-1018I10 | RRWIRVAVIIRV |
| 403 | RI-1018I11 | RRWIRVAVILIV |
| 404 | RI-1018I12 | RRWIRVAVILRI |
| 405 | RI-1018L1 | LRWIRVAVILRV |
| 406 | RI-1018L2 | RLWIRVAVILRV |
| 407 | RI-1018L3 | RRLIRVAVILRV |
| 408 | RI-1018L4 | RRWLRVAVILRV |
| 409 | RI-1018L5 | RRWILVAVILRV |
| 410 | RI-1018L6 | RRWIRLAVILRV |
| 411 | RI-1018L7 | RRWIRVLVILRV |
| 412 | RI-1018L8 | RRWIRVALILRV |
| 413 | RI-1018L9 | RRWIRVAVLLRV |
| 414 | RI-1018L11 | RRWIRVAVILLV |
| 415 | RI-1018L12 | RRWIRVAVILRL |
| 416 | RI-1018W1 | WRWIRVAVILRV |
| 417 | RI-1018W2 | RWWIRVAVILRV |
| 418 | RI-1018W4 | RRWWRVAVILRV |
| 419 | RI-1018W5 | RRWIWVAVILRV |
| 420 | RI-1018W6 | RRWIRWAVILRV |
| 421 | RI-1018W7 | RRWIRVWVILRV |
| 422 | RI-1018W8 | RRWIRVAWILRV |
| 423 | RI-1018W9 | RRWIRVAVWLRV |
| 424 | RI-1018W10 | RRWIRVAVIWRV |
| 425 | RI-1018W11 | RRWIRVAVILWV |
| 426 | RI-1018W12 | RRWIRVAVILRW |
| 427 | RI-1018Q1 | QRWIRVAVILRV |
| 428 | RI-1018Q2 | RQWIRVAVILRV |
| 429 | RI-1018Q3 | RRQIRVAVILRV |
| 430 | RI-1018Q4 | RRWQRVAVILRV |
| 431 | RI-1018Q5 | RRWIQVAVILRV |
| 432 | RI-1018Q6 | RRWIRQAVILRV |
| 433 | RI-1018Q7 | RRWIRVQVILRV |
| 434 | RI-1018Q8 | RRWIRVAQILRV |
| 435 | RI-1018Q9 | RRWIRVAVQLRV |
| 436 | RI-1018Q10 | RRWIRVAVIQRV |
| 437 | RI-1018Q11 | RRWIRVAVILQV |
| 438 | RI-1018Q12 | RRWIRVAVILRQ |
| 439 | DJK6G1 | GQWRRIRVWVIR |
| 440 | DJK6G2 | VGWRRIRVWVIR |
| 441 | DJK6G3 | VQGRRIRVWVIR |
| 442 | DJK6G4 | VQWGRIRVWVIR |
| 443 | DJK6G5 | VQWRGIRVWVIR |
| 444 | DJK6G6 | VQWRRGRVWVIR |
| 445 | DJK6G7 | VQWRRIGVWVIR |
| 446 | DJK6G8 | VQWRRIRGWVIR |
| 447 | DJK6G9 | VQWRRIRVGVIR |
| 448 | DJK6G10 | VQWRRIRVWGIR |
| 449 | DJK6G11 | VQWRRIRVWVGR |
| 450 | DJK6G12 | VQWRRIRVWVIG |
| 451 | DJK6A1 | AQWRRIRVWVIR |
| 452 | DJK6A2 | VAWRRIRVWVIR |
| 453 | DJK6A3 | VQARRIRVWVIR |
| 454 | DJK6A4 | VQWARIRVWVIR |
| 455 | DJK6A5 | VQWRAIRVWVIR |
| 456 | DJK6A6 | VQWRRARVWVIR |
| 457 | DJK6A7 | VQWRRIAVWVIR |
| 458 | DJK6A8 | VQWRRIRAWVIR |
| 459 | DJK6A9 | VQWRRIRVAVIR |
| 460 | DJK6A10 | VQWRRIRVWAIR |
| 461 | DJK6A11 | VQWRRIRVWVAR |
| 462 | DJK6Al2 | VQWRRIRVWVIA |
| 463 | DJK6R1 | RQWRRIRVWVIR |
| 464 | DJK6R2 | VRWRRIRVWVIR |
| 465 | DJK6R3 | VQRRRIRVWVIR |
| 466 | DJK6R6 | VQWRRRRVWVIR |
| 467 | DJK6R8 | VQWRRIRRWVIR |
| 468 | DJK6R9 | VQWRRIRVRVIR |
| 469 | DJK6R10 | VQWRRIRVWRIR |
| 470 | DJK6R11 | VQWRRIRVWVRR |
| 471 | DJK6K1 | KQWRRIRVWVIR |
| 472 | DJK6K2 | VKWRRIRVWVIR |
| 473 | DJK6K3 | VQKRRIRVWVIR |
| 474 | DJK6K4 | VQWKRIRVWVIR |
| 475 | DJK6K5 | VQWRKIRVWVIR |
| 476 | DJK6K6 | VQWRRKRVWVIR |
| 477 | DJK6K7 | VQWRRIKVWVIR |
| 478 | DJK6K8 | VQWRRIRKWVIR |
| 479 | DJK6K9 | VQWRRIRVKVIR |
| 480 | DJK6K10 | VQWRRIRVWKIR |
| 481 | DJK6K11 | VQWRRIRVWVKR |
| 482 | DJK6K12 | VQWRRIRVWVIK |
| 483 | DJK6V2 | VVWRRIRVWVIR |
| 484 | DJK6V3 | VQVRRIRVWVIR |
| 485 | DJK6V4 | VQWVRIRVWVIR |
| 486 | DJK6V5 | VQWRVIRVWVIR |
| 487 | DJK6V6 | VQWRRVRVWVIR |
| 488 | DJK6V7 | VQWRRIVVWVIR |
| 489 | DJK6V9 | VQWRRIRVVVIR |
| 490 | DJK6V11 | VQWRRIRVWVVR |
| 491 | DJK6V12 | VQWRRIRVWVIV |
| 492 | DJK6I1 | IQWRRIRVWVIR |
| 493 | DJK6I2 | VIWRRIRVWVIR |
| 494 | DJK6I3 | VQIRRIRVWVIR |
| 495 | DJK6I4 | VQWIRIRVWVIR |
| 496 | DJK6I5 | VQWRIIRVWVIR |
| 497 | DJK6I7 | VQWRRIIVWVIR |
| 498 | DJK6I8 | VQWRRIRIWVIR |
| 499 | DJK6I9 | VQWRRIRVIVIR |
| 500 | DJK6I10 | VQWRRIRVWIIR |
| 501 | DJK6I12 | VQWRRIRVWVII |
| 502 | DJK6L1 | LQWRRIRVWVIR |
| 503 | DJK6L2 | VLWRRIRVWVIR |
| 504 | DJK6L3 | VQLRRIRVWVIR |
| 505 | DJK6L4 | VQWLRIRVWVIR |
| 506 | DJK6L5 | VQWRLIRVWVIR |
| 507 | DJK6L6 | VQWRRLRVWVIR |
| 508 | DJK6L7 | VQWRRILVWVIR |
| 509 | DJK6L8 | VQWRRIRLWVIR |
| 510 | DJK6L9 | VQWRRIRVLVIR |
| 511 | DJK6L10 | VQWRRIRVWLIR |
| 512 | DJK6L11 | VQWRRIRVWVLR |
| 513 | DJK6L12 | VQWRRIRVWVIL |
| 514 | DJK6W1 | WQWRRIRVWVIR |
| 515 | DJK6W2 | VWWRRIRVWVIR |
| 516 | DJK6W4 | VQWWRIRVWVIR |
| 517 | DJK6W5 | VQWRWIRVWVIR |
| 518 | DJK6W6 | VQWRRWRVWVIR |
| 519 | DJK6W7 | VQWRRIWVWVIR |
| 520 | DJK6W8 | VQWRRIRWWVIR |
| 521 | DJK6W10 | VQWRRIRVWWIR |
| 522 | DJK6W11 | VQWRRIRVWVWR |
| 523 | DJK6W12 | VQWRRIRVWVIW |
| 524 | DJK6Q1 | QQWRRIRVWVIR |
| 525 | DJK6Q3 | VQQRRIRVWVIR |
| 526 | DJK6Q4 | VQWQRIRVWVIR |
| 527 | DJK6Q5 | VQWRQIRVWVIR |
| 528 | DJK6Q6 | VQWRRQRVWVIR |
| 529 | DJK6Q7 | VQWRRIQVWVIR |
| 530 | DJK6Q8 | VQWRRIRQWVIR |
| 531 | DJK6Q9 | VQWRRIRVQVIR |
| 532 | DJK6Q10 | VQWRRIRVWQIR |
| 533 | DJK6Q11 | VQWRRIRVWVQR |
| 534 | DJK6Q12 | VQWRRIRVWVIQ |
| 535 | RI-1002G1 | GRIRWVILWRQV |
| 536 | RI-1002G2 | KGIRWVILWRQV |
| 537 | RI-1002G3 | KRGRWVILWRQV |
| 538 | RI-1002G4 | KRIGWVILWRQV |
| 539 | RI-1002G5 | KRIRGVILWRQV |
| 540 | RI-1002G6 | KRIRWGILWRQV |
| 541 | RI-1002G7 | KRIRWVGLWRQV |
| 542 | RI-1002G8 | KRIRWVIGWRQV |
| 543 | RI-1002G9 | KRIRWVILGRQV |
| 544 | RI-1002G10 | KRIRWVILWGQV |
| 545 | RI-1002G11 | KRIRWVILWRGV |
| 546 | RI-1002G12 | KRIRWVILWRQG |
| 547 | RI-1002A1 | ARIRWVILWRQV |
| 548 | RI-1002A2 | KAIRWVILWRQV |
| 549 | RI-1002A3 | KRARWVILWRQV |
| 550 | RI-1002A4 | KRIAWVILWRQV |
| 551 | RI-1002A5 | KRIRAVILWRQV |
| 552 | RI-1002A6 | KRIRWAILWRQV |
| 553 | RI-1002A7 | KRIRWVALWRQV |
| 554 | RI-1002A8 | KRIRWVIAWRQV |
| 555 | RI-1002A9 | KRIRWVILARQV |
| 556 | RI-1002A10 | KRIRWVILWAQV |
| 557 | RI-1002A11 | KRIRWVILWRAV |
| 558 | RI-1002Al2 | KRIRWVILWRQA |
| 559 | RI-1002R1 | RRIRWVILWRQV |
| 560 | RI-1002R3 | KRRRWVILWRQV |
| 561 | RI-1002R5 | KRIRRVILWRQV |
| 562 | RI-1002R6 | KRIRWRILWRQV |
| 563 | RI-1002R7 | KRIRWVRLWRQV |
| 564 | RI-1002R8 | KRIRWVIRWRQV |
| 565 | RI-1002R9 | KRIRWVILRRQV |
| 566 | RI-1002R11 | KRIRWVILWRRV |
| 567 | RI-1002R12 | KRIRWVILWRQR |
| 568 | RI-1002K2 | KKIRWVILWRQV |
| 569 | RI-1002K3 | KRKRWVILWRQV |
| 570 | RI-1002K4 | KRIKWVILWRQV |
| 571 | RI-1002K5 | KRIRKVILWRQV |
| 572 | RI-1002K6 | KRIRWKILWRQV |
| 573 | RI-1002K7 | KRIRWVKLWRQV |
| 574 | RI-1002-K8 | KRIRWVIKWRQV |
| 575 | RI-1002K9 | KRIRWVILKRQV |
| 576 | RI-1002K10 | KRIRWVILWKQV |
| 577 | RI-1002K11 | KRIRWVILWRKV |
| 578 | RI-1002K12 | KRIRWVILWRQK |
| 579 | RI-1002V1 | VRIRWVILWRQV |
| 580 | RI-1002V2 | KVIRWVILWRQV |
| 581 | RI-1002V3 | KRVRWVILWRQV |
| 582 | RI-1002V4 | KRIVWVILWRQV |
| 583 | RI-1002V5 | KRIRVVILWRQV |
| 584 | RI-1002V7 | KRIRWVVLWRQV |
| 585 | RI-1002V8 | KRIRWVIVWRQV |
| 586 | RI-1002V9 | KRIRWVILVRQV |
| 587 | RI-1002V10 | KRIRWVILWVQV |
| 588 | RI-1002V11 | KRIRWVILWRVV |
| 589 | RI-1002I1 | IRIRWVILWRQV |
| 590 | RI-1002I2 | KIIRWVILWRQV |
| 591 | RI-1002I4 | KRIIWVILWRQV |
| 592 | RI-1002I5 | KRIRIVILWRQV |
| 593 | RI-1002I6 | KRIRWIILWRQV |
| 594 | RI-1002I8 | KRIRWVIIWRQV |
| 595 | RI-1002I9 | KRIRWVILIRQV |
| 596 | RI-1002I10 | KRIRWVILWIQV |
| 597 | RI-1002I11 | KRIRWVILWRVV |
| 598 | RI-1002I12 | KRIRWVILWRQI |
| 599 | RI-1002L1 | LRIRWVILWRQV |
| 600 | RI-1002L2 | KLIRWVILWRQV |
| 601 | RI-1002L3 | KRLRWVILWRQV |
| 602 | RI-1002L4 | KRILWVILWRQV |
| 603 | RI-1002L5 | KRIRLVILWRQV |
| 604 | RI-1002L6 | KRIRWLILWRQV |
| 605 | RI-1002L7 | KRIRWVLLWRQV |
| 606 | RI-1002L9 | KRIRWVILLRQV |
| 607 | RI-1002L10 | KRIRWVILWLQV |
| 608 | RI-1002L11 | KRIRWVILWRLV |
| 609 | RI-1002L12 | KRIRWVILWRQL |
| 610 | RI-1002W1 | WRIRWVILWRQV |
| 611 | RI-1002W2 | KWIRWVILWRQV |
| 612 | RI-1002W3 | KRWRWVILWRQV |
| 613 | RI-1002W4 | KRIWWVILWRQV |
| 614 | RI-1002W6 | KRIRWWILWRQV |
| 615 | RI-1002W7 | KRIRWVWLWRQV |
| 616 | RI-1002W8 | KRIRWVIWWRQV |
| 617 | RI-1002W10 | KRIRWVILWWQV |
| 618 | RI-1002W11 | KRIRWVILWRWV |
| 619 | RI-1002W12 | KRIRWVILWRQW |
| 620 | RI-1002Q1 | QRIRWVILWRQV |
| 621 | RI-1002Q2 | KQIRWVILWRQV |
| 622 | RI-1002Q3 | KRQRWVILWRQV |
| 623 | RI-1002Q4 | KRIQWVILWRQV |
| 624 | RI-1002Q5 | KRIRQVILWRQV |
| 625 | RI-1002Q6 | KRIRWQILWRQV |
| 626 | RI-1002Q7 | KRIRWVQLWRQV |
| 627 | RI-1002Q8 | KRIRWVIQWRQV |
| 628 | RI-1002Q9 | KRIRWVILQRQV |
| 629 | RI-1002Q10 | KRIRWVILWQQV |
| 630 | RI-1002Q12 | KRIRWVILWRQQ |
| 631 | RH8N-RI02C | RRWIRVILWRQV |
| 632 | RH8N-DJK6C | RRWIRVRVWVIR |
| 633 | RI02N-R118C | KRIRWVAVILRV |
| 634 | RI02N-DJK6C | KRIRWVRVWVIR |
| 635 | DJK6N-RI18C | VQWRRIAVILRV |
| 636 | DJK6N-RI02C | VQWRRIILWRQV |
| 637 | RI02C-RI18N | AVILRVKRIRWV |
| 638 | DJK6C-R118N | RVWVIRRRWIRV |
| 639 | RI18C- | AVILRVKRIRWV |
| | RI-02N | |
| 640 | DJK6C-RI02N | RVWVIRKRIRWV |
| 641 | RI18C-DJK6N | AVILRVVQWRRI |
| 642 | RI02C-DJK6N | ILWRQVVQWRRI |
| 643 | RI18C-RI18N | AVILRVRRWIRV |
| 644 | RI02C-RI02N | ILWRQVKRIRWV |
| 645 | DJK6C-DJK6N | RVWVIRVQWRRI |
| 646 | RI18N4- | RRWIWVILWRQV |
| | RI02C8 | |
| 647 | RI18N4- | RRWIRIRVWVIR |
| | DJK6C8 | |
| 648 | RI02N4- | KRIRRVAVILRV |
| | RI18C8 | |
| 649 | RI02N4- | KRIRRIRVWVIR |
| | DJK6C8 | |
| 650 | DJK6N4- | VQWRRVAVILRV |
| | RI18C8 | |
| 651 | DJK6N4- | VQWRWVILWRQV |
| | RI02C8 | |
| 652 | RI18N8- | RRWIRVAVWRQV |
| | RI02C4 | |
| 653 | RI18N8- | RRWIRVAVWVIR |
| | DJK6C4 | |
| 654 | RI02N8- | KRIRWVILILRV |
| | RI18C4 | |
| 655 | RI02N8- | KRIRWVILWVIR |
| | DJK6C4 | |
| 656 | DJK6N8- | VQWRRIRVILRV |
| | RI18C4 | |
| 657 | DJK6N8- | VQWRRIRVWRQV |
| | RI02C4 | |
| 658 | D-1018 | VRLIVAVRIWRR |
| 659 | D-1002 | VQRWLIVWRIRK |
| 660 | DJK6Rev | RIVWVRIRRWQV |
| 661 | RI18C- | VRIWRRVRLIVA |
| | RI18NRev | |
| 662 | RI02C- | VWRIRKVQRWLI |
| | RI02NRev | |
| 663 | DJK6C- | IRRWQVRIVWVR |
| | DJK6NRev | |
| 664 | DJK1 | VFLRRIRVIVIR |
| 665 | DJK2 | VFWRRIRVWVIR |
| 666 | DJK3 | VQLRAIRVRVIR |
| 667 | DJK4b | VQLRRIRVWVIR |
| 668 | DJK5 | VQWRAIRVRVIR |
| 669 | DJK6 | VQWRRIRVWVIR |
| 670 | 1005 | VQLRIRVAV |
| 671 | 1002 | VQRWLIVWRIRK |
| 672 | HH2 | VQLRIRVAVIRA |
| 673 | 1018 | VRLIVAVRIWRR |
| 674 | 1020 | VRLRIRWWVLRK |
| 675 | 1021 | VRLRIRVAV |
| 676 | 1032 | IRVRVIWRK |
| 677 | 1041 | VIWIRWR |
| 678 | 1043 | WIVIWRR |
| 679 | 1044 | IRWVIRW |
| 680 | HHC 53 | FRRWWKWFK |
| 681 | HHC 75 | RKWIWRWFL |
| 682 | Bac241 | RLERIVVIRVAR |
| | (D1) | |
| 683 | Bac263 | RLAGIVVIRVAR |
| | (D2) | |
| 684 | K7 (D6) | RLARIVKIRVAR |
| 685 | 1021 | VRLRIRVAV |
| 686 | IN62 | ILRWKWRWWVWRR |
| 687 | HH18 | IWVIWRR |
| 688 | 1005 | VQLRIRVAV |
| 689 | 1011 | RRWVVWRIVQRR |
| 690 | 1010 | IRWRIRVWVRRI |
| 691 | HH5 | VRLWIRVAVIRA |
| 692 | VKJ11 | VQWRIRVRV |
| 693 | Kai-39 | ILPWWWPWWPWRR |
| 694 | IN65 | ILVWKWRWWVWRR |
| 695 | Kai-10 | RLWRIVVIRVKR |
| 696 | HH17 | KIWVRWK |
| 697 | Kai-38 | RLWRIVVIRVAR |
| 698 | Kai-30 | RWTISFKRS-CONH2 |
| 699 | HH7 | VRLRIRVAVRRA |
| 700 | Kai-22 | (RRWRIVVIRVRR)4-K2-K |
| 701 | LJK6 | VQWRRIRVWVIR |
| 702 | VKJ7 | VRFRIRVRVWIK |
| 703 | IN66 | ILVWKWVWWVWRR |
| 704 | Kai-49 | HQFRFRFRVRRK |
| 705 | 1027 | KKQVSRVKVWRK |
| 706 | 1001 | LVRAIQVRAVIR |
| 707 | R-E2 | RRWIVWIR |
| 708 | 1013 | VRLRIRVAV |
| 709 | Kai-3 | QRLRIRVAVIRA |
| 710 | VKJ12 | VRFRIRVRV |
| 711 | 1014 | RQVIVRRW |
| 712 | VKJ13 | FRIRVRF |
| 713 | CP26 | KWKSFIKKLTSAAKKVVTTAKPLISS |
| 714 | HH2 | VQLRIRVAVIRA |
| 715 | 1051 | VQLRIRVWVIRK |
| 716 | Kai-48 | KQFRIRVRVIRK |
| 717 | C3 | RGARIVVIRVAR |
| 718 | VKJ14 | RFRIRVRV |
| 719 | 1022 | LRIRVIVWR |
| 720 | E6 | RRWRIVVIRVRR |
| 721 | 1019 | IVVWRRQLVKNK |
| 722 | E1 | RLARIVVFRVAR |
| 723 | 1004 | RFWKVRVKYIRF |
| 724 | 1009 | AIRVVRARLVRR |
| 725 | W3 | VRWRIRVAVIRA |
| 726 | 1003 | IVWKIKRWWVGR |
| 727 | Kai-27 | KRWIVKWVK |
| 728 | HH14 | HQWRIRVAVRRH |
| 729 | 1023 | IRVWVLRQR |
| 730 | E3 | RLARIVVIRVRR |
| 731 | 1008 | RIKWIVRFR |
| 732 | 1029 | KQFRIRVRV |
| 733 | 11CN | ILKKWPWWPWRRK |
| 734 | C2 | GLARIVVIRVAR |
| 735 | 1024 | RIRVIVLKK |
| 736 | LJK2 | VFWRRIRVWVIR |
| 737 | Kai-24 | RVRWYRIFY |
| 738 | HH16 | KRWRIRVRVIRK |
| 739 | C6 | RLRRIVVIRVAR |
| 740 | 2003; 1018 | VRLIVKVRIRRR |
| | derivative | |
| 741 | 2004; 1018 | VRVIVKVRIRRR |
| | derivative | |
| 742 | 2006; 1018 | VRWIVKVRIRRR |
| | derivative | |
| 743 | 2007; 1018 | RRLIVKVRIWRR |
| | derivative | |
| 744 | 2008; 1018 | RRWIVKVRIRRR |
| | derivative | |
| 745 | 2009; 1002 | KWRLLIRWRIQK |
| | derivative | |
| 746 | 2010; 1002 | KQRWLIRWRIRK |
| | derivative | |
| 747 | 2011; HH2 | VQLRIRVKVIRK |
| | derivative | |
| 748 | 2012; HH2 | WQLRIRVKVIRK |
| | derivative | |
| 749 | 2013; HH2 | WQRVRRVKVIRK |
| | derivative | |
| 750 | LL37 | LLGDFFRKSKEKIGKEFKRIVQRIKDFLRNLVPRT |
| | | ES |
| 751 | MX226^{a} | ILRWPWPWRRK |
| 752 | CALL | KWKLFKKIFKRIVQRIKDFLR |
| 753 | Indolicidin | ILPWKWPWWPWRR |

### Example 2

### Anti-Biofilm Activity

Methods of Assessment of Anti-Biofilm Activity—

Biofilm formation was initially analyzed using a static abiotic solid surface assay as described elsewhere (de la Fuente-Nunez et al., 2012). Dilutions (1/100) of overnight cultures were incubated in BM2 biofilm-adjusted medium [62 mM potassium phosphate buffer (pH 7), 7 mM (NH4)2SO4, 2 mM MgSO4, 10 μM FeSO4, 0.4% (wt/vol) glucose, 0.5% (wt/vol) Casamino Acids], or a nutrient medium such as Luria Broth, in polypropylene microtiter plates (Falcon, United States) in the absence (control) or presence of peptide. Peptide was added at time zero (prior to adding the diluted, overnight cultures) in varying concentrations, and the decrease in biofilm formation was recorded at 22-46 h for most bacteria. Planktonic cells were removed, biofilm cells adhering to the side of the tubes were stained with crystal violet, and absorbance at 595 nm was measured using a microtiter plate reader (Bio-Tek Instruments Inc., United States). Some peptides were screened against two Gram negative organisms, P. aeruginosa and K. pneumoniae using a Bioflux apparatus (AutoMate Scientific, Berkeley, Calif.; http://www.autom8.com/bioflux_biofilm.html), which allows for the high-throughput, real-time analysis of biofilms.

Antibiofilm Activity—

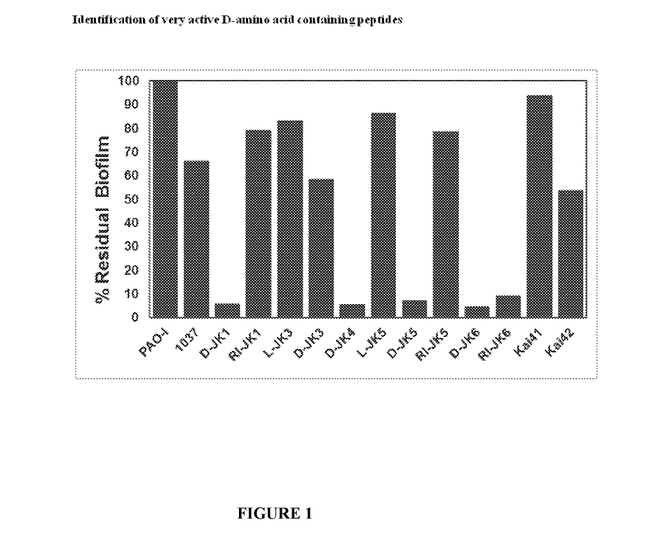

As can be seen in FIG. 1, screening of a series of L-D- and retro-inverso (RI) peptides indicated clearly that peptides differed widely in their activity. Peptides ranged from very active to inactive and the most active peptides were clearly superior to previously investigated peptides such as 1037 (de la Fuente-Nunez et al, 2011).

Broader screening revealed a substantial number of active peptides (Table 2).

We have also observed activity for 1018, DJK5 and DJK6 against multiple multidrug resistant isolates of many Gram negative and Gram positive including MDR strains of Pseudomonas aeruginosa and Acinetobacter baumannii, carbapenemase expressing Klebsiella pneumoniae, Enterobacter cloacae with de-repressed chromosomal β-lactamase, and vancomycin resistant Enterococcus, in addition to activity vs. oral biofilms formed on hydroxyapatite disks.

Using peptide array methods, >300 derivatives of HH2, 1002 and 1018 were made on peptide arrays by SPOT synthesis using single amino acid substitutions, and screened for their ability to inhibit MRSA biofilms at a concentration of 2.5 μM (approximately 3-4 μg/ml) (Table 2A). Many peptides showed similar or improved activities, compared to their parent peptides, and are indicated by bold typeface in Table 2A. Other peptides were rationally and iteratively designed based on the results of the single amino acid substitutions and are described in Table 2B.

TABLE 2
Activity of anti-biofilm peptides: All
sequences were amidated and sequences
with D or RI in front of them contain
all D-amino-acids. Pseudomonas
aeruginosa (Pa) and Klebsiella
pneumoniae (Kp) were tested at 10
μg/ml; methicillin resistant S.
aureus (Sa) at 5 μg/ml. “—” means
not tested. The peptides varied in
activity with the first group of
peptides representing broad spectrum
anti-biofilm peptides that were as
good, or better than, the control
peptides tested, while the group
under other peptides had lesser or
narrow spectrum activity.

| | Sequences (all pep- | | | |
|---|---|---|---|---|
| | tides amidated); D | % Biofilm | | |
| Peptide | and RI peptides com- | Inhibition | | |
| name | posed of D amino acids | Pa | Kp | Sa |

Broad spectrum anti-biofilm peptides

| | | | | |
|---|---|---|---|---|
| RIJK3 | RIVRVRIARLQV | 100 | 99 | — |
| Bac241 | RLERIVVIRVAR | 99.8 | — | — |
| RI1035 | RRINRVIWRWRK | 99.8 | 85 | — |
| RIJK4 | RIVWVRIRRLQV | 99.8 | 71 | — |
| DJK5 | VQWRAIRVRVIR | 99.7 | 99.8 | 95 |
| DJK4 | VQLRRIRVWVIR | 99 | 97 | 95 |
| 1018 | VRLIVAVRIWRR | 99 | 99 | 67 |
| 1021 | VRLRIRVAV | 99 | — | 73 |
| HE4 | VRLIWAVRIWRR | 99 | — | 88 |
| DJK6 | VQWRRIRVWVIR | 98.4 | 98 | 95 |
| 1005 | VQLRIRVAV | 96 | — | 83 |
| Bac263 | RLAGIVVIRVAR | 96 | 97 | — |
| 1040 | FQVVKIKVR | 95 | 86 | 38 |
| HE12 | RFKRVARVIW | 95 | — | 14 |
| RI1012 | FKKVIVIRRWFI | 95 | — | 95 |
| RI1018 | RRWIRVAVILRV | 95 | — | 95 |
| 1032 | IRVRVIWRK | 94 | 94 | — |
| HE1 | RRWIRVAVILRV | 93 | — | — |
| RIJK2 | RIVWVRIRRWFV | 91 | — | — |
| 1044 | IRWVIRW | 88 | 6 | 42 |
| DJK2 | VFWRRIRVWVIR | 87 | — | 95 |
| DJK1 | VFLRRIRVIVIR | 85 | — | 95 |
| RIJK6 | RIVWVRIRRWQV | 74 | 92 | 98 |
| RI1002 | KRIRWVILWRQV | 72 | 73 | 95 |
| 1041 | VIWIRWR | 64 | 68 | 31 |
| K7 | RLARIVKIRVAR | 63 | 49 | — |
| R-E2 | RRWIVWIR | 63 | 95 | 63 |
| 1043 | WIVIWRR | 57 | 44 | — |
| VKJ15 | RFRIRVRR | 46 | 8 | — |
| DJK3 | VQLRAIRVRVIR | 45 | — | — |
| HHC 10 | KRWWKWIRW | 40 | — | 65 |
| HE10 | VRLIVRIWRR | 39 | — | 75 |
| 1039 | IWVIRRVWR | 37 | 86 | — |
| 1048 | IRWVIRW | 31 | 61 | — |
| 1020 | VRLRIRWWVLRK | 22 | — | 76 |
| HHC 53 | FRRWWKWFK | — | — | 85 |
| HHC 75 | RKWIWRWFL | — | — | 94 |

Control peptides: Seq ID 749-753

| | | | | |
|---|---|---|---|---|
| LL37 | LLGDFFRKSKEKIGKEFKRIVQ | 88 | 78 | 76 |
| | RIKDFLRNLVPRTES | | | |
| MX226 | ILRWPWPWRRK | 18 | 18 | — |
| CALL | KWKLFKKIFKRIVQRIKDFLR | 84 | 50 | — |
| Indolici- | ILPWKWPWWPWRR | 29 | — | 48 |
| din | | | | |

Other peptides

| | | | | |
|---|---|---|---|---|
| IN62 | ILRWKWRWWVWRR | 98 | — | — |
| HH18 | IWVIWRR | 97 | — | — |
| 1011 | RRWVVWRIVQRR | 96 | — | 20 |
| 1010 | IRWRIRVWVRRI | 96 | — | — |
| HH5 | VRLWIRVAVIRA | 94 | — | — |
| Bac2a | RLARIVVIRVAR | 91 | — | 20 |
| E4 | RLARIVVIRVAG | 89 | 8 | — |
| VKJ11 | VQWRIRVRV | 89 | — | — |
| IN65 | ILVWKWRWWVWRR | 83 | — | — |
| Kai-39 | ILPWWWPWWPWRR | 83 | 10 | — |
| Kai-10 | RLWRIVVIRVKR | 81 | — | — |
| Kai-3 | QRLRIRVAVIRA | 81 | — | — |
| Kai-38 | RLWRIVVIRVAR | 79 | 2 | — |
| HH7 | VRLRIRVAVRRA | 75 | — | — |
| Kai-30 | RWTISFKRS-CONH2 | 75 | 5 | — |
| Kai-22 | (RRWRIVVIRVRR)₄-K2-K | 70 | — | — |
| LJK6 | VQWRRIRVWVIR | 69 | — | — |
| VKJ7 | VRFRIRVRVWIK | 68 | — | — |
| IN66 | ILVWKWVWWVWRR | 67 | 50 | — |
| 1027 | KKQVSRVKVWRK | 66 | — | 0 |
| Kai-49 | HQFRFRFRVRRK | 66 | 22 | — |
| 1001 | LVRAIQVRAVIR | 65 | — | 27 |
| VKJ12 | VRFRIRVRV | 64 | — | — |
| 1014 | RQVIVRRW | 62 | 0 | 0 |
| CP26 | KWKSFIKKLTSAAKKVVTTA | 62 | — | — |
| | KPLISS | | | |
| VKJ13 | FRIRVRF | 62 | — | — |
| 1051 | VQLRIRVWVIRK | 61 | — | — |
| C3 | RGARIVVIRVAR | 59 | — | — |
| Kai-48 | KQFRIRVRVIRK | 59 | 0 | — |
| VKJ14 | RFRIRVRV | 58 | — | — |
| E6 | RRWRIVVIRVRR | 57 | 0 | — |
| HH17 | KIWVRWK | 56 | — | — |
| 1022 | LRIRVIVWR | 56 | — | 15 |
| E1 | RLARIVVFRVAR | 55 | — | — |
| 1004 | RFWKVRVKYIRF | 55 | — | 24 |
| 1019 | IVVWRRQLVKNK | 55 | — | — |
| 1009 | AIRVVRARLVRR | 54 | — | — |
| 1003 | IVWKIKRWWVGR | 53 | 1 | — |
| Kai-27 | KRWIVKWVK | 52 | 9 | — |
| 1023 | IRVWVLRQR | 51 | — | 7 |
| HH14 | HQWRIRVAVRRH | 51 | — | — |
| HH2 | VQLRIRVAVIRA | 47 | — | 30 |
| E3 | RLARIVVIRVRR | 47 | — | — |
| 1008 | RIKWIVRFR | 47 | — | — |
| 1029 | KQFRIRVRV | 46 | — | 32 |
| 11CN | ILKKWPWWPWRRK | 44 | 43 | 32 |
| LJK2 | VFWRRIRVWVIR | 43 | — | — |
| 1024 | RIRVIVLKK | 43 | — | 0 |
| C2 | GLARIVVIRVAR | 43 | — | — |
| HE2 | VRLIRAVRAWRV | 42 | 0 | — |
| Kai-24 | RVRWYRIFY | 42 | — | — |
| 1012 | IFWRRIVIVKKF | 41 | 1 | — |
| C6 | RLRRIVVIRVAR | 40 | — | — |
| HH16 | KRWRIRVRVIRK | 40 | — | — |
| HHC10 | KRWWKWIRW | 40 | 0 | 65 |
| HE10 | VRLIVRIWRR-NH2 | 39 | — | — |
| 1031 | WRWRVRVWR | 38 | 20 | 38 |
| IDR-1 | KSRIVPAIPVSLL | 37 | 19 | 3 |
| VKJ10 | KQFRIRVRVWIK | 35 | 22 | — |
| W3 | VRWRIRVAVIRA | 32 | 41 | 66 |
| HE3 | VRWARVARILRV | 31 | — | — |
| 1048 | IRWVIRW | 31 | 61 | — |
| 1028 | LIQRIRVRNIVK | 31 | — | 0 |
| 1016 | LRIRWIFKR | 30 | — | 54 |
| D5 | RLARIVPIRVAR | 29 | 15 | — |
| 1017 | KRIVRRLVARIV | 26 | — | 21 |
| VK-J8 | KRFRIRVRWVIK | 25 | 0 | — |
| 1013 | VRLRIRVAV | 24 | — | 65 |
| D4 | RLARICVIRVAR | 21 | 12 | — |
| VK-J16 | VFRIRVRVR | 21 | 0 | — |
| VKJ18 | VRIVRRVI | 19 | 1 | — |
| D3 | RLARRVVIRVAR | 17 | 8 | — |
| 1007 | WNRVKWIRR | 15 | — | 15 |
| E5 | RLRRIVVIRVRR | 8 | 8 | — |
| 1002 | VQRWLIVWRIRK | 7 | — | 69 |
| KaiE2 | RIWVIWRR | 5 | — | 5 |
| 1038 | IVVRRVIRK | 4 | 23 | 0 |
| 1045 | RWWRIVI | 3 | 64 | — |
| 1042 | IVWIWRR | 3 | 66 | 0 |
| HHC36 | KRWWKWWRR | 2 | — | 48 |
| F3 | RLARIVVIRVA | 1 | 13 | — |
| 1015 | VLIRWNGKK | 1 | — | 19 |
| 1037 | KRFRIRVRV | 0 | 33 | — |
| 1046 | WIRVIRW | 0 | 63 | 0 |
| 1034 | KQFRNRLRIVKK | 0 | 30 | 0 |
| 1033 | RRVIVKKFRIRR | 0 | 47 | 0 |
| 1047 | IIRRWWV | 0 | 24 | — |
| LJK5 | VQWRAIRVRVIR | 0 | — | — |
| L-JK3 | VQLRAIRVRVIR | 0 | — | — |
| RI-JK5 | RIVRVRAIRWQV | 0 | — | — |
| RI-JK1 | RIVIVRIRRLFV | 0 | — | — |
| PMXB | C₅₆H₁₀₀N₁₆O₁₇S | 0 | — | — |
| 1030 | FRIRVRVIR | — | — | 26 |

TABLE 2A
Screening of single substituted peptides for antibiofilm activity against
methicillin resistant Staphylococcus aureus (MRSA).
The percentage of biofilm growth is reported compared to untreated
samples. All peptides that reduced the biofilm by more than 50% are
highlighted in bold. All peptides were tested at an approximate peptide
concentration of 2.5 μM (around 3 μg/ml) and appear as 3 sets
of two columns in the Table.

| | | | | | |
|---|---|---|---|---|---|
| HH2-K11 | 43 | 1018-L11 | 4 | 1002-I11 | 67 |
| HH2-K12 | 67 | 1018-L12 | 44 | 1002-I12 | 66 |
| HH2-L1 | 72 | 1018-I1 | 56 | 1002-V2 | 72 |
| HH2-L2 | 69 | 1018-I2 | 61 | 1002-V3 | 49 |
| HH2-L4 | 54 | 1018-I3 | 68 | 1002-V4 | 69 |
| HH2-L5 | 58 | 1018-I5 | 60 | 1002-V5 | 75 |
| HH2-L6 | 18 | 1018-I6 | 65 | 1002-V6 | 71 |
| HH2-L7 | 68 | 1018-I7 | 63 | 1002-V8 | 56 |
| HH2-L8 | 50 | 1018-I8 | 39 | 1002-V9 | 47 |
| HH2-L9 | 65 | 1018-I10 | 58 | 1002-V10 | 63 |
| HH2-L10 | 63 | 1018-I11 | 16 | 1002-V11 | 68 |
| HH2-L11 | 47 | 1018-I12 | 57 | 1002-V12 | 67 |
| HH2-L12 | 51 | 1018-V2 | 67 | 1002-W1 | 76 |
| HH2-I1 | 73 | 1018-V3 | 76 | 1002-W2 | 61 |
| HH2-I2 | 54 | 1018-V4 | 75 | 1002-W3 | 68 |
| HH2-I3 | 62 | 1018-V6 | 70 | 1002-W5 | 76 |
| HH2-I4 | 47 | 1018-V8 | 34 | 1002-W6 | 53 |
| HH2-I6 | 45 | 1018-V9 | 53 | 1002-W7 | 62 |
| HH2-I7 | 69 | 1018-V10 | 50 | 1002-W9 | 73 |
| HH2-I8 | 74 | 1018-V11 | 29 | 1002-W10 | 71 |
| HH2-I9 | 73 | 1018-V12 | 56 | 1002-W11 | 37 |
| HH2-I11 | 55 | 1018-W1 | 68 | 1002-W12 | 71 |
| HH2-I12 | 65 | 1018-W2 | 68 | 1002-Q1 | 77 |
| HH2-V2 | 77 | 1018-W3 | 66 | 1002-Q3 | 70 |
| HH2-V3 | 57 | 1018-W4 | 67 | 1002-Q4 | 36 |
| HH2-V4 | 60 | 1018-W5 | 55 | 1002-Q5 | 48 |
| HH2-V5 | 50 | 1018-W6 | 75 | 1002-Q6 | 22 |
| HH2-V6 | 39 | 1018-W7 | 55 | 1002-Q7 | 45 |
| HH2-V8 | 66 | 1018-W8 | 43 | 1002-Q8 | 20 |
| HH2-V10 | 68 | 1018-W9 | 69 | 1002-Q9 | 41 |
| HH2-V11 | 51 | 1018-W11 | 53 | 1002-Q10 | 39 |
| HH2-V12 | 65 | 1018-W12 | 65 | 1002-Q11 | 67 |
| HH2-W1 | 67 | 1018-Q1 | 46 | 1002-Q12 | 65 |
| HH2-W2 | 70 | 1018-Q2 | 52 | 1018N-1002C | 60 |
| HH2-W3 | 62 | 1018-Q3 | 12 | 1018N-HH2C | 46 |
| HH2-W4 | 58 | 1018-Q4 | 39 | 1002N-1018C | 73 |
| HH2-W5 | 63 | 1018-Q5 | 13 | 1002N-HH2C | 54 |
| HH2-W6 | 56 | 1018-Q6 | 55 | HH2N-1018C | 70 |
| HH2-W7 | 70 | 1018-Q7 | 28 | HH2N-1002C | 60 |
| HH2-W8 | 75 | 1018-Q8 | 51 | 1002C-1018N | 92 |
| HH2-W9 | 75 | 1018-Q9 | 38 | HH2C-1018N | 28 |
| HH2-W10 | 48 | 1018-Q10 | 38 | 1018C-1002N | 85 |
| HH2-W11 | 45 | 1018-Q11 | 51 | HH2C-1002N | 0 |
| HH2-W12 | 58 | 1018-Q12 | 57 | 1018C-HH2N | 19 |
| HH2-Q1 | 57 | 1002 | 73 | 1002C-HH2N | 22 |
| HH2-Q3 | 28 | 1002-G1 | 68 | 1018C-1018N | 31 |
| HH2-Q4 | 45 | 1002-G2 | 74 | 1002C-1002N | 45 |
| HH2-Q5 | 25 | 1002-G3 | 56 | HH2C-HH2N | 61 |
| HH2-Q6 | 35 | 1002-G4 | 42 | 1018N4-1002C8 | 68 |
| HH2-Q7 | 21 | 1002-G5 | 59 | 1018N4-HH2C8 | 35 |
| HH2-Q8 | 65 | 1002-G6 | 26 | 1002N4-1018C8 | 27 |
| HH2-Q9 | 43 | 1002-G7 | 54 | 1002N4-HH2C8 | 8 |
| HH2-Q10 | 61 | 1002-G8 | 42 | HH2N4-1018C8 | 47 |
| HH2-Q11 | 63 | 1002-G9 | 44 | HH2N4-1002C8 | 55 |
| HH2-Q12 | 72 | 1002-G10 | 35 | 1018N8-1002C4 | 42 |
| 1018 | 72 | 1002-G11 | 60 | 1018N8-HH2C4 | 50 |
| 1018-G1 | 38 | 1002-G12 | 50 | 1002N8-1018C4 | 55 |
| 1018-G2 | 59 | 1002-A1 | 55 | 1002N8-HH2C4 | 30 |
| 1018-G3 | 42 | 1002-A2 | 68 | HH2N8-1018C4 | 48 |
| 1018-G4 | 63 | 1002-A3 | 65 | HH2N8-1002C4 | 39 |
| 1018-G5 | 38 | 1002-A4 | 52 | 1018Reverse | 35 |
| 1018-G6 | 36 | 1002-A5 | 70 | 1002Reverse | 72 |
| 1018-G7 | 47 | 1002-A6 | 28 | HH2Reverse | 21 |
| 1018-G8 | 54 | 1002-A7 | 58 | 1018C-1018NRev | 30 |
| 1018-G9 | 67 | 1002-A8 | 52 | 1002C-1002NRev | 18 |
| | | | | HH2C-HH2NRev | 0 |

TABLE 2B
Antibiofilm activity of 1018, 1002 and HH2
derived peptides. Values are reported as the
minimal biofilm inhibitory concentration
(MBIC) that reduced biofilm growth by 50%
compared to growth control samples. These
peptides were rationally designed based on
the results of the immunomodulatory screen
of single amino acid substituted peptides
(Table 2A) of the three parent peptides.
Residues that have been changed relative to
the parent sequence are highlighted in bold.

| | | MBIC₅₀ | MBIC₅₀ |
|---|---|---|---|
| | | S. | P. |
| | | aureus | aeruginosa |
| Peptide | Sequences | (μg/ml) | (μg/ml) |
| 1018 | VRLIVAVRIWRR-NH2 | 20 | 2.5 |
| 2001 | VRLIVKVRIWRR-NH2 | 10 | 2.5 |
| 2002 | VRLIVAVRIRRR-NH2 | 20 | 2.5 |
| 2003 | VRLIVKVRIRRR-NH2 | 20 | 2.5 |
| 2004 | VRVIVKVRIRRR-NH2 | 20 | 2.5 |
| 2005 | VRLIVRVRIWRR-NH2 | 10 | 2.5 |
| 2006 | VRWIVKVRIRRR-NH2 | 10 | 20 |
| 2007 | RRLIVKVRIWRR-NH2 | 10 | 5 |
| 2008 | RRWIVKVRIRRR-NH2 | 10 | 10 |
| 1002 | VQRWLIVWRIRK-NH2 | 10 | 5 |
| 2009 | KWRLLIRWRIQK-NH2 | 5 | 2.5 |
| 2010 | KQRWLIRWRIRK-NH2 | 20 | 2.5 |
| HH2 | VQLRIRVAVIRA-NH2 | 40 | >80 |
| 2011 | VQLRIRVKVIRK-NH2 | 80 | 10 |
| 2012 | WQLRIRVKVIRK-NH2 | 40 | 20 |
| 2013 | WQRVRRVKVIRK-NH2 | >80 | 20 |

Flow Cell Confirmation—

Biofilms were cultivated for 72 h in the presence of 2-20 μg/mL of peptides at 37° C. in flow chambers with channel dimensions of 1×4×40 mm, as previously described (62) but with minor modifications. Silicone tubing (VWR, 0.062 ID×0.125 OD×0.032 wall) was autoclaved and the system was assembled and sterilized by pumping a 0.5% hypochlorite solution through the system at 6 rpm for 1 hour using a Watson Marlow 205S peristaltic pump. The system was then rinsed at 6 rpm with sterile water and medium for 30 min each. Flow chambers were inoculated by injecting 400 μl of mid-log culture diluted to an OD₆₀₀ of 0.02 with a syringe. After inoculation, chambers were left without flow for 2 h after which medium was pumped though the system at a constant rate of 0.75 rpm (3.6 ml/h). Microscopy was done with a Leica DMI 4000 B widefield fluorescence microscope equipped with filter sets for monitoring of blue [Excitation (Ex) 390/40, Emission (Em) 455/50], green (Ex 490/20, Em 525/36), red (Ex 555/25, Em 605/52) and far red (Ex 645/30, Em 705/72) fluorescence, using the Quorum Angstrom Optigrid (MetaMorph) acquisition software. Images were obtained with a 63×1.4 objective. Deconvolution was done with Huygens Essential (Scientific Volume Imaging B.V.) and 3D reconstructions were generated using the Imaris software package (Bitplane AG).

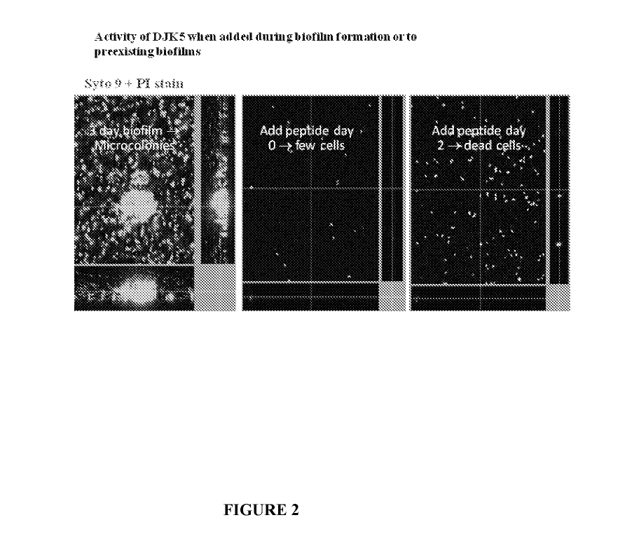

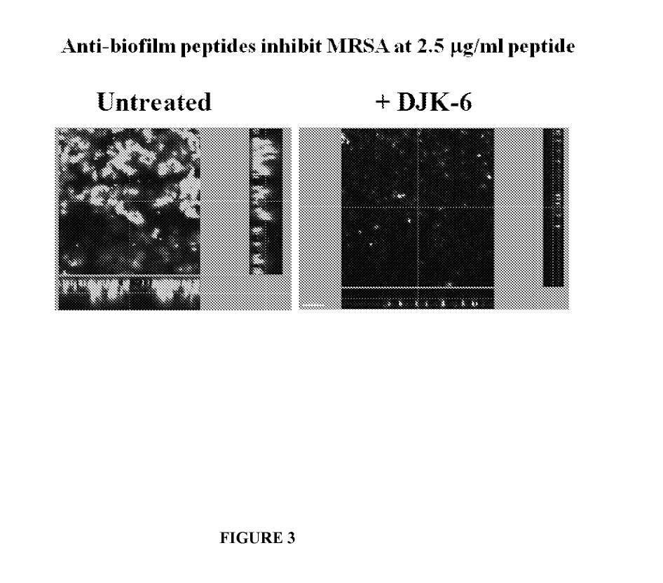

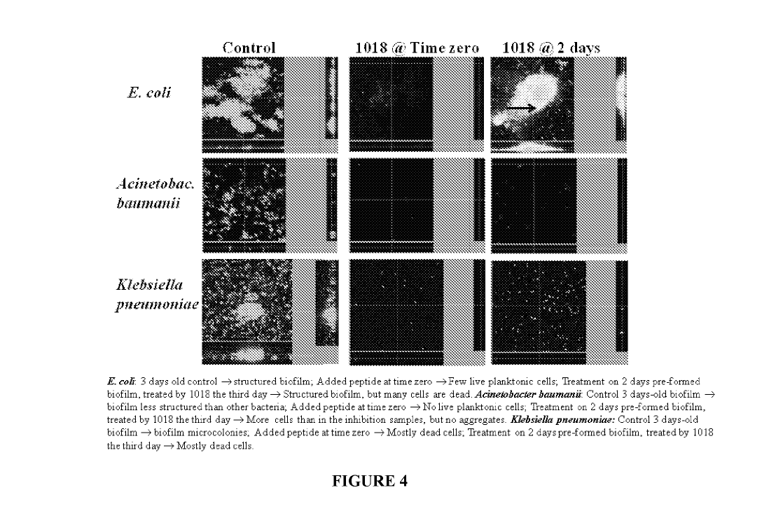

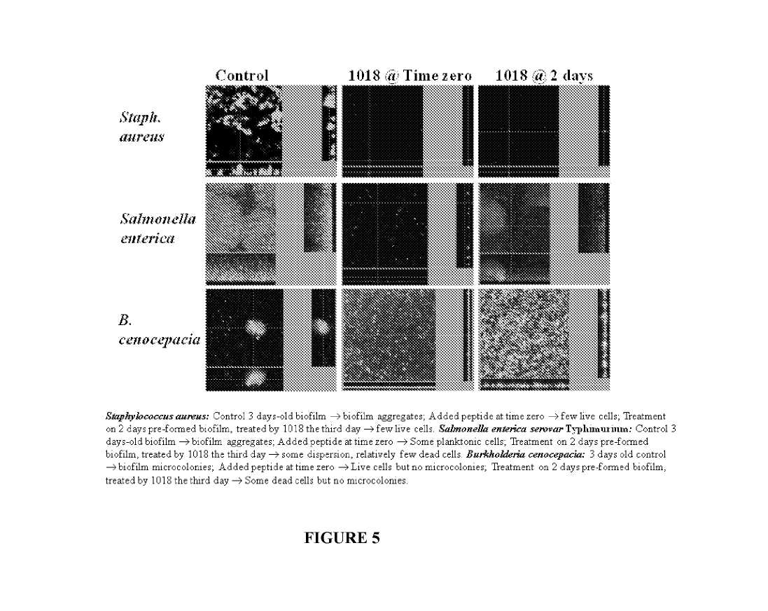

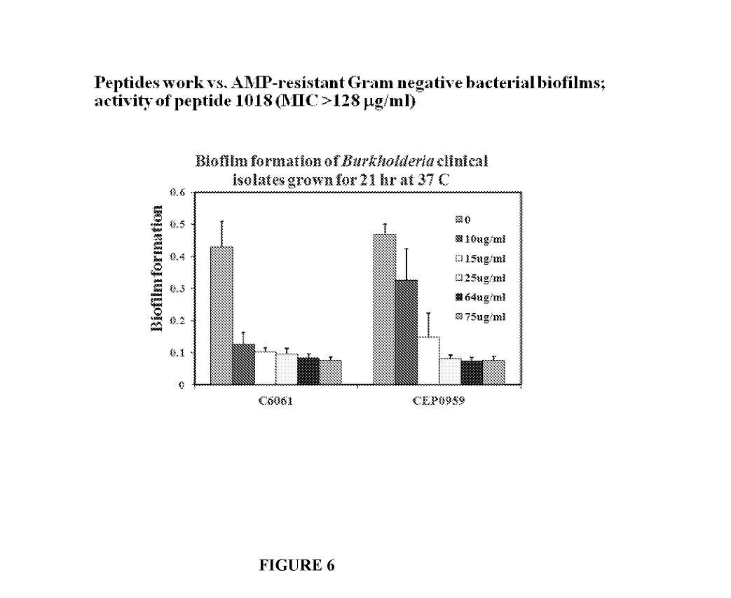

FIGS. 2, 3, 4 and 5 show representative images with peptides DJK-5 vs. Pseudomonas biofilms (FIG. 2), DJK-6 vs. methicillin resistant S. aureus (MRSA) biofilms (FIG. 3) and peptide 1018 vs. E. coli, Acinetobacter baumannii, Klebsiella pneumoniae (FIG. 4), S. aureus, Salmonella enterica ssp. Typhimurium and Burkholderia cenocepacia (FIG. 5) biofilms. The excellent activity of peptide 1018 against two further clinical isolates of Burkholderia cepacia complex in simple biofilm assays is shown in FIG. 6. FIGS. 2, 4, and 5 all show that the peptides can work against biofilms when added prior to initiation of biofilm formation or after biofilms had been growing for 2 days (i.e. pre-formed biofilms). FIGS. 2 and 4 shows that the peptides works well against the Gram positive superbug MRSA as well as several Gram negative Species that are amongst the most feared multi-resistant pathogens (FIG. 1,3,4). FIGS. 5 and 6 demonstrate that the peptide works against Burkholderia cenocepacia that is completely resistant to all antimicrobial peptides in its planktonic form due to its altered outer membrane [Moore, R. A., and R. E. W. Hancock. 1986. Involvement of outer membrane of Pseudomonas cepacia in aminoglycoside and polymyxin resistance. Antimicrob. Agents Chemother. 30: 923-926, also confirmed here with minimal inhibitory concentrations (MIC)>128 μg/ml]. This demonstrates that anti-biofilm activity is completely independent of antimicrobial activity vs. planktonic (free swimming) cells. This is almost certainly related to the fact that the biofilm growth state causes very large changes in bacterial gene expression, a subset of which are likely required for making the biofilms resistant to antibiotics, while another subset are likely required for making the biofilms susceptible to inhibition by these peptides. Furthermore most peptides were shown to be active against biofilms at concentrations well below their MIC vs. planktonic cells. In contrast, the highly active known antimicrobial peptide HHC-36 [Cherkasov et al, 2009] was completely inactive vs. biofilms [Table 2].

### Example 3

### Synergy with Conventional Antibiotics

Peptides and various conventional antibiotics were analyzed by checkerboard titration into microtiter trays using CLSI methods (Wiegand, I., K. Hilpert, and R. E. W. Hancock. 2008. Nature Protocols 3:163-175), bacteria added and, after overnight incubation at 37° C., the residual biofilm assessed by the crystal violet method mentioned in the body of the grant, with an A₅₉₅ of 0.2 considered as 100% biofilm inhibition. The effects of the peptide in reducing the minimal biofilm inhibitory concentration (MBIC) of the antibiotic and vice versa were assessed using the Fractional Inhibitory Concentration (FIC) method whereby ΣFIC=FIC_{A}+FIC_{B}=(C_{A}/MIC_{A})+(C_{B}/MIC_{B}), where MBIC_{A} and MBIC_{B} are the MBICs of peptide A and antibiotic B alone, respectively, and C_{A} and C_{B} (expressed in μg/ml) are the MICs of the drugs in combination. This conventional clinical microbiology assay is interpreted as follows

FIC≦0.5=synergy (4-fold decrease in MIC of each compound); shown as bold below for easy viewing.

FIC of 1=additive activity (2-fold decrease in MIC of each compound)

FIC≧4=antagonism

Results are presented in Tables 3-9 and in Tables 3, 4, 6, and 9 were also expressed in terms of the reduction in MIC of the conventional drug in the presence of the anti-biofilm peptide.

TABLE 3
Synergy of anti-biofilm peptides 1018 and DJK5 with conventional
antibiotics vs. E. coli biofilms:

| | | | | |
|---|---|---|---|---|
| | | | Concentration | |
| | | | of peptide - | |
| | | | concentration of | Fold decrease in |
| | MBIC | | antibiotic at FIC | antibiotic |
| Antibiotic | (μg/ml) | FIC | (μg/ml) | concentration |

Synergy for peptide 1018; MBIC = 32 μg/ml

| | | | | |
|---|---|---|---|---|
| Ceftazidime | 0.4 | 1 | 16-0.1 | 4X |
| Ciprofloxacin | 0.02 | 0.6875 | 16-0.005 | 4X |
| Imipenem | 6.4 | 0.75 | 16-1.6 | 4X |
| Tobramycin | 6.4 | 0.375 | 8-1.6 | 4X |

Synergy for peptide DJK5; MBIC = 1.6 μg/ml

| | | | | |
|---|---|---|---|---|
| Ceftazidime | 0.4 | 0.542 | 0.1-0.2 | 2X |
| Ciprofloxacin | 0.02 | 1 | 1.6-0.00125 | 16X |
| Imipenem | 6.4 | 1 | 1.6-0.1 | 64X |
| Tobramycin | 6.4 | 0.5625 | 0.8-0.4 | 16X |

TABLE 4
Synergy of anti-biofilm peptides 1018 and DJK5 with conventional
antibiotics vs. S. aureus biofilms:

| | | | | |
|---|---|---|---|---|
| | | | Concentration | |
| | | | of peptide - | |
| | | | concentration of | Fold decrease in |
| | MBIC | | antibiotic at FIC | antibiotic |
| Antibiotic | (μg/ml) | FIC | (μg/ml) | concentration |

Synergy for peptide 1018; MBIC = 64 μg/ml

| | | | | |
|---|---|---|---|---|
| Ceftazidime | 256 | 0.16 | 8-8 | 32X |
| Ciprofloxacin | >25.6 | 0.25 | 16-0.4 | 64X |
| Imipenem | >102.4 | 0.25 | 8-25.6 | 4X |
| Tobramycin | >102.4 | 0.52 | 32-1.6 | 64X |

Synergy for peptide DJK5; MBIC = 25.6 μg/ml

| | | | | |
|---|---|---|---|---|
| Ceftazidime | 256 | 0.375 | 6.4-32 | 8X |
| Ciprofloxacin | >25.6 | 0.5 | 12.8-6.4 | 4X |
| Imipenem | >102.4 | 0.52 | 12.8-3.2 | 32X |
| Tobramycin | >102.4 | 1 | 25.6-1.6 | 64X |

TABLE 5
Synergy of anti-biofilm peptides 1018 and DJK5 with conventional
antibiotics vs. P. aeruginosa biofilms:

| | | |
|---|---|---|
| | | Concentration of peptide - |
| | | Concentration of antibiotic at the |
| Antibiotic | FIC | FIC (μg/ml) |

Synergy for peptide 1018

| | | |
|---|---|---|
| Ceftazidime | 0.38 | 6.4-1.4 |
| Ciprofloxacin | 0.14 | 0.8-0.04 |
| Imipenem | 0.502 | 0.1-0.8 |
| Tobramycin | 0.502 | 0.1-1.6 |

Synergy for peptide DJK5

| | | |
|---|---|---|
| Ceftazidime | 0.51 | 0.1-0.8 |
| Ciprofloxacin | 0.14 | 0.1-0.04 |
| Imipenem | 0.51 | 0.1-0.8 |
| Tobramycin | 0.51 | 0.1-1.6 |

TABLE 6
Synergy of anti-biofilm peptide DJK6 with conventional
antibiotics vs. S. aureus biofilms:

| | | Fold decrease in antibiotic |
|---|---|---|
| Antibiotic | FIC | concentration |
| Vancomycin | 0.4 | 16 |
| Ceftazidime | 0.4 | 8 |
| Ciprofloxacin | 0.3 | 16 |

TABLE 7
Synergy of anti-biofilm peptide 1018 with conventional
antibiotics vs. various biofilms expressed as FIC:

| | FIC | | | |
|---|---|---|---|---|
| Biofilm | Ceftazidime | Ciprofloxacin | Imipenem | Tobramycin |
| P. aeruginosa | 0.38 | 0.14 | 0.5 | 0.5 |
| E. coli | 1 | 0.69 | 0.75 | 0.38 |
| A. baumannii | 0.37 | 0.52 | 0.53 | 0.38 |
| S. aureus | 0.16 | 0.25 | 0.25 | 0.5 |
| K. pneumoniae | 0.75 | 0.63 | 0.53 | 0.31 |
| Salmonella | 0.31 | 1 | 1 | 0.75 |

TABLE 8
Synergy of anti-biofilm peptide DJK5 with conventional
antibiotics vs. various biofilms expressed as FIC:

| | FIC | | | |
|---|---|---|---|---|
| Biofilm | Ceftazidime | Ciprofloxacin | Imipenem | Tobramycin |
| P. aeruginosa | 0.5 | 0.14 | 0.5 | 0.5 |
| E. coli | 0.54 | 1 | 1 | 0.56 |
| A. baumannii | 0.75 | 1 | 0.75 | 0.56 |
| S. aureus | 0.38 | 0.5 | 0.52 | 1 |
| K. pneumoniae | 0.89 | 0.75 | 1 | 0.75 |
| Salmonella | 0.75 | 0.56 | 1 | 1.03 |

TABLE 9
Synergy of anti-biofilm peptide DJK5 with conventional
antibiotics vs. various biofilms expressed as fold decrease
in MIC of the conventional antibiotic:

| | Fold decrease in antibiotic | | | |
|---|---|---|---|---|
| | MIC in the presence of peptide | | | |
| Biofilm | Ceftazidime | Ciprofloxacin | Imipenem | Tobramycin |
| E. coli | 2X | 16X | 64X | 16X |
| A. baumannii | 2X | 1 | 2X | 16X |
| S. aureus | 8X | 4X | 32X | 64X |
| K. pneumoniae | 16X | 2X | 64X | 4X |
| Salmonella | 4X | 2X | 2X | 32X |

The results demonstrate either synergy or near synergy for many situations. This was due in part to a substantial lowering of the MIC for peptides or the antibiotics; for example, especially DJK5 has an MIC for complete inhibition of Pseudomonas aeruginosa of 1 μg/ml in the absence of antibiotics, and 0.1 μg/ml in the presence of antibiotics. For ciprofloxacin in P. aeruginosa, the MIC in the presence of peptide was reduced from 500 to 40 ng/ml.

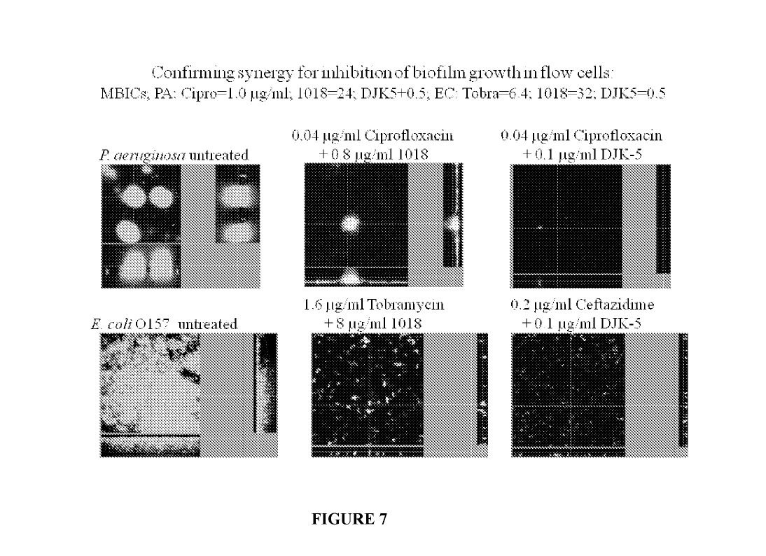

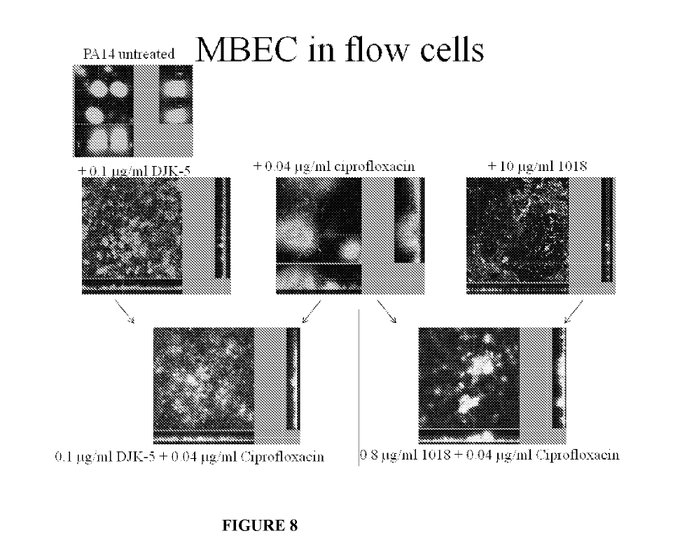

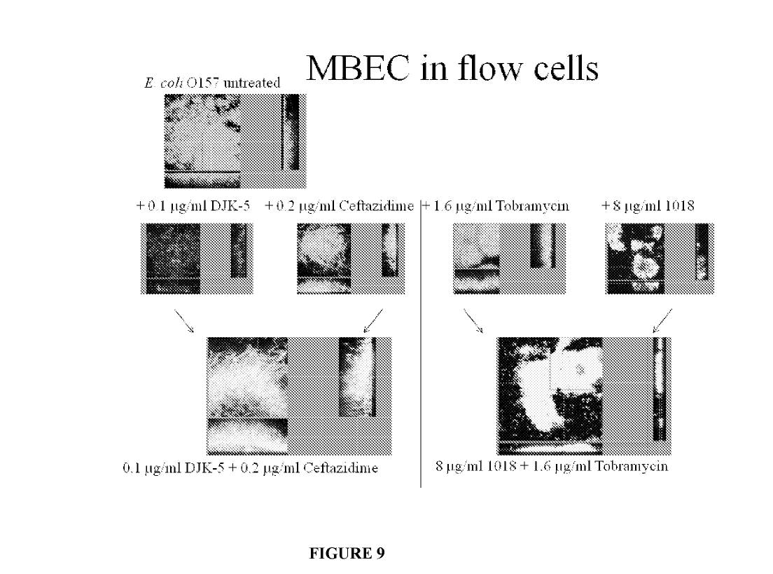

This was also confirmed by flow cell experiments (FIGS. 7, 8, and 9). When anti-biofilm peptide and antibiotic were added together to biofilms, at concentrations that caused minimal effects on biofilms when added separately, the combinations caused substantial disruption of biofilms and/or massive death (demonstrated by the yellow staining which is due to an overlay of the green color of the general stain Cyto-9 and the red color of the dead cell stain propidium iodide. Thus it is clear that the anti-biofilm peptides promote the activities of conventional antibiotics and vice versa.

### Example 5

### Mechanism of Action Studies

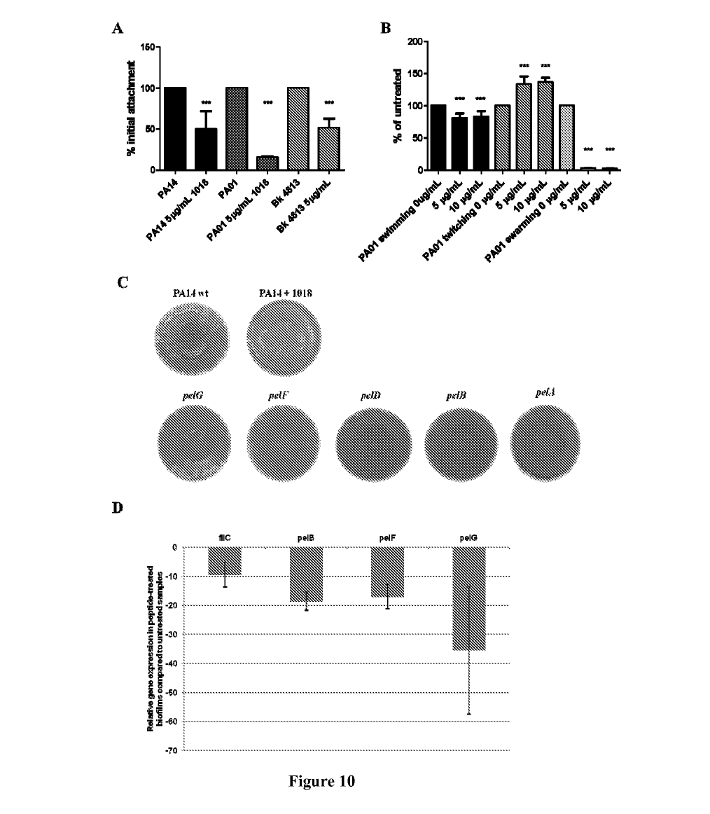

Biofilm formation depends on the initial attachment of planktonic cells to surfaces. Therefore, blocking this early event in biofilm development is key for efficient biofilm treatment. Based on this notion, we decided to test whether 1018 (SEQ ID No 8) interfered with early surface attachment. For this, bacterial cells were treated with the peptide and allowed to bind to the surface of polypropylene plates for 3 hours. Initial attachment was reduced by at least 50% in P. aeruginosa (PAO1 and PA14) and B. cenocepacia clinical isolate 4813 (FIG. 10A).

Bacterial translocation on surfaces also significantly contributes to the proper development and stability of biofilms. Swimming motility depends on the activity of flagella, which propel cells across semi-liquid surfaces (such as 0.3% agar). Planktonic cells depend on their ability to swim towards a surface in order to initiate the development of biofilms and thus represent an interesting target. Peptide 1018 significantly reduced the ability of bacteria to swim on surfaces (FIG. 10B). Furthermore, the flagellin gene fliC was significantly down-regulated (−9.44±4.2) in biofilms treated with 10 μg/mL 1018 (FIG. 10D).

Type-IV pili-dependent twitching motility allows bacteria to translocate on solid surfaces (e.g., 1% agar). These pili are composed primarily of a single small protein subunit, termed PilA or pilin. Stimulation of this type of motility has been shown to lead to both inability to form biofilms and biofilm dispersion. Low levels of the peptide induced twitching motility (FIG. 10B). In addition, the P. aeruginosa gene pilA that encodes for PilA was up-regulated by 5.26±0.23 fold in biofilm cells treated with sub-MIC levels of 1018 (1 μg/mL), as determined by RT-qPCR assays. These results suggest that the peptide may activate this process resulting in both inhibition of biofilm formation and dispersal of cells from biofilms.

In P. aeruginosa, the products of seven adjacent genes commonly referred to as the pel operon synthesize Pel polysaccharide, which is involved in the formation of the protective extracellular matrix in pellicle biofilms and is required for the formation of solid surface-associated biofilms. Indeed, expression of the pel genes is associated with the production of the matrix component Pel, that allows binding of Congo red. In fact, a standard experimental procedure to identify Pel polysaccharide is based on its ability to bind to Congo red. When grown on agar plates containing Congo red, P. aeruginosa and B. cenocepacia biofilm colonies were dark red whereas the pel mutants were pale pink-white (FIG. 10C). The wild type colonies also had a wrinkled or ‘rugose’ morphology, whereas the pel mutant colonies were smooth (FIG. 10C). The smooth phenotype of the pel mutant colonies is known to be due to the loss of the extracellular matrix component Pel polysaccharide. Addition of low levels of peptide 1018 to Congo red plates led to colony biofilms similar to those formed by pel mutants (FIG. 10C). Further RT-qPCR experiments revealed that treatment of cells undergoing early biofilm development with 10 μg/mL 1018 led to down-regulation of pelG (−35.5±21.98), pelB (−18.63±3.09) and pelF (−17.04±4.13), all genes involved in Pel synthesis (FIG. 10D).

These mechanisms described above were unsatisfying since the anti-biofim activity was very broad spectrum while the above mechanisms were somewhat specific for Pseudomonas. To provide a more general explanation for the broad spectrum anti-biofilm action we turned to the stringent response as a potential explanation.

Bacteria are known to respond to stressful environmental conditions (such as starvation) by activating the stringent response. As a consequence, the stressed cell synthesizes two small signaling nucleotides—guanosine 5′-diphosphate 3′-diphosphate (ppGpp) and guanosine 5′-triphosphate 3′-diphosphate (pppGpp), together denoted (p)ppGpp—which serve as a second messenger that regulate the expression of many genes in both Gram-negative and Gram-positive species (Magnusson L U, Farewell A, Nyström T (2005) ppGpp: a global regulator in Escherichia coli. Trends Microbiol 13:236-42.; Potrykus K, Cashel M. 2008 (p)ppGpp: still magical? Annu Rev Microbiol. 62:35-51). (p)ppGpp is synthesized by the ribosome-dependent pyrophosphate transfer of the β and γ phosphates from an ATP donor to the 3′ hydroxyl group of GTP or GDP. In Gram negative bacteria (p)ppGpp production mostly depends on synthetase RelA; the enzyme SpoT contributes to both synthesis and hydrolysis of (p)ppGpp. Likewise, in Gram positive bacteria, a bifunctional enzyme, RelA/SpoT homolog (Rsh), is responsible for both synthesis and hydrolysis of (p)ppGpp.

Since, bacteria predominantly exist as biofilms rather than free-swimming (planktonic) cells in most environments, we hypothesized that a universal environmental stress signal could be responsible for the transition to the biofilm phenotype. Most environments are known to encounter periods of nutrient limitation or starvation that expose the population to a life or death situation. In bacteria, the nucleotide (p)ppGpp is produced intracellularly in response to a variety of environmental stresses, a process commonly referred to as the stringent response. We argued that, upon starvation, bacterial cells could induce (p)ppGpp synthesis, which in turn would lead to the development of biofilms.

If our hypothesis were correct, mutants lacking the ability to produce (p)ppGpp should be unable to develop biofilms under conditions that enable planktonic growth. We confirmed this prediction in a series of experiments.

In addition, we found that starvation led to biofilm formation through the activation of (p)ppGpp. Notably, peptide 1018 with potent, broad-spectrum anti-biofilm activity was found to inhibit (p)ppGpp synthesis. Conversely, (p)ppGpp overproduction led to peptide resistance. Taken together, our results suggest the peptide repressed (p)ppGpp accumulation thus blocking the universal signal for biofilm development.

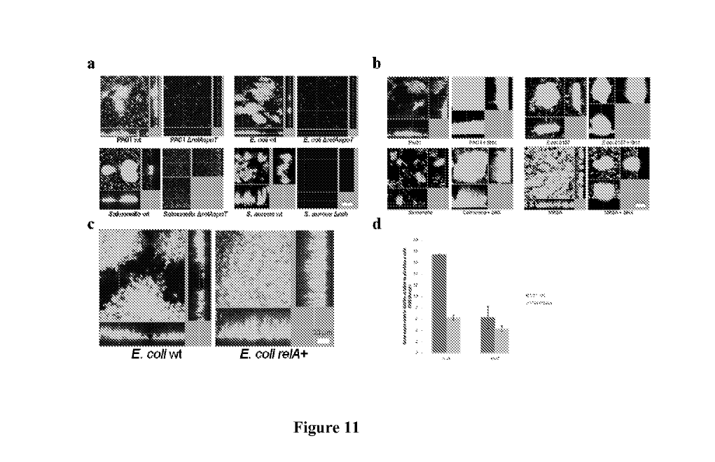

We first monitored biofilm formation of wild-type strains of P. aeruginosa, Salmonella, Escherichia coli and the Gram-positive Staphylococcus aureus and their respective (p)ppGpp mutants. Cells unable to synthesize (p)ppGpp did not adhere tightly to the plastic surface of flow cell chambers and were unable to develop structured biofilms (FIG. 11a). Indeed, (p)ppGpp-negative cells appeared to be in the planktonic (free swimming) state, as they underwent continuous swimming around the chamber as opposed to adhering to its surface. These swimming cells were easily cleared by stresses as mild as increased flow rate. Genetic complementation restored the ability to form biofilms. These results are consistent with the hypothesis that (p)ppGpp plays a fundamental role in initiating the biofilm developmental process.

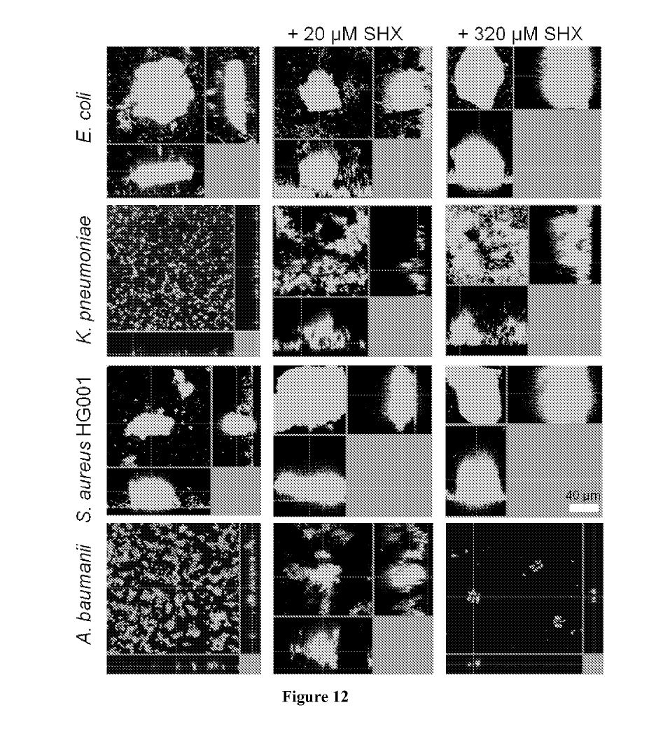

To further confirm the hypothesis, we evaluated the effect of chemically-induced starvation on biofilm formation. Starvation was artificially achieved by using serine hydroxamate (SHX), a structural analogue of L-serine, which induces the stringent response by inhibiting charging of seryl-tRNA synthetase and is known to promote growth arrest of planktonic cells. To evaluate the effect of SHX on biofilms, wild-type cells of the different bacterial strains were grown in flow cell chambers and treated with different concentrations of SHX. Interestingly, we noticed that cells tended to aggregate and developed large, structured microcolonies in certain regions of the flow cells (FIG. 11b). In other words, in the presence of SHX, bacteria were driven to form more robust multicellular biofilms. The degree of biofilm induction depended on the concentration of SHX used (FIG. 12) and the minimum concentration of SHX required to trigger biofilm development varied among bacterial species.

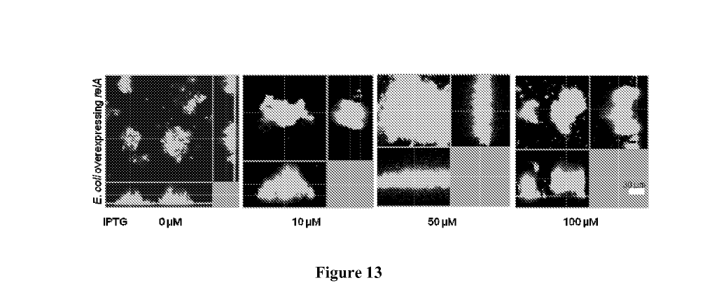

In addition, overexpression of the major (p)ppGpp synthetase gene relA in E. coli resulted in a hyper-biofilm phenotype (FIG. 11c) and this was indeed dependent on the relative overexpression of relA, controlled by increasing concentrations of Isopropyl β-D-1-thiogalactopyranoside (IPTG) (FIG. 13). To assess whether biofilm cells synthesized more (p)ppGpp than planktonic cells, the expression of the two (p)ppGpp synthetase genes relA and spoT present in P. aeruginosa was evaluated by qRT-PCR. These genes were significantly up-regulated in biofilm cells compared to both stationary and mid-log phase bacteria (FIG. 11d).

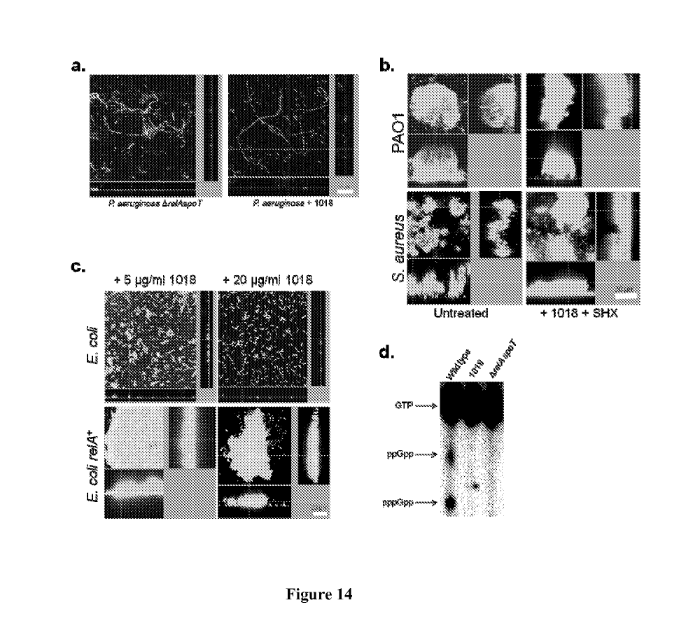

We then investigated how anti-biofilm peptide 1018 affected the formation of biofilms. While performing flow cell biofilm experiments, we noticed that both mutations in (p)ppGpp and peptide-treated samples induced bacterial cell filiamentation and cell death (FIG. 14a). Based on these and the previously described observations, we hypothesized that the peptide exerted its potent broad-spectrum anti-biofilm activity by repressing (p)ppGpp production or targeting (p)ppGpp for degradation. A commonly used strategy to identify potential antimicrobial targets is to overexpress them and see if that leads to resistance to the particular antimicrobial agent used. To determine if overproduction of (p)ppGpp led to peptide resistance, we used both the E. coli strain overexpressing wild-type relA under the control of an IPTG-inducible promoter and wild type E. coli treated with SHX to induce (p)ppGpp. In both scenarios, biofilms became resistant to the presence of the peptide (FIG. 14b,c), thus indicating that (p)ppGpp overexpression suppressed 1018 anti-biofilm activity and suggesting that it was the likely target of the peptide. To directly demonstrate this, we examined, by thin layer chromatography, the levels of (p)ppGpp produced by biofilms in the presence and absence of peptide 1018. These experiments revealed that cells treated with peptide 1018 did not accumulate (p)ppGpp (FIG. 14d) indicating that 1018 acted by suppressing the effects of (p)ppGpp in promoting biofilm formation. Indeed adding peptide to cells that had accumulated (p)ppGpp led to raid degradation (as judged by thin layer chromatography or NMR within 30 minutes) We were also able to demonstrate by NMR that peptide 1018 was able to directly bind to synthetic ppGpp, suppressing the NMR signal. Together this indicates that 1018 binds to (p)ppGpp and marks it for degradation by enzymes like SpoT.

Similar results to those shown with 1018 (FIG. 14) were also observed with peptide DJK5.

### Example 6

### Effect on Swarming

Motility is strongly involved in the virulence of bacteria since it plays an important role in the attachment of bacteria to surfaces, including those in the body and on indwelling medical devices, and in colonization of these surfaces and biofilm formation. P. aeruginosa is known to utilize at least 4 different types of motility: (a) flagellum-mediated swimming in aqueous environments and at low agar concentrations (<0.3% agar), (b) type IV pilus-mediated twitching on solid surfaces or interfaces, (c) swarming on semi-solid media (0.5-0.7% agar) in poor nitrogen (N) sources such as amino acids (AA) and (d) surfing on low agar concentrations containing mucin.

Swarming motility is a social phenomenon (a complex adaptation) involving the coordinated and rapid movement of bacteria across a semi-solid (viscous) surface, and is widespread among flagellated pathogenic bacteria. With specific reference to Pseudomonas virulence, the mucous environment of the lung, especially in the case of chronic (mucoid) infections of CF patients, can be considered to be a viscous environment with amino acids serving as the main N source, which might equate to swarming motility conditions. Swarming in P. aeruginosa leads to dendritic (strain PA14) or solar flare like (strain PAO1) colonial structures. Comparing the leading edge of tendrils to the center of swarming zones revealed coordinated (aligned) cells that are resistant to all tested antibiotic classes except polymyxins. Microarray analysis under these conditions revealed that the leading edge cells demonstrated dysregulation of 417 genes (309 up- and 108 down-regulated), including 18 regulators, and numerous genes involved in energy metabolism, nitrogen assimilation, fatty acid biosynthesis, transport and phenazine production [Overhage, J, M Bains, M D Brazas, and R E W Hancock. 2008. Swarming of Pseudomonas aeruginosa is a complex adaptation leading to increased production of virulence factors and antibiotic resistance. J Bacteriol 190:2671-2679]. Under swarming conditions there was also upregulation of virtually all known virulence factors (by 2- to 11-fold) and many antibiotic resistance genes. Mutant library screening [Yeung A. T. Y., E. C. W. Torfs, F. Jamshidi, M. Bains, I. Wiegand, R. E. W. Hancock, and J. Overhage. 2009. Swarming of Pseudomonas aeruginosa is controlled by a broad spectrum of transcriptional regulators including MetR. 2009. J Bacteriol 191:5591-5602] revealed 233 genes that were essential to this process, including 35 regulators that when mutated inhibited or blocked swarming (two caused hyperswarming), but generally did not affect swimming or twitching motility.

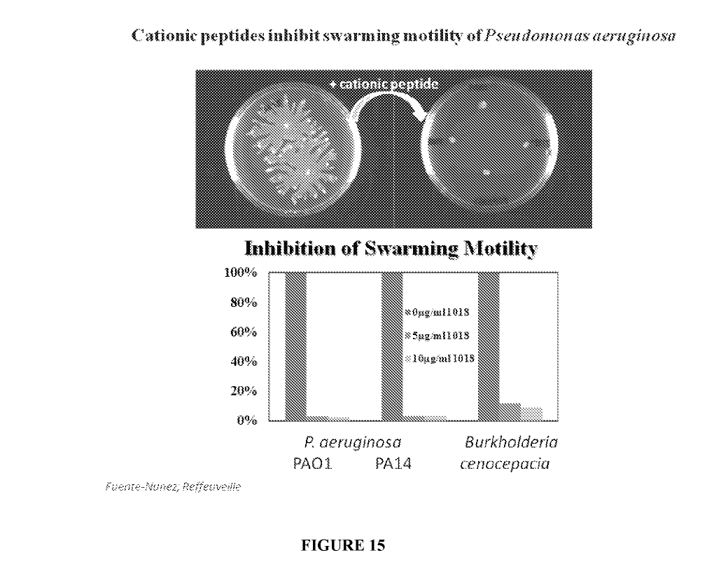

These data clearly indicate that swarming is not just a third kind of motility but an alternative growth state (complex adaptation) and due to the massive complexity involved we have focused on specific regulators that affect metabolism. Evidence was obtained that peptide 1018 and other anti-biofilm peptides are able to completely knock down swarming motility at low concentrations (FIG. 15). This was independent of bacterial killing (antimicrobial peptide action measured by MIC) since these peptides were able to inhibit the swarming of Burkholderia cenocepacia, which as mentioned above is completely resistant to all cationic antimicrobial peptides due to its altered outer membrane. The peptide also inhibited surfing motility on mucin-containing plates. In contrast the conventional cationic antibiotic, polymyxin B, does not have anti-swarming or anti-surfing activity.

### Example 7

### Animal Models

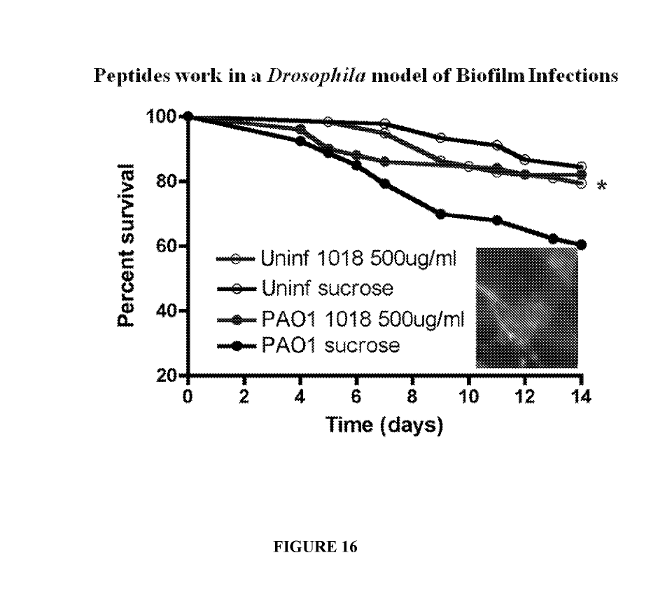

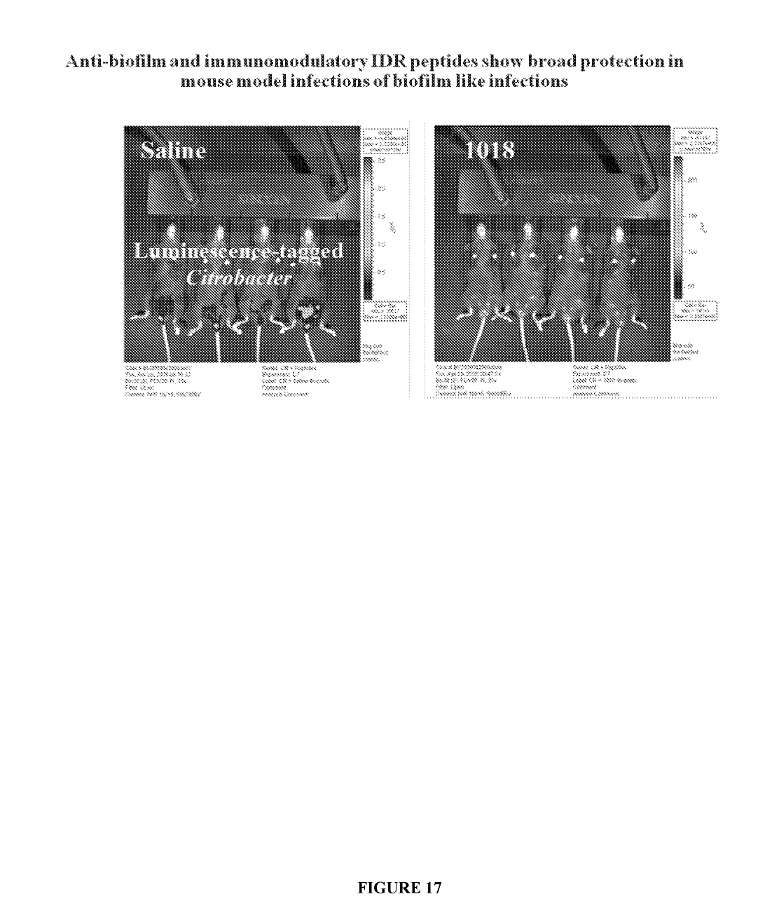

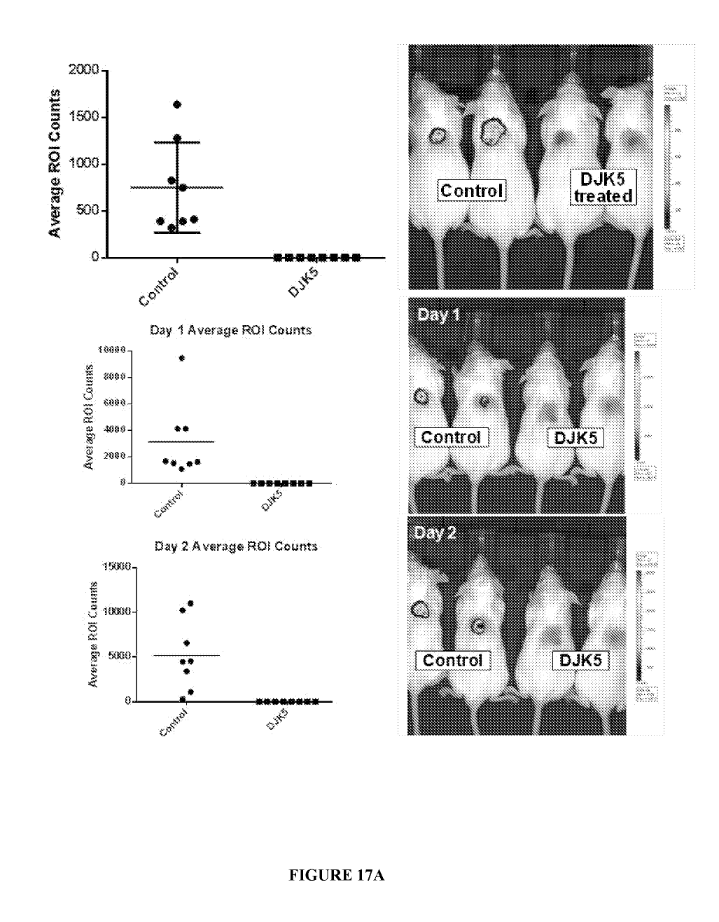

To confirm the potential utility of these peptides in treating infections, two models were initially utilized. The first examined protection by an anti-biofilm peptide in a Drosophila model of Pseudomonas aeruginosa biofilm infection [Mulcahy, H., C. D. Sibley, M. G. Surette, and S. Lewenza. 2011. Drosophila melanogaster as an animal model for the study of Pseudomonas aeruginosa biofilm infections in vivo. PLoS Pathogens 7(10):e1002299]. The inset to FIG. 16 shows the in vivo biofilm growth mode of Pseudomonas (stained green in this model). Protection was observed due to injection of anti-biofilm peptide 1018 (FIG. 16), and was equivalent to protection seen due to injection of 5 μg/ml tobramycin (not shown). Anti-biofilm peptide 1018 also demonstrated anti-infective activity in a Citrobacter rodentium (luxCDABE) mouse model (FIG. 17), where the Citrobacter appeared to form biofilms in the gastrointestinal tract of mice. The Citrobacter was imaged by IVIS imaging of light production at day 7 after application of a single dose of peptides (8 mg/kg) at time −4 hr. Peptide 1018 led to the complete loss of all bacteria.

Using a surface abrasion model (FIG. 17A) we were also able to clearly show the protective nature of these peptides in a murine biofilm infection model.

D-Enantiomeric Peptides Protected Caenorhabditis elegans and Galleria mellonella from P. aeruginosa Biofilm Infections.

D-enantiomeric peptides DJK-5, DJK-6 and RI-1018 were tested in vivo for their ability to protect the nematode C. elegans and the moth G. mellonella from biofilm infections induced by P. aeruginosa PAO1, using previously-described models (Brackman G, Cos P, Maes L, Nelis H J, and Coenye T. 2011. Quorum sensing inhibitors increase the susceptibility of bacterial biofilms to antibiotics in vitro and in vivo. Antimicrobial Agents Chemotherapy 55:2655-61).

The C. elegans survival assay was carried out as previously described (Brackman et al., 2011). In brief, synchronized worms (L4 stage) were suspended in a medium containing 95% M9 buffer (3 g of KH₂PO₄, 6 g of Na₂HPO₄, 5 g of NaCl, and 1 ml of 1 M MgSO₄.7H₂O in 1 liter of water), 5% brain heart infusion broth (Oxoid), and 10 μg of cholesterol (Sigma-Aldrich) per ml. 0.5 ml of this suspension of nematodes was transferred to the wells of a 24-well microtiter plate. An overnight bacterial culture was centrifuged, resuspended in the assay medium, and standardized to 10⁸ CFU/ml. Next, 250 μl of this standardized suspension were added to each well, while 250 μl of sterile medium was added to the positive control. Peptides were added to the test wells at a final concentration of 20 μg/ml. The assay plates were incubated at 25° C. for up to 2 days. The fraction of dead worms was determined by counting the number of dead worms and the total number of worms in each well, using a dissecting microscope. Peptides were tested at least four times in each assay, and each assay was repeated at least three times (n≧12).

The peptides did not display any toxic activity against C. elegans, since no significant differences in survival were observed after 24 h and 48 h in uninfected C. elegans nematodes treated with peptides compared to untreated animals (Table 10). Untreated controls infected with P. aeruginosa PAO1 demonstrated 100% death after 48 h in both biofilm infection models (Table 10). We tested 4 anti-biofilm peptides 1018, its D-enantiomeric retro-inverso version RI-1018, and DJK-5 and DJK-6. In the C. elegans experiments, all peptides significantly (p<0.001) protected the nematodes against P. aeruginosa PAO1-induced mortality after 24 h, with DJK-5 and DJK-6 giving nearly complete protection (Table 10). After 48 h of infection, significant protection (p<0.001) was still observed for groups treated with peptides DJK-5 and DJK-6, while mortality was close to 100% (and not significantly different from the peptide untreated control) for RI-1018 and 1018 (Table 10).

The G. mellonella survival assay was carried out as previously described (Brackman et al., 2011). In brief, prior to injection in G. mellonella, bacterial cells were washed with PBS and then diluted to either 10⁴ or 10⁵ CFU per 10 μl. A Hamilton syringe was used to inject 10 μl in the G. mellonella last left proleg. The peptides (20 μg/10 μl) were administered by injecting 10 μl into a different proleg within 1 h after injecting the bacteria. Two control groups were used: the first group included uninfected larvae injected with PBS to monitor killing due to physical trauma; the second group included uninfected larvae receiving no treatment at all. Results from experiments in which one or more larvae in either control group died were discarded and the experiments were repeated. To evaluate the toxicity of the peptides, uninfected larvae were injected with peptides. Larvae were placed in the dark at 37° C. and were scored as dead or alive 24 h and 48 h post-infection. Larvae were considered dead when they displayed no movement in response to shaking or touch. At least 20 larvae were injected for each treatment. For each treatment, data from at least six independent experiments were combined.

In experiments performed using the Galleria biofilm model, in which moths were infected with 10⁴ CFU, no protective effect was observed after 24 h with peptide 1018, a moderate but significant protective effect was observed for RI-1018 and DJK-6, and a strong and significant protective effect was conferred by DJK-5 (Table 10). After 48 h, RI-1018 and particularly peptides DJK-5 and DJK-6 resulted in increased survival (18-42% survival cf. complete killing in the control group) (Table 10).

TABLE 10
In vivo anti-biofilm activity of D-enantiomeric peptides. C. elegans and
G. mellonella biofilm survival assays. Percent survival of infected
C. elegans and G. mellonella (average ± the SD) after treatment
with peptides D-enantiomeric peptides RI-1018 (and its L-version 1018),
DJK-5 and DJK-6 and P. aeruginosa strain PAO1. The results are
expressed as the percent survival after both 24 h and 48 h of infection
and peptide treatment. Statistical significance comparing peptide-treated
groups to untreated was determined (*, P < 0.001).

| | 24 h | | 48 h post infection | |
|---|---|---|---|---|
| | | P. aeruginosa | | P. aeruginosa |
| Peptide | No infection | PAO1 | No infection | PAO1 |
| | C. elegans survival (%) | | | |
| None | 100 ± 0 | 61 ± 21 | 95 ± 4 | 1 ± 2 |
| RI1018 | 99 ± 1 | 83 ± 13* | 81 ± 23 | 4 ± 6 |
| 1018 | 97 ± 4 | 91 ± 12* | 88 ± 9 | 1 ± 3 |
| DJK5 | 99 ± 2 | 99 ± 2* | 99 ± 2 | 96 ± 4* |
| DJK6 | 99 ± 2 | 99 ± 2* | 97 ± 4 | 90 ± 5* |
| | G. mellonella survival (%) | | | |
| CTRL | 100 ± 0 | 13 ± 11 | 100 ± 0 | 0 ± 0 |
| RI1018 | 90 ± 14 | 50 ± 8* | 80 ± 10 | 18 ± 7* |
| 1018 | 90 ± 14 | 27 ± 11 | 90 ± 14 | 3 ± 5 |
| DJK5 | 100 ± 0 | 90 ± 6* | 100 ± 0 | 42 ± 7* |
| DJK6 | 100 ± 0 | 50 ± 8* | 100 ± 0 | 30 ± 6* |

*survival significantly different from untreated control (p < 0.001)

### Example 8

### Enhancement of Innate Immunity

The natural human peptide LL-37 is able to protect against bacterial infections despite having no antimicrobial activity under physiological conditions (Bowdish, D. M. E., D. J. Davidson, Y. E. Lau, K. Lee, M. G. Scott, and R. E. W. Hancock. 2005. Impact of LL-37 on anti-infective immunity. J. Leukocyte Biol. 77:451-459). Innate defence regulator peptide (IDR)-1 that had no direct antibiotic activity was nevertheless able, in mouse models, to protect against infections by major Gram-positive and -negative pathogens, including MRSA, VRE and Salmonella [Scott M G, E Dullaghan, N Mookherjee, N Glavas, M Waldbrook, A. Thompson, A Wang, K Lee, S Doria, P Hamill, J Yu, Y Li, O Donini, M M Guarna, B B Finlay, J R North, and R E W Hancock. 2007. An anti-infective peptide that selectively modulates the innate immune response. Nature Biotech. 25: 465-472]. IDR-1 peptide functioned by selectively modulating innate immunity, i.e. by suppressing potentially harmful inflammation while stimulating protective mechanisms such as recruitment of phagocytes and cell differentiation. This was also true of peptide 1018 which demonstrated superior protection in models of cerebral malaria and Staph aureus [Achtman, A H, S Pilat, C W Law, D J Lynn, L Janot, M Mayer, S Ma, J Kindrachuk, B B Finlay, F S L Brinkman, G K Smyth, R E W Hancock and L Schofield. 2012. Effective adjunctive therapy by an innate defense regulatory peptide in a pre-clinical model of severe malaria. Science Translational Medicine 4:135ra64] and tuberculosis [Rivas-Santiago, B., J. E. Castañeda-Delgado, C. E. Rivas Santiago, M. Waldbrook, I. González-Curiel, J. C. León-Contreras, A. Enciso-Moreno, V. del Villar, J. Méndez-Ramos, R. E. W. Hancock, R. Hernandez-Pando. 2013. Ability of innate defence regulator peptides IDR-1002, IDR-HH2 and IDR-1018 to protect against Mycobacterium tuberculosis infections in animal models. PLoS One 8:e59119], as well as wound healing [Steinstraesser, L., T. Hirsch, M. Schulte, M. Kueckelhaus, F. Jacobsen, E. A. Mersch, I. Stricker, N. Afacan, H. Jenssen, R. E. W. Hancock and J. Kindrachuk. 2012. Innate defense regulator peptide 1018 in wound healing and wound infection. PLoS ONE 7:e39373]. LL-37 and 1018 appear to manifest this activity due to their ability to induce the production of certain chemokines which are able to recruit subsets of cells of innate immunity to infected tissues and to cause differentiation of recruited monocytes into particular subsets of macrophages with superior phagocytic activity [Pena O. M., N. Afacan, J. Pistolic, C. Chen, L. Madera, R. Falsafi, C. D. Fjell, and R. E. W. Hancock. 2013. Synthetic cationic peptide IDR-1018 modulates human macrophage differentiation. PLoS One 8:e52449]. Therefore we tested if the novel peptides described here also had the ability to induce chemokine production in human peripheral blood mononuclear cells.

Venous blood (20 ml) from healthy volunteers was collected in Vacutainer® collection tubes containing sodium heparin as an anticoagulant (Becton Dickinson, Mississauga, ON) in accordance with UBC ethical approval and guidelines. Blood was diluted 1:1 with complete RPMI 1640 medium and separated by centrifugation over a Ficoll-Paque® Plus (Amersham Biosciences, Piscataway, N.J., USA) density gradient. White blood cells were isolated from the buffy coat, washed twice in RPMI 1640 complete medium, and the number of peripheral blood mononuclear cells (PBMC) was determined by Trypan blue exclusion. PBMC (5×10⁵) were seeded into 12-well tissue culture dishes (Falcon; Becton Dickinson) at 0.75 to 1×10⁶ cells/ml at 37° C. in 5% CO₂. The above conditions were chosen to mimic conditions for circulating blood monocytes entering tissues at the site of infection via extravasation.

Following incubation of the cells under various treatment regimens, the tissue culture supernatants were centrifuged at 1000×g for 5 min, then at 10,000×g for 2 min to obtain cell-free samples. Supernatants were aliquoted and then stored at −20° C. prior to assay for various chemokines by capture ELISA (eBioscience and BioSource International Inc., CA, USA respectively)

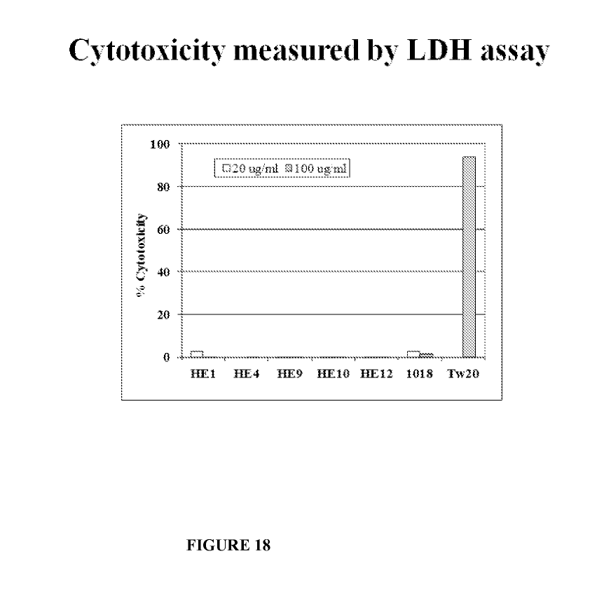

Cytotoxicity was assessed using the Lactate dehydrogenase assay. This was done using the same cell-free supernatants as for cytokine detection except that the supernatants were tested the same day as they were obtained to avoid freeze-thawing. Lactate dehydrogenase (LDH) assay (Roche cat#11644793001) is a colorimetric method of measuring cytotoxicity/cytolysis based on measurement of LHD activity released from cytosol of damaged cells into the supernatant. LDH released from permeable cells into the tissue culture supernatant will act to reduce the soluble pale yellow tetrazolium salt in the LDH assay reagent mixture into the soluble red coloured formazan salt product. Amount of colour formed is detected as increased absorbance measured at ˜500 nm. The calculations were done using the following formula Cytotoxicity %=(exp value−CTR)/(Triton−CTR)*100%. Anything under 10% is considered acceptable. None of the tested peptides showed any LDH release even at 100 μg/ml (FIG. 18).

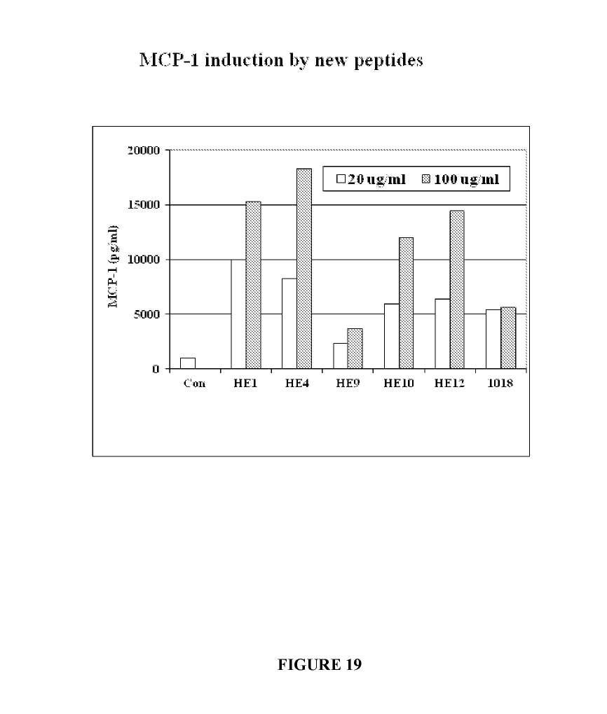

As shown in FIG. 19, most of the peptides stimulated the expression of the macrophage chemokine MCP-1 even at the lowest peptide concentration utilized (20 μg/ml). Indeed peptides HE1, HE4, HE10, and HE12 were clearly superior to peptide 1018 in inducing MCP-1. The basis for the design of these next generation peptides relative to 1018 is shown in Table 11.

TABLE 11
Basis for the design of most active HE peptides.

| Peptide | Design | Sequence |
|---|---|---|
| 1018 | Native 1018 | VRLIVAVRIWRR- |
| | | NH₂ |
| HE1 | Retro 1018 | RRWIRVAVILRV- |
| | | NH₂ |
| HE4 | Substitute in another W | VRLIWAVRIWRR- |
| | | NH₂ |
| HE10 | Truncate to remove hydrophobic patch | VRLIVRIWRR-NH₂ |
| HE12 | Add RFK entry sequence and truncate | RFKRVARVIW-NH₂ |

Based on these results, new peptides were iteratively designed from our best immunomodulatory peptides by substitution analysis of peptide sequences using SPOT synthesis on cellulose, and tested for immunomodulatory activity (production of chemokine MCP-1 from human peripheral blood mononuclear cells treated with at ˜18-24 μM concentrations. Results are shown in columns 2 and 3 of Tables 12 and 12A with results in bold showing very substantial changes relative to control (parent) peptides.

TABLE 12
Screening of substituted derivatives for enhanced immunomodulatory and
anti-inflammatory activity. Results in column 2 are background subtracted (139 and
170 pg/ml for the HH2 and 1018 derivatives respectively. Results shown in bold led to very
substantial changes relative to the control peptides HH2 and 1018 respectfully. Many other
peptides were at least equivalent to or better than parent peptides HH2 and 1018 in MCP-1
induction are not marked.

| | | | Cells stimulated | |
|---|---|---|---|---|
| | No LPS | | with 10 ng/ml LPS | |
| | | Fold | | Fold change in |
| | | increase in | | IL1β relative to |
| | | MCP1 cf. | IL1β Production | no peptide LPS |
| Peptide | MCP1 (pg/ml)^{a} | untreated cells | (pg/ml)^{b} | stimulated cells |
| No Peptide | 0 | 1.0 | 1313 (LPS alone) | 1.00 |
| HH2 | 450 | 3.6 | 1307 | 1.00 |
| HH2-G1 | 157 | 1.9 | 1533 | 1.17 |
| HH2-G2 | 471 | 3.8 | 1400 | 1.07 |
| HH2-G3 | 117 | 1.7 | 1687 | 1.29 |
| HH2-G4 | 25045 | 148 | 1582 | 1.21 |
| HH2-G5 | 321 | 2.9 | 1672 | 1.27 |
| HH2-G6 | 1287 | 8.6 | 1423 | 1.08 |
| HH2-G7 | 78 | 1.5 | 1344 | 1.02 |
| HH2-G8 | 157 | 1.9 | 1344 | 1.02 |
| HH2-G9 | 342 | 3.0 | 1363 | 1.04 |
| HH2-G10 | 12063 | 72 | 1391 | 1.06 |
| HH2-G11 | 177 | 2.0 | 1303 | 0.99 |
| HH2-G12 | 11442 | 68 | 1433 | 1.09 |
| HH2-A1 | 2254 | 14 | 1587 | 1.21 |
| HH2-A2 | 471 | 3.8 | 1612 | 1.23 |
| HH2-A3 | 59 | 1.3 | 1886 | 1.44 |
| HH2-A4 | 672 | 5.0 | 2000 | 1.52 |
| HH2-A5 | 59 | 1.3 | 2095 | 1.60 |
| HH2-A6 | 137 | 1.8 | 1391 | 1.06 |
| HH2-A7 | 59 | 1.3 | 1713 | 1.30 |
| HH2-A9 | 157 | 1.9 | 1923 | 1.46 |
| HH2-A10 | 258 | 2.5 | 2234 | 1.70 |
| HH2-A11 | 833 | 5.9 | 2105 | 1.60 |
| HH2-R1 | 4034 | 25 | 1965 | 1.50 |
| HH2-R2 | 604 | 4.6 | 1438 | 1.10 |
| HH2-R3 | 9987 | 60 | 1746 | 1.33 |
| HH2-R5 | 515 | 4.0 | 2186 | 1.66 |
| HH2-R7 | 98 | 1.6 | 1959 | 1.49 |
| HH2-R8 | 1890 | 121 | 1042 | 0.79 |
| HH2-R9 | 406 | 3.4 | 2099 | 1.60 |
| HH2-R10 | 701 | 5.1 | 2003 | 1.53 |
| HH2-R12 | 8574 | 51 | 1618 | 1.23 |
| HH2-K1 | 968 | 6.7 | 1423 | 1.08 |
| HH2-K2 | 1168 | 7.9 | 937 | 0.71 |
| HH2-K3 | 1763 | 11 | 1761 | 1.34 |
| HH2-K4 | 1553 | 10 | 759 | 0.58 |
| HH2-K5 | 1923 | 12 | 1559 | 1.19 |
| HH2-K6 | 23989 | 142 | 1055 | 0.80 |
| HH2-K7 | 117 | 1.7 | 1782 | 1.36 |
| HH2-K8 | 1501 | 9.8 | 851 | 0.65 |
| HH2-K9 | 180 | 2.1 | 1520 | 1.16 |
| HH2-K10 | 797 | 5.7 | 1878 | 1.43 |
| HH2-K11 | 2884 | 18 | 1593 | 1.21 |
| HH2-K12 | 2329 | 15 | 1203 | 0.92 |
| HH2-L1 | 654 | 4.8 | 1234 | 0.94 |
| HH2-L2 | 1950 | 13 | 1172 | 0.89 |
| HH2-L4 | 2884 | 18 | 800 | 0.61 |
| HH2-L5 | 97 | 1.6 | 1156 | 0.88 |
| HH2-L6 | 138 | 1.8 | 901 | 0.69 |
| HH2-L7 | 3138 | 19 | 1378 | 1.05 |
| HH2-L8 | 17953 | 107 | 725 | 0.55 |
| HH2-L9 | 1527 | 10 | 1359 | 1.04 |
| HH2-L10 | 3338 | 21 | 1359 | 1.04 |
| HH2-L11 | 6545 | 40 | 1425 | 1.09 |
| HH2-L12 | 3916 | 24 | 301 | 0.23 |
| HH2-I1 | 8573 | 51 | 1103 | 0.84 |
| HH2-I2 | 1748 | 11 | 1060 | 0.81 |
| HH2-I3 | 982 | 6.8 | 1336 | 1.02 |
| HH2-I4 | 1669 | 11 | 760 | 0.58 |
| HH2-I6 | 1206 | 8.1 | 984 | 0.75 |
| HH2-I7 | 1332 | 8.8 | 897 | 0.68 |
| HH2-I8 | 2789 | 17 | 701 | 0.53 |
| HH2-I9 | 8276 | 50 | 980 | 0.75 |
| HH2-I11 | 9977 | 60 | 1402 | 1.07 |
| HH2-I12 | 2845 | 18 | 667 | 0.51 |
| HH2-V2 | 3945 | 24 | 1267 | 0.97 |
| HH2-V3 | 35750 | 211 | 1665 | 1.27 |
| HH2-V4 | 3216 | 20 | 877 | 0.67 |
| HH2-V5 | 1409 | 9.3 | 1696 | 1.29 |
| HH2-V6 | 6624 | 40 | 694 | 0.53 |
| HH2-V8 | 7460 | 45 | 691 | 0.53 |
| HH2-V10 | 5929 | 36 | 1390 | 1.06 |
| HH2-V11 | 788 | 5.6 | 1515 | 1.15 |
| HH2-V12 | 5492 | 33 | 1119 | 0.85 |
| HH2-W1 | 4725 | 29 | 866 | 0.66 |
| HH2-W2 | 1802 | 12 | 1293 | 0.98 |
| HH2-W3 | 3418 | 21 | 874 | 0.67 |
| HH2-W4 | 3945 | 24 | 399 | 0.30 |
| HH2-W5 | 2198 | 14 | 1007 | 0.77 |
| HH2-W6 | 534 | 4.1 | 718 | 0.55 |
| HH2-W7 | 1395 | 9.2 | 1154 | 0.88 |
| HH2-W8 | 13556 | 81 | 553 | 0.42 |
| HH2-W9 | 4995 | 31 | 1050 | 0.80 |
| HH2-W10 | 2448 | 15 | 1344 | 1.02 |
| HH2-W11 | 2309 | 15 | 1362 | 1.04 |
| HH2-W12 | 7325 | 44 | 420 | 0.32 |
| HH2-Q1 | 10838 | 65 | 1171 | 0.89 |
| HH2-Q3 | 989 | 6.8 | 1141 | 0.87 |
| HH2-Q4 | 246 | 2.4 | 817 | 0.62 |
| HH2-Q5 | 603 | 4.5 | 1284 | 0.98 |
| HH2-Q6 | 32306 | 191 | 1088 | 0.83 |
| HH2-Q7 | 488 | 3.9 | 1316 | 1.00 |
| HH2-Q8 | 1421 | 9.4 | 1279 | 0.97 |
| HH2-Q9 | 5588 | 34 | 1321 | 1.01 |
| HH2-Q10 | 5057 | 31 | 1180 | 0.90 |
| HH2-Q11 | 2759 | 17 | 1020 | 0.78 |
| HH2-Q12 | 2034 | 13 | 965 | 0.73 |
| No Peptide | 0 | 1.0 | 1927 (LPS alone) | 1.47 |
| 1018 | 1040 | 7.1 | 127 | 0.10 |
| 1018-G1 | 36611 | 216 | 127 | 0.10 |
| 1018-G2 | 267 | 2.6 | 127 | 0.10 |
| 1018-G3 | 83 | 1.5 | 748 | 0.57 |
| 1018-G4 | 32 | 1.2 | 511 | 0.39 |
| 1018-G5 | 2090 | 13 | 1620 | 1.23 |
| 1018-G6 | 3560 | 22 | 598 | 0.46 |
| 1018-G7 | 4098 | 25 | 1610 | 1.23 |
| 1018-G8 | 57 | 1.3 | 127 | 0.10 |
| 1018-G9 | −17 | 0.9 | 1481 | 1.13 |
| 1018-G10 | −41 | 0.8 | 605 | 0.46 |
| 1018-G11 | 7922 | 48 | 1189 | 0.91 |
| 1018-G12 | 7 | 1.0 | 490 | 0.37 |
| 1018-A1 | 161 | 1.9 | 170 | 0.13 |
| 1018-A2 | −65 | 0.6 | 127 | 0.10 |
| 1018-A3 | −110 | 0.4 | 748 | 0.57 |
| 1018-A4 | −88 | 0.5 | 521 | 0.40 |
| 1018-A5 | 57 | 1.3 | 752 | 0.57 |
| 1018-A7 | −88 | 0.5 | 891 | 0.68 |
| 1018-A8 | 460 | 3.7 | 127 | 0.10 |
| 1018-A9 | −88 | 0.5 | 1176 | 0.90 |
| 1018-A10 | 7 | 1.0 | 369 | 0.28 |
| 1018-A11 | −65 | 0.6 | 1148 | 0.87 |
| 1018-A12 | −41 | 0.8 | 658 | 0.50 |
| 1018-R1 | 832 | 5.9 | 176 | 0.13 |
| 1018-R3 | 349 | 3.0 | 1636 | 1.25 |
| 1018-R4 | −65 | 0.6 | 615 | 0.47 |
| 1018-R5 | −41 | 0.8 | 1973 | 1.50 |
| 1018-R6 | 2122 | 14 | 1258 | 0.96 |
| 1018-R7 | 161 | 1.9 | 2201 | 1.68 |
| 1018-R9 | −10 | 0.9 | 310 | 0.24 |
| 1018-R10 | 12628 | 75 | 332 | 0.25 |
| 1018-K1 | 40 | 1.2 | 173 | 0.13 |
| 1018-K2 | 92 | 1.5 | 127 | 0.10 |
| 1018-K3 | 8203 | 49 | 1489 | 1.13 |
| 1018-K4 | 40 | 1.2 | 393 | 0.30 |
| 1018-K5 | −10 | 0.9 | 1722 | 1.31 |
| 1018-K6 | 31733 | 188 | 705 | 0.54 |
| 1018-K7 | 92 | 1.5 | 1222 | 0.93 |
| 1018-K8 | 1237 | 8.3 | 242 | 0.18 |
| 1018-K9 | 15 | 1.1 | 300 | 0.23 |
| 1018-K10 | 390 | 3.3 | 310 | 0.24 |
| 1018-K11 | 1419 | 9.3 | 170 | 0.13 |
| 1018-K12 | 531 | 4.1 | 127 | 0.10 |
| 1018-L1 | 531 | 4.1 | 282 | 0.21 |
| 1018-L2 | −35 | 0.8 | 426 | 0.32 |
| 1018-L4 | −10 | 0.9 | 127 | 0.10 |
| 1018-L5 | 335 | 3.0 | 162 | 0.12 |
| 1018-L6 | −60 | 0.7 | 340 | 0.26 |
| 1018-L7 | −10 | 0.9 | 132 | 0.10 |
| 1018-L8 | −83 | 0.5 | 553 | 0.42 |
| 1018-L9 | 66 | 1.4 | 171 | 0.13 |
| 1018-L10 | −60 | 0.7 | 127 | 0.10 |
| 1018-L11 | 118 | 1.7 | 1467 | 1.12 |
| 1018-L12 | 29 | 1.2 | 220 | 0.17 |
| 1018-I1 | 2873 | 18 | 138 | 0.11 |
| 1018-I2 | 7342 | 44 | 557 | 0.42 |
| 1018-I3 | −50 | 0.7 | 156 | 0.12 |
| 1018-I5 | 2103 | 13 | 127 | 0.10 |
| 1018-I6 | 110 | 1.6 | 582 | 0.44 |
| 1018-I7 | 714 | 5.2 | 127 | 0.10 |
| 1018-I8 | 2 | 1.0 | 1125 | 0.86 |
| 1018-I10 | 56 | 1.3 | 135 | 0.10 |
| 1018-I11 | 83 | 1.5 | 1775 | 1.35 |
| 1018-I12 | 3003 | 19 | 314 | 0.24 |
| 1018-V2 | 56 | 1.3 | 159 | 0.12 |
| 1018-V3 | 2550 | 16 | 295 | 0.22 |
| 1018-V4 | 29 | 1.2 | 144 | 0.11 |
| 1018-V6 | 110 | 1.6 | 226 | 0.17 |
| 1018-V8 | 29 | 1.2 | 463 | 0.35 |
| 1018-V9 | 17711 | 105 | 142 | 0.11 |
| 1018-V10 | 29 | 1.2 | 370 | 0.28 |
| 1018-V11 | 2 | 1.0 | 1240 | 0.94 |
| 1018-V12 | 56 | 1.3 | 673 | 0.51 |
| 1018-W1 | 684 | 5.0 | 204 | 0.16 |
| 1018-W2 | 83 | 1.5 | 962 | 0.73 |
| 1018-W3 | 953 | 6.6 | 127 | 0.10 |
| 1018-W4 | 29 | 1.2 | 182 | 0.14 |
| 1018-W5 | −28 | 0.8 | 127 | 0.10 |
| 1018-W6 | −54 | 0.7 | 310 | 0.24 |
| 1018-W7 | −28 | 0.8 | 483 | 0.37 |
| 1018-W8 | −28 | 0.8 | 448 | 0.34 |
| 1018-W9 | 247 | 2.5 | 127 | 0.10 |
| 1018-W11 | −28 | 0.8 | 814 | 0.62 |
| 1018-W12 | 79 | 1.5 | 441 | 0.34 |
| 1018-Q1 | 363 | 3.1 | 158 | 0.12 |
| 1018-Q2 | 422 | 3.5 | 127 | 0.10 |
| 1018-Q3 | −2 | 1.0 | 1359 | 1.03 |
| 1018-Q4 | −104 | 0.4 | 458 | 0.35 |
| 1018-Q5 | −54 | 0.7 | 1354 | 1.03 |
| 1018-Q6 | −54 | 0.7 | 186 | 0.14 |
| 1018-Q7 | −54 | 0.7 | 1368 | 1.04 |
| 1018-Q8 | −104 | 0.4 | 127 | 0.10 |
| 1018-Q9 | −28 | 0.8 | 1313 | 1.00 |
| 1018-Q10 | −54 | 0.7 | 154 | 0.12 |
| 1018-Q11 | 1773 | 11 | 1625 | 1.24 |
| 1018-Q12 | −28 | 0.8 | 655 | 0.50 |
| No Peptide | 0 | 1.0 | 2455 (LPS alone) | 1.0 |
| 1002 | 603.2 | 3.5 | 127 | 0.05 |
| 1002-G1 | 3335.7 | 14.8 | 127 | 0.05 |
| 1002-G2 | 891.9 | 4.7 | 127 | 0.05 |
| 1002-G3 | 3157.8 | 14.0 | 127 | 0.05 |
| 1002-G4 | 33.0 | 1.1 | 723 | 0.29 |
| 1002-G5 | −36.2 | 0.9 | 621 | 0.25 |
| 1002-G6 | −36.2 | 0.9 | 1098 | 0.45 |
| 1002-G7 | −36.2 | 0.9 | 353 | 0.14 |
| 1002-G8 | −36.2 | 0.9 | 692 | 0.28 |
| 1002-G9 | 444.4 | 2.8 | 127 | 0.05 |
| 1002-G10 | 91.3 | 1.4 | 222 | 0.09 |
| 1002-G11 | 465.0 | 2.9 | 127 | 0.05 |
| 1002-G12 | −36.2 | 0.9 | 240 | 0.10 |
| 1002-A1 | 4559.2 | 19.8 | 127 | 0.05 |
| 1002-A2 | 245.4 | 2.0 | 127 | 0.05 |
| 1002-A3 | 787.2 | 4.2 | 127 | 0.05 |
| 1002-A4 | −36.2 | 0.9 | 655 | 0.27 |
| 1002-A5 | −36.2 | 0.9 | 182 | 0.07 |
| 1002-A6 | 485.3 | 3.0 | 382 | 0.16 |
| 1002-A7 | −36.2 | 0.9 | 132 | 0.05 |
| 1002-A8 | −36.2 | 0.9 | 219 | 0.09 |
| 1002-A9 | 3239.9 | 14.4 | 127 | 0.05 |
| 1002-A10 | 245.4 | 2.0 | 127 | 0.05 |
| 1002-A11 | 485.3 | 3.0 | 127 | 0.05 |
| 1002-A12 | 91.3 | 1.4 | 127 | 0.05 |
| 1002-R1 | 1043.2 | 5.3 | 127 | 0.05 |
| 1002-R2 | 840.0 | 4.5 | 127 | 0.05 |
| 1002-R4 | −36.2 | 0.9 | 834 | 0.34 |
| 1002-R5 | −36.2 | 0.9 | 349 | 0.14 |
| 1002-R6 | 1.8 | 1.0 | 605 | 0.25 |
| 1002-R7 | 270.8 | 2.1 | 382 | 0.16 |
| 1002-R8 | −11.7 | 1.0 | 892 | 0.36 |
| 1002-R10 | 375.3 | 2.5 | 127 | 0.05 |
| 1002-R12 | 270.8 | 2.1 | 127 | 0.05 |
| 1002-K1 | 6146.9 | 26.4 | 127 | 0.05 |
| 1002-K2 | 392.4 | 2.6 | 127 | 0.05 |
| 1002-K3 | 576.2 | 3.4 | 137 | 0.06 |
| 1002-K4 | 106.3 | 1.4 | 1065 | 0.43 |
| 1002-K5 | 592.6 | 3.4 | 339 | 0.14 |
| 1002-K6 | −36.2 | 0.9 | 808 | 0.33 |
| 1002-K7 | −36.2 | 0.9 | 302 | 0.12 |
| 1002-K8 | −36.2 | 0.9 | 1632 | 0.66 |
| 1002-K9 | 143.9 | 1.6 | 127 | 0.05 |
| 1002-K10 | −32.5 | 0.9 | 127 | 0.05 |
| 1002-K11 | 543.3 | 3.2 | 127 | 0.05 |
| 1002-L1 | 2988.6 | 13.3 | 127 | 0.05 |
| 1002-L2 | 1968.2 | 9.1 | 127 | 0.05 |
| 1002-L3 | 106.3 | 1.4 | 127 | 0.05 |
| 1002-L4 | 493.6 | 3.0 | 283 | 0.12 |
| 1002-L6 | 68.0 | 1.3 | 127 | 0.05 |
| 1002-L7 | 199.1 | 1.8 | 127 | 0.05 |
| 1002-L8 | 2367.1 | 10.8 | 135 | 0.06 |
| 1002-L9 | 199.1 | 1.8 | 127 | 0.05 |
| 1002-L10 | 493.6 | 3.0 | 127 | 0.05 |
| 1002-L11 | 2048.4 | 9.5 | 127 | 0.05 |
| 1002-L12 | 905.7 | 4.7 | 127 | 0.05 |
| 1002-I1 | 4059.0 | 17.7 | 127 | 0.05 |
| 1002-I2 | 508.6 | 3.1 | 127 | 0.05 |
| 1002-I3 | 2953.7 | 13.2 | 127 | 0.05 |
| 1002-I4 | 273.6 | 2.1 | 213 | 0.09 |
| 1002-I5 | 5633.1 | 24.2 | 186 | 0.08 |
| 1002-I7 | 557.5 | 3.3 | 127 | 0.05 |
| 1002-I8 | 1393.2 | 6.7 | 144 | 0.06 |
| 1002-I9 | 605.9 | 3.5 | 158 | 0.06 |
| 1002-I11 | 238.8 | 2.0 | 127 | 0.05 |
| 1002-I12 | −36.2 | 0.9 | 127 | 0.05 |
| 1002-V2 | 5209.2 | 22.5 | 127 | 0.05 |
| 1002-V3 | 359.1 | 2.5 | 127 | 0.05 |
| 1002-V4 | 2300.2 | 10.5 | 190 | 0.08 |
| 1002-V5 | 9022.6 | 38.2 | 440 | 0.18 |
| 1002-V6 | 113.5 | 1.5 | 127 | 0.05 |
| 1002-V8 | 1468.2 | 7.1 | 198 | 0.08 |
| 1002-V9 | 131.8 | 1.5 | 134 | 0.05 |
| 1002-V10 | 308.0 | 2.3 | 127 | 0.05 |
| 1002-V11 | 541.2 | 3.2 | 127 | 0.05 |
| 1002-V12 | 76.4 | 1.3 | 127 | 0.05 |
| 1002-W1 | 622.0 | 3.6 | 127 | 0.05 |
| 1002-W2 | 2611.5 | 11.8 | 127 | 0.05 |
| 1002-W3 | 90.4 | 1.4 | 127 | 0.05 |
| 1002-W5 | −36.2 | 0.9 | 127 | 0.05 |
| 1002-W6 | −36.2 | 0.9 | 127 | 0.05 |
| 1002-W7 | 128.3 | 1.5 | 127 | 0.05 |
| 1002-W9 | 12.0 | 1.0 | 127 | 0.05 |
| 1002-W10 | 1889.1 | 8.8 | 127 | 0.05 |
| 1002-W11 | −36.2 | 0.9 | 127 | 0.05 |
| 1002-W12 | 412.5 | 2.7 | 127 | 0.05 |
| 1002-Q1 | 1423.5 | 6.9 | 127 | 0.05 |
| 1002-Q3 | 343.8 | 2.4 | 127 | 0.05 |
| 1002-Q4 | −28.8 | 0.9 | 1165 | 0.47 |
| 1002-Q5 | −36.2 | 0.9 | 180 | 0.07 |
| 1002-Q6 | −36.2 | 0.9 | 859 | 0.35 |
| 1002-Q7 | −36.2 | 0.9 | 138 | 0.06 |
| 1002-Q8 | −36.2 | 0.9 | 1608 | 0.66 |
| 1002-Q9 | 1514.2 | 7.2 | 127 | 0.05 |
| 1002-Q10 | 71.2 | 1.3 | 127 | 0.05 |
| 1002-Q11 | 3022.9 | 13.5 | 127 | 0.05 |
| 1002-Q12 | 1225.7 | 6.1 | 127 | 0.05 |
| 1018N-1002C | 32.0 | 1.1 | 203 | 0.08 |
| 1018N-HH2C | −36.2 | 0.9 | 780 | 0.32 |
| 1002N-1018C | −36.2 | 0.9 | 127 | 0.05 |
| 1002N-HH2C | −8.2 | 1.0 | 157 | 0.06 |
| HH2N-1018C | −36.2 | 0.9 | 171 | 0.07 |
| HH2N-1002C | −36.2 | 0.9 | 127 | 0.05 |
| 1002C-1018N | 51.7 | 1.2 | 127 | 0.05 |
| HH2C-1018N | 12797.7 | 53.8 | 1611 | 0.66 |
| 1018C-1002N | −34.7 | 0.9 | 129 | 0.05 |
| HH2C-1002N | 257.2 | 2.1 | 1471 | 0.60 |
| 1018C-HH2N | 381.4 | 2.6 | 1185 | 0.48 |
| 1002C-HH2N | 293.2 | 2.2 | 528 | 0.21 |
| 1018C-1018N | 450.3 | 2.9 | 632 | 0.26 |
| 1002C-1002N | 381.4 | 2.6 | 1174 | 0.48 |
| HH2C-HH2N | 239.0 | 2.0 | 769 | 0.31 |
| 1018N4-1002C8 | 1018.5 | 5.2 | 190 | 0.08 |
| 1018N4-HH2C8 | 48.4 | 1.2 | 411 | 0.17 |
| 1002N4-1018C8 | 239.0 | 2.0 | 693 | 0.28 |
| 1002N4-HH2C8 | 126.9 | 1.5 | 464 | 0.19 |
| HH2N4-1018C8 | 88.1 | 1.4 | 325 | 0.13 |
| HH2N4-1002C8 | 220.7 | 1.9 | 127 | 0.05 |
| 1018N8-1002C4 | 202.3 | 1.8 | 718 | 0.29 |
| 1018N8-HH2C4 | 700.0 | 3.9 | 587 | 0.24 |
| 1002N8-1018C4 | −36.2 | 0.9 | 127 | 0.05 |
| 1002N8-HH2C4 | 202.3 | 1.8 | 234 | 0.10 |
| HH2N8-1018C4 | 202.3 | 1.8 | 903 | 0.37 |
| HH2N8-1002C4 | 202.3 | 1.8 | 1089 | 0.44 |
| 1018 Reverse | 202.3 | 1.8 | 127 | 0.05 |
| 1002 Reverse | 3383.7 | 15.0 | 199 | 0.08 |
| HH2Rev | 585.0 | 3.4 | 239 | 0.10 |
| 1018C-1018N Rev | 257.2 | 2.1 | 692 | 0.28 |
| 1002C-1002N Rev | −36.2 | 0.9 | 614 | 0.25 |
| HH2C-HH2N Rev | −74.9 | 0.7 | 1667 | 0.68 |

^{a}background subtracted 139 (for HH2 derivatives) or 170 (for 1018 derivatives) and 242 (for 1002 and hybrid peptides) pg/ml.
^{b}IL1β production by PBMCs in the absence of peptide varied between donors, ranging from 1313 (for HH2 derivatives) to 1927 (for 1018 derivatives) and 2455 (for 1002 and hybrid peptides) pg/ml.

TABLE 12A
Screening of 1018, 1002 and HH2 derived peptides for immunomodulatory
activity. Results in column 2 have been background subtracted for the production of MCP1
(21.3 pg/ml). Peptides with enhanced MCP1 production or increased IL1β knockdown
relative to their respective parent peptide are shown in bold.

| | No LPS | | Cells stimulated with 10 ng/ml LPS | |
|---|---|---|---|---|
| | | Fold | | Fold change in |
| | | increase in | | IL1β relative to no |
| | | MCP1 cf. | IL1β Production | peptide LPS |
| Peptide | MCP1 (pg/ml)^{a} | untreated cells | (pg/ml) | stimulated cells |
| No Peptide | 0.0 | 1.0 | 984 | 1.00 |
| 1018 | 8915 | 419 | 172 | 0.17 |
| 2001 | 16300 | 765 | 76 | 0.08 |
| 2002 | 3215 | 152 | 293 | 0.30 |
| 2003 | 10848 | 509 | 1051 | 1.07 |
| 2004 | 7226 | 340 | 549 | 0.56 |
| 2005 | 17826 | 837 | 101 | 0.10 |
| 2006 | 6714 | 316 | 322 | 0.33 |
| 2007 | 7954 | 386 | 867 | 0.88 |
| 2008 | 45524 | 2146 | 250 | 0.25 |
| 1002 | 11994 | 576 | 111* | 0.13 |
| 2009 | 12475 | 586 | 74* | 0.08 |
| 2010 | 5649 | 266 | 377* | 0.42 |
| HH2 | 896 | 43 | 469* | 0.53 |
| 2011 | 796 | 38 | 550* | 0.62 |
| 2012 | 4824 | 227 | 381* | 0.43 |
| 2013 | 645 | 31 | 830* | 0.93 |

*Note - IL1β production for 1002-2013 peptides were determined separately and compared to untreated cells stimulated with LPS that produced 889 pg/ml of IL1β.

Other IDR peptides had much weaker activities than the peptides described above as shown in Table 12.

TABLE 12
Other IDR peptides designed as immunomoudulators.

| | Sequences (all peptides amidated; | |
|---|---|---|
| Peptide | sequences with D or RI in front of | |
| name | them are D amino acid containing) | Notes |
| EH1 | VRRIWRR | Weaker activity than 1018 |
| EH2 | VRFRIWRR | Weaker activity than 1018 |
| HE8 | VRRIVRVLIRWA | Toxic |
| HE3 | VRWARVARILRV | Weaker activity than 1018 |
| HE9 | RVLIRVARRVIW | Weaker activity than 1018 |
| HE7 | VRLIRVWRVIRK | No secretion of MCP-1 |

### Example 9

### Anti-Inflammatory Impact on Innate Immunity

It is well known that cationic antimicrobial peptides have the ability to boost immunity while suppressing inflammatory responses to bacterial signaling molecules like lipopolysaccharide and lipoteichoic acids as well as reducing inflammation and endotoxaemia (Hancock, R. E. W., A. Nijnik and D. J. Philpott. 2012. Modulating immunity as a therapy for bacterial infections. Nature Rev. Microbiol. 10:243-254). This suppression of inflammatory responses has stand-alone potential as it can result in protection in the neuro-inflammatory cerebral malaria model [Achtman et al, 2012] and with hyperinflammatory responses induced by flagellin in cystic fibrosis epithelial cells [Mayer, M. L., C. J. Blohmke, R. Falsafi, C. D. Fjell, L. Madera, S. E. Turvey, and R. E. W. Hancock. 2013. Rescue of dysfunctional autophagy by IDR-1018 attenuates hyperinflammatory responses from cystic fibrosis cells. J. Immunol. 190:1227-1238].

LPS from P. aeruginosa strain H103 was highly purified free of proteins and lipids using the Darveau-Hancock method. Briefly, P. aeruginosa was grown overnight in LB broth at 37° C. Cells were collected and washed and the isolated LPS pellets were extracted with a 2:1 chloroform:methanol solution to remove contaminating lipids. Purified LPS samples were quantitated using an assay for the specific sugar 2-keto-3-deoxyoctosonic acid (KDO assay) and then resuspended in endotoxin-free water (Sigma-Aldrich).

Human PBMC were obtained as described above and treated with P. aeruginosa LPS (10 or 100 ng/ml) with or without peptides for 24 hr after which supernatants were collected and TNFα assessed by ELISA.

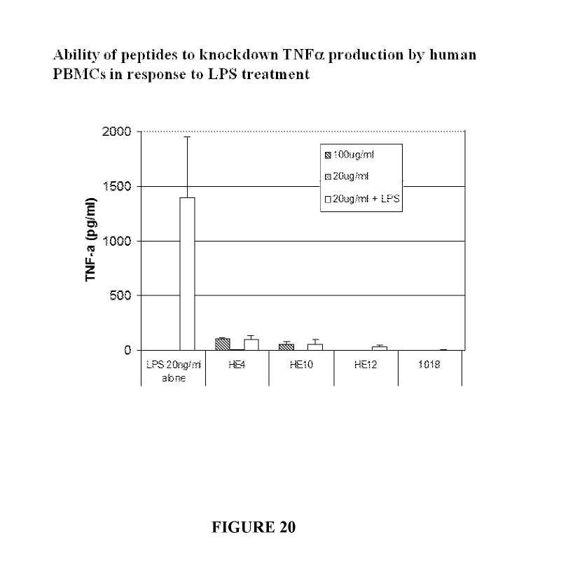

The data in FIG. 20 demonstrate that LPS as expected induced large levels of TNFα. This was strongly suppressed by peptides HE4, HE10, HE12 and 1018. By themselves, these peptides caused no significant increase in TNFα production.

Based on these results, new peptides were iteratively designed from our best immunomodulatory IDR peptides by substitution analysis of peptide sequences using SPOT synthesis on cellulose, and tested for immunomodulatory activity (reduction in the expression of the pro-inflammatory cytokine IL1-β in LPS-stimulated human peripheral blood mononuclear cells treated with at ˜18-24 μM concentrations of peptides). Results are shown in columns 4 and 5 of Table 12 and 12A above. Results shown in bold led to very substantial changes relative to the control peptide HH2 or equivalent to the more anti-inflammatory peptide 1018 respectively.

### Example 10

### Adjuvanticity as a Result of Enhancement of Innate Immunity

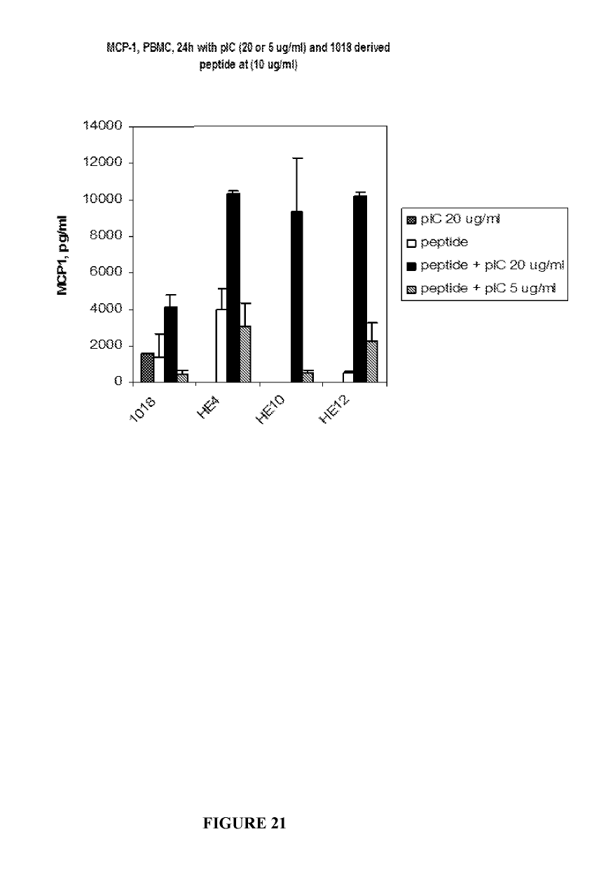

It is well accepted that vaccine immunization is best achieved by co-administration of an adjuvant. The precise mechanism by which these adjuvants work has eluded immunologists but appears to work in part by upregulating elements of innate immunity that smooth the transition to adaptive (antigen-specific) immunity (Bendelac A and R. Medzhitov. 2002. Adjuvants of immunity: Harnessing innate immunity to promote adaptive immunity J. Exp. Med. 195:F19-F23). Within this concept there are several possible avenues by which adjuvants might work including the attraction of immune cells into the site at which a particular antigen is injected, through e.g. upregulation of chemokines, the appropriate activation of cells when they reach that site, which can be caused by local cell or tissue damage releasing endogenous adjuvants or through specific cell activation by the adjuvants, and the compartmentalization of immune responses to the site of immunization (the so-called “depot” effect). Due to their ability to selectively modulate cell responses, including induction of chemokine expression, cationic host defence peptides such as human LL-37 and defensins, have been examined for adjuvant activity and demonstrated to enhance adaptive immune responses to a variety of antigens [Nicholls, E. F., L. Madera and R. E. W. Hancock. 2010. Immunomodulators as adjuvants for vaccines and antimicrobial therapy. Ann. NY Acad. Sci. 1213:46-61]. Peptides were shown to upregulate chemokines in human PBMC (FIG. 19; Table 12, Table 12A), consistent with an ability to act as adjuvants. They also showed synergy in inducing chemokines in combination with other proposed adjuvant agents that might work through other mechanisms such as poly(I:C). For example peptides 1018, HE4, HE10 and HE12 all showed synergy with 20 μg/ml of poly(I:C) (FIG. 21).

1. An isolated antibiofilm or immunomodulatory peptide having 7 to 12 amino acids, wherein the peptide has an amino acid sequence of SEQ ID NOS: 1-663, or analogs, derivatives, enantiomers, amidated and unamidated variations and conservative variations thereof.

2. An isolated polynucleotide that encodes a peptide of claim 1.

3. The peptide of claim 1 comprising any contiguous sequence of amino acids having the formula: AA1-AA2-AA3-AA4-AA5-AA6-AA7-AA8-AA9-AA10-AA11-AA12 and containing only the residues K, R, F, L, I, A, V, W and no more than a single Q or G residue.

4. A polypeptide X1-A-X2 or a functional variant or mimetic thereof, wherein A represents at least one peptide having an amino acid sequence of SEQ ID NOS: 1-749 or analogs, derivatives, enantiomers, amidated and unamidated variations and conservative variations thereof; and wherein each X1 and X2 independently of one another represents any amino acid sequence of n amino acids, n varying from 0 to 50, and n being identical or different in X1 and X2.

5. The polypeptide of claim 4 wherein the functional variant or mimetic is a conservative amino acid substitution or peptide mimetic substitution.

6. The polypeptide of claim 4 wherein the functional variant has about 66% or greater amino acid identity.

7. The polypeptide of claim 4, wherein the amino acids are non-natural amino acid equivalents.

8. The polypeptide of claim 4 wherein n is zero the peptide has the amino acid sequence VX₁X₂X₃X₄X₅X₆X₇X₈X₉X₁₀X₁₁; wherein X₁ is F, Q, or R; X₂ is L, W, or R; X₃ is R, I, or W; X₄ is R, A, V, or L; X₅ is I or A; X₆ is R or V; X₇ is V, R, or W; X₈ is W, I, or R; X₉ is V, W, or I; X₁₀ is I or R; X₁₁ is R or K or the reversed sequences.

9. A method of inhibiting the growth of bacterial biofilms comprising contacting a bacterial biofilm with an inhibiting effective amount of:
(i) a peptide having an amino acid sequence of SEQ ID NOS: 1-749, or any combination thereof, or analogs, derivatives, enantiomers, amidated and unamidated variations and conservative variations thereof, and/or
(ii) an isolated anti-biofilm polypeptide X1-A-X2, or a functional variant or mimetic thereof, wherein A represents at least one peptide having an amino acid sequence of SEQ ID NOS: 1-749 or analogs, derivatives, enantiomers, amidated and unamidated variations and conservative variations thereof, each X1 and X2 independently of one another represents any amino acid sequence of n amino acids, n varying from 0 to 5, and n being identical or different in X1 and X2.

10-13. (canceled)

14. The method of claim 9, wherein the peptide or the isolated anti-biofilm polypeptide is provided in combination with at least one antibiotic.

15-16. (canceled)

17. The method of claim 9, wherein the peptide is bound to a solid support or surface.

18. (canceled)

19. The peptide of claim 1, wherein the peptide is capable of selectively enhancing innate immunity as determined by contacting a cell containing one or more genes that encode a polypeptide involved in innate immunity and protection against an infection, with the peptide of interest, wherein expression of the one or more genes or polypeptides in the presence of the peptide is modulated as compared with expression of the one or more genes or polypeptides in the absence of the peptide, and wherein the modulated expression results in enhancement of innate immunity.

20. The peptide of claim 12, wherein the peptide:
(i) does not stimulate a septic reaction, or
(ii) stimulates expression of the one or more genes or proteins, thereby selectively enhancing innate immunity.

21-24. (canceled)

25. The peptide of claim 1, wherein the peptide selectively suppresses proinflammatory responses, whereby the peptide can contact a cell treated with an inflammatory stimulus and containing a polynucleotide or polynucleotides that encode a polypeptide involved in inflammation and sepsis and which is normally upregulated in response to this inflammatory stimulus, and wherein the peptides suppresses the expression of this gene or polypeptide as compared with expression of the inflammatory gene in the absence of the peptide and wherein the modulated expression results in enhancement of innate immunity.

26. The peptide of claim 25, wherein the peptide inhibits or blocks the inflammatory or septic response or inhibits the expression of a pro-inflammatory gene or molecule.

27-44. (canceled)

45. The peptide of claim 4, wherein the peptide is capable of selectively enhancing innate immunity as determined by contacting a cell containing one or more genes that encode a polypeptide involved in innate immunity and protection against an infection, with the peptide of interest, wherein expression of the one or more genes or polypeptides in the presence of the peptide is modulated as compared with expression of the one or more genes or polypeptides in the absence of the peptide, and wherein the modulated expression results in enhancement of innate immunity.

46. The method of claim 45, wherein the peptide does not stimulate a septic reaction or stimulates expression of the one or more genes or proteins, thereby selectively enhancing innate immunity.

47-49. (canceled)

50. The peptide of claim 4, wherein the peptide selectively suppresses proinflammatory responses, whereby the peptide can contact a cell treated with an inflammatory stimulus and containing a polynucleotide or polynucleotides that encode a polypeptide involved in inflammation and sepsis and which is normally upregulated in response to this inflammatory stimulus, and wherein the peptides suppresses the expression of this gene or polypeptide as compared with expression of the inflammatory gene in the absence of the peptide and wherein the modulated expression results in enhancement of innate immunity.

51. The method of claim 50, wherein the peptide inhibits or blocks the inflammatory or septic response or inhibits the expression of a pro-inflammatory gene or molecule.

52-56. (canceled)

57. An isolated molecule that has anti-biofilm activity by virtue of inhibiting (p)ppGpp synthesis or causing (p)ppGpp degradation.

58-59. (canceled)
